(19) United States
(12) Patent Application Publication
(10) Pub. No.: US 2019/0292155 A1
(43) Pub. Date: Sep. 26, 2019

(54) THERAPEUTIC INHIBITORY COMPOUNDS

(71) Applicant: LifeSci Pharmaceuticals, Inc., St. Michael (BB)

(72) Inventors: Andrew MCDONALD, New York, NY (US); Shawn QIAN, New York, NY (US)

(21) Appl. No.: 16/317,768

(22) PCT Filed: Jul. 14, 2017
(86) PCT No.: PCT/IB17/01342

§ 371 Date: Jan. 14, 2019

Related U.S. Application Data
(60) Provisional application No. 62/362,795, filed on Jul. 15, 2016.

(51) Int. Cl. C07D 231/56; A61K 9/00; A61K 9/20; A61P 7/06; C07D 401/12; C07D 401/14; C07D 405/14; C07D 409/12; C07D 405/12; C07D 471/04

(52) U.S. Cl. CPC C07D 231/56; A61K 9/0053; A61K 9/2054; A61K 9/2013; A61P 7/06; C07D 471/04; C07D 401/14; C07D 405/14; C07D 409/12; C07D 405/12; C07D 401/12

(57) ABSTRACT

Provided herein are heterocyclic derivative compounds and pharmaceutical compositions comprising said compounds. Said heterocyclic derivative compounds are complement factor D inhibitors. Such compounds are useful for treating complement related disorders including, but are not limited to, autoimmune, inflammatory, and neurodegenerative diseases.

### CROSS-REFERENCE

This application claims the benefit of U.S. provisional patent application No. 62/362,795 filed on Jul. 15, 2016 which is incorporated herein by reference in its entirety.

### BACKGROUND

A need exists in the medicinal arts for the effective treatment of diseases and disorders mediated by complement factor D. Such diseases and disorders include, but are not limited to, autoimmune, inflammatory, and neurodegenerative diseases.

### BRIEF SUMMARY OF THE INVENTION

Provided herein are heterocyclic derivative compounds and pharmaceutical compositions comprising said compounds. The subject compounds and compositions are useful for inhibiting complement factor D activity.

One embodiment provides a compound, or a pharmaceutically acceptable salt thereof, having the structure of Formula (I):

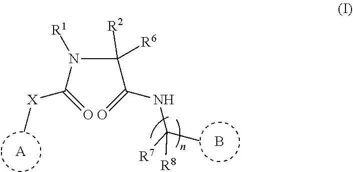

wherein
Ring A is an optionally substituted heteroaryl ring;
Ring B is an optionally substituted aryl, optionally substituted heteroaryl, optionally substituted 3-, 4-, 5-, or 6-membered cycloalkyl, or optionally substituted 3-, 4-, 5-, or 6-membered heterocyclyl;
X is —CR⁹R¹⁰— or —NR¹¹—;
R¹ is optionally substituted alkyl, optionally substituted 3-, 4-, 5-, or 6-membered cycloalkyl, optionally substituted carbocyclylalkyl, optionally substituted 3-, 4-, 5-, or 6-membered heterocyclyl, optionally substituted aryl, optionally substituted aralkyl, optionally substituted heteroaryl, optionally substituted heteroarylalkyl, or optionally substituted dicyclopropylmethyl;
R², R⁶, R⁹, R¹⁰, each R⁷, or each R⁸, is independently selected from hydrogen, halo, hydroxy, amino, —CO₂H, —S(O)—R²⁰, —S—R²⁰, —S(O)₂—R²⁰, optionally substituted alkoxy, optionally substituted aryloxy, optionally substituted heteroaryloxy, optionally substituted (heterocyclyl)-O—, optionally substituted alkyl, optionally substituted cycloalkyl, optionally substituted alkenyl, optionally substituted aryl, optionally substituted heteroaryl, optionally substituted heterocyclyl, optionally substituted alkylamino, optionally substituted dialkylamino, —CO—R²⁰, —CO₂—R²⁰, —CON(R²¹)₂, —SO₂N(R²¹)₂, —C(═NR²²)—N(R²¹)₂, optionally substituted alkynyl, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂;
each R²⁰ is independently optionally substituted alkyl, optionally substituted cycloalkyl, optionally substituted aryl, or optionally substituted heteroaryl;
each R²¹ is independently hydrogen, optionally substituted alkyl, optionally substituted cycloalkyl, optionally substituted aryl, or optionally substituted heteroaryl;
each R²² is independently hydrogen, optionally substituted alkyl, or optionally substituted cycloalkyl;
R¹ is hydrogen, or optionally substituted alkyl; and
n is 0, 1, 2, or 3.

One embodiment provides a compound, or a pharmaceutically acceptable salt thereof, chosen from:
(S)-1-(2-((2-((3-chloro-2-fluorobenzyl)amino)-1-cyclopropyl-2-oxoethyl)amino)-2-oxoethyl)-1H-indazole-3-carboxamide;
(S)-1-(2-((1-((3-chloro-2-fluorophenyl)amino)-1-oxopropan-2-yl)(methyl)amino)-2-oxoethyl)-1H-indazole-3-carboxamide;
(S)-1-(2-((1-((3-chloro-2-fluorobenzyl)amino)-1-oxopropan-2-yl)(methyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(isopropyl)amino)-2-oxoethyl)-1H-indazole-3-carboxamide;
1-(2-((2-((3-chloro-2-fluorobenzyl)amino)-2-oxoethyl)(ethyl)amino)-2-oxoethyl)-1H-indazole-3-carboxamide;
1-(2-((2-((3-chloro-2-fluorophenyl)amino)-2-oxoethyl)(cyclopropyl)amino)-2-oxoethyl)-1H-indazole-3-carboxamide;
3-(2-((2-((3-chloro-2-fluorobenzyl)amino)-2-oxoethyl)(cyclopropyl)amino)-2-oxoethyl)-1H-indazole-1-carboxamide;
1-(2-(cyclopropyl(2-((5-hydroxy-3,3-dimethylcyclohexyl)amino)-2-oxoethyl)amino)-2-oxoethyl)-1H-indazole-3-carboxamide;
1-(2-(cyclopropyl(2-(((1S,5S)-5-hydroxy-3,3-dimethylcyclohexyl)amino)-2-oxoethyl)amino)-2-oxoethyl)-1H-indazole-3-carboxamide;
1-(2-((2-((3-chloro-2-fluorophenyl)amino)-2-oxoethyl)(isopropyl)amino)-2-oxoethyl)-1H-indazole-3-carboxamide;
1-(2-((2-((6-chloropyridin-2-yl)amino)-2-oxoethyl)(isopropyl)amino)-2-oxoethyl)-1H-indazole-3-carboxamide;
1-(2-((2-((6-chloropyridin-2-yl)amino)-2-oxoethyl)(2-hydroxyethyl)amino)-2-oxoethyl)-1H-indazole-3-carboxamide;
1-(2-((2-((6-chloropyridin-2-yl)amino)-2-oxoethyl)(methyl)amino)-2-oxoethyl)-1H-indazole-3-carboxamide;
1-(2-((2-((6-chloropyridin-2-yl)amino)-2-oxoethyl)(ethyl)amino)-2-oxoethyl)-1H-indazole-3-carboxamide;
1-(2-((2-((6-chloropyridin-2-yl)amino)-2-oxoethyl)(cyclopropyl)amino)-2-oxoethyl)-1H-indazole-3-carboxamide;
1-(2-((2-((6-chloropyridin-2-yl)amino)-2-oxoethyl)(piperidin-4-yl)amino)-2-oxoethyl)-1H-indazole-3-carboxamide hydrochloride;
3-(2-((2-aminoethyl)(2-((6-chloropyridin-2-yl)amino)-2-oxoethyl)amino)-2-oxoethyl)-1H-indazole-1-carboxamide;
3-(2-((2-((6-chloropyridin-2-yl)amino)-2-oxoethyl)(cyclopentyl)amino)-2-oxoethyl)-1H-indazole-1-carboxamide;
3-(2-((2-((6-chloropyridin-2-yl)amino)-2-oxoethyl)(cyclobutyl)amino)-2-oxoethyl)-1H-indazole-1-carboxamide;
3-(2-((2-((6-chloropyridin-2-yl)amino)-2-oxoethyl)(tetrahydro-2H-pyran-4-yl)amino)-2-oxoethyl)-1H-indazole-1-carboxamide;
1-(2-(azetidin-3-yl(2-((6-chloropyridin-2-yl)amino)-2-oxoethyl)amino)-2-oxoethyl)-1H-indazole-3-carboxamide;
1-(2-((2-((6-chloropyridin-2-yl)amino)-2-oxoethyl)(2-(methylamino)ethyl)amino)-2-oxoethyl)-1H-indazole-3-carboxamide;
1-(2-((2-acetamidoethyl)(2-((6-chloropyridin-2-yl)amino)-2-oxoethyl)amino)-2-oxoethyl)-1H-indazole-3-carboxamide;
1-(2-((2-((6-chloropyridin-2-yl)amino)-2-oxoethyl)(pyridin-4-ylmethyl)amino)-2-oxoethyl)-1H-indazole-3-carboxamide;
1-(2-((2-((6-chloropyridin-2-yl)amino)-2-oxoethyl)(pyrrolidin-3-yl)amino)-2-oxoethyl)-1H-indazole-3-carboxamide;
1-(2-((2-((3-chloro-2-fluorobenzyl)amino)-2-oxoethyl)(thiophen-3-ylmethyl)amino)-2-oxoethyl)-1H-indazole-3-carboxamide;
1-(2-((2-((3-chloro-2-fluorobenzyl)amino)-2-oxoethyl)(cyclobutyl)amino)-2-oxoethyl)-1H-indazole-3-carboxamide;
1-(2-((2-((3-chloro-2-fluorobenzyl)amino)-2-oxoethyl)(tetrahydro-2H-pyran-4-yl)amino)-2-oxoethyl)-1H-indazole-3-carboxamide;
1-(2-((2-((3-chloro-2-fluorobenzyl)amino)-2-oxoethyl)(cyclopentyl)amino)-2-oxoethyl)-1H-indazole-3-carboxamide;
1-(2-((2-aminoethyl)(2-((3-chloro-2-fluorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(pyridin-4-ylmethyl)amino)-2-oxoethyl)-1H-indazole-3-carboxamide;
1-(2-((2-((3-chloro-2-fluorobenzyl)amino)-2-oxoethyl)(methyl)amino)-2-oxoethyl)-1H-indazole-3-carboxamide;
1-(2-((2-((3-chloro-2-fluorobenzyl)amino)-2-oxoethyl)(piperidin-4-yl)amino)-2-oxoethyl)-1H-indazole-3-carboxamide;
1-(2-((2-((3-chloro-2-fluorobenzyl)amino)-2-oxoethyl)(pyridin-3-ylmethyl)amino)-2-oxoethyl)-1H-indazole-3-carboxamide;
1-(2-((2-((3-chloro-2-fluorobenzyl)amino)-2-oxoethyl)(phenyl)amino)-2-oxoethyl)-1H-indazole-3-carboxamide;
1-(2-((2-((3-chloro-2-fluorobenzyl)amino)-2-oxoethyl)(pyrrolidin-3-yl)amino)-2-oxoethyl)-1H-indazole-3-carboxamide;
(R)-1-(2-((1-((3-chloro-2-fluorobenzyl)amino)-1-oxopropan-2-yl)(cyclopropyl)amino)-2-oxoethyl)-1H-indazole-3-carboxamide;
(S)-1-(2-((1-((3-chloro-2-fluorobenzyl)amino)-1-oxopropan-2-yl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-1H-pyrazolo[3,4-c]pyridine-3-carboxamide;
3-(2-((2-((3-chloro-2-fluorobenzyl)amino)-2-oxoethyl)(cyclopropyl)amino)-2-oxoethyl)-1H-indole-1-carboxamide;
3-(2-((2-((3-chloro-2-fluorobenzyl)amino)-2-oxoethyl)(cyclopropyl)amino)-2-oxoethyl)imidazo[1,5-a]pyridine-1-carboxamide;
1-(2-((2-((3-chloro-2-fluorobenzyl)amino)-2-oxoethyl)(cyclopropyl)amino)-2-oxoethyl)-5-(trifluoromethyl)-1H-pyrazole-3-carboxamide;
3-carbamoyl-1-(2-((2-((3-chloro-2-fluorobenzyl)amino)-2-oxoethyl)(cyclopropyl)amino)-2-oxoethyl)-1H-pyrazole-5-carboxylic acid;
1-(2-((2-((3-chloro-2-fluorobenzyl)amino)-2-oxoethyl)(cyclopropyl)amino)-2-oxoethyl)-1H-pyrazole-3,5-dicarboxamide;
4-bromo-1-(2-((2-((3-chloro-2-fluorobenzyl)amino)-2-oxoethyl)(cyclopropyl)amino)-2-oxoethyl)-1H-pyrazole-3-carboxamide;
2-(3-acetyl-1H-indazol-1-yl)-N-(2-((3-chloro-2-fluorobenzyl)amino)-2-oxoethyl)-N-cyclopropylacetamide;
2-(1-acetylimidazo[1,5-a]pyridin-3-yl)-N-(2-((3-chloro-2-fluorobenzyl)amino)-2-oxoethyl)-N-cyclopropylacetamide; N-(2-((3-chloro-2-fluorobenzyl)amino)-2-oxoethyl)-N-cyclopropyl-2-(3-(1-hydroxyethyl)-1H-indazol-1-yl)acetamide;
3-(3-(2-((3-chloro-2-fluorobenzyl)amino)-2-oxoethyl)-3-isopropylureido)-1H-indole-1-carboxamide;
3-(3-(2-((3-chloro-2-fluorobenzyl)amino)-2-oxoethyl)-3-cyclopropylureido)-1H-indole-1-carboxamide;
3-(3-(2-((3-chloro-2-fluorobenzyl)amino)-2-oxoethyl)-3-(pyrrolidin-3-yl)ureido)-1H-indole-1-carboxamide;
1-(3-(2-((3-chloro-2-fluorobenzyl)amino)-2-oxoethyl)-3-cyclopropylureido)imidazo[1,5-a]pyridine-3-carboxamide;
1-(2-((2-((3-chloro-2-fluorobenzyl)amino)-2-oxoethyl)(3-methylbutan-2-yl)amino)-2-oxoethyl)-1H-indazole-3-carboxamide;
1-(2-((2-((3-chloro-2-fluorobenzyl)amino)-2-oxoethyl)(1-cyclopropylethyl)amino)-2-oxoethyl)-1H-indazole-3-carboxamide;
1-(2-(sec-butyl(2-((3-chloro-2-fluorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(4-hydroxybutan-2-yl)amino)-2-oxoethyl)-1H-indazole-3-carboxamide;
1-(2-((2-((3-chloro-2-fluorobenzyl)amino)-2-oxoethyl)(1-hydroxypropan-2-yl)amino)-2-oxoethyl)-1H-indazole-3-carboxamide;
methyl 2-(2-(3-carbamoyl-1H-indazol-1-yl)-N-(2-((3-chloro-2-fluorobenzyl)amino)-2-oxoethyl)acetamido)propanoate;
1-(2-((2-((3-chloro-2-fluorobenzyl)amino)-2-oxoethyl)(4-fluorobutan-2-yl)amino)-2-oxoethyl)-1H-indazole-3-carboxamide;
1-(2-((2-((3-chloro-2-fluorobenzyl)amino)-2-oxoethyl)(1-methoxybutan-2-yl)amino)-2-oxoethyl)-1H-indazole-3-carboxamide;
1-(2-((1-amino-1-oxopropan-2-yl)(2-((3-chloro-2-fluorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(2-methylcyclopropyl)amino)-2-oxoethyl)-1H-indazole-3-carboxamide;
1-(2-((2-((3-chloro-2-fluorobenzyl)amino)-2-oxoethyl)(3,3-dimethylbutan-2-yl)amino)-2-oxoethyl)-1H-indazole-3-carboxamide;
1-(2-((2-((3-chloro-2-fluorobenzyl)amino)-2-oxoethyl)(1-phenylethyl)amino)-2-oxoethyl)-1H-indazole-3-carboxamide;
1-(2-((2-((3-chloro-2-fluorobenzyl)amino)-2-oxoethyl)(1-fluoropropan-2-yl)amino)-2-oxoethyl)-1H-indazole-3-carboxamide;
1-(2-((2-((3-chloro-2-fluorobenzyl)amino)-2-oxoethyl)(1,1,1-trifluoropropan-2-yl)amino)-2-oxoethyl)-1H-indazole-3-carboxamide;
1-(2-((2-((3-chloro-2-fluorobenzyl)amino)-2-oxoethyl)(1-methoxypropan-2-yl)amino)-2-oxoethyl)-1H-indazole-3-carboxamide;
1-(2-((2-((3-chloro-2-fluorobenzyl)amino)-2-oxoethyl)(1-cyclopropyl-2-hydroxyethyl)amino)-2-oxoethyl)-1H-indazole-3-carboxamide;
1-(2-((2-((2-chloro-3-fluorobenzyl)amino)-2-oxoethyl)(1,3-difluoropropan-2-yl)amino)-2-oxoethyl)-1H-indazole-3-carboxamide;
1-(2-((4-aminobutan-2-yl)(2-((2-chloro-3-fluorobenzyl)amino)-2-oxoethyl)amino)-2-oxoethyl)-1H-indazole-3-carboxamide;
ethyl 3-(2-(3-carbamoyl-1H-indazol-1-yl)-N-(2-((3-chloro-2-fluorobenzyl)amino)-2-oxoethyl)acetamido)butanoate;
1-(2-((2-((3-chloro-2-fluorobenzyl)amino)-2-oxoethyl)(1-cyclopropyl-3-hydroxypropyl)amino)-2-oxoethyl)-1H-indazole-3-carboxamide;
3-(2-(3-carbamoyl-1H-indazol-1-yl)-N-(2-((3-chloro-2-fluorobenzyl)amino)-2-oxoethyl)acetamido)butanoic acid;
1-(2-((3-amino-1-cyclopropyl-3-oxopropyl)(2-((3-chloro-2-fluorobenzyl)amino)-2-oxoethyl)amino)-2-oxoethyl)-1H-indazole-3-carboxamide;
1-(2-((4-amino-4-oxobutan-2-yl)(2-((3-chloro-2-fluorobenzyl)amino)-2-oxoethyl)amino)-2-oxoethyl)-1H-indazole-3-carboxamide;
ethyl 3-(2-(3-carbamoyl-1H-indazol-1-yl)-N-(2-((3-chloro-2-fluorobenzyl)amino)-2-oxoethyl)acetamido)-4,4,4-trifluorobutanoate;
1-(2-((2-((3-chloro-2-fluorobenzyl)amino)-2-oxoethyl)(cyclopropyl)amino)-2-oxoethyl)-5-fluoro-1H-indazole-3-carboxamide;
3-(2-(3-carbamoyl-1H-indazol-1-yl)-N-(2-((3-chloro-2-fluorobenzyl)amino)-2-oxoethyl)acetamido)-4,4,4-trifluorobutanoic acid;
5-chloro-1-(2-((2-((3-chloro-2-fluorobenzyl)amino)-2-oxoethyl)(cyclopropyl)amino)-2-oxoethyl)-1H-indazole-3-carboxamide;
7-chloro-1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-2,6-difluorobenzyl)amino)-2-oxoethyl)(cyclopropyl)amino)-2-oxoethyl)-1H-indazole-3-carboxamide;
1-(2-((2-((3-chloro-2,4-difluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-6-fluoro-1H-indazole-3-carboxamide;
6-chloro-1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-6-cyano-1H-indazole-3-carboxamide;
1-(2-((2-((3-chloro-6-cyano-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-7-fluoro-1H-indazole-3-carboxamide;
6-(aminomethyl)-1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-5-cyano-2-fluorobenzyl)amino)-2-oxoethyl)(cyclopropyl)amino)-2-oxoethyl)-1H-indazole-3-carboxamide;
1-(2-((2-(((6-chloro-1H-indazol-4-yl)methyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-5-cyano-1H-indazole-3-carboxamide;
1-(2-((2-((3-chloro-2-fluorobenzyl)amino)-2-oxoethyl)(cyclopropyl)amino)-2-oxoethyl)-1H-indazole-3,5-dicarboxamide;
1-(2-((2-((3-chloro-2-fluorobenzyl)amino)-2-oxoethyl)(cyclopropyl)amino)-2-oxoethyl)-6-(trifluoromethyl)-1H-indazole-3-carboxamide;
1-(2-((2-((3-chloro-2-fluorobenzyl)amino)-2-oxoethyl)(cyclopropyl)amino)-2-oxoethyl)-5-(trifluoromethyl)-1H-indazole-3-carboxamide;
1-(2-((2-((3-chloro-4-cyano-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-4-fluoro-1H-indazole-3-carboxamide;
2-(3-(1H-imidazol-2-yl)-1H-indazol-1-yl)-N-(2-((3-chloro-2-fluorobenzyl)amino)-2-oxoethyl)-N-cyclopropylacetamide;
1-(2-((2-((3-chloro-2-fluoro-4-(hydroxymethyl)benzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-1H-indazole-3,6-dicarboxamide;
1-(2-((2-((5-(aminomethyl)-3-chloro-2-fluorobenzyl)amino)-2-oxoethyl)(cyclopropyl)amino)-2-oxoethyl)-1H-indazole-3-carboxamide;
1-(2-((2-(((4-chloro-1H-indazol-6-yl)methyl)amino)-2-oxoethyl)(cyclopropyl)amino)-2-oxoethyl)-1H-indazole-3-carboxamide;
1-(2-((2-((3-chloro-5-fluorobenzyl)amino)-2-oxoethyl)(cyclopropyl)amino)-2-oxoethyl)-1H-indazole-3-carboxamide;
1-(2-(cyclopropyl(2-((3,4-dichlorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-7-cyano-1H-indazole-3-carboxamide;
1-(2-(cyclopropyl(2-((2,3-dichlorobenzyl)amino)-2-oxoethyl)amino)-2-oxoethyl)-1H-indazole-3-carboxamide;
1-(2-((2-(((6-chloropyridin-2-yl)methyl)amino)-2-oxoethyl)(cyclopropyl)amino)-2-oxoethyl)-1H-indazole-3-carboxamide;
1-(2-((2-((3-chloro-4-fluorobenzyl)amino)-2-oxoethyl)(cyclopropyl)amino)-2-oxoethyl)-1H-indazole-3-carboxamide;
1-(2-((2-(((5-chloro-1H-indazol-7-yl)methyl)amino)-2-oxoethyl)(cyclopropyl)amino)-2-oxoethyl)-1H-indazole-3-carboxamide;
1-(2-((2-(((5-chlorobenzofuran-7-yl)methyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-7-(trifluoromethyl)-1H-indazole-3-carboxamide;
1-(2-((2-((3-chlorobenzyl)amino)-2-oxoethyl)(cyclopropyl)amino)-2-oxoethyl)-1H-indazole-3-carboxamide;
1-(2-((2-((3-chloro-4-methylbenzyl)amino)-2-oxoethyl)(cyclopropyl)amino)-2-oxoethyl)-1H-indazole-3-carboxamide;
1-(2-((2-(((7-chloro-1H-indazol-5-yl)methyl)amino)-2-oxoethyl)(cyclopropyl)amino)-2-oxoethyl)-1H-indazole-3-carboxamide;
1-(2-(cyclopropyl(2-((2-fluorobenzyl)amino)-2-oxoethyl)amino)-2-oxoethyl)-1H-indazole-3-carboxamide;
1-(2-((2-((3-chloro-2-methoxybenzyl)amino)-2-oxoethyl)(cyclopropyl)amino)-2-oxoethyl)-1H-indazole-3-carboxamide;
1-(2-((2-((4-carbamoyl-2-fluorobenzyl)amino)-2-oxoethyl)(cyclopropyl)amino)-2-oxoethyl)-1H-indazole-3-carboxamide;
1-(2-((2-((3-amino-2-fluorobenzyl)amino)-2-oxoethyl)(cyclopropyl)amino)-2-oxoethyl)-1H-indazole-3-carboxamide;
1-(2-(cyclopropyl(2-((3,5-dichlorobenzyl)amino)-2-oxoethyl)amino)-2-oxoethyl)-1H-indazole-3-carboxamide;
1-(2-((2-((3-bromo-2-fluorobenzyl)amino)-2-oxoethyl)(cyclopropyl)amino)-2-oxoethyl)-1H-indazole-3-carboxamide;
1-(2-((2-((4-amino-2-fluorobenzyl)amino)-2-oxoethyl)(cyclopropyl)amino)-2-oxoethyl)-1H-indazole-3-carboxamide;
1-(2-(cyclopropyl(2-((2-fluoro-3-methoxybenzyl)amino)-2-oxoethyl)amino)-2-oxoethyl)-1H-indazole-3-carboxamide;
1-(2-((2-((3-cyano-2-fluorobenzyl)amino)-2-oxoethyl)(cyclopropyl)amino)-2-oxoethyl)-1H-indazole-3-carboxamide;
1-(2-((2-((3-chloro-5-(trifluoromethyl)benzyl)amino)-2-oxoethyl)(cyclopropyl)amino)-2-oxoethyl)-1H-indazole-3-carboxamide;
1-(2-((2-((3-chloro-4-methoxybenzyl)amino)-2-oxoethyl)(cyclopropyl)amino)-2-oxoethyl)-1H-indazole-3-carboxamide;
1-(2-(cyclopropyl(2-((2-fluoro-3-(hydroxymethyl)benzyl)amino)-2-oxoethyl)amino)-2-oxoethyl)-1H-indazole-3-carboxamide;
1-(2-(cyclopropyl(2-((3-cyclopropyl-2-fluorobenzyl)amino)-2-oxoethyl)amino)-2-oxoethyl)-1H-indazole-3-carboxamide;
1-(2-(cyclopropyl(2-((2-fluoro-3-methylbenzyl)amino)-2-oxoethyl)amino)-2-oxoethyl)-1H-indazole-3-carboxamide;
1-(2-((2-((3-chloro-5-methylbenzyl)amino)-2-oxoethyl)(cyclopropyl)amino)-2-oxoethyl)-1H-indazole-3-carboxamide;
1-(2-((2-(((5-chloropyridin-3-yl)methyl)amino)-2-oxoethyl)(cyclopropyl)amino)-2-oxoethyl)-1H-indazole-3-carboxamide;
1-(2-((2-((3-chloro-2-cyclopropylbenzyl)amino)-2-oxoethyl)(cyclopropyl)amino)-2-oxoethyl)-1H-indazole-3-carboxamide;
1-(2-(cyclopropyl(2-((2,3-difluorobenzyl)amino)-2-oxoethyl)amino)-2-oxoethyl)-1H-indazole-3-carboxamide;
1-(2-((2-((3-chloro-5-cyanobenzyl)amino)-2-oxoethyl)(cyclopropyl)amino)-2-oxoethyl)-1H-indazole-3-carboxamide;
1-(2-((2-((3-chloro-2-methylbenzyl)amino)-2-oxoethyl)(cyclopropyl)amino)-2-oxoethyl)-1H-indazole-3-carboxamide;
1-(2-((2-((3-chloro-2-(trifluoromethyl)benzyl)amino)-2-oxoethyl)(cyclopropyl)amino)-2-oxoethyl)-1H-indazole-3-carboxamide;
1-(2-(cyclopropyl(2-((2-fluoro-3-(trifluoromethyl)benzyl)amino)-2-oxoethyl)amino)-2-oxoethyl)-1H-indazole-3-carboxamide;
2-(3-acetylimidazo[1,5-a]pyridin-1-yl)-N-(2-((3-chloro-2-fluorobenzyl)amino)-2-oxoethyl)-N-cyclopropylacetamide;
1-(2-((2-((3-chloro-2-fluorobenzyl)amino)-2-oxoethyl)(cyclopropyl)amino)-2-oxoethyl)imidazo[1,5-a]pyridine-3-carboxamide;
2-(1-acetylimidazo[1,5-a]pyridin-3-yl)-N-(2-((3-chloro-2-fluorobenzyl)amino)-2-oxoethyl)-N-cyclopropylacetamide;
3-(3-(2-((3-chloro-2-fluorobenzyl)amino)-2-oxoethyl)-3-cyclopropylureido)imidazo[1,5-a]pyridine-1-carboxamide; or
2-(3-(3-acetylimidazo[1,5-a]pyridin-1-yl)-1-cyclopropylureido)-N-(3-chloro-2-fluorobenzyl)acetamide.

One embodiment provides a pharmaceutical composition comprising a compound of Formula (I), or a pharmaceutically acceptable salt thereof, and a pharmaceutically acceptable excipient.

One embodiment provides a method of inhibiting complement factor D comprising contacting the complement factor D protein with a compound of Formula (I).

One embodiment provides a method for treating paraoxysmal nocturnal hemoglobinuria in a patient in need thereof comprising administering to the patient a composition comprising a compound of Formula (I), or a pharmaceutically acceptable salt thereof.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O— alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group is through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atom (e.g., C₂ alkylene). In other embodiments, an alkynylene comprises five to eight carbon atoms (e.g., C₅-C₅ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) t-electron system in accordance with the Hickel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

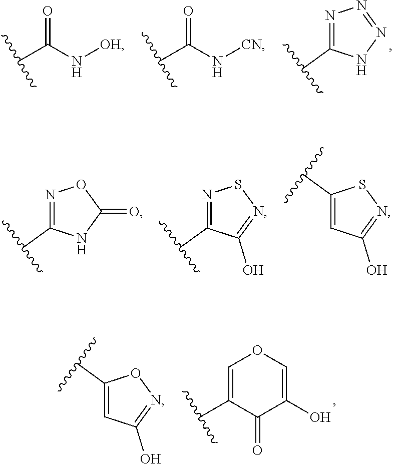

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) n-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolin yl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

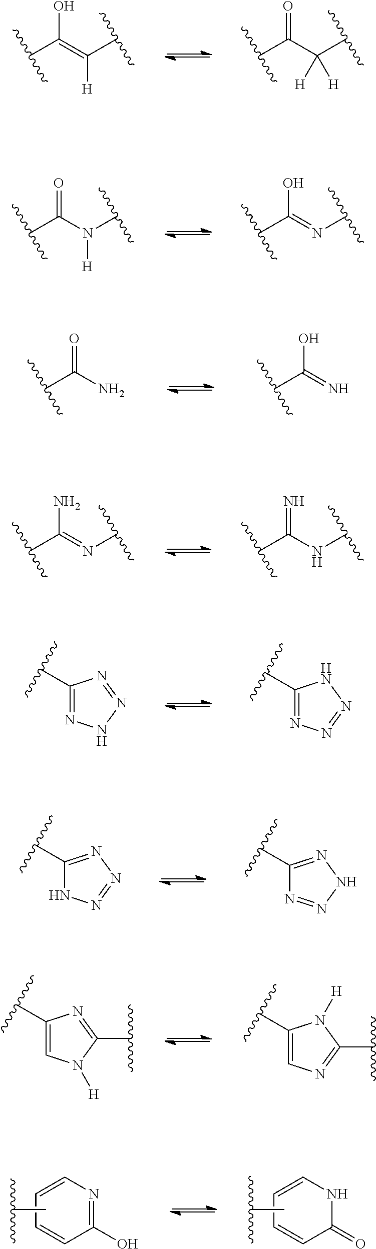

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commerically from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

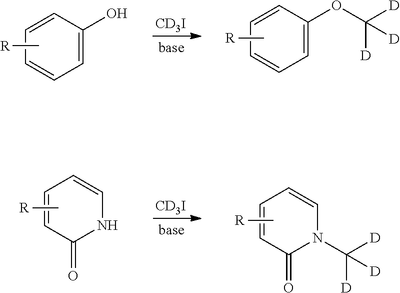

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

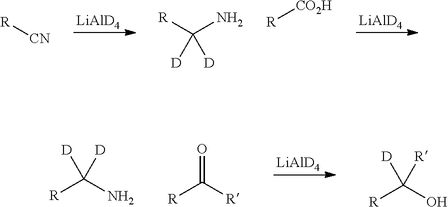

Deuterium gas and palladium catalyst are employed to reduce unsaturated carbon-carbon linkages and to perform a reductive substitution of aryl carbon-halogen bonds as illustrated, by way of example only, in the reaction schemes below.

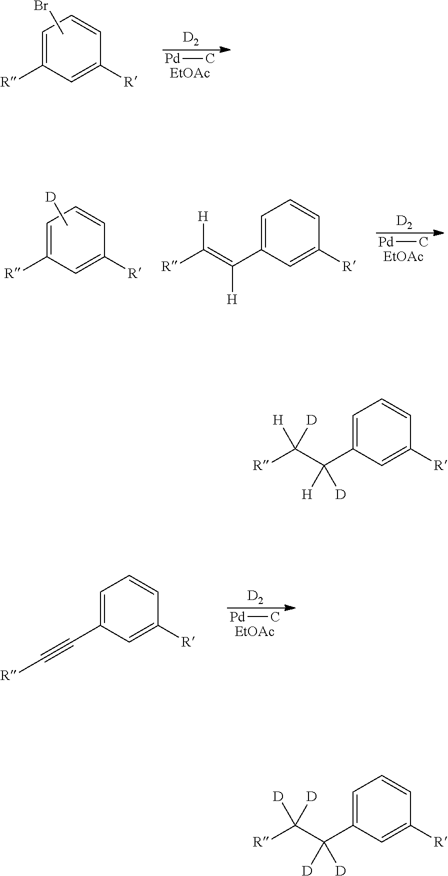

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the kallikrein inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

“Prodrug” is meant to indicate a compound that is, in some embodiments, converted under physiological conditions or by solvolysis to a biologically active compound described herein. Thus, the term “prodrug” refers to a precursor of a biologically active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

### Complement Factor D Inhibitory Compounds

Provided herein are heterocyclic derivative compounds and pharmaceutical compositions comprising said compounds. The subject compounds and compositions are useful for inhibiting complement factor D activity.

One embodiment provides a compound, or a pharmaceutically acceptable salt thereof, having the structure of Formula (I):

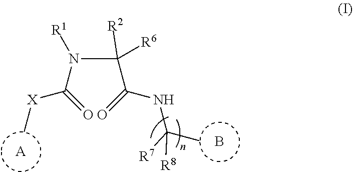

wherein
Ring A is an optionally substituted heteroaryl ring;
Ring B is an optionally substituted aryl, optionally substituted heteroaryl, optionally substituted 3-, 4-, 5-, or 6-membered cycloalkyl, or optionally substituted 3-, 4-, 5-, or 6-membered heterocyclyl;
X is —CR⁹R¹⁰— or —NR¹¹—;
R¹ is optionally substituted alkyl, optionally substituted 3-, 4-, 5-, or 6-membered cycloalkyl, optionally substituted carbocyclylalkyl, optionally substituted 3-, 4-, 5-, or 6-membered heterocyclyl, optionally substituted aryl, optionally substituted aralkyl, optionally substituted heteroaryl, optionally substituted heteroarylalkyl, or optionally substituted dicyclopropylmethyl;
R², R⁶, R⁹, R¹⁰, each R⁷, or each R⁸, is independently selected from hydrogen, halo, hydroxy, amino, —CO₂H, —S(O)—R²⁰, —S—R²⁰, —S(O)₂—R²⁰, optionally substituted alkoxy, optionally substituted aryloxy, optionally substituted heteroaryloxy, optionally substituted (heterocyclyl)-O—, optionally substituted alkyl, optionally substituted cycloalkyl, optionally substituted alkenyl, optionally substituted aryl, optionally substituted heteroaryl, optionally substituted heterocyclyl, optionally substituted alkylamino, optionally substituted dialkylamino, —CO—R²⁰, —CO₂—R²⁰, —CON(R²¹)₂, —SO₂N(R²¹)₂, —C(═NR²²)—N(R²¹)₂, optionally substituted alkynyl, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂;
each R²⁰ is independently optionally substituted alkyl, optionally substituted cycloalkyl, optionally substituted aryl, or optionally substituted heteroaryl;
each R²¹ is independently hydrogen, optionally substituted alkyl, optionally substituted cycloalkyl, optionally substituted aryl, or optionally substituted heteroaryl;
each R²² is independently hydrogen, optionally substituted alkyl, or optionally substituted cycloalkyl;
R¹¹ is hydrogen, or optionally substituted alkyl; and
n is 0, 1, 2, or 3.

Another embodiment provides a compound of Formula (I), or a pharmaceutically acceptable salt thereof, wherein Ring A is

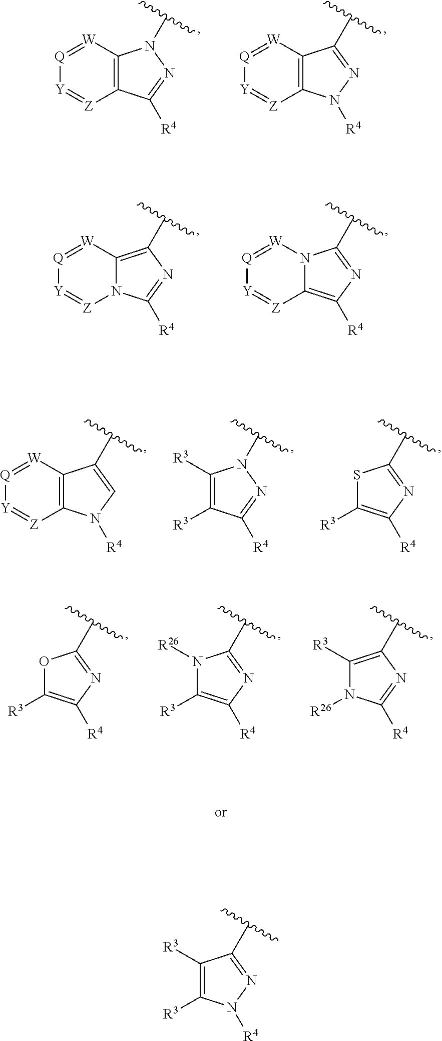

wherein
W, Q, Y, and Z are each independently selected from N or C—R³;
each R³ is independently selected from hydrogen, cyano, halo, hydroxy, azido, amino, nitro, —CO₂H, —S(O)—R²³, —S—R²³, —S(O)₂—R²³, optionally substituted alkoxy, optionally substituted aryloxy, optionally substituted heteroaryloxy, optionally substituted (heterocyclyl)-O—, optionally substituted alkyl, optionally substituted alkynyl, optionally substituted carbocyclyl, optionally substituted alkenyl, optionally substituted aryl, optionally substituted heteroaryl, optionally substituted heterocyclyl, optionally substituted aralkoxy, optionally substituted heteroarylalkoxy, optionally substituted (carbocyclyl)-O—, optionally substituted heterocyclylalkoxy, —N(R²⁴), —CO—R²³, —CO₂—R²³, —CO(R²⁴)₂, —NR²⁴CO—R²³, —NR²⁴CO₂—R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, —O—CO₂—R²³;
each R²³ is independently optionally substituted alkyl, optionally substituted cycloalkyl, optionally substituted aryl, or optionally substituted heteroaryl;
each R²⁴ is independently hydrogen, optionally substituted alkyl, optionally substituted cycloalkyl, optionally substituted aryl, or optionally substituted heteroaryl;
each R²⁵ is independently hydrogen, optionally substituted alkyl, or optionally substituted cycloalkyl;
each R²⁶ is independently hydrogen or optionally substituted alkyl;
R⁴ is optionally substituted alkyl, —C(═O)R⁵, or heteroaryl;
R⁵ is selected from NH₂, optionally substituted alkylamino, optionally substituted dialkylamino, optionally substituted alkyl, or optionally substituted cycloalkyl.

Another embodiment provides a compound of Formula (I), or a pharmaceutically acceptable salt thereof, wherein Ring A is

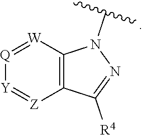

Another embodiment provides a compound of Formula (I), or a pharmaceutically acceptable salt thereof, wherein W, Q, Y, and Z are C—R³.

Another embodiment provides a compound of Formula (I), or a pharmaceutically acceptable salt thereof, wherein W, Q, Y, and Z are C—R³ and each R³ is independently selected from hydrogen, CO₂H, —S(O)—R²³, —S—R²³, —S(O)₂—R²³, —N(R²⁴), —CO—R²³, —CO₂—R²³, —CO(NR)₂, —NR²⁴CO—R²³, —NR²⁴CO₂—R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, or —O—CO₂—R²³.

Another embodiment provides a compound of Formula (I), or a pharmaceutically acceptable salt thereof, wherein W, Q, Y, and Z are C—R³ and each R³ is independently selected from hydrogen, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

Another embodiment provides a compound of Formula (I), or a pharmaceutically acceptable salt thereof, wherein W, Q, Y, and Z are C—R³ and each R³ is independently selected from hydrogen, halogen, optionally substituted alkyl, optionally substituted cycloalkyl, or optionally substituted alkoxy.

Another embodiment provides a compound of Formula (I), or a pharmaceutically acceptable salt thereof, wherein W, Q, Y, and Z are C—R³ and each R³ is hydrogen.

Another embodiment provides a compound of Formula (I), or a pharmaceutically acceptable salt thereof, wherein Q is N; W, Y, and Z are C—R³.

Another embodiment provides a compound of Formula (I), or a pharmaceutically acceptable salt thereof, wherein Q is N; W, Y, and Z are C—R³; and each R³ is independently selected from hydrogen, CO₂H, —S(O)—R²³, —S—R²³, —S(O)₂—R²³, —N(R²⁴), —CO—R²³, —CO₂—R²³, —CO(NR²⁴)₂, —NR²⁴CO—R²³, —NR²⁴CO₂—R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, or —O—CO₂—R²³.

Another embodiment provides a compound of Formula (I), or a pharmaceutically acceptable salt thereof, wherein Q is N; W, Y, and Z are C—R³; and each R³ is independently selected from hydrogen, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

Another embodiment provides a compound of Formula (I), or a pharmaceutically acceptable salt thereof, wherein Q is N; W, Y, and Z are C—R³; and each R³ is independently selected from hydrogen, halogen, optionally substituted alkyl, optionally substituted cycloalkyl, or optionally substituted alkoxy.

Another embodiment provides a compound of Formula (I), or a pharmaceutically acceptable salt thereof, wherein Q is N; W, Y, and Z are C—R³; and each R³ is hydrogen.

Another embodiment provides a compound of Formula (I), or a pharmaceutically acceptable salt thereof, wherein W is N; Q, Y, and Z are C—R³.

Another embodiment provides a compound of Formula (I), or a pharmaceutically acceptable salt thereof, wherein W is N; Q, Y, and Z are C—R³; and each R³ is independently selected from hydrogen, CO₂H, —S(O)—R²³, —S—R²³, —S(O)₂—R²³, —N(R²⁴), —CO—R²³, —CO₂—R²³, —CO(NR²⁴)₂, —NR²⁴CO—R²³, —NR²⁴CO₂—R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, or —O—CO₂—R²³.

Another embodiment provides a compound of Formula (I), or a pharmaceutically acceptable salt thereof, wherein W is N; Q, Y, and Z are C—R³; and each R³ is independently selected from hydrogen, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

Another embodiment provides a compound of Formula (I), or a pharmaceutically acceptable salt thereof, wherein W is N; Q, Y, and Z are C—R³; and each R³ is independently selected from hydrogen, halogen, optionally substituted alkyl, optionally substituted cycloalkyl, or optionally substituted alkoxy.

Another embodiment provides a compound of Formula (I), or a pharmaceutically acceptable salt thereof, wherein W is N; Q, Y, and Z are C—R³; and each R³ is hydrogen.

Another embodiment provides a compound of Formula (I), or a pharmaceutically acceptable salt thereof, wherein Y is N; W, Q, and Z are C—R³.

Another embodiment provides a compound of Formula (I), or a pharmaceutically acceptable salt thereof, wherein Y is N; W, Q, and Z are C—R³; and each R³ is independently selected from hydrogen, CO₂H, —S(O)—R²³, —S—R²³, —S(O)₂—R²³, —N(R²⁴), —CO—R²³, —CO₂—R²³, —CO(NR)₂, —NR²⁴CO—R²³, —NR²⁴CO₂—R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, or —O—CO₂—R²³.

Another embodiment provides a compound of Formula (I), or a pharmaceutically acceptable salt thereof, wherein Y is N; W, Q, and Z are C—R³; and each R³ is independently selected from hydrogen, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

Another embodiment provides a compound of Formula (I), or a pharmaceutically acceptable salt thereof, wherein Y is N; W, Q, and Z are C—R³; and each R³ is independently selected from hydrogen, halogen, optionally substituted alkyl, optionally substituted cycloalkyl, or optionally substituted alkoxy.

Another embodiment provides a compound of Formula (I), or a pharmaceutically acceptable salt thereof, wherein Y is N; W, Q, and Z are C—R³; and each R³ is hydrogen.

Another embodiment provides a compound of Formula (I), or a pharmaceutically acceptable salt thereof, wherein Z is N; W, Q, and Y are C—R³.

Another embodiment provides a compound of Formula (I), or a pharmaceutically acceptable salt thereof, wherein Z is N; W, Q, and Y are C—R³; and each R³ is independently selected from hydrogen, CO₂H, —S(O)—R²³, —S—R²³, —S(O)₂—R²³, —N(R²⁴), —CO—R²³, —CO₂—R²³, —CO(NR)₂, —NR²⁴CO—R²³, —NR²⁴CO₂—R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, or —O—CO₂—R²³.

Another embodiment provides a compound of Formula (I), or a pharmaceutically acceptable salt thereof, wherein Z is N; W, Q, and Y are C—R³; and each R³ is independently selected from hydrogen, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

Another embodiment provides a compound of Formula (I), or a pharmaceutically acceptable salt thereof, wherein Z is N; W, Q, and Y are C—R³; and each R³ is independently selected from hydrogen, halogen, optionally substituted alkyl, optionally substituted cycloalkyl, or optionally substituted alkoxy.

Another embodiment provides a compound of Formula (I), or a pharmaceutically acceptable salt thereof, wherein Z is N; W, Q, and Y are C—R³; and each R³ is hydrogen.

Another embodiment provides a compound of Formula (I), or a pharmaceutically acceptable salt thereof, wherein R⁴ is —CONH₂.

Another embodiment provides a compound of Formula (I), or a pharmaceutically acceptable salt thereof, wherein Ring A is

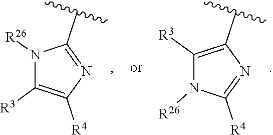

Another embodiment provides a compound of Formula (I), or a pharmaceutically acceptable salt thereof, wherein Ring A is

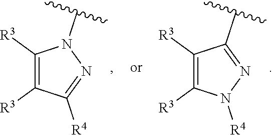

Another embodiment provides a compound of Formula (I), or a pharmaceutically acceptable salt thereof, wherein Ring A is

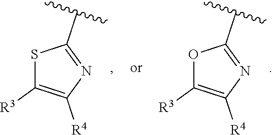

Another embodiment provides a compound of Formula (I), or a pharmaceutically acceptable salt thereof, wherein Ring B is optionally substituted aryl.

Another embodiment provides a compound of Formula (I), or a pharmaceutically acceptable salt thereof, wherein Ring B is optionally substituted phenyl.

Another embodiment provides a compound of Formula (I), or a pharmaceutically acceptable salt thereof, wherein Ring B is optionally substituted heteroaryl.

Another embodiment provides a compound of Formula (I), or a pharmaceutically acceptable salt thereof, wherein Ring B is optionally substituted pyridyl.

Another embodiment provides a compound of Formula (I), or a pharmaceutically acceptable salt thereof, wherein Ring B is optionally substituted 6-membered cycloalkyl.

Another embodiment provides a compound of Formula (I), or a pharmaceutically acceptable salt thereof, wherein Ring B is optionally substituted with at least one substituent selected from halogen, cyano, halo, hydroxy, azido, amino, nitro, —CO₂H, cycloalkyl, —CONH₂, —SO₂Me, or alkoxy.

Another embodiment provides a compound of Formula (I), or a pharmaceutically acceptable salt thereof, wherein Ring B is a phenyl substituted with at least one halogen and n is 1.

Another embodiment provides a compound of Formula (I), or a pharmaceutically acceptable salt thereof, wherein n is 0.

Another embodiment provides a compound of Formula (I), or a pharmaceutically acceptable salt thereof, wherein n is 1.

Another embodiment provides a compound of Formula (I), or a pharmaceutically acceptable salt thereof, wherein R¹ is optionally substituted alkyl.

Another embodiment provides a compound of Formula (I), or a pharmaceutically acceptable salt thereof, wherein R¹ is optionally substituted cycloalkyl.

Another embodiment provides a compound of Formula (I), or a pharmaceutically acceptable salt thereof, wherein R¹ is optionally substituted cyclopropyl.

Another embodiment provides a compound of Formula (I), or a pharmaceutically acceptable salt thereof, wherein R¹ is optionally substituted 3-, 4-, 5-, or 6-membered heterocyclyl, optionally substituted aryl, or optionally substituted heteroaryl.

Another embodiment provides a compound of Formula (I), or a pharmaceutically acceptable salt thereof, wherein R¹ is optionally substituted carbocyclylalkyl, optionally substituted aralkyl, or optionally substituted heteroarylalkyl.

Another embodiment provides a compound of Formula (I), or a pharmaceutically acceptable salt thereof, wherein R² is hydrogen.

Another embodiment provides a compound of Formula (I), or a pharmaceutically acceptable salt thereof, wherein R² is optionally substituted alkyl.

Another embodiment provides a compound of Formula (I), or a pharmaceutically acceptable salt thereof, wherein R² is —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂—NR²⁴CO—R²³, —NR²⁴CO₂—R²³, —NR²⁴CO—N(R²⁴)₂, or —NR²⁴SO₂—N(R²⁴)₂.

Another embodiment provides a compound of Formula (I), or a pharmaceutically acceptable salt thereof, wherein X is —CH₂—.

Another embodiment provides a compound of Formula (I), or a pharmaceutically acceptable salt thereof, wherein X is —NH—.

Another embodiment provides a compound of Formula (I), or a pharmaceutically acceptable salt thereof, wherein R⁹ or R¹⁰ is hydrogen or optionally substituted alkyl.

Another embodiment provides a compound of Formula (I), or a pharmaceutically acceptable salt thereof, wherein R⁹ or R¹⁰ is —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

Another embodiment provides a compound of Formula (I), or a pharmaceutically acceptable salt thereof, wherein R⁹ or R¹⁰ is hydrogen.

Another embodiment provides a compound of Formula (I), or a pharmaceutically acceptable salt thereof, wherein R⁶ is hydrogen.

Another embodiment provides a compound of Formula (I), or a pharmaceutically acceptable salt thereof, wherein R⁶ is —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂—NR²⁴CO—R²³, —NR²⁴CO₂—R²³, —NR²⁴CO—N(R²⁴)₂, or —NR²⁴SO₂—N(R²⁴)₂.

Another embodiment provides a compound of Formula (I), or a pharmaceutically acceptable salt thereof, wherein R⁷ is hydrogen.

Another embodiment provides a compound of Formula (I), or a pharmaceutically acceptable salt thereof, wherein R⁷ and R⁸ are hydrogen.

Another embodiment provides a compound of Formula (I), or a pharmaceutically acceptable salt thereof, wherein R⁷ is —N(R²⁴), —NR²⁴CO—R²³, —NR²⁴CO₂—R²³, —NR²⁴CO—R²³, —NR²⁴CO₂R²³, —NR²⁴CO—N(R²³)₂, —NR²⁴SO₂—N(R²³)₂—NR²⁴CO—R²³, —NR²⁴CO₂—R²³, —NR²⁴CO—N(R²⁴)₂, or —NR²⁴SO₂—N(R²⁴)₂.

Another embodiment provides a compound of Formula (I), or a pharmaceutically acceptable salt thereof, wherein Ring B is a pyridine substituted with at least one halogen and n is 0.

One embodiment provides a compound of Formula (I), or pharmaceutically acceptable salt thereof, chosen from:
(S)-1-(2-((2-((3-chloro-2-fluorobenzyl)amino)-1-cyclopropyl-2-oxoethyl)amino)-2-oxoethyl)-1H-indazole-3-carboxamide;
(S)-1-(2-((1-((3-chloro-2-fluorophenyl)amino)-1-oxopropan-2-yl)(methyl)amino)-2-oxoethyl)-1H-indazole-3-carboxamide;
(S)-1-(2-((1-((3-chloro-2-fluorobenzyl)amino)-1-oxopropan-2-yl)(methyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(isopropyl)amino)-2-oxoethyl)-1H-indazole-3-carboxamide;
1-(2-((2-((3-chloro-2-fluorobenzyl)amino)-2-oxoethyl)(ethyl)amino)-2-oxoethyl)-1H-indazole-3-carboxamide;
1-(2-((2-((3-chloro-2-fluorophenyl)amino)-2-oxoethyl)(cyclopropyl)amino)-2-oxoethyl)-1H-indazole-3-carboxamide;
3-(2-((2-((3-chloro-2-fluorobenzyl)amino)-2-oxoethyl)(cyclopropyl)amino)-2-oxoethyl)-1H-indazole-1-carboxamide;
1-(2-(cyclopropyl(2-((5-hydroxy-3,3-dimethylcyclohexyl)amino)-2-oxoethyl)amino)-2-oxoethyl)-1H-indazole-3-carboxamide;
1-(2-(cyclopropyl(2-(((1S,5S)-5-hydroxy-3,3-dimethylcyclohexyl)amino)-2-oxoethyl)amino)-2-oxoethyl)-1H-indazole-3-carboxamide;
1-(2-((2-((3-chloro-2-fluorophenyl)amino)-2-oxoethyl)(isopropyl)amino)-2-oxoethyl)-1H-indazole-3-carboxamide;
1-(2-((2-((6-chloropyridin-2-yl)amino)-2-oxoethyl)(isopropyl)amino)-2-oxoethyl)-1H-indazole-3-carboxamide;
1-(2-((2-((6-chloropyridin-2-yl)amino)-2-oxoethyl)(2-hydroxyethyl)amino)-2-oxoethyl)-1H-indazole-3-carboxamide;
1-(2-((2-((6-chloropyridin-2-yl)amino)-2-oxoethyl)(methyl)amino)-2-oxoethyl)-1H-indazole-3-carboxamide;
1-(2-((2-((6-chloropyridin-2-yl)amino)-2-oxoethyl)(ethyl)amino)-2-oxoethyl)-1H-indazole-3-carboxamide;
1-(2-((2-((6-chloropyridin-2-yl)amino)-2-oxoethyl)(cyclopropyl)amino)-2-oxoethyl)-1H-indazole-3-carboxamide;
1-(2-((2-((6-chloropyridin-2-yl)amino)-2-oxoethyl)(piperidin-4-yl)amino)-2-oxoethyl)-1H-indazole-3-carboxamide hydrochloride;
3-(2-((2-aminoethyl)(2-((6-chloropyridin-2-yl)amino)-2-oxoethyl)amino)-2-oxoethyl)-1H-indazole-1-carboxamide;
3-(2-((2-((6-chloropyridin-2-yl)amino)-2-oxoethyl)(cyclopentyl)amino)-2-oxoethyl)-1H-indazole-1-carboxamide;
3-(2-((2-((6-chloropyridin-2-yl)amino)-2-oxoethyl)(cyclobutyl)amino)-2-oxoethyl)-1H-indazole-1-carboxamide;
3-(2-((2-((6-chloropyridin-2-yl)amino)-2-oxoethyl)(tetrahydro-2H-pyran-4-yl)amino)-2-oxoethyl)-1H-indazole-1-carboxamide;
1-(2-(azetidin-3-yl(2-((6-chloropyridin-2-yl)amino)-2-oxoethyl)amino)-2-oxoethyl)-1H-indazole-3-carboxamide;
1-(2-((2-((6-chloropyridin-2-yl)amino)-2-oxoethyl)(2-(methylamino)ethyl)amino)-2-oxoethyl)-1H-indazole-3-carboxamide;
1-(2-((2-acetamidoethyl)(2-((6-chloropyridin-2-yl)amino)-2-oxoethyl)amino)-2-oxoethyl)-1H-indazole-3-carboxamide;
1-(2-((2-((6-chloropyridin-2-yl)amino)-2-oxoethyl)(pyridin-4-ylmethyl)amino)-2-oxoethyl)-1H-indazole-3-carboxamide;
1-(2-((2-((6-chloropyridin-2-yl)amino)-2-oxoethyl)(pyrrolidin-3-yl)amino)-2-oxoethyl)-1H-indazole-3-carboxamide;
1-(2-((2-((3-chloro-2-fluorobenzyl)amino)-2-oxoethyl)(thiophen-3-ylmethyl)amino)-2-oxoethyl)-1H-indazole-3-carboxamide;
1-(2-((2-((3-chloro-2-fluorobenzyl)amino)-2-oxoethyl)(cyclobutyl)amino)-2-oxoethyl)-1H-indazole-3-carboxamide;
1-(2-((2-((3-chloro-2-fluorobenzyl)amino)-2-oxoethyl)(tetrahydro-2H-pyran-4-yl)amino)-2-oxoethyl)-1H-indazole-3-carboxamide;
1-(2-((2-((3-chloro-2-fluorobenzyl)amino)-2-oxoethyl)(cyclopentyl)amino)-2-oxoethyl)-1H-indazole-3-carboxamide;
1-(2-((2-aminoethyl)(2-((3-chloro-2-fluorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(pyridin-4-ylmethyl)amino)-2-oxoethyl)-1H-indazole-3-carboxamide;
1-(2-((2-((3-chloro-2-fluorobenzyl)amino)-2-oxoethyl)(methyl)amino)-2-oxoethyl)-1H-indazole-3-carboxamide;
1-(2-((2-((3-chloro-2-fluorobenzyl)amino)-2-oxoethyl)(piperidin-4-yl)amino)-2-oxoethyl)-1H-indazole-3-carboxamide;
1-(2-((2-((3-chloro-2-fluorobenzyl)amino)-2-oxoethyl)(pyridin-3-ylmethyl)amino)-2-oxoethyl)-1H-indazole-3-carboxamide;
1-(2-((2-((3-chloro-2-fluorobenzyl)amino)-2-oxoethyl)(phenyl)amino)-2-oxoethyl)-1H-indazole-3-carboxamide;
1-(2-((2-((3-chloro-2-fluorobenzyl)amino)-2-oxoethyl)(pyrrolidin-3-yl)amino)-2-oxoethyl)-1H-indazole-3-carboxamide;
(R)-1-(2-((1-((3-chloro-2-fluorobenzyl)amino)-1-oxopropan-2-yl)(cyclopropyl)amino)-2-oxoethyl)-1H-indazole-3-carboxamide;
(S)-1-(2-((1-((3-chloro-2-fluorobenzyl)amino)-1-oxopropan-2-yl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-1H-pyrazolo[3,4-c]pyridine-3-carboxamide;
3-(2-((2-((3-chloro-2-fluorobenzyl)amino)-2-oxoethyl)(cyclopropyl)amino)-2-oxoethyl)-1H-indole-1-carboxamide;
3-(2-((2-((3-chloro-2-fluorobenzyl)amino)-2-oxoethyl)(cyclopropyl)amino)-2-oxoethyl)imidazo[1,5-a]pyridine-1-carboxamide;
1-(2-((2-((3-chloro-2-fluorobenzyl)amino)-2-oxoethyl)(cyclopropyl)amino)-2-oxoethyl)-5-(trifluoromethyl)-1H-pyrazole-3-carboxamide;
3-carbamoyl-1-(2-((2-((3-chloro-2-fluorobenzyl)amino)-2-oxoethyl)(cyclopropyl)amino)-2-oxoethyl)-1H-pyrazole-5-carboxylic acid;
1-(2-((2-((3-chloro-2-fluorobenzyl)amino)-2-oxoethyl)(cyclopropyl)amino)-2-oxoethyl)-1H-pyrazole-3,5-dicarboxamide;
4-bromo-1-(2-((2-((3-chloro-2-fluorobenzyl)amino)-2-oxoethyl)(cyclopropyl)amino)-2-oxoethyl)-1H-pyrazole-3-carboxamide;
2-(3-acetyl-1H-indazol-1-yl)-N-(2-((3-chloro-2-fluorobenzyl)amino)-2-oxoethyl)-N-cyclopropylacetamide;
2-(1-acetylimidazo[1,5-a]pyridin-3-yl)-N-(2-((3-chloro-2-fluorobenzyl)amino)-2-oxoethyl)-N-cyclopropylacetamide;
N-(2-((3-chloro-2-fluorobenzyl)amino)-2-oxoethyl)-N-cyclopropyl-2-(3-(1-hydroxyethyl)-1H-indazol-1-yl)acetamide;
3-(3-(2-((3-chloro-2-fluorobenzyl)amino)-2-oxoethyl)-3-isopropylureido)-1H-indole-1-carboxamide;
3-(3-(2-((3-chloro-2-fluorobenzyl)amino)-2-oxoethyl)-3-cyclopropylureido)-1H-indole-1-carboxamide;
3-(3-(2-((3-chloro-2-fluorobenzyl)amino)-2-oxoethyl)-3-(pyrrolidin-3-yl)ureido)-1H-indole-1-carboxamide;
1-(3-(2-((3-chloro-2-fluorobenzyl)amino)-2-oxoethyl)-3-cyclopropylureido)imidazo[1,5-a]pyridine-3-carboxamide;
1-(2-((2-((3-chloro-2-fluorobenzyl)amino)-2-oxoethyl)(3-methylbutan-2-yl)amino)-2-oxoethyl)-1H-indazole-3-carboxamide;
1-(2-((2-((3-chloro-2-fluorobenzyl)amino)-2-oxoethyl)(1-cyclopropylethyl)amino)-2-oxoethyl)-1H-indazole-3-carboxamide;
1-(2-(sec-butyl(2-((3-chloro-2-fluorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(4-hydroxybutan-2-yl)amino)-2-oxoethyl)-1H-indazole-3-carboxamide;
1-(2-((2-((3-chloro-2-fluorobenzyl)amino)-2-oxoethyl)(1-hydroxypropan-2-yl)amino)-2-oxoethyl)-1H-indazole-3-carboxamide;
methyl 2-(2-(3-carbamoyl-1H-indazol-1-yl)-N-(2-((3-chloro-2-fluorobenzyl)amino)-2-oxoethyl)acetamido)propanoate;
1-(2-((2-((3-chloro-2-fluorobenzyl)amino)-2-oxoethyl)(4-fluorobutan-2-yl)amino)-2-oxoethyl)-1H-indazole-3-carboxamide;
1-(2-((2-((3-chloro-2-fluorobenzyl)amino)-2-oxoethyl)(1-methoxybutan-2-yl)amino)-2-oxoethyl)-1H-indazole-3-carboxamide;
1-(2-((1-amino-1-oxopropan-2-yl)(2-((3-chloro-2-fluorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(2-methylcyclopropyl)amino)-2-oxoethyl)-1H-indazole-3-carboxamide;
1-(2-((2-((3-chloro-2-fluorobenzyl)amino)-2-oxoethyl)(3,3-dimethylbutan-2-yl)amino)-2-oxoethyl)-1H-indazole-3-carboxamide;
1-(2-((2-((3-chloro-2-fluorobenzyl)amino)-2-oxoethyl)(1-phenylethyl)amino)-2-oxoethyl)-1H-indazole-3-carboxamide;
1-(2-((2-((3-chloro-2-fluorobenzyl)amino)-2-oxoethyl)(1-fluoropropan-2-yl)amino)-2-oxoethyl)-1H-indazole-3-carboxamide;
1-(2-((2-((3-chloro-2-fluorobenzyl)amino)-2-oxoethyl)(1,1,1-trifluoropropan-2-yl)amino)-2-oxoethyl)-1H-indazole-3-carboxamide;
1-(2-((2-((3-chloro-2-fluorobenzyl)amino)-2-oxoethyl)(1-methoxypropan-2-yl)amino)-2-oxoethyl)-1H-indazole-3-carboxamide;
1-(2-((2-((3-chloro-2-fluorobenzyl)amino)-2-oxoethyl)(1-cyclopropyl-2-hydroxyethyl)amino)-2-oxoethyl)-1H-indazole-3-carboxamide;
1-(2-((2-((2-chloro-3-fluorobenzyl)amino)-2-oxoethyl)(1,3-difluoropropan-2-yl)amino)-2-oxoethyl)-1H-indazole-3-carboxamide;
1-(2-((4-aminobutan-2-yl)(2-((2-chloro-3-fluorobenzyl)amino)-2-oxoethyl)amino)-2-oxoethyl)-1H-indazole-3-carboxamide;
ethyl 3-(2-(3-carbamoyl-1H-indazol-1-yl)-N-(2-((3-chloro-2-fluorobenzyl)amino)-2-oxoethyl)acetamido)butanoate;
1-(2-((2-((3-chloro-2-fluorobenzyl)amino)-2-oxoethyl)(1-cyclopropyl-3-hydroxypropyl)amino)-2-oxoethyl)-1H-indazole-3-carboxamide;
3-(2-(3-carbamoyl-1H-indazol-1-yl)-N-(2-((3-chloro-2-fluorobenzyl)amino)-2-oxoethyl)acetamido)butanoic acid;
1-(2-((3-amino-1-cyclopropyl-3-oxopropyl)(2-((3-chloro-2-fluorobenzyl)amino)-2-oxoethyl)amino)-2-oxoethyl)-1H-indazole-3-carboxamide;
1-(2-((4-amino-4-oxobutan-2-yl)(2-((3-chloro-2-fluorobenzyl)amino)-2-oxoethyl)amino)-2-oxoethyl)-1H-indazole-3-carboxamide;
ethyl 3-(2-(3-carbamoyl-1H-indazol-1-yl)-N-(2-((3-chloro-2-fluorobenzyl)amino)-2-oxoethyl)acetamido)-4,4,4-trifluorobutanoate;
1-(2-((2-((3-chloro-2-fluorobenzyl)amino)-2-oxoethyl)(cyclopropyl)amino)-2-oxoethyl)-5-fluoro-1H-indazole-3-carboxamide;
3-(2-(3-carbamoyl-1H-indazol-1-yl)-N-(2-((3-chloro-2-fluorobenzyl)amino)-2-oxoethyl)acetamido)-4,4,4-trifluorobutanoic acid;
5-chloro-1-(2-((2-((3-chloro-2-fluorobenzyl)amino)-2-oxoethyl)(cyclopropyl)amino)-2-oxoethyl)-1H-indazole-3-carboxamide;
7-chloro-1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-2,6-difluorobenzyl)amino)-2-oxoethyl)(cyclopropyl)amino)-2-oxoethyl)-1H-indazole-3-carboxamide;
1-(2-((2-((3-chloro-2,4-difluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-6-fluoro-1H-indazole-3-carboxamide;
6-chloro-1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-6-cyano-1H-indazole-3-carboxamide;
1-(2-((2-((3-chloro-6-cyano-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-7-fluoro-1H-indazole-3-carboxamide;
6-(aminomethyl)-1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-5-cyano-2-fluorobenzyl)amino)-2-oxoethyl)(cyclopropyl)amino)-2-oxoethyl)-1H-indazole-3-carboxamide;
1-(2-((2-(((6-chloro-1H-indazol-4-yl)methyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-5-cyano-1H-indazole-3-carboxamide;
1-(2-((2-((3-chloro-2-fluorobenzyl)amino)-2-oxoethyl)(cyclopropyl)amino)-2-oxoethyl)-1H-indazole-3,5-dicarboxamide;
1-(2-((2-((3-chloro-2-fluorobenzyl)amino)-2-oxoethyl)(cyclopropyl)amino)-2-oxoethyl)-6-(trifluoromethyl)-1H-indazole-3-carboxamide;
1-(2-((2-((3-chloro-2-fluorobenzyl)amino)-2-oxoethyl)(cyclopropyl)amino)-2-oxoethyl)-5-(trifluoromethyl)-1H-indazole-3-carboxamide;
1-(2-((2-((3-chloro-4-cyano-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-4-fluoro-1H-indazole-3-carboxamide;
2-(3-(1H-imidazol-2-yl)-1H-indazol-1-yl)-N-(2-((3-chloro-2-fluorobenzyl)amino)-2-oxoethyl)-N-cyclopropylacetamide;
1-(2-((2-((3-chloro-2-fluoro-4-(hydroxymethyl)benzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-1H-indazole-3,6-dicarboxamide;
1-(2-((2-((5-(aminomethyl)-3-chloro-2-fluorobenzyl)amino)-2-oxoethyl)(cyclopropyl)amino)-2-oxoethyl)-1H-indazole-3-carboxamide;
1-(2-((2-(((4-chloro-1H-indazol-6-yl)methyl)amino)-2-oxoethyl)(cyclopropyl)amino)-2-oxoethyl)-1H-indazole-3-carboxamide;
1-(2-((2-((3-chloro-5-fluorobenzyl)amino)-2-oxoethyl)(cyclopropyl)amino)-2-oxoethyl)-1H-indazole-3-carboxamide;
1-(2-(cyclopropyl(2-((3,4-dichlorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-7-cyano-1H-indazole-3-carboxamide;
1-(2-(cyclopropyl(2-((2,3-dichlorobenzyl)amino)-2-oxoethyl)amino)-2-oxoethyl)-1H-indazole-3-carboxamide;
1-(2-((2-(((6-chloropyridin-2-yl)methyl)amino)-2-oxoethyl)(cyclopropyl)amino)-2-oxoethyl)-1H-indazole-3-carboxamide;
1-(2-((2-((3-chloro-4-fluorobenzyl)amino)-2-oxoethyl)(cyclopropyl)amino)-2-oxoethyl)-1H-indazole-3-carboxamide;
1-(2-((2-(((5-chloro-1H-indazol-7-yl)methyl)amino)-2-oxoethyl)(cyclopropyl)amino)-2-oxoethyl)-1H-indazole-3-carboxamide;
1-(2-((2-(((5-chlorobenzofuran-7-yl)methyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-7-(trifluoromethyl)-1H-indazole-3-carboxamide;
1-(2-((2-((3-chlorobenzyl)amino)-2-oxoethyl)(cyclopropyl)amino)-2-oxoethyl)-1H-indazole-3-carboxamide;
1-(2-((2-((3-chloro-4-methylbenzyl)amino)-2-oxoethyl)(cyclopropyl)amino)-2-oxoethyl)-1H-indazole-3-carboxamide;
1-(2-((2-(((7-chloro-1H-indazol-5-yl)methyl)amino)-2-oxoethyl)(cyclopropyl)amino)-2-oxoethyl)-1H-indazole-3-carboxamide;
1-(2-(cyclopropyl(2-((2-fluorobenzyl)amino)-2-oxoethyl)amino)-2-oxoethyl)-1H-indazole-3-carboxamide;
1-(2-((2-((3-chloro-2-methoxybenzyl)amino)-2-oxoethyl)(cyclopropyl)amino)-2-oxoethyl)-1H-indazole-3-carboxamide;
1-(2-((2-((4-carbamoyl-2-fluorobenzyl)amino)-2-oxoethyl)(cyclopropyl)amino)-2-oxoethyl)-1H-indazole-3-carboxamide;
1-(2-((2-((3-amino-2-fluorobenzyl)amino)-2-oxoethyl)(cyclopropyl)amino)-2-oxoethyl)-1H-indazole-3-carboxamide;
1-(2-(cyclopropyl(2-((3,5-dichlorobenzyl)amino)-2-oxoethyl)amino)-2-oxoethyl)-1H-indazole-3-carboxamide;
1-(2-((2-((3-bromo-2-fluorobenzyl)amino)-2-oxoethyl)(cyclopropyl)amino)-2-oxoethyl)-1H-indazole-3-carboxamide;
1-(2-((2-((4-amino-2-fluorobenzyl)amino)-2-oxoethyl)(cyclopropyl)amino)-2-oxoethyl)-1H-indazole-3-carboxamide;
1-(2-(cyclopropyl(2-((2-fluoro-3-methoxybenzyl)amino)-2-oxoethyl)amino)-2-oxoethyl)-1H-indazole-3-carboxamide;
1-(2-((2-((3-cyano-2-fluorobenzyl)amino)-2-oxoethyl)(cyclopropyl)amino)-2-oxoethyl)-1H-indazole-3-carboxamide;
1-(2-((2-((3-chloro-5-(trifluoromethyl)benzyl)amino)-2-oxoethyl)(cyclopropyl)amino)-2-oxoethyl)-1H-indazole-3-carboxamide;
1-(2-((2-((3-chloro-4-methoxybenzyl)amino)-2-oxoethyl)(cyclopropyl)amino)-2-oxoethyl)-1H-indazole-3-carboxamide;
1-(2-(cyclopropyl(2-((2-fluoro-3-(hydroxymethyl)benzyl)amino)-2-oxoethyl)amino)-2-oxoethyl)-1H-indazole-3-carboxamide;
1-(2-(cyclopropyl(2-((3-cyclopropyl-2-fluorobenzyl)amino)-2-oxoethyl)amino)-2-oxoethyl)-1H-indazole-3-carboxamide;
1-(2-(cyclopropyl(2-((2-fluoro-3-methylbenzyl)amino)-2-oxoethyl)amino)-2-oxoethyl)-1H-indazole-3-carboxamide;
1-(2-((2-((3-chloro-5-methylbenzyl)amino)-2-oxoethyl)(cyclopropyl)amino)-2-oxoethyl)-1H-indazole-3-carboxamide;
1-(2-((2-(((5-chloropyridin-3-yl)methyl)amino)-2-oxoethyl)(cyclopropyl)amino)-2-oxoethyl)-1H-indazole-3-carboxamide;
1-(2-((2-((3-chloro-2-cyclopropylbenzyl)amino)-2-oxoethyl)(cyclopropyl)amino)-2-oxoethyl)-1H-indazole-3-carboxamide;
1-(2-(cyclopropyl(2-((2,3-difluorobenzyl)amino)-2-oxoethyl)amino)-2-oxoethyl)-1H-indazole-3-carboxamide;
1-(2-((2-((3-chloro-5-cyanobenzyl)amino)-2-oxoethyl)(cyclopropyl)amino)-2-oxoethyl)-1H-indazole-3-carboxamide;
1-(2-((2-((3-chloro-2-methylbenzyl)amino)-2-oxoethyl)(cyclopropyl)amino)-2-oxoethyl)-1H-indazole-3-carboxamide;
1-(2-((2-((3-chloro-2-(trifluoromethyl)benzyl)amino)-2-oxoethyl)(cyclopropyl)amino)-2-oxoethyl)-1H-indazole-3-carboxamide;
1-(2-(cyclopropyl(2-((2-fluoro-3-(trifluoromethyl)benzyl)amino)-2-oxoethyl)amino)-2-oxoethyl)-1H-indazole-3-carboxamide;
2-(3-acetylimidazo[1,5-a]pyridin-1-yl)-N-(2-((3-chloro-2-fluorobenzyl)amino)-2-oxoethyl)-N-cyclopropylacetamide;
1-(2-((2-((3-chloro-2-fluorobenzyl)amino)-2-oxoethyl)(cyclopropyl)amino)-2-oxoethyl)imidazo[1,5-a]pyridine-3-carboxamide;
2-(1-acetylimidazo[1,5-a]pyridin-3-yl)-N-(2-((3-chloro-2-fluorobenzyl)amino)-2-oxoethyl)-N-cyclopropylacetamide;
3-(3-(2-((3-chloro-2-fluorobenzyl)amino)-2-oxoethyl)-3-cyclopropylureido)imidazo[1,5-a]pyridine-1-carboxamide; or
2-(3-(3-acetylimidazo[1,5-a]pyridin-1-yl)-1-cyclopropylureido)-N-(3-chloro-2-fluorobenzyl)acetamide.

One embodiments provides a pharmaceutical composition comprising a pharmaceutically acceptable excipient and a compound of Formula (I), or a pharmaceutically acceptable salt thereof.

One embodiment provides a method of treating an autoimmune, inflammatory, or neurodegenerative disease in a patient in need thereof comprising administering to the patient a pharmaceutical composition comprising a compound Formula (I), or a pharmaceutically acceptable salt thereof.

Another embodiment provides a method treating an autoimmune, inflammatory, or neurodegenerative disease in a patient in need thereof comprising administering to the patient a pharmaceutical composition comprising a compound Formula (I), or a pharmaceutically acceptable salt thereof, wherein the autoimmune, inflammatory, or neurodegenerative disease is paraoxysmal nocturnal hemoglobinuria.

One embodiment provides a compound, or a pharmaceutically acceptable salt thereof, having the structure of Formula (Ia):

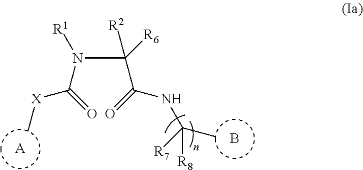

wherein,
Ring A is an optionally substituted heteroaryl ring;
Ring B is an optionally substituted aryl, optionally substituted heteroaryl, optionally substituted 3-, 4-, 5-, or 6-membered cycloalkyl, or optionally substituted 3-, 4-, 5-, or 6-membered heterocyclyl;
X is —CR⁹R¹⁰— or —NR¹¹—;
R¹ is optionally substituted alkyl, optionally substituted 3-, 4-, 5-, or 6-membered cycloalkyl, optionally substituted 3-, 4-, 5-, or 6-membered heterocyclyl, optionally substituted aryl, optionally substituted aralkyl, optionally substituted heteroaryl, or optionally substituted heteroarylalkyl;
R², R⁶, R⁹, R¹⁰, each R⁷, or each R⁸, is independently selected from hydrogen, halo, hydroxy, amino, —CO₂H, —S(O)—R²⁰, —S—R²⁰, —S(O)₂—R²⁰, optionally substituted alkoxy, optionally substituted aryloxy, optionally substituted heteroaryloxy, optionally substituted (heterocyclyl)-O—, optionally substituted alkyl, optionally substituted cycloalkyl, optionally substituted alkenyl, optionally substituted aryl, optionally substituted heteroaryl, optionally substituted heterocyclyl, optionally substituted alkylamino, optionally substituted dialkylamino, —CO—R²⁰, —CO₂—R²⁰, —CO(NR²¹)₂, —SO₂(NR²¹)₂, —C(═NR²²)—(NR²¹)₂, or optionally substituted alkynyl;
each R²⁰ is independently optionally substituted alkyl, optionally substituted cycloalkyl, optionally substituted aryl, or optionally substituted heteroaryl;
each R²¹ is independently hydrogen, optionally substituted alkyl, optionally substituted cycloalkyl, optionally substituted aryl, or optionally substituted heteroaryl;
each R²² is independently hydrogen, optionally substituted alkyl, or optionally substituted cycloalkyl;
R¹¹ is hydrogen, or optionally substituted alkyl; and
n is 0, 1, 2, or 3.

Another embodiment provides the compound of Formula (Ia), or a pharmaceutically acceptable salt thereof, wherein Ring A is

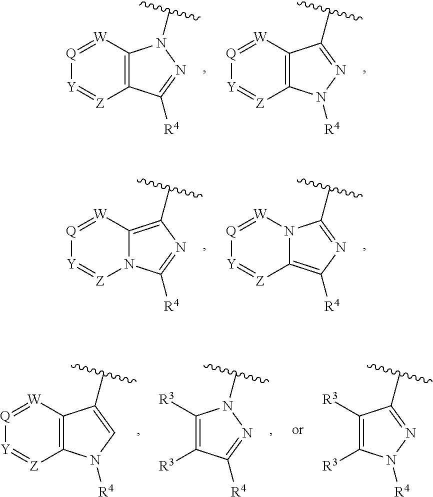

wherein
W, Q, Y, and Z are each independently selected from N or C—R³;
each R³ is independently selected from hydrogen, cyano, halo, hydroxy, azido, amino, nitro, —CO₂H, —S(O)—R²³, —S—R²³, —S(O)₂—R²³, optionally substituted alkoxy, optionally substituted aryloxy, optionally substituted heteroaryloxy, optionally substituted (heterocyclyl)-O—, optionally substituted alkyl, optionally substituted cycloalkyl, optionally substituted alkenyl, optionally substituted aryl, optionally substituted heteroaryl, optionally substituted heterocyclyl, optionally substituted alkylamino, optionally substituted dialkylamino, —CO—R²³, —CO₂—R²³, —CO(NR²⁴)₂, —NR²⁴CO—R²³, —NR²⁴CO₂—R²³, —SO₂(NR²⁴)₂, —C(═NR²⁵)—(NR²⁴)₂, or optionally substituted alkynyl;
each R²³ is independently optionally substituted alkyl, optionally substituted cycloalkyl, optionally substituted aryl, or optionally substituted heteroaryl;
each R²⁴ is independently hydrogen, optionally substituted alkyl, optionally substituted cycloalkyl, optionally substituted aryl, or optionally substituted heteroaryl;
each R²⁵ is independently hydrogen, optionally substituted alkyl, or optionally substituted cycloalkyl;
R⁴ is optionally substituted alkyl or —C(═O)R⁵;
R⁵ is selected from NH₂, optionally substituted alkylamino, optionally substituted dialkylamino, optionally substituted alkyl, or optionally substituted cycloalkyl.

Another embodiment provides the compound of Formula (Ia), or a pharmaceutically acceptable salt thereof, wherein W, Q, Y, and Z are C—R³ and each R³ is independently selected from hydrogen, halogen, optionally substituted alkyl, optionally substituted cycloalkyl, or optionally substituted alkoxy.

Another embodiment provides the compound of Formula (Ia), or a pharmaceutically acceptable salt thereof, wherein W, Q, Y, and Z are C—R³ and each R³ is hydrogen.

Another embodiment provides the compound of Formula (Ia), or a pharmaceutically acceptable salt thereof, wherein Q is N; W, Y, and Z are C—R³; and each R³ is independently selected from hydrogen, halogen, optionally substituted alkyl, optionally substituted cycloalkyl, or optionally substituted alkoxy.

Another embodiment provides the compound of Formula (Ia), or a pharmaceutically acceptable salt thereof, wherein Q is N; W, Y, and Z are C—R³; and each R³ is hydrogen.

Another embodiment provides the compound of Formula (Ia), or a pharmaceutically acceptable salt thereof, wherein Q is N; W, Y, and Z are C—R³; and each R³ is independently selected from hydrogen, halogen, optionally substituted alkyl, optionally substituted cycloalkyl, or optionally substituted alkoxy.

Another embodiment provides the compound of Formula (Ia), or a pharmaceutically acceptable salt thereof, wherein Q is N; W, Y, and Z are C—R³; and each R³ is hydrogen.

Another embodiment provides the compound of Formula (Ia), or a pharmaceutically acceptable salt thereof, wherein W is N; Q, Y, and Z are C—R³; and each R³ is independently selected from hydrogen, halogen, optionally substituted alkyl, optionally substituted cycloalkyl, or optionally substituted alkoxy.

Another embodiment provides the compound of Formula (Ia), or a pharmaceutically acceptable salt thereof, wherein W is N; Q, Y, and Z are C—R³; and each R³ is hydrogen.

Another embodiment provides the compound of Formula (Ia), or a pharmaceutically acceptable salt thereof, wherein Y is N; W, Q, and Z are C—R³; and each R³ is independently selected from hydrogen, halogen, optionally substituted alkyl, optionally substituted cycloalkyl, or optionally substituted alkoxy.

Another embodiment provides the compound of Formula (Ia), or a pharmaceutically acceptable salt thereof, wherein Y is N; W, Q, and Z are C—R³; and each R³ is hydrogen.

Another embodiment provides the compound of Formula (Ia), or a pharmaceutically acceptable salt thereof, wherein Z is N; W, Q, and Y are C—R³; and each R³ is independently selected from hydrogen, halogen, optionally substituted alkyl, optionally substituted cycloalkyl, or optionally substituted alkoxy.

Another embodiment provides the compound of Formula (Ia), or a pharmaceutically acceptable salt thereof, wherein Z is N; W, Q, and Y are C—R³; and each R³ is hydrogen.

Another embodiment provides the compound of Formula (Ia), or a pharmaceutically acceptable salt thereof, wherein Ring B is optionally substituted aryl. Another embodiment provides the compound of Formula (Ia), or a pharmaceutically acceptable salt thereof, wherein Ring B is optionally substituted heteroaryl. Another embodiment provides the compound of Formula (Ia), or a pharmaceutically acceptable salt thereof, wherein Ring B is optionally substituted 6-membered cycloalkyl.

Another embodiment provides the compound of Formula (Ia), or a pharmaceutically acceptable salt thereof, wherein n is 0. Another embodiment provides the compound of Formula (I), or a pharmaceutically acceptable salt thereof, wherein n is 1.

Another embodiment provides the compound of Formula (Ia), or a pharmaceutically acceptable salt thereof, wherein R¹ is optionally substituted alkyl. Another embodiment provides the compound of Formula (Ia), or a pharmaceutically acceptable salt thereof, wherein R¹ is optionally substituted cycloalkyl. Another embodiment provides the compound of Formula (Ia), or a pharmaceutically acceptable salt thereof, wherein R¹ is optionally substituted heterocyclylalkyl.

Another embodiment provides the compound of Formula (Ia), or a pharmaceutically acceptable salt thereof, wherein R² is hydrogen. Another embodiment provides the compound of Formula (Ia), or a pharmaceutically acceptable salt thereof, wherein R² is optionally substituted alkyl.

Another embodiment provides the compound of Formula (Ia), or a pharmaceutically acceptable salt thereof, wherein X is —CH₂—.

Another embodiment provides the compound of Formula (Ia), or a pharmaceutically acceptable salt thereof, wherein R⁹ or R¹⁰ is hydrogen or optionally substituted alkyl. Another embodiment provides the compound of Formula (Ia), or a pharmaceutically acceptable salt thereof, wherein R⁹ or R¹⁰ is hydrogen. Another embodiment provides the compound of Formula (Ia), or a pharmaceutically acceptable salt thereof, wherein R⁶ is hydrogen. Another embodiment provides the compound of Formula (Ia), or a pharmaceutically acceptable salt thereof, wherein R⁷ is hydrogen. Another embodiment provides the compound of Formula (Ia), or a pharmaceutically acceptable salt thereof, wherein R⁷ and R⁸ are hydrogen. Another embodiment provides the compound of Formula (Ia), or a pharmaceutically acceptable salt thereof, wherein R¹¹ is hydrogen.

In some embodiments, the complement factor D inhibitory compound described by Formula (I) or (Ia) has a structure provided in Table 1.

TABLE 1

| Example | Structure | Name |
|---|---|---|
| 1 | 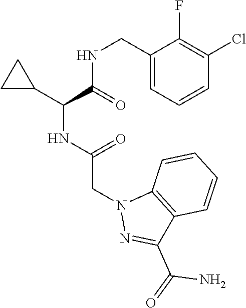 [SMILES: NC(=O)c1nn(CC(=O)N[C@H](C(=O)NCc2cccc(Cl)c2F)C2CC2)c2ccccc12] | (S)-1-(2-((2-((3-chloro-2- fluorobenzyl)amino)-1- cyclopropyl-2- oxoethyl)amino)-2- oxoethyl)-1H-indazole-3- carboxamide |
| 2 | 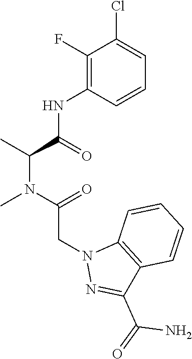 [SMILES: C[C@@H](C(=O)Nc1cccc(Cl)c1F)N(C)C(=O)Cn1nc(C(N)=O)c2ccccc21] | (S)-1-(2-((1-((3-chloro-2- fluorophenyl)amino)-1- oxopropan-2- yl)(methyl)amino)-2- oxoethyl)-1H-indazole-3- carboxamide |
| 3 | 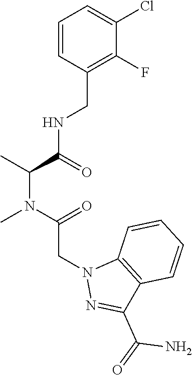 [SMILES: C[C@@H](C(=O)NCc1cccc(Cl)c1F)N(C)C(=O)Cn1nc(C(N)=O)c2ccccc21] | (S)-1-(2-((1-((3-chloro-2- fluorobenzyl)amino)-1- oxopropan-2- yl)(methyl)amino)-2- oxoethyl)-1H-indazole-3- carboxamide |
| 4 | 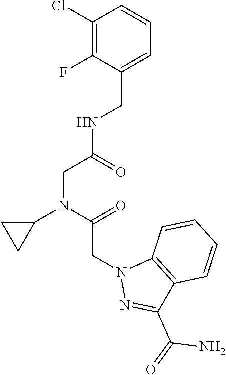 [SMILES: NC(=O)c1nn(CC(=O)N(CC(=O)NCc2cccc(Cl)c2F)C2CC2)c2ccccc12] | 1-(2-((2-((3-chloro-2- fluorobenzyl)amino)-2- oxoethyl)(cyclopropyl)amino)- 2-oxoethyl)-1H-indazole- 3-carboxamide |
| 5 | 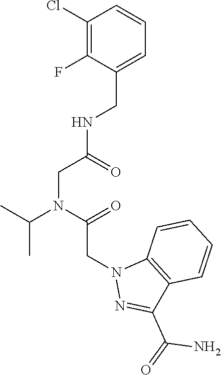 [SMILES: CC(C)N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(isopropyl)amino)- 2-oxoethyl)-1H-indazole-3- carboxamide |
| 6 | 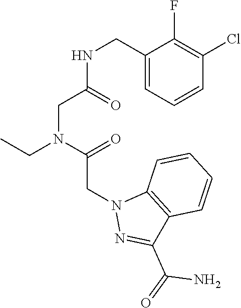 [SMILES: CCN(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(ethyl)amino)-2- oxoethyl)-1H-indazole-3- carboxamide |
| 7 | 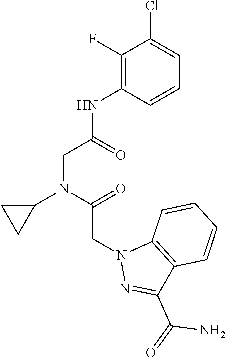 [SMILES: NC(=O)c1nn(CC(=O)N(CC(=O)Nc2cccc(Cl)c2F)C2CC2)c2ccccc12] | 1-(2-((2-((3-chloro-2- fluorophenyl)amino)-2- oxoethyl)(cyclopropyl)amino)- 2-oxoethyl)-1H-indazole- 3-carboxamide |
| 8 | 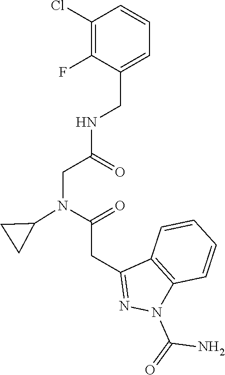 [SMILES: NC(=O)n1nc(CC(=O)N(CC(=O)NCc2cccc(Cl)c2F)C2CC2)c2ccccc21] | 3-(2-((2-((3-chloro-2- fluorobenzyl)amino)-2- oxoethyl)(cyclopropyl)amino)- 2-oxoethyl)-1H-indazole- 1-carboxamide |
| 9 | 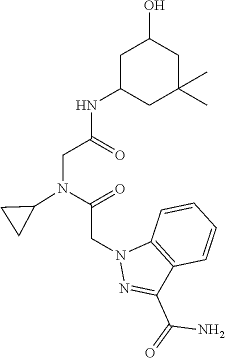 [SMILES: CC1(C)CC(O)CC(NC(=O)CN(C(=O)Cn2nc(C(N)=O)c3ccccc32)C2CC2)C1] | 1-(2-(cyclopropyl(2-((5- hydroxy-3,3- dimethylcyclohexyl)amino)- 2-oxoethyl)amino)-2- oxoethyl)-1H-indazole-3- carboxamide |
| 10 | 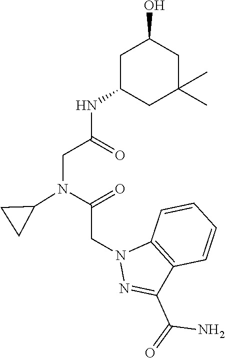 [SMILES: CC1(C)C[C@H](O)C[C@@H](NC(=O)CN(C(=O)Cn2nc(C(N)=O)c3ccccc32)C2CC2)C1] | 1-(2-(cyclopropyl(2- (((1S,5S)-5-hydroxy-3,3- dimethylcyclohexyl)amino)- 2-oxoethyl)amino)-2- oxoethyl)-1H-indazole-3- carboxamide |
| 11 | 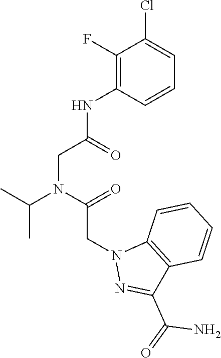 [SMILES: CC(C)N(CC(=O)Nc1cccc(Cl)c1F)C(=O)Cn1nc(C(N)=O)c2ccccc21] | 1-(2-((2-((3-chloro-2- fluorophenyl)amino)-2- oxoethyl)(isopropyl)amino)- 2-oxoethyl)-1H-indazole-3- carboxamide |
| 12 | 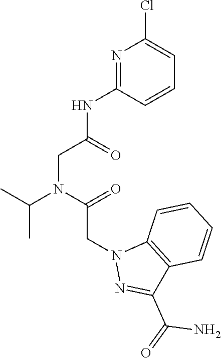 [SMILES: CC(C)N(CC(=O)Nc1cccc(Cl)n1)C(=O)Cn1nc(C(N)=O)c2ccccc21] | 1-(2-((2-((6-chloropyridin- 2-yl)amino)-2- oxoethyl)(isopropyl)amino)- 2-oxoethyl)-1H-indazole-3- carboxamide |
| 13 | 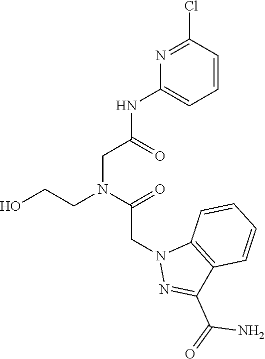 [SMILES: NC(=O)c1nn(CC(=O)N(CCO)CC(=O)Nc2cccc(Cl)n2)c2ccccc12] | 1-(2-((2-((6-chloropyridin- 2-yl)amino)-2-oxoethyl)(2- hydroxyethyl)amino)-2- oxoethyl)-1H-indazole-3- carboxamide |
| 14 | 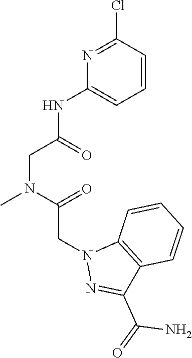 [SMILES: CN(CC(=O)Nc1cccc(Cl)n1)C(=O)Cn1nc(C(N)=O)c2ccccc21] | 1-(2-((2-((6-chloropyridin- 2-yl)amino)-2- oxoethyl)(methyl)amino)-2- oxoethyl)-1H-indazole-3- carboxamide |
| 15 | 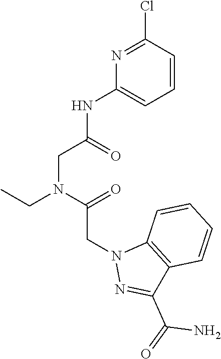 [SMILES: CCN(CC(=O)Nc1cccc(Cl)n1)C(=O)Cn1nc(C(N)=O)c2ccccc21] | 1-(2-((2-((6-chloropyridin- 2-yl)amino)-2- oxoethyl)(ethyl)amino)-2- oxoethyl)-1H-indazole-3- carboxamide |
| 16 | 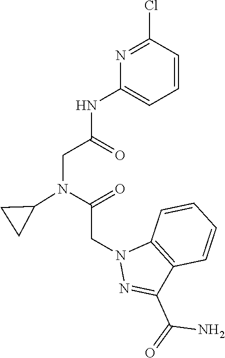 [SMILES: NC(=O)c1nn(CC(=O)N(CC(=O)Nc2cccc(Cl)n2)C2CC2)c2ccccc12] | 1-(2-((2-((6-chloropyridin- 2-yl)amino)-2- oxoethyl)(cyclopropyl)amino)- 2-oxoethyl)-1H-indazole- 3-carboxamide |
| 17 | 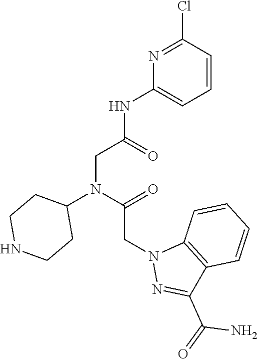 [SMILES: NC(=O)c1nn(CC(=O)N(CC(=O)Nc2cccc(Cl)n2)C2CCNCC2)c2ccccc12] | 1-(2-((2-((6-chloropyridin- 2-yl)amino)-2- oxoethyl)(piperidin-4- yl)amino)-2-oxoethyl)-1H- indazole-3-carboxamide |
| 18 | 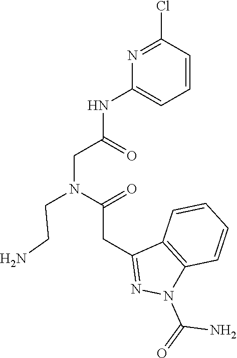 [SMILES: NCCN(CC(=O)Nc1cccc(Cl)n1)C(=O)Cc1nn(C(N)=O)c2ccccc12] | 3-(2-((2-aminoethyl)(2-((6- chloropyridin-2-yl)amino)- 2-oxoethyl)amino)-2- oxoethyl)-1H-indazole-1- carboxamide |
| 19 | 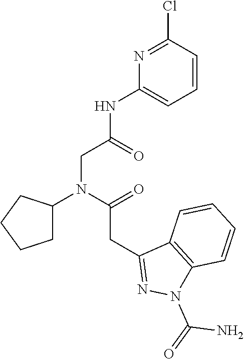 [SMILES: NC(=O)n1nc(CC(=O)N(CC(=O)Nc2cccc(Cl)n2)C2CCCC2)c2ccccc21] | 3-(2-((2-((6-chloropyridin- 2-yl)amino)-2- oxoethyl)(cyclopentyl)amino)- 2-oxoethyl)-1H-indazole- 1-carboxamide |
| 20 | 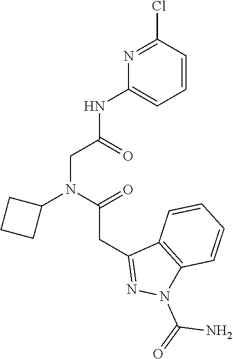 [SMILES: NC(=O)n1nc(CC(=O)N(CC(=O)Nc2cccc(Cl)n2)C2CCC2)c2ccccc21] | 3-(2-((2-((6-chloropyridin- 2-yl)amino)-2- oxoethyl)(cyclobutyl)amino)- 2-oxoethyl)-1H-indazole- 1-carboxamide |
| 21 | 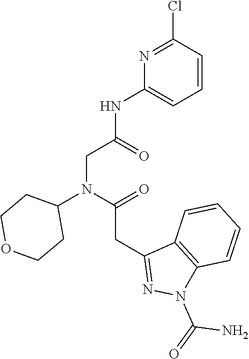 [SMILES: NC(=O)n1nc(CC(=O)N(CC(=O)Nc2cccc(Cl)n2)C2CCOCC2)c2ccccc21] | 3-(2-((2-((6-chloropyridin- 2-yl)amino)-2- oxoethyl)(tetrahydro-2H- pyran-4-yl)amino)-2- oxoethyl)-1H-indazole-1- carboxamide |
| 22 | 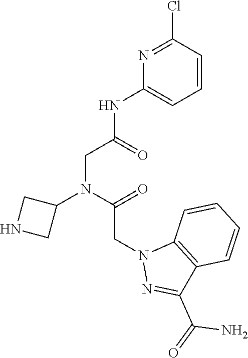 [SMILES: NC(=O)c1nn(CC(=O)N(CC(=O)Nc2cccc(Cl)n2)C2CNC2)c2ccccc12] | 1-(2-(azetidin-3-yl(2-((6- chloropyridin-2-yl)amino)- 2-oxoethyl)amino)-2- oxoethyl)-1H-indazole-3- carboxamide |
| 23 | 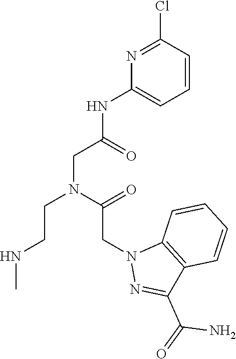 [SMILES: CNCCN(CC(=O)Nc1cccc(Cl)n1)C(=O)Cn1nc(C(N)=O)c2ccccc21] | 1-(2-((2-((6-chloropyridin- 2-yl)amino)-2-oxoethyl)(2- (methylamino)ethyl)amino)- 2-oxoethyl)-1H-indazole-3- carboxamide |
| 24 | 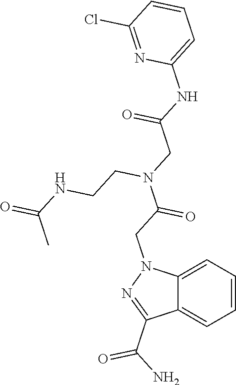 [SMILES: CC(=O)NCCN(CC(=O)Nc1cccc(Cl)n1)C(=O)Cn1nc(C(N)=O)c2ccccc21] | 1-(2-((2-acetamidoethyl)(2- ((6-chloropyridin-2- yl)amino)-2- oxoethyl)amino)-2- oxoethyl)-1H-indazole-3- carboxamide |
| 25 | 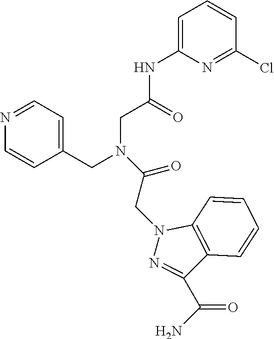 [SMILES: NC(=O)c1nn(CC(=O)N(CC(=O)Nc2cccc(Cl)n2)Cc2ccncc2)c2ccccc12] | 1-(2-((2-((6-chloropyridin- 2-yl)amino)-2- oxoethyl)(pyridin-4- ylmethyl)amino)-2- oxoethyl)-1H-indazole-3- carboxamide |
| 26 | 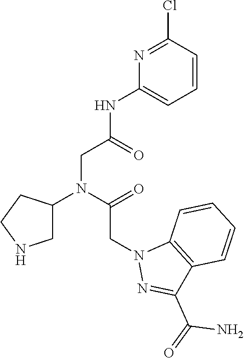 [SMILES: NC(=O)c1nn(CC(=O)N(CC(=O)Nc2cccc(Cl)n2)C2CCNC2)c2ccccc12] | 1-(2-((2-((6-chloropyridin- 2-yl)amino)-2- oxoethyl)(pyrrolidin-3- yl)amino)-2-oxoethyl)-1H- indazole-3-carboxamide |
| 27 | 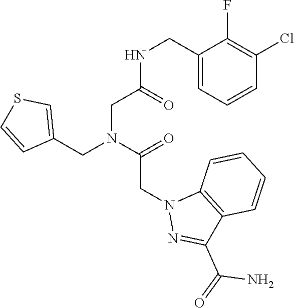 [SMILES: NC(=O)c1nn(CC(=O)N(CC(=O)NCc2cccc(Cl)c2F)Cc2ccsc2)c2ccccc12] | 1-(2-((2-((3-chloro-2- fluorobenzyl)amino)-2- oxoethyl)(thiophen-3- ylmethyl)amino)-2- oxoethyl)-1H-indazole-3- carboxamide |
| 28 | 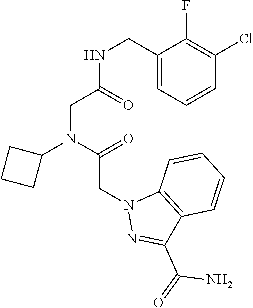 [SMILES: NC(=O)c1nn(CC(=O)N(CC(=O)NCc2cccc(Cl)c2F)C2CCC2)c2ccccc12] | 1-(2-((2-((3-chloro-2- fluorobenzyl)amino)-2- oxoethyl)(cyclobutyl)amino)- 2-oxoethyl)-1H-indazole- 3-carboxamide |
| 29 | 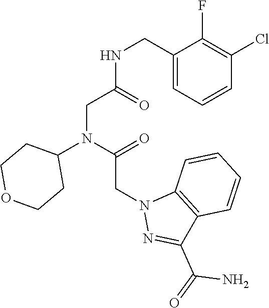 [SMILES: NC(=O)c1nn(CC(=O)N(CC(=O)NCc2cccc(Cl)c2F)C2CCOCC2)c2ccccc12] | 1-(2-((2-((3-chloro-2- fluorobenzyl)amino)-2- oxoethyl)(tetrahydro-2H- pyran-4-yl)amino)-2- oxoethyl)-1H-indazole-3- carboxamide |
| 30 | 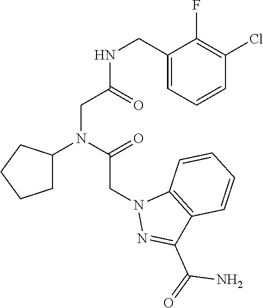 [SMILES: NC(=O)c1nn(CC(=O)N(CC(=O)NCc2cccc(Cl)c2F)C2CCCC2)c2ccccc12] | 1-(2-((2-((3-chloro-2- fluorobenzyl)amino)-2- oxoethyl)(cyclopentyl)amino)- 2-oxoethyl)-1H-indazole- 3-carboxamide |
| 31 | 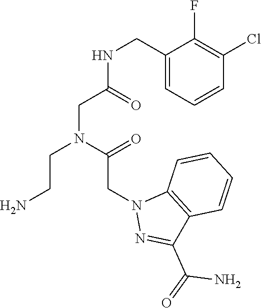 [SMILES: NCCN(CC(=O)NCc1cccc(Cl)c1F)C(=O)Cn1nc(C(N)=O)c2ccccc21] | 1-(2-((2-aminoethyl)(2-((3- chloro-2- fluorobenzyl)amino)-2- oxoethyl)amino)-2- oxoethyl)-1H-indazole-3- carboxamide |
| 32 | 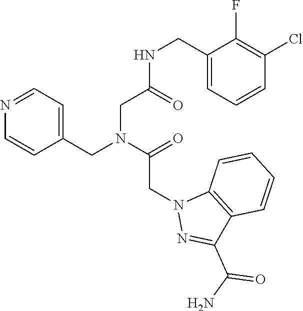 [SMILES: NC(=O)c1nn(CC(=O)N(CC(=O)NCc2cccc(Cl)c2F)Cc2ccncc2)c2ccccc12] | 1-(2-((2-((3-chloro-2- fluorobenzyl)amino)-2- oxoethyl)(pyridin-4- ylmethyl)amino)-2- oxoethyl)-1H-indazole-3- carboxamide |
| 33 | 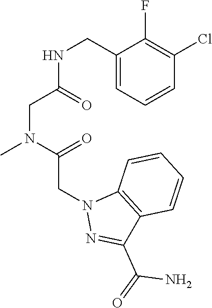 [SMILES: CN(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(methyl)amino)-2- oxoethyl)-1H-indazole-3- carboxamide |
| 34 | 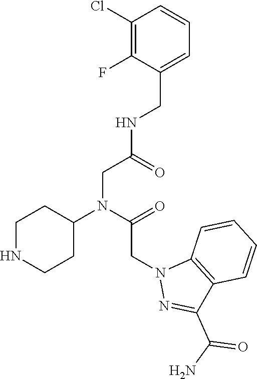 [SMILES: NC(=O)c1nn(CC(=O)N(CC(=O)NCc2cccc(Cl)c2F)C2CCNCC2)c2ccccc12] | 1-(2-((2-((3-chloro-2- fluorobenzyl)amino)-2- oxoethyl)(piperidin-4- yl)amino)-2-oxoethyl)-1H- indazole-3-carboxamide |
| 35 | 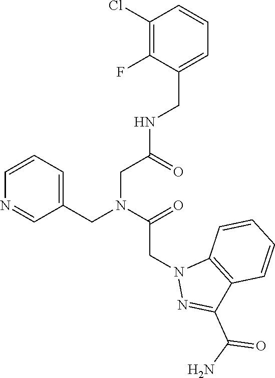 [SMILES: NC(=O)c1nn(CC(=O)N(CC(=O)NCc2cccc(Cl)c2F)Cc2cccnc2)c2ccccc12] | 1-(2-((2-((3-chloro-2- fluorobenzyl)amino)-2- oxoethyl)(pyridin-3- ylmethyl)amino)-2- oxoethyl)-1H-indazole-3- carboxamide |
| 36 | 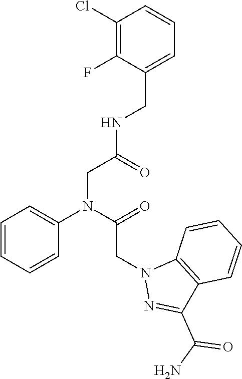 [SMILES: NC(=O)c1nn(CC(=O)N(CC(=O)NCc2cccc(Cl)c2F)c2ccccc2)c2ccccc12] | 1-(2-((2-((3-chloro-2- fluorobenzyl)amino)-2- oxoethyl)(phenyl)amino)-2- oxoethyl)-1H-indazole-3- carboxamide |
| 37 | 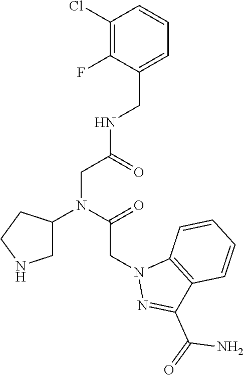 [SMILES: NC(=O)c1nn(CC(=O)N(CC(=O)NCc2cccc(Cl)c2F)C2CCNC2)c2ccccc12] | 1-(2-((2-((3-chloro-2- fluorobenzyl)amino)-2- oxoethyl)(pyrrolidin-3- yl)amino)-2-oxoethyl)-1H- indazole-3-carboxamide |
| 38 | 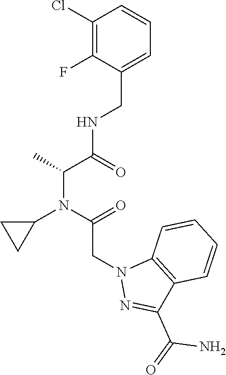 [SMILES: C[C@H](C(=O)NCc1cccc(Cl)c1F)N(C(=O)Cn1nc(C(N)=O)c2ccccc21)C1CC1] | (R)-1-(2-((1-((3-chloro-2- fluorobenzyl)amino)-1- oxopropan-2- yl)(cyclopropyl)amino)-2- oxoethyl)-1H-indazole-3- carboxamide |
| 39 | 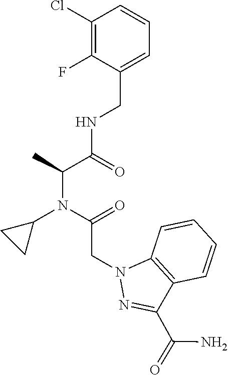 [SMILES: C[C@@H](C(=O)NCc1cccc(Cl)c1F)N(C(=O)Cn1nc(C(N)=O)c2ccccc21)C1CC1] | (S)-1-(2-((1-((3-chloro-2- fluorobenzyl)amino)-1- oxopropan-2- yl)(cyclopropyl)amino)-2- oxoethyl)-1H-indazole-3- carboxamide |
| 40 | 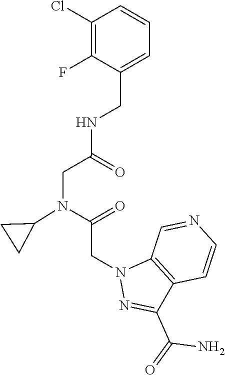 [SMILES: NC(=O)c1nn(CC(=O)N(CC(=O)NCc2cccc(Cl)c2F)C2CC2)c2cnccc12] | 1-(2-((2-((3-chloro-2- fluorobenzyl)amino)-2- oxoethyl)(cyclopropyl)amino)- 2-oxoethyl)-1H- pyrazolo[3,4-c]pyridine-3- carboxamide |
| 41 | 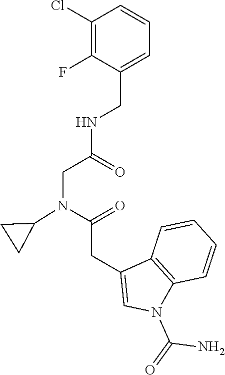 [SMILES: NC(=O)n1cc(CC(=O)N(CC(=O)NCc2cccc(Cl)c2F)C2CC2)c2ccccc21] | 3-(2-((2-((3-chloro-2- fluorobenzyl)amino)-2- oxoethyl)(cyclopropyl)amino)- 2-oxoethyl)-1H-indole-1- carboxamide |
| 42 | 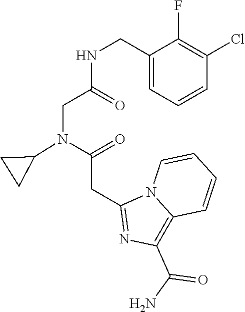 [SMILES: NC(=O)c1nc(CC(=O)N(CC(=O)NCc2cccc(Cl)c2F)C2CC2)n2ccccc12] | 3-(2-((2-((3-chloro-2- fluorobenzyl)amino)-2- oxoethyl)(cyclopropyl)amino)- 2-oxoethyl)imidazo[1,5- a]pyridine-1-carboxamide |
| 43 | 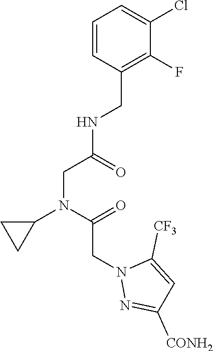 [SMILES: NC(=O)c1cc(C(F)(F)F)n(CC(=O)N(CC(=O)NCc2cccc(Cl)c2F)C2CC2)n1] | 1-(2-((2-((3-chloro-2- fluorobenzyl)amino)-2- oxoethyl)(cyclopropyl)amino)- 2-oxoethyl)-5- (trifluoromethyl)-1H- pyrazole-3-carboxamide |
| 44 | 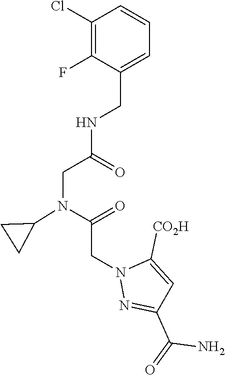 [SMILES: NC(=O)c1cc(C(=O)O)n(CC(=O)N(CC(=O)NCc2cccc(Cl)c2F)C2CC2)n1] | 3-carbamoyl-1-(2-((2-((3- chloro-2- fluorobenzyl)amino)-2- oxoethyl)(cyclopropyl)amino)- 2-oxoethyl)-1H- pyrazole-5-carboxylic acid |
| 45 | 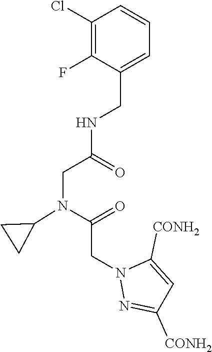 [SMILES: NC(=O)c1cc(C(N)=O)n(CC(=O)N(CC(=O)NCc2cccc(Cl)c2F)C2CC2)n1] | 1-(2-((2-((3-chloro-2- fluorobenzyl)amino)-2- oxoethyl)(cyclopropyl)amino)- 2-oxoethyl)-1H- pyrazole-3,5-dicarboxamide |
| 46 | 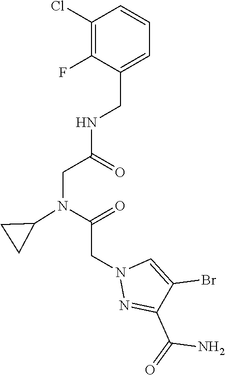 [SMILES: NC(=O)c1nn(CC(=O)N(CC(=O)NCc2cccc(Cl)c2F)C2CC2)cc1Br] | 4-bromo-1-(2-((2-((3- chloro-2- fluorobenzyl)amino)-2- oxoethyl)(cyclopropyl)amino)- 2-oxoethyl)-1H- pyrazole-3-carboxamide |
| 47 | 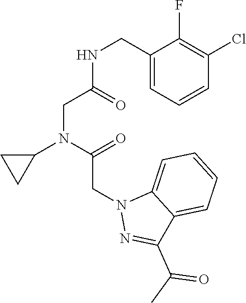 [SMILES: CC(=O)c1nn(CC(=O)N(CC(=O)NCc2cccc(Cl)c2F)C2CC2)c2ccccc12] | 2-(3-acetyl-1H-indazol-1- yl)-N-(2-((3-chloro-2- fluorobenzyl)amino)-2- oxoethyl)-N- cyclopropylacetamide |
| 48 | 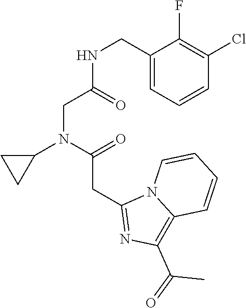 [SMILES: CC(=O)c1nc(CC(=O)N(CC(=O)NCc2cccc(Cl)c2F)C2CC2)n2ccccc12] | 2-(1-acetylimidazo[1,5- a]pyridin-3-yl)-N-(2-((3- chloro-2- fluorobenzyl)amino)-2- oxoethyl)-N- cyclopropylacetamide |
| 49 | 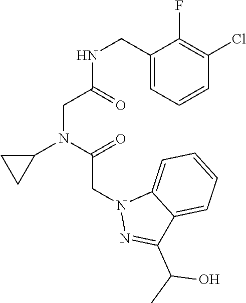 [SMILES: CC(O)c1nn(CC(=O)N(CC(=O)NCc2cccc(Cl)c2F)C2CC2)c2ccccc12] | N-(2-((3-chloro-2- fluorobenzyl)amino)-2- oxoethyl)-N-cyclopropyl-2- (3-(1-hydroxyethyl)-1H- indazol-1-yl)acetamide |
| 50 | 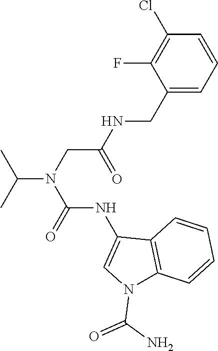 [SMILES: CC(C)N(CC(=O)NCc1cccc(Cl)c1F)C(=O)Nc1cn(C(N)=O)c2ccccc12] | 3-(3-(2-((3-chloro-2- fluorobenzyl)amino)-2- oxoethyl)-3- isopropylureido)-1H-indole- 1-carboxamide |
| 51 | 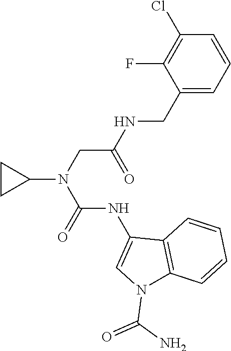 [SMILES: NC(=O)n1cc(NC(=O)N(CC(=O)NCc2cccc(Cl)c2F)C2CC2)c2ccccc21] | 3-(3-(2-((3-chloro-2- fluorobenzyl)amino)-2- oxoethyl)-3- cyclopropylureido)-1H- indole-1-carboxamide |
| 52 | 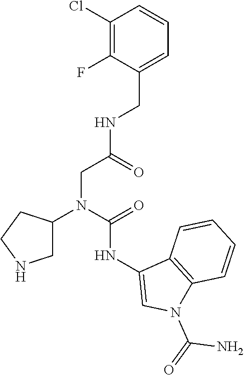 [SMILES: NC(=O)n1cc(NC(=O)N(CC(=O)NCc2cccc(Cl)c2F)C2CCNC2)c2ccccc21] | 3-(3-(2-((3-chloro-2- fluorobenzyl)amino)-2- oxoethyl)-3-(pyrrolidin-3- yl)ureido)-1H-indole-1- carboxamide |
| 53 | 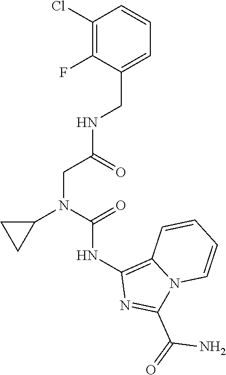 [SMILES: NC(=O)c1nc(NC(=O)N(CC(=O)NCc2cccc(Cl)c2F)C2CC2)c2ccccn12] | 1-(3-(2-((3-chloro-2- fluorobenzyl)amino)-2- oxoethyl)-3- cyclopropylureido)imidazo [1,5-a]pyridine-3- carboxamide |
| 54 | 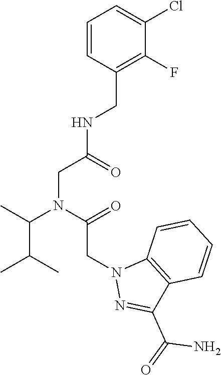 [SMILES: CC(C)C(C)N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(3-methylbutan-2- yl)amino)-2-oxoethyl)-1H- indazole-3-carboxamide |
| 55 | 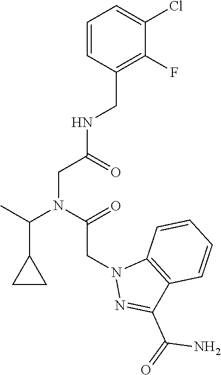 [SMILES: CC(C1CC1)N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(1- cyclopropylethyl)amino)-2- oxoethyl)-1H-indazole-3- carboxamide |
| 56 | 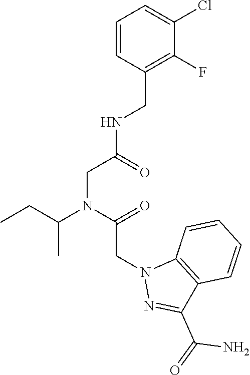 [SMILES: CCC(C)N(CC(=O)NCc1cccc(Cl)c1F)C(=O)Cn1nc(C(N)=O)c2ccccc21] | 1-(2-(sec-butyl(2-((3- chloro-2- fluorobenzyl)amino)-2- oxoethyl)amino)-2- oxoethyl)-1H-indazole-3- carboxamide |
| 57 | 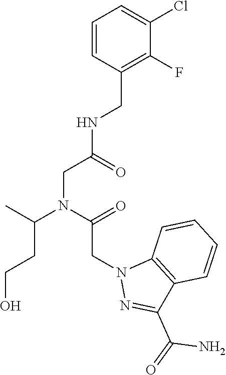 [SMILES: CC(CCO)N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(4-hydroxybutan- 2-yl)amino)-2-oxoethyl)- 1H-indazole-3-carboxamide |
| 58 | 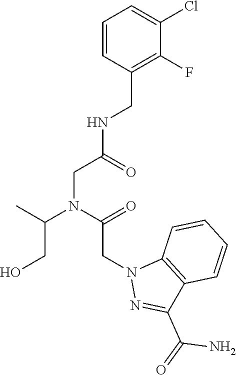 [SMILES: CC(CO)N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(1-hydroxypropan- 2-yl)amino)-2-oxoethyl)- 1H-indazole-3-carboxamide |
| 59 | 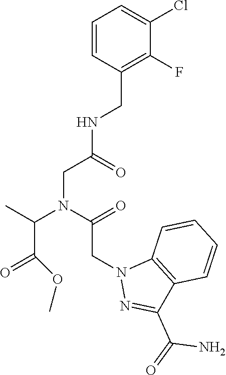 [SMILES: COC(=O)C(C)N(CC(=O)NCc1cccc(Cl)c1F)C(=O)Cn1nc(C(N)=O)c2ccccc21] | methyl 2-(2-(3-carbamoyl- 1H-indazol-1-yl)-N-(2-((3- chloro-2- fluorobenzyl)amino)-2- oxoethyl)acetamido)propan- oate |
| 60 | 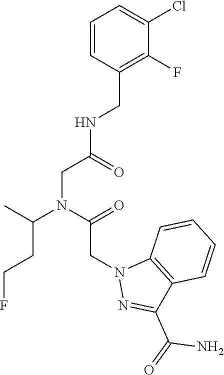 [SMILES: CC(CCF)N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(4-fluorobutan-2- yl)amino)-2-oxoethyl)-1H- indazole-3-carboxamide |
| 61 | 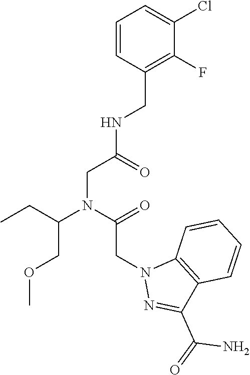 [SMILES: CCC(COC)N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(1-methoxybutan- 2-yl)amino)-2-oxoethyl)- 1H-indazole-3-carboxamide |
| 62 | 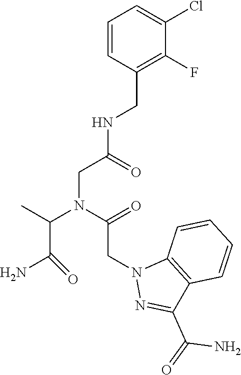 [SMILES: CC(C(N)=O)N(CC(=O)NCc1cccc(Cl)c1F)C(=O)Cn1nc(C(N)=O)c2ccccc21] | 1-(2-((1-amino-1- oxopropan-2-yl)(2-((3- chloro-2- fluorobenzyl)amino)-2- oxoethyl)amino)-2- oxoethyl)-1H-indazole-3- carboxamide |
| 63 | 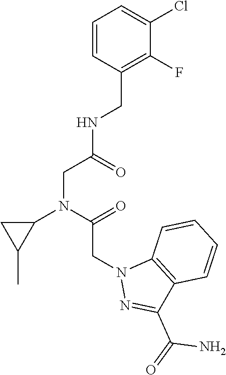 [SMILES: CC1CC1N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(2- methylcyclopropyl)amino)- 2-oxoethyl)-1H-indazole-3- carboxamide |
| 64 | 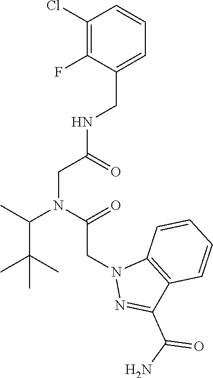 [SMILES: CC(N(CC(=O)NCc1cccc(Cl)c1F)C(=O)Cn1nc(C(N)=O)c2ccccc21)C(C)(C)C] | 1-(2-((2-((3-chloro-2- fluorobenzyl)amino)-2- oxoethyl)(3,3- dimethylbutan-2-yl)amino)- 2-oxoethyl)-1H-indazole-3- carboxamide |
| 65 | 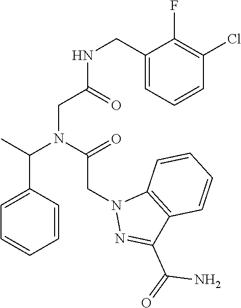 [SMILES: CC(c1ccccc1)N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(1- phenylethyl)amino)-2- oxoethyl)-1H-indazole-3- carboxamide |
| 66 | 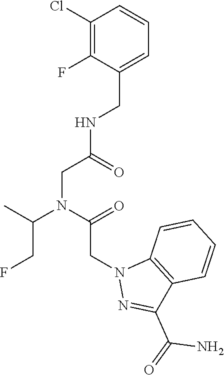 [SMILES: CC(CF)N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(1-fluoropropan-2- yl)amino)-2-oxoethyl)-1H- indazole-3-carboxamide |
| 67 | 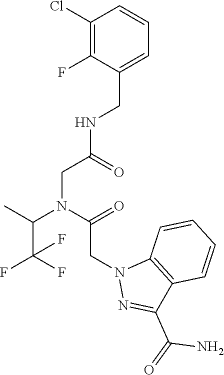 [SMILES: CC(N(CC(=O)NCc1cccc(Cl)c1F)C(=O)Cn1nc(C(N)=O)c2ccccc21)C(F)(F)F] | 1-(2-((2-((3-chloro-2- fluorobenzyl)amino)-2- oxoethyl)(1,1,1- trifluoropropan-2- yl)amino)-2-oxoethyl)-1H- indazole-3-carboxamide |
| 68 | 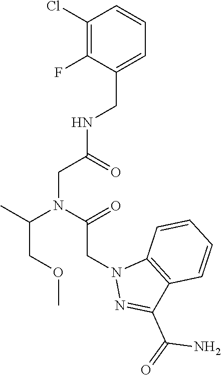 [SMILES: COCC(C)N(CC(=O)NCc1cccc(Cl)c1F)C(=O)Cn1nc(C(N)=O)c2ccccc21] | 1-(2-((2-((3-chloro-2- fluorobenzyl)amino)-2- oxoethyl)(1- methoxypropan-2- yl)amino)-2-oxoethyl)-1H- indazole-3-carboxamide |
| 69 | 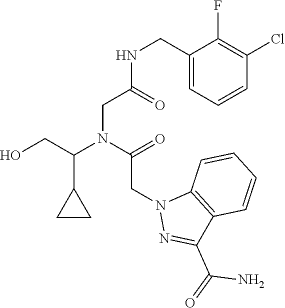 [SMILES: NC(=O)c1nn(CC(=O)N(CC(=O)NCc2cccc(Cl)c2F)C(CO)C2CC2)c2ccccc12] | 1-(2-((2-((3-chloro-2- fluorobenzyl)amino)-2- oxoethyl)(1-cyclopropyl-2- hydroxyethyl)amino)-2- oxoethyl)-1H-indazole-3- carboxamide |
| 70 | 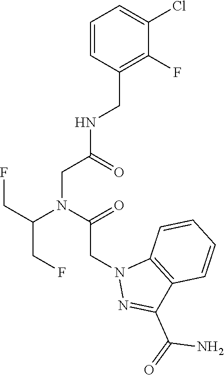 [SMILES: NC(=O)c1nn(CC(=O)N(CC(=O)NCc2cccc(Cl)c2F)C(CF)CF)c2ccccc12] | 1-(2-((2-((2-chloro-3- fluorobenzyl)amino)-2- oxoethyl)(1,3- difluoropropan-2-yl)amino)- 2-oxoethyl)-1H-indazole-3- carboxamide |
| 71 | 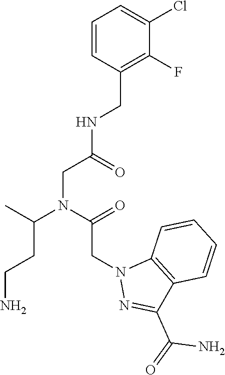 [SMILES: CC(CCN)N(CC(=O)NCc1cccc(Cl)c1F)C(=O)Cn1nc(C(N)=O)c2ccccc21] | 1-(2-((4-aminobutan-2- yl)(2-((2-chloro-3- fluorobenzyl)amino)-2- oxoethyl)amino)-2- oxoethyl)-1H-indazole-3- carboxamide |
| 72 | 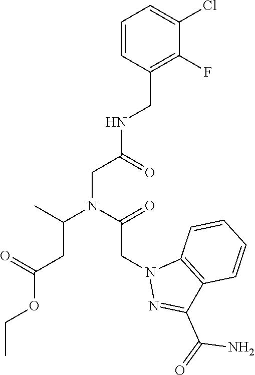 [SMILES: CCOC(=O)CC(C)N(CC(=O)NCc1cccc(Cl)c1F)C(=O)Cn1nc(C(N)=O)c2ccccc21] | ethyl 3-(2-(3-carbamoyl- 1H-indazol-1-yl)-N-(2-((3- chloro-2- fluorobenzyl)amino)-2- oxoethyl)acetamido)butanoate |
| 73 | 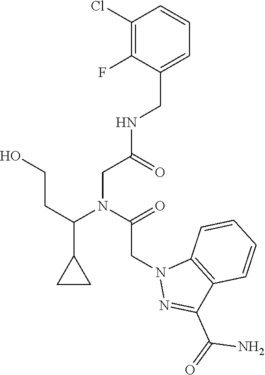 [SMILES: NC(=O)c1nn(CC(=O)N(CC(=O)NCc2cccc(Cl)c2F)C(CCO)C2CC2)c2ccccc12] | 1-(2-((2-((3-chloro-2- fluorobenzyl)amino)-2- oxoethyl)(1-cyclopropyl-3- hydroxypropyl)amino)-2- oxoethyl)-1H-indazole-3- carboxamide |
| 74 | 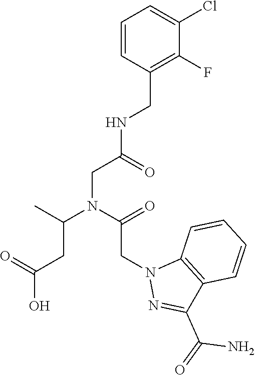 [SMILES: CC(CC(=O)O)N(CC(=O)NCc1cccc(Cl)c1F)C(=O)Cn1nc(C(N)=O)c2ccccc21] | 3-(2-(3-carbamoyl-1H- indazol-1-yl)-N-(2-((3- chloro-2- fluorobenzyl)amino)-2- oxoethyl)acetamido)butanoic acid |
| 75 | 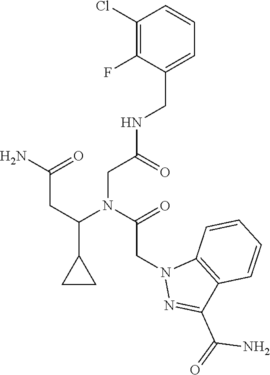 [SMILES: NC(=O)CC(C1CC1)N(CC(=O)NCc1cccc(Cl)c1F)C(=O)Cn1nc(C(N)=O)c2ccccc21] | 1-(2-((3-amino-1- cyclopropyl-3- oxopropyl)(2-((3-chloro-2- fluorobenzyl)amino)-2- oxoethyl)amino)-2- oxoethyl)-1H-indazole-3- carboxamide |
| 76 | 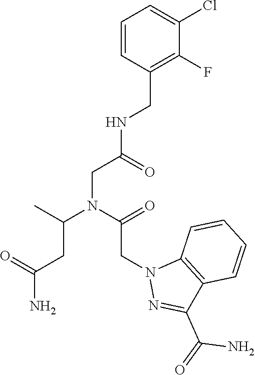 [SMILES: CC(CC(N)=O)N(CC(=O)NCc1cccc(Cl)c1F)C(=O)Cn1nc(C(N)=O)c2ccccc21] | 1-(2-((4-amino-4-oxobutan- 2-yl)(2-((3-chloro-2- fluorobenzyl)amino)-2- oxoethyl)amino)-2- oxoethyl)-1H-indazole-3- carboxamide |
| 77 | 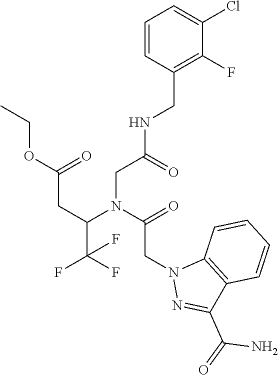 [SMILES: CCOC(=O)CC(N(CC(=O)NCc1cccc(Cl)c1F)C(=O)Cn1nc(C(N)=O)c2ccccc21)C(F)(F)F] | ethyl 3-(2-(3-carbamoyl- 1H-indazol-1-yl)-N-(2-((3- chloro-2- fluorobenzyl)amino)-2- oxoethyl)acetamido)-4,4,4- trifluorobutanoate |
| 78 | 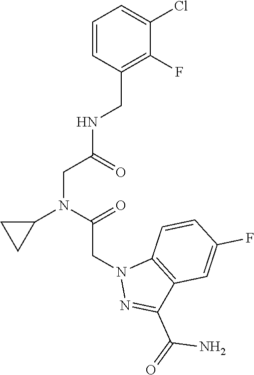 [SMILES: NC(=O)c1nn(CC(=O)N(CC(=O)NCc2cccc(Cl)c2F)C2CC2)c2ccc(F)cc12] | 1-(2-((2-((3-chloro-2- fluorobenzyl)amino)-2- oxoethyl)(cyclopropyl)amino)- 2-oxoethyl)-5-fluoro-1H- indazole-3-carboxamide |
| 79 | 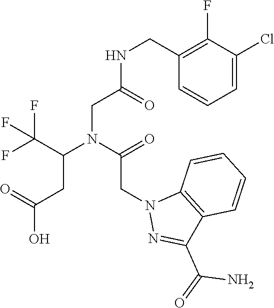 [SMILES: NC(=O)c1nn(CC(=O)N(CC(=O)NCc2cccc(Cl)c2F)C(CC(=O)O)C(F)(F)F)c2ccccc12] | 3-(2-(3-carbamoyl-1H- indazol-1-yl)-N-(2-((3- chloro-2- fluorobenzyl)amino)-2- oxoethyl)acetamido)-4,4,4- trifluorobutanoic acid |
| 80 | 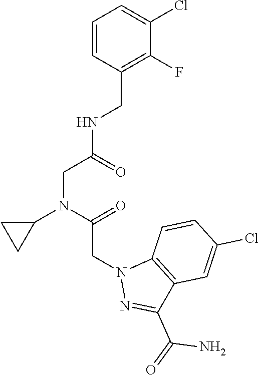 [SMILES: NC(=O)c1nn(CC(=O)N(CC(=O)NCc2cccc(Cl)c2F)C2CC2)c2ccc(Cl)cc12] | 5-chloro-1-(2-((2-((3- chloro-2- fluorobenzyl)amino)-2- oxoethyl)(cyclopropyl)amino)- 2-oxoethyl)-1H-indazole- 3-carboxamide |
| 81 | 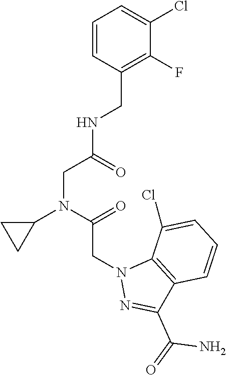 [SMILES: NC(=O)c1nn(CC(=O)N(CC(=O)NCc2cccc(Cl)c2F)C2CC2)c2c(Cl)cccc12] | 7-chloro-1-(2-((2-((3- chloro-2- fluorobenzyl)amino)-2- oxoethyl)(cyclopropyl)amino)- 2-oxoethyl)-1H-indazole- 3-carboxamide |
| 82 | 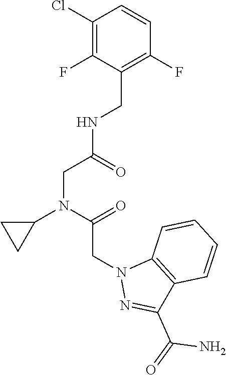 [SMILES: NC(=O)c1nn(CC(=O)N(CC(=O)NCc2c(F)ccc(Cl)c2F)C2CC2)c2ccccc12] | 1-(2-((2-((3-chloro-2,6- difluorobenzyl)amino)-2- oxoethyl)(cyclopropyl)amino)- 2-oxoethyl)-1H-indazole- 3-carboxamide |
| 83 | 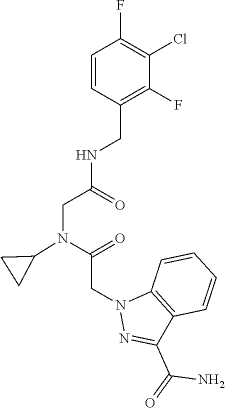 [SMILES: NC(=O)c1nn(CC(=O)N(CC(=O)NCc2ccc(F)c(Cl)c2F)C2CC2)c2ccccc12] | 1-(2-((2-((3-chloro-2,4- difluorobenzyl)amino)-2- oxoethyl)(cyclopropyl)amino)- 2-oxoethyl)-1H-indazole- 3-carboxamide |
| 84 | 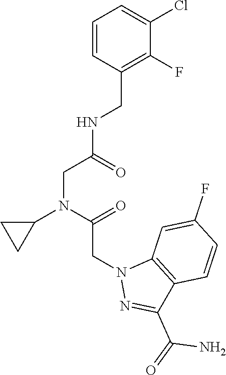 [SMILES: NC(=O)c1nn(CC(=O)N(CC(=O)NCc2cccc(Cl)c2F)C2CC2)c2cc(F)ccc12] | 1-(2-((2-((3-chloro-2- fluorobenzyl)amino)-2- oxoethyl)(cyclopropyl)amino)- 2-oxoethyl)-6-fluoro-1H- indazole-3-carboxamide |
| 85 | 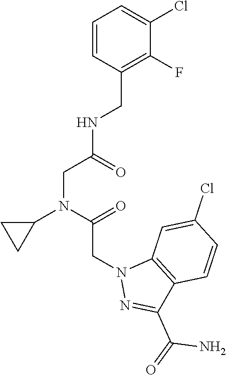 [SMILES: NC(=O)c1nn(CC(=O)N(CC(=O)NCc2cccc(Cl)c2F)C2CC2)c2cc(Cl)ccc12] | 6-chloro-1-(2-((2-((3- chloro-2- fluorobenzyl)amino)-2- oxoethyl)(cyclopropyl)amino)- 2-oxoethyl)-1H-indazole- 3-carboxamide |
| 86 | 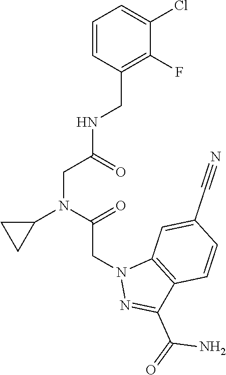 [SMILES: N#Cc1ccc2c(C(N)=O)nn(CC(=O)N(CC(=O)NCc3cccc(Cl)c3F)C3CC3)c2c1] | 1-(2-((2-((3-chloro-2- fluorobenzyl)amino)-2- oxoethyl)(cyclopropyl)amino)- 2-oxoethyl)-6-cyano-1H- indazole-3-carboxamide |
| 87 | 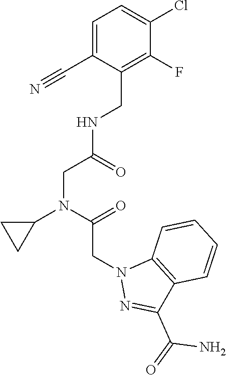 [SMILES: N#Cc1ccc(Cl)c(F)c1CNC(=O)CN(C(=O)Cn1nc(C(N)=O)c2ccccc21)C1CC1] | 1-(2-((2-((3-chloro-6-cyano- 2-fluorobenzyl)amino)-2- oxoethyl)(cyclopropyl)amino)- 2-oxoethyl)-1H-indazole- 3-carboxamide |
| 88 | 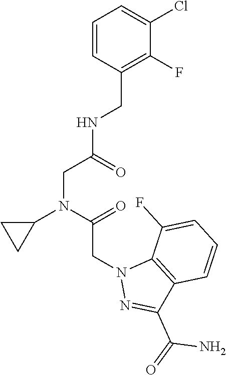 [SMILES: NC(=O)c1nn(CC(=O)N(CC(=O)NCc2cccc(Cl)c2F)C2CC2)c2c(F)cccc12] | 1-(2-((2-((3-chloro-2- fluorobenzyl)amino)-2- oxoethyl)(cyclopropyl)amino)- 2-oxoethyl)-7-fluoro-1H- indazole-3-carboxamide |
| 89 | 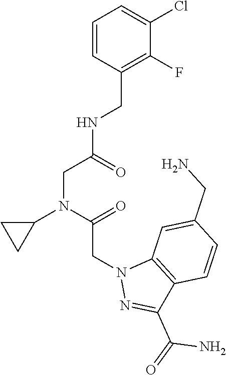 [SMILES: NCc1ccc2c(C(N)=O)nn(CC(=O)N(CC(=O)NCc3cccc(Cl)c3F)C3CC3)c2c1] | 6-(aminomethyl)-1-(2-((2- ((3-chloro-2- fluorobenzyl)amino)-2- oxoethyl)(cyclopropyl)amino)- 2-oxoethyl)-1H-indazole- 3-carboxamide |
| 90 | 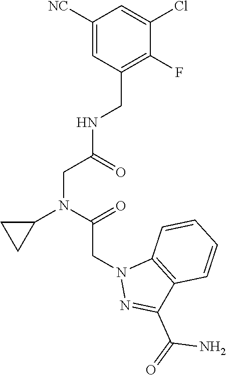 [SMILES: N#Cc1cc(Cl)c(F)c(CNC(=O)CN(C(=O)Cn2nc(C(N)=O)c3ccccc32)C2CC2)c1] | 1-(2-((2-((3-chloro-5-cyano- 2-fluorobenzyl)amino)-2- oxoethyl)(cyclopropyl)amino)- 2-oxoethyl)-1H-indazole- 3-carboxamide |
| 91 | 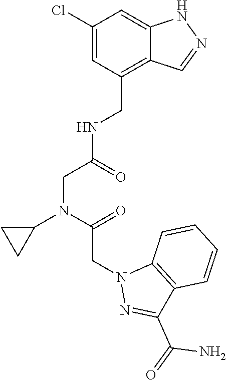 [SMILES: NC(=O)c1nn(CC(=O)N(CC(=O)NCc2cc(Cl)cc3[nH]ncc23)C2CC2)c2ccccc12] | 1-(2-((2-(((6-chloro-1H- indazol-4- yl)methyl)amino)-2- oxoethyl)(cyclopropyl)amino)- 2-oxoethyl)-1H-indazole- 3-carboxamide |
| 92 | 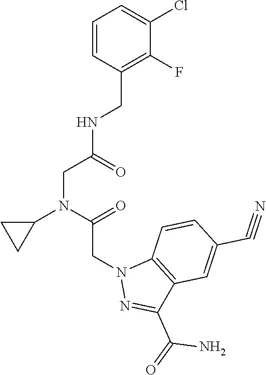 [SMILES: N#Cc1ccc2c(c1)c(C(N)=O)nn2CC(=O)N(CC(=O)NCc1cccc(Cl)c1F)C1CC1] | 1-(2-((2-((3-chloro-2- fluorobenzyl)amino)-2- oxoethyl)(cyclopropyl)amino)- 2-oxoethyl)-5-cyano-1H- indazole-3-carboxamide |
| 93 | 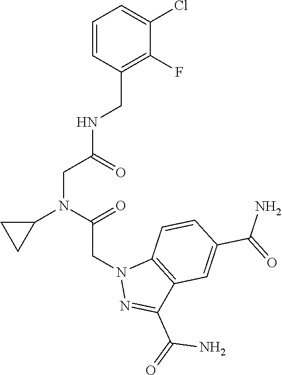 [SMILES: NC(=O)c1ccc2c(c1)c(C(N)=O)nn2CC(=O)N(CC(=O)NCc1cccc(Cl)c1F)C1CC1] | 1-(2-((2-((3-chloro-2- fluorobenzyl)amino)-2- oxoethyl)(cyclopropyl)amino)- 2-oxoethyl)-1H-indazole- 3,5-dicarboxamide |
| 94 | 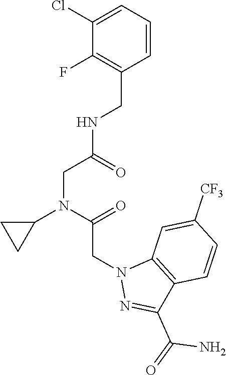 [SMILES: NC(=O)c1nn(CC(=O)N(CC(=O)NCc2cccc(Cl)c2F)C2CC2)c2cc(C(F)(F)F)ccc12] | 1-(2-((2-((3-chloro-2- fluorobenzyl)amino)-2- oxoethyl)(cyclopropyl)amino)- 2-oxoethyl)-6- (trifluoromethyl)-1H- indazole-3-carboxamide |
| 95 | 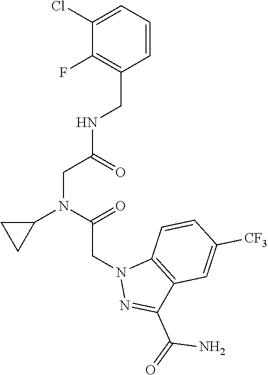 [SMILES: NC(=O)c1nn(CC(=O)N(CC(=O)NCc2cccc(Cl)c2F)C2CC2)c2ccc(C(F)(F)F)cc12] | 1-(2-((2-((3-chloro-2- fluorobenzyl)amino)-2- oxoethyl)(cyclopropyl)amino)- 2-oxoethyl)-5- (trifluoromethyl)-1H- indazole-3-carboxamide |
| 96 | 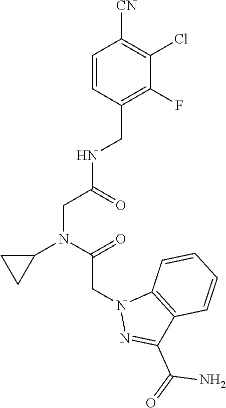 [SMILES: N#Cc1ccc(CNC(=O)CN(C(=O)Cn2nc(C(N)=O)c3ccccc32)C2CC2)c(F)c1Cl] | 1-(2-((2-((3-chloro-4-cyano- 2-fluorobenzyl)amino)-2- oxoethyl)(cyclopropyl)amino)- 2-oxoethyl)-1H-indazole- 3-carboxamide |
| 97 | 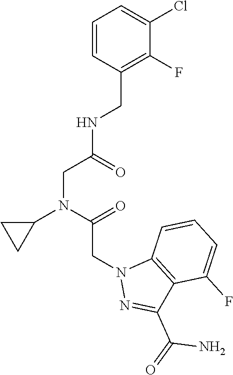 [SMILES: NC(=O)c1nn(CC(=O)N(CC(=O)NCc2cccc(Cl)c2F)C2CC2)c2cccc(F)c12] | 1-(2-((2-((3-chloro-2- fluorobenzyl)amino)-2- oxoethyl)(cyclopropyl)amino)- 2-oxoethyl)-4-fluoro-1H- indazole-3-carboxamide |
| 98 | 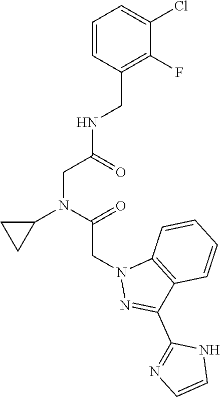 [SMILES: O=C(CN(C(=O)Cn1nc(-c2ncc[nH]2)c2ccccc21)C1CC1)NCc1cccc(Cl)c1F] | 2-(3-(1H-imidazol-2-yl)- 1H-indazol-1-yl)-N-(2-((3- chloro-2- fluorobenzyl)amino)-2- oxoethyl)-N- cyclopropylacetamide |
| 99 | 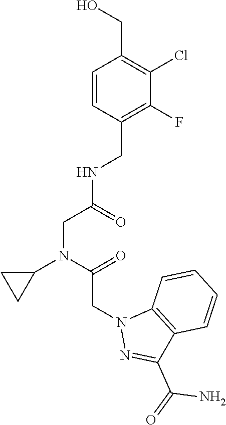 [SMILES: NC(=O)c1nn(CC(=O)N(CC(=O)NCc2ccc(CO)c(Cl)c2F)C2CC2)c2ccccc12] | 1-(2-((2-((3-chloro-2- fluoro-4- (hydroxymethyl)benzyl) amino)-2- oxoethyl)(cyclopropyl)amino)- 2-oxoethyl)-1H-indazole- 3-carboxamide |
| 100 | 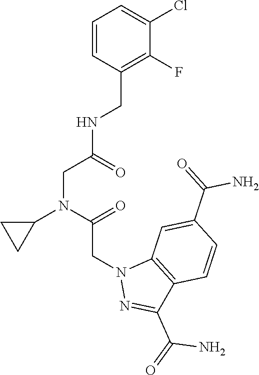 [SMILES: NC(=O)c1ccc2c(C(N)=O)nn(CC(=O)N(CC(=O)NCc3cccc(Cl)c3F)C3CC3)c2c1] | 1-(2-((2-((3-chloro-2- fluorobenzyl)amino)-2- oxoethyl)(cyclopropyl)amino)- 2-oxoethyl)-1H-indazole- 3,6-dicarboxamide |
| 101 | 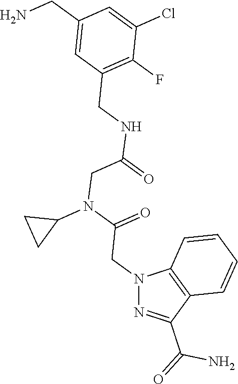 [SMILES: NCc1cc(Cl)c(F)c(CNC(=O)CN(C(=O)Cn2nc(C(N)=O)c3ccccc32)C2CC2)c1] | 1-(2-((2-((5-(aminomethyl)- 3-chloro-2- fluorobenzyl)amino)-2- oxoethyl)(cyclopropyl)amino)- 2-oxoethyl)-1H-indazole- 3-carboxamide |
| 102 | 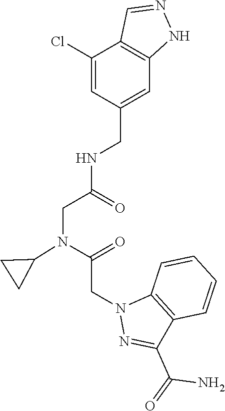 [SMILES: NC(=O)c1nn(CC(=O)N(CC(=O)NCc2cc(Cl)c3cn[nH]c3c2)C2CC2)c2ccccc12] | 1-(2-((2-(((4-chloro-1H- indazol-6- yl)methyl)amino)-2- oxoethyl)(cyclopropyl)amino)- 2-oxoethyl)-1H-indazole- 3-carboxamide |
| 103 | 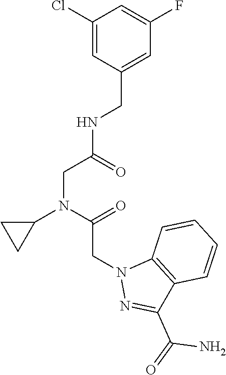 [SMILES: NC(=O)c1nn(CC(=O)N(CC(=O)NCc2cc(F)cc(Cl)c2)C2CC2)c2ccccc12] | 1-(2-((2-((3-chloro-5- fluorobenzyl)amino)-2- oxoethyl)(cyclopropyl)amino)- 2-oxoethyl)-1H-indazole- 3-carboxamide |
| 104 | 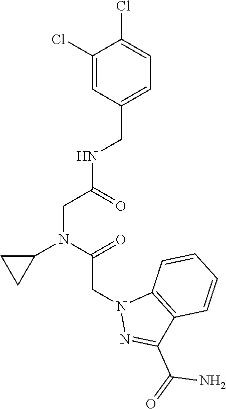 [SMILES: NC(=O)c1nn(CC(=O)N(CC(=O)NCc2ccc(Cl)c(Cl)c2)C2CC2)c2ccccc12] | 1-(2-(cyclopropyl(2-((3,4- dichlorobenzyl)amino)-2- oxoethyl)amino)-2- oxoethyl)-1H-indazole-3- carboxamide |
| 105 | 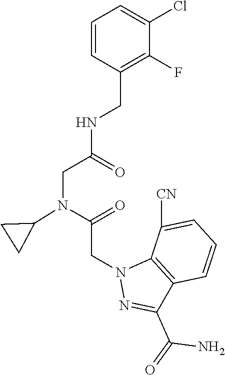 [SMILES: N#Cc1cccc2c(C(N)=O)nn(CC(=O)N(CC(=O)NCc3cccc(Cl)c3F)C3CC3)c12] | 1-(2-((2-((3-chloro-2- fluorobenzyl)amino)-2- oxoethyl)(cyclopropyl)amino)- 2-oxoethyl)-7-cyano-1H- indazole-3-carboxamide |
| 106 | 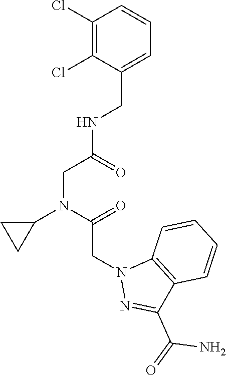 [SMILES: NC(=O)c1nn(CC(=O)N(CC(=O)NCc2cccc(Cl)c2Cl)C2CC2)c2ccccc12] | 1-(2-(cyclopropyl(2-((2,3- dichlorobenzyl)amino)-2- oxoethyl)amino)-2- oxoethyl)-1H-indazole-3- carboxamide |
| 107 | 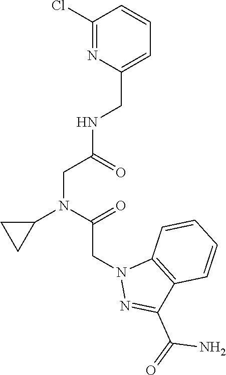 [SMILES: NC(=O)c1nn(CC(=O)N(CC(=O)NCc2cccc(Cl)n2)C2CC2)c2ccccc12] | 1-(2-((2-(((6-chloropyridin- 2-yl)methyl)amino)-2- oxoethyl)(cyclopropyl)amino)- 2-oxoethyl)-1H-indazole- 3-carboxamide |
| 108 | 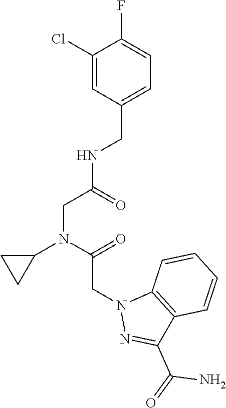 [SMILES: NC(=O)c1nn(CC(=O)N(CC(=O)NCc2ccc(F)c(Cl)c2)C2CC2)c2ccccc12] | 1-(2-((2-((3-chloro-4- fluorobenzyl)amino)-2- oxoethyl)(cyclopropyl)amino)- 2-oxoethyl)-1H-indazole- 3-carboxamide |
| 109 | 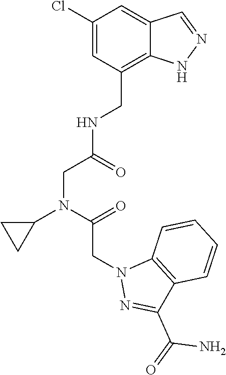 [SMILES: NC(=O)c1nn(CC(=O)N(CC(=O)NCc2cc(Cl)cc3cn[nH]c23)C2CC2)c2ccccc12] | 1-(2-((2-(((5-chloro-1H- indazol-7- yl)methyl)amino)-2- oxoethyl)(cyclopropyl)amino)- 2-oxoethyl)-1H-indazole- 3-carboxamide |
| 110 | 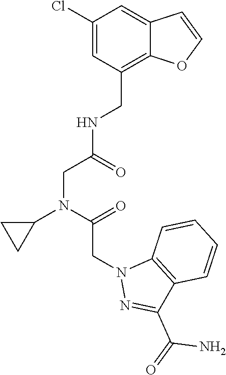 [SMILES: NC(=O)c1nn(CC(=O)N(CC(=O)NCc2cc(Cl)cc3ccoc23)C2CC2)c2ccccc12] | 1-(2-((2-(((5- chlorobenzofuran-7- yl)methyl)amino)-2- oxoethyl)(cyclopropyl)amino)- 2-oxoethyl)-1H-indazole- 3-carboxamide |
| 111 | 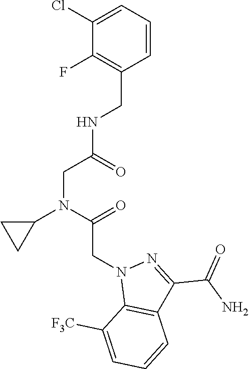 [SMILES: NC(=O)c1nn(CC(=O)N(CC(=O)NCc2cccc(Cl)c2F)C2CC2)c2c(C(F)(F)F)cccc12] | 1-(2-((2-((3-chloro-2- fluorobenzyl)amino)-2- oxoethyl)(cyclopropyl)amino)- 2-oxoethyl)-7- (trifluoromethyl)-1H- indazole-3-carboxamide |
| 112 | 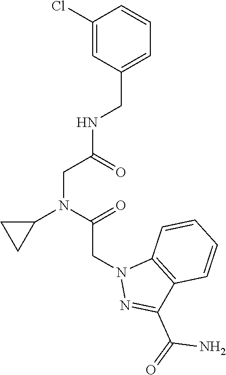 [SMILES: NC(=O)c1nn(CC(=O)N(CC(=O)NCc2cccc(Cl)c2)C2CC2)c2ccccc12] | 1-(2-((2-((3- chlorobenzyl)amino)-2- oxoethyl)(cyclopropyl)amino)- 2-oxoethyl)-1H-indazole- 3-carboxamide |
| 113 | 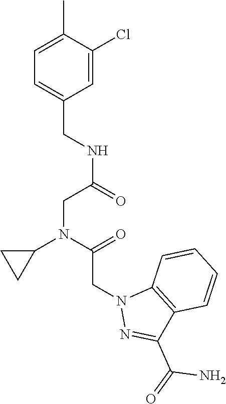 [SMILES: Cc1ccc(CNC(=O)CN(C(=O)Cn2nc(C(N)=O)c3ccccc32)C2CC2)cc1Cl] | 1-(2-((2-((3-chloro-4- methylbenzyl)amino)-2- oxoethyl)(cyclopropyl)amino)- 2-oxoethyl)-1H-indazole- 3-carboxamide |
| 114 | 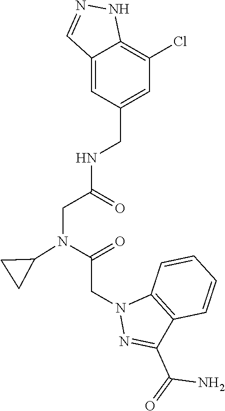 [SMILES: NC(=O)c1nn(CC(=O)N(CC(=O)NCc2cc(Cl)c3[nH]ncc3c2)C2CC2)c2ccccc12] | 1-(2-((2-(((7-chloro-1H- indazol-5- yl)methyl)amino)-2- oxoethyl)(cyclopropyl)amino)- 2-oxoethyl)-1H-indazole- 3-carboxamide |
| 115 | 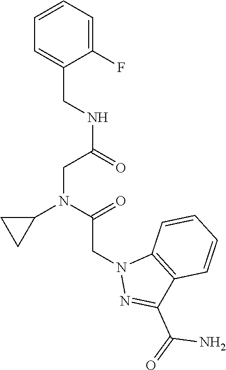 [SMILES: NC(=O)c1nn(CC(=O)N(CC(=O)NCc2ccccc2F)C2CC2)c2ccccc12] | 1-(2-(cyclopropyl(2-((2- fluorobenzyl)amino)-2- oxoethyl)amino)-2- oxoethyl)-1H-indazole-3- carboxamide |
| 116 | 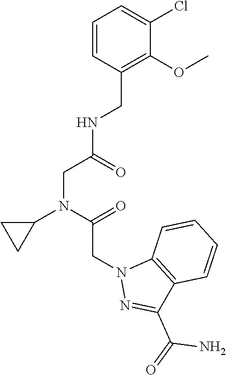 [SMILES: COc1c(Cl)cccc1CNC(=O)CN(C(=O)Cn1nc(C(N)=O)c2ccccc21)C1CC1] | 1-(2-((2-((3-chloro-2- methoxybenzyl)amino)-2- oxoethyl)(cyclopropyl)amino)- 2-oxoethyl)-1H-indazole- 3-carboxamide |
| 117 | 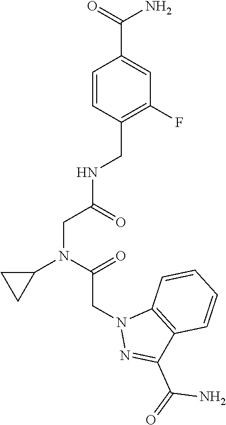 [SMILES: NC(=O)c1ccc(CNC(=O)CN(C(=O)Cn2nc(C(N)=O)c3ccccc32)C2CC2)c(F)c1] | 1-(2-((2-((4-carbamoyl-2- fluorobenzyl)amino)-2- oxoethyl)(cyclopropyl)amino)- 2-oxoethyl)-1H-indazole- 3-carboxamide |
| 118 | 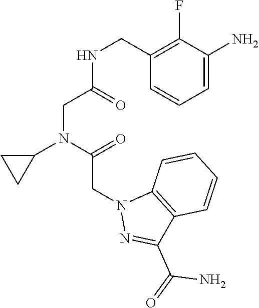 [SMILES: NC(=O)c1nn(CC(=O)N(CC(=O)NCc2cccc(N)c2F)C2CC2)c2ccccc12] | 1-(2-((2-((3-amino-2- fluorobenzyl)amino)-2- oxoethyl)(cyclopropyl)amino)- 2-oxoethyl)-1H-indazole- 3-carboxamide |
| 119 | 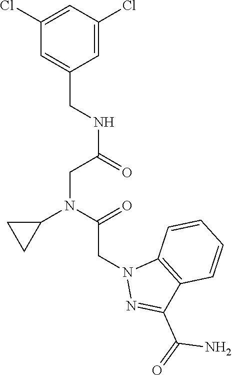 [SMILES: NC(=O)c1nn(CC(=O)N(CC(=O)NCc2cc(Cl)cc(Cl)c2)C2CC2)c2ccccc12] | 1-(2-(cyclopropyl(2-((3,5- dichlorobenzyl)amino)-2- oxoethyl)amino)-2- oxoethyl)-1H-indazole-3- carboxamide |
| 120 | 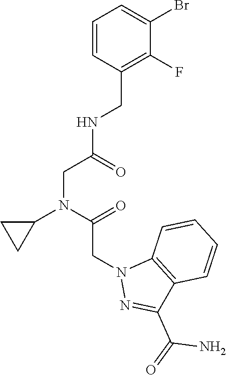 [SMILES: NC(=O)c1nn(CC(=O)N(CC(=O)NCc2cccc(Br)c2F)C2CC2)c2ccccc12] | 1-(2-((2-((3-bromo-2- fluorobenzyl)amino)-2- oxoethyl)(cyclopropyl)amino)- 2-oxoethyl)-1H-indazole- 3-carboxamide |
| 121 | 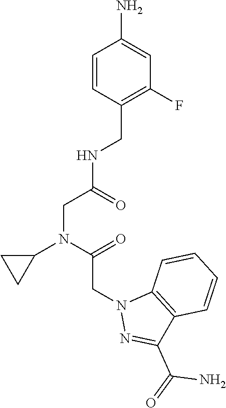 [SMILES: NC(=O)c1nn(CC(=O)N(CC(=O)NCc2ccc(N)cc2F)C2CC2)c2ccccc12] | 1-(2-((2-((4-amino-2- fluorobenzyl)amino)-2- oxoethyl)(cyclopropyl)amino)- 2-oxoethyl)-1H-indazole- 3-carboxamide |
| 122 | 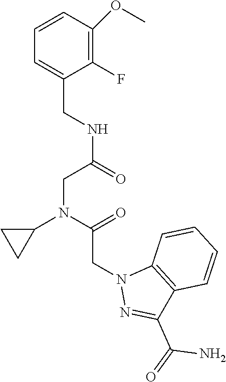 [SMILES: COc1cccc(CNC(=O)CN(C(=O)Cn2nc(C(N)=O)c3ccccc32)C2CC2)c1F] | 1-(2-(cyclopropyl(2-((2- fluoro-3- methoxybenzyl)amino)-2- oxoethyl)amino)-2- oxoethyl)-1H-indazole-3- carboxamide |
| 123 | 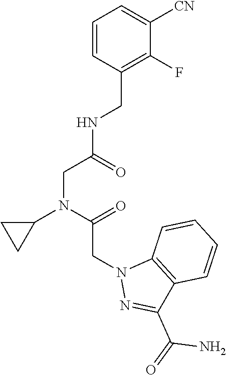 [SMILES: N#Cc1cccc(CNC(=O)CN(C(=O)Cn2nc(C(N)=O)c3ccccc32)C2CC2)c1F] | 1-(2-((2-((3-cyano-2- fluorobenzyl)amino)-2- oxoethyl)(cyclopropyl)amino)- 2-oxoethyl)-1H-indazole- 3-carboxamide |
| 124 | 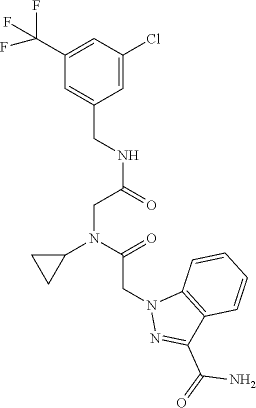 [SMILES: NC(=O)c1nn(CC(=O)N(CC(=O)NCc2cc(Cl)cc(C(F)(F)F)c2)C2CC2)c2ccccc12] | 1-(2-((2-((3-chloro-5- (trifluoromethyl)benzyl) amino)-2- oxoethyl)(cyclopropyl)amino)- 2-oxoethyl)-1H-indazole- 3-carboxamide |
| 125 | 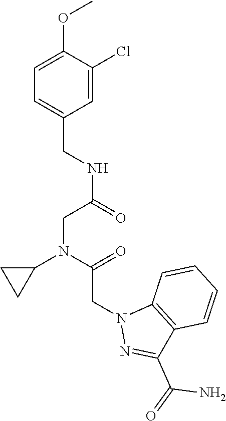 [SMILES: COc1ccc(CNC(=O)CN(C(=O)Cn2nc(C(N)=O)c3ccccc32)C2CC2)cc1Cl] | 1-(2-((2-((3-chloro-4- methoxybenzyl)amino)-2- oxoethyl)(cyclopropyl)amino)- 2-oxoethyl)-1H-indazole- 3-carboxamide |
| 126 | 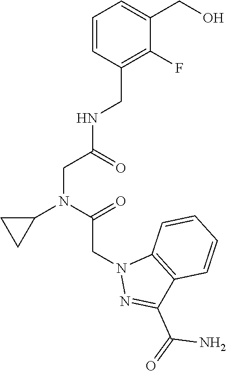 [SMILES: NC(=O)c1nn(CC(=O)N(CC(=O)NCc2cccc(CO)c2F)C2CC2)c2ccccc12] | 1-(2-(cyclopropyl(2-((2- fluoro-3- (hydroxymethyl)benzyl) amino)-2-oxoethyl)amino)-2- oxoethyl)-1H-indazole-3- carboxamide |
| 127 | 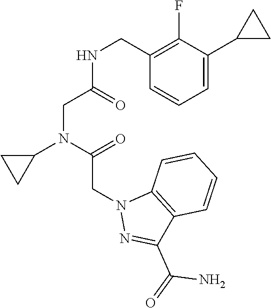 [SMILES: NC(=O)c1nn(CC(=O)N(CC(=O)NCc2cccc(C3CC3)c2F)C2CC2)c2ccccc12] | 1-(2-(cyclopropyl(2-((3- cyclopropyl-2- fluorobenzyl)amino)-2- oxoethyl)amino)-2- oxoethyl)-1H-indazole-3- carboxamide |
| 128 | 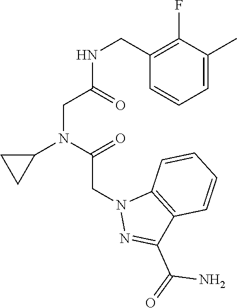 [SMILES: Cc1cccc(CNC(=O)CN(C(=O)Cn2nc(C(N)=O)c3ccccc32)C2CC2)c1F] | 1-(2-(cyclopropyl(2-((2- fluoro-3- methylbenzyl)amino)-2- oxoethyl)amino)-2- oxoethyl)-1H-indazole-3- carboxamide |
| 129 | 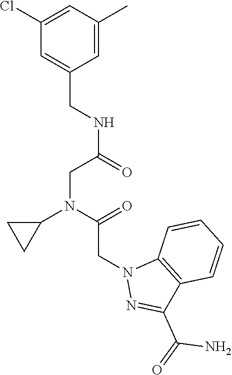 [SMILES: Cc1cc(Cl)cc(CNC(=O)CN(C(=O)Cn2nc(C(N)=O)c3ccccc32)C2CC2)c1] | 1-(2-((2-((3-chloro-5- methylbenzyl)amino)-2- oxoethyl)(cyclopropyl)amino)- 2-oxoethyl)-1H-indazole- 3-carboxamide |
| 130 | 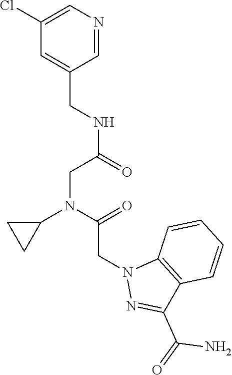 [SMILES: NC(=O)c1nn(CC(=O)N(CC(=O)NCc2cncc(Cl)c2)C2CC2)c2ccccc12] | 1-(2-((2-(((5-chloropyridin- 3-yl)methyl)amino)-2- oxoethyl)(cyclopropyl)amino)- 2-oxoethyl)-1H-indazole- 3-carboxamide |
| 131 | 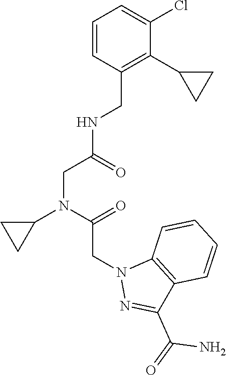 [SMILES: NC(=O)c1nn(CC(=O)N(CC(=O)NCc2cccc(Cl)c2C2CC2)C2CC2)c2ccccc12] | 1-(2-((2-((3-chloro-2- cyclopropylbenzyl)amino)- 2- oxoethyl)(cyclopropyl)amino)- 2-oxoethyl)-1H-indazole- 3-carboxamide |
| 132 | 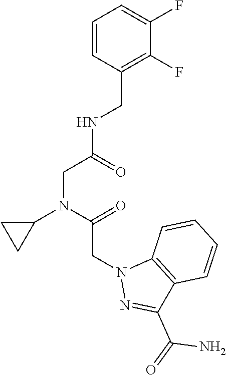 [SMILES: NC(=O)c1nn(CC(=O)N(CC(=O)NCc2cccc(F)c2F)C2CC2)c2ccccc12] | 1-(2-(cyclopropyl(2-((2,3- difluorobenzyl)amino)-2- oxoethyl)amino)-2- oxoethyl)-1H-indazole-3- carboxamide |
| 133 | 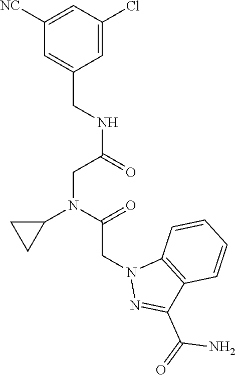 [SMILES: N#Cc1cc(Cl)cc(CNC(=O)CN(C(=O)Cn2nc(C(N)=O)c3ccccc32)C2CC2)c1] | 1-(2-((2-((3-chloro-5- cyanobenzyl)amino)-2- oxoethyl)(cyclopropyl)amino)- 2-oxoethyl)-1H-indazole- 3-carboxamide |
| 134 | 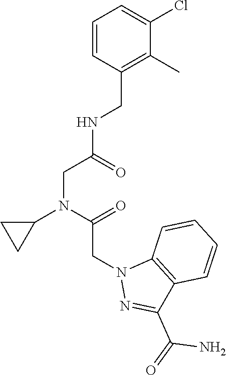 [SMILES: Cc1c(Cl)cccc1CNC(=O)CN(C(=O)Cn1nc(C(N)=O)c2ccccc21)C1CC1] | 1-(2-((2-((3-chloro-2- methylbenzyl)amino)-2- oxoethyl)(cyclopropyl)amino)- 2-oxoethyl)-1H-indazole- 3-carboxamide |
| 135 | 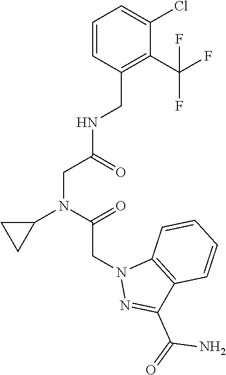 [SMILES: NC(=O)c1nn(CC(=O)N(CC(=O)NCc2cccc(Cl)c2C(F)(F)F)C2CC2)c2ccccc12] | 1-(2-((2-((3-chloro-2- (trifluoromethyl)benzyl) amino)-2- oxoethyl)(cyclopropyl)amino)- 2-oxoethyl)-1H-indazole- 3-carboxamide |
| 136 | 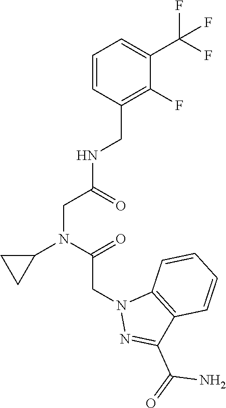 [SMILES: NC(=O)c1nn(CC(=O)N(CC(=O)NCc2cccc(C(F)(F)F)c2F)C2CC2)c2ccccc12] | 1-(2-(cyclopropyl(2-((2- fluoro-3- (trifluoromethyl)benzyl) amino)-2-oxoethyl)amino)-2- oxoethyl)-1H-indazole-3- carboxamide |
| 137 | 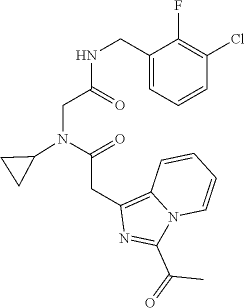 [SMILES: CC(=O)c1nc(CC(=O)N(CC(=O)NCc2cccc(Cl)c2F)C2CC2)c2ccccn12] | 2-(3-acetylimidazo[1,5- a]pyridin-1-yl)-N-(2-((3- chloro-2- fluorobenzyl)amino)-2- oxoethyl)-N- cyclopropylacetamide |
| 138 | 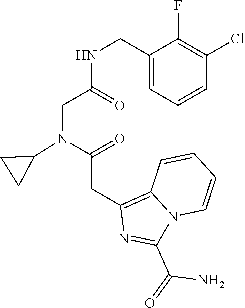 [SMILES: NC(=O)c1nc(CC(=O)N(CC(=O)NCc2cccc(Cl)c2F)C2CC2)c2ccccn12] | 1-(2-((2-((3-chloro-2- fluorobenzyl)amino)-2- oxoethyl)(cyclopropyl)amino)- 2-oxoethyl)imidazo[1,5- a]pyridine-3-carboxamide |
| 139 | 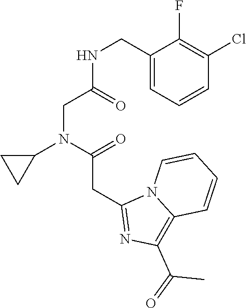 [SMILES: CC(=O)c1nc(CC(=O)N(CC(=O)NCc2cccc(Cl)c2F)C2CC2)n2ccccc12] | 2-(1-acetylimidazo[1,5- a]pyridin-3-yl)-N-(2-((3- chloro-2- fluorobenzyl)amino)-2- oxoethyl)-N- cyclopropylacetamide |
| 140 | 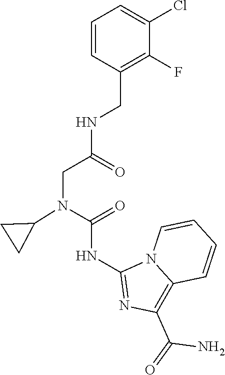 [SMILES: NC(=O)c1nc(NC(=O)N(CC(=O)NCc2cccc(Cl)c2F)C2CC2)n2ccccc12] | 3-(3-(2-((3-chloro-2- fluorobenzyl)amino)-2- oxoethyl)-3- cyclopropylureido)imidazo [1,5-a]pyridine-1- carboxamide |
| 141 | 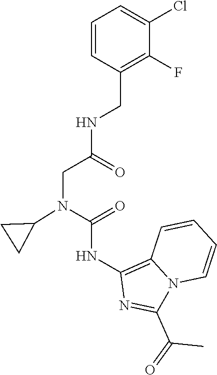 [SMILES: CC(=O)c1nc(NC(=O)N(CC(=O)NCc2cccc(Cl)c2F)C2CC2)c2ccccn12] | 2-(3-(3-acetylimidazo[1,5- a]pyridin-1-yl)-1- cyclopropylureido)-N-(3- chloro-2- fluorobenzyl)acetamide |

In some embodiments, the complement factor D inhibitory compound described by Formula (I) or Formula (Ia) has a structure provided in Table 2.

TABLE 2

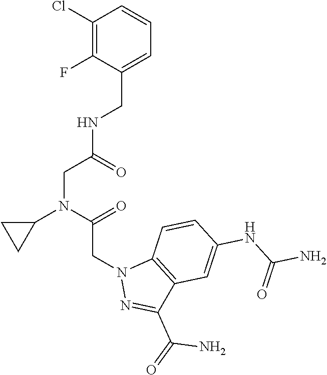 [SMILES: NC(=O)Nc1ccc2c(c1)c(C(N)=O)nn2CC(=O)N(CC(=O)NCc1cccc(Cl)c1F)C1CC1]

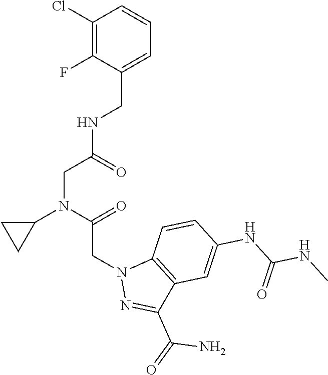 [SMILES: CNC(=O)Nc1ccc2c(c1)c(C(N)=O)nn2CC(=O)N(CC(=O)NCc1cccc(Cl)c1F)C1CC1]

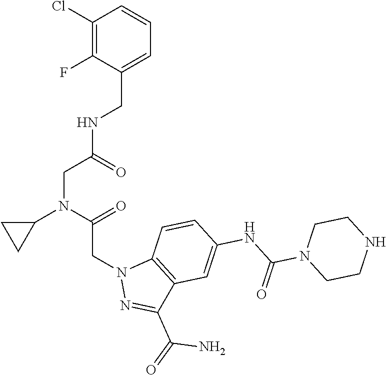 [SMILES: NC(=O)c1nn(CC(=O)N(CC(=O)NCc2cccc(Cl)c2F)C2CC2)c2ccc(NC(=O)N3CCNCC3)cc12]

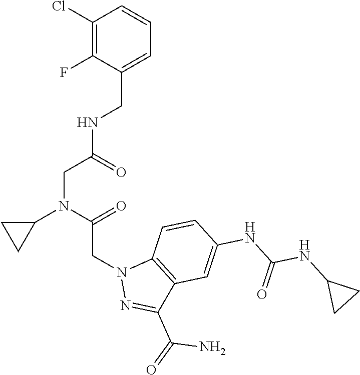 [SMILES: NC(=O)c1nn(CC(=O)N(CC(=O)NCc2cccc(Cl)c2F)C2CC2)c2ccc(NC(=O)NC3CC3)cc12]

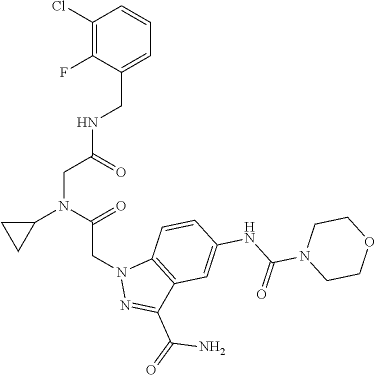 [SMILES: NC(=O)c1nn(CC(=O)N(CC(=O)NCc2cccc(Cl)c2F)C2CC2)c2ccc(NC(=O)N3CCOCC3)cc12]

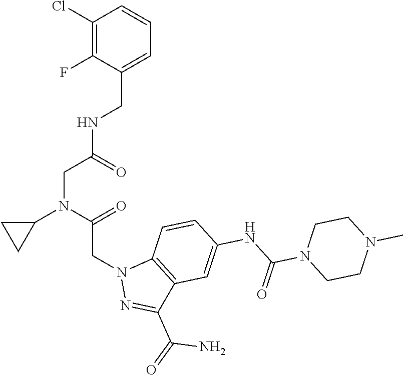 [SMILES: CN1CCN(C(=O)Nc2ccc3c(c2)c(C(N)=O)nn3CC(=O)N(CC(=O)NCc2cccc(Cl)c2F)C2CC2)CC1]

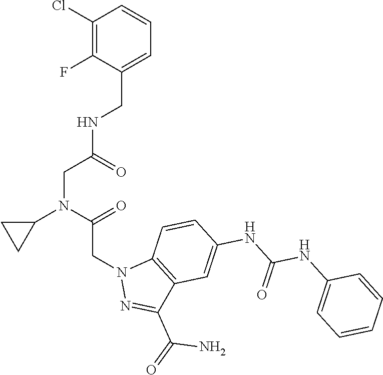 [SMILES: NC(=O)c1nn(CC(=O)N(CC(=O)NCc2cccc(Cl)c2F)C2CC2)c2ccc(NC(=O)Nc3ccccc3)cc12]

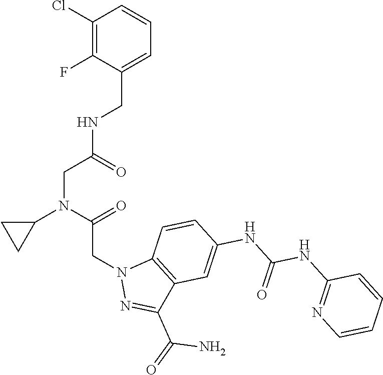 [SMILES: NC(=O)c1nn(CC(=O)N(CC(=O)NCc2cccc(Cl)c2F)C2CC2)c2ccc(NC(=O)Nc3ccccn3)cc12]

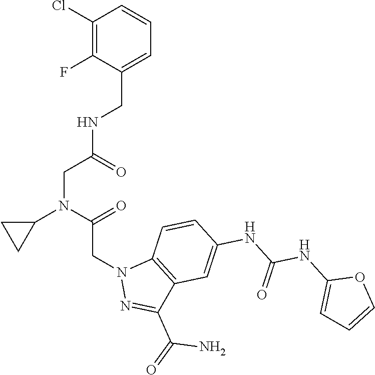 [SMILES: NC(=O)c1nn(CC(=O)N(CC(=O)NCc2cccc(Cl)c2F)C2CC2)c2ccc(NC(=O)Nc3ccco3)cc12]

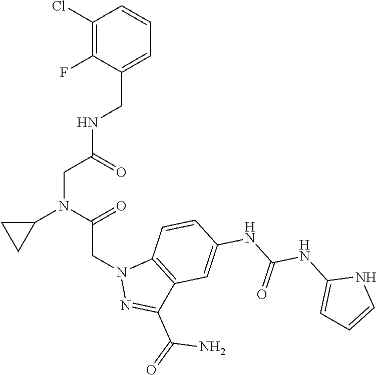 [SMILES: NC(=O)c1nn(CC(=O)N(CC(=O)NCc2cccc(Cl)c2F)C2CC2)c2ccc(NC(=O)Nc3ccc[nH]3)cc12]

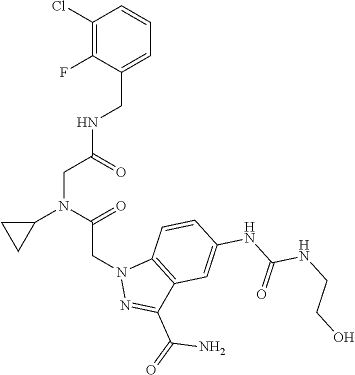 [SMILES: NC(=O)c1nn(CC(=O)N(CC(=O)NCc2cccc(Cl)c2F)C2CC2)c2ccc(NC(=O)NCCO)cc12]

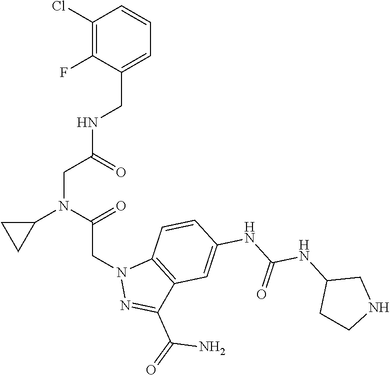 [SMILES: NC(=O)c1nn(CC(=O)N(CC(=O)NCc2cccc(Cl)c2F)C2CC2)c2ccc(NC(=O)NC3CCNC3)cc12]

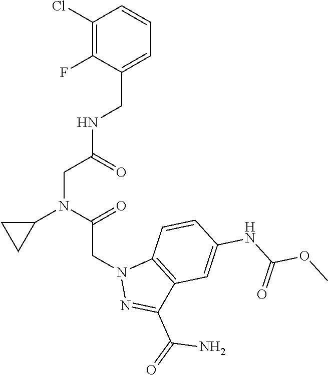 [SMILES: COC(=O)Nc1ccc2c(c1)c(C(N)=O)nn2CC(=O)N(CC(=O)NCc1cccc(Cl)c1F)C1CC1]

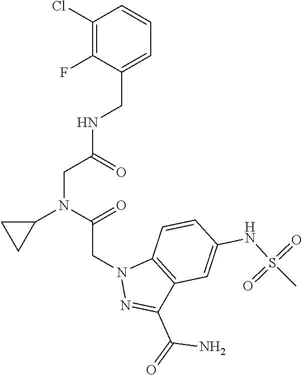 [SMILES: CS(=O)(=O)Nc1ccc2c(c1)c(C(N)=O)nn2CC(=O)N(CC(=O)NCc1cccc(Cl)c1F)C1CC1]

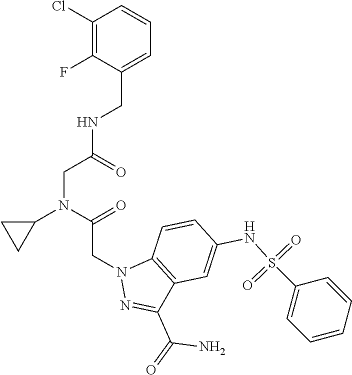 [SMILES: NC(=O)c1nn(CC(=O)N(CC(=O)NCc2cccc(Cl)c2F)C2CC2)c2ccc(NS(=O)(=O)c3ccccc3)cc12]

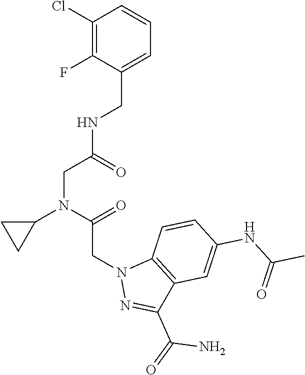 [SMILES: CC(=O)Nc1ccc2c(c1)c(C(N)=O)nn2CC(=O)N(CC(=O)NCc1cccc(Cl)c1F)C1CC1]

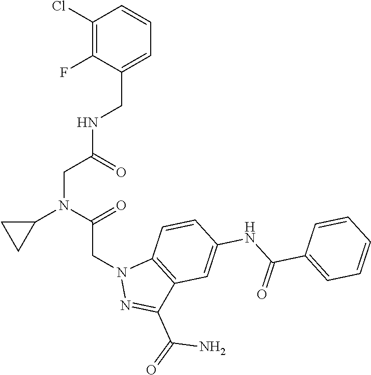 [SMILES: NC(=O)c1nn(CC(=O)N(CC(=O)NCc2cccc(Cl)c2F)C2CC2)c2ccc(NC(=O)c3ccccc3)cc12]

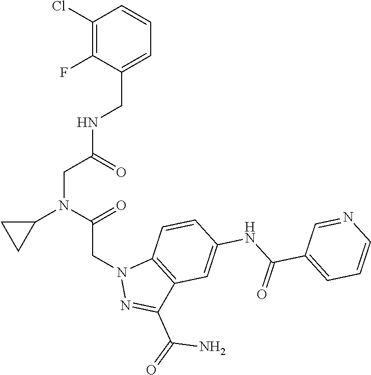 [SMILES: NC(=O)c1nn(CC(=O)N(CC(=O)NCc2cccc(Cl)c2F)C2CC2)c2ccc(NC(=O)c3cccnc3)cc12]

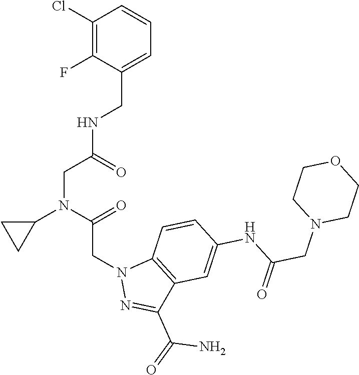 [SMILES: NC(=O)c1nn(CC(=O)N(CC(=O)NCc2cccc(Cl)c2F)C2CC2)c2ccc(NC(=O)CN3CCOCC3)cc12]

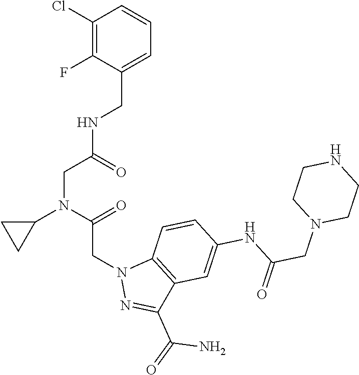 [SMILES: NC(=O)c1nn(CC(=O)N(CC(=O)NCc2cccc(Cl)c2F)C2CC2)c2ccc(NC(=O)CN3CCNCC3)cc12]

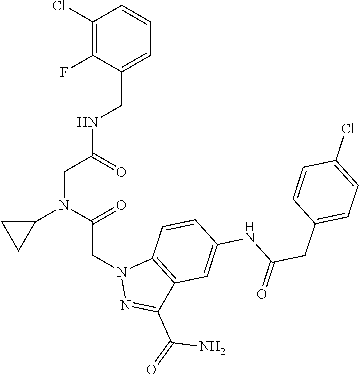 [SMILES: NC(=O)c1nn(CC(=O)N(CC(=O)NCc2cccc(Cl)c2F)C2CC2)c2ccc(NC(=O)Cc3ccc(Cl)cc3)cc12]

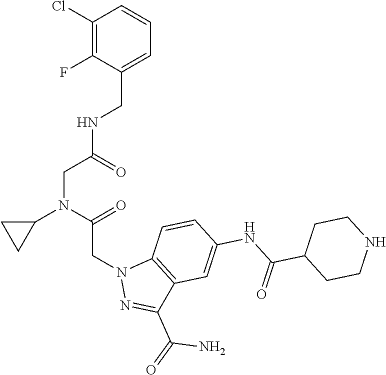 [SMILES: NC(=O)c1nn(CC(=O)N(CC(=O)NCc2cccc(Cl)c2F)C2CC2)c2ccc(NC(=O)C3CCNCC3)cc12]

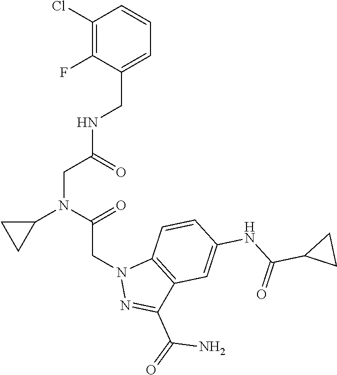 [SMILES: NC(=O)c1nn(CC(=O)N(CC(=O)NCc2cccc(Cl)c2F)C2CC2)c2ccc(NC(=O)C3CC3)cc12]

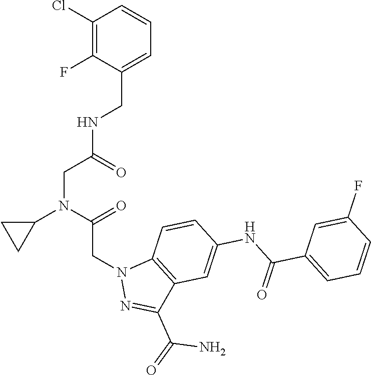 [SMILES: NC(=O)c1nn(CC(=O)N(CC(=O)NCc2cccc(Cl)c2F)C2CC2)c2ccc(NC(=O)c3cccc(F)c3)cc12]

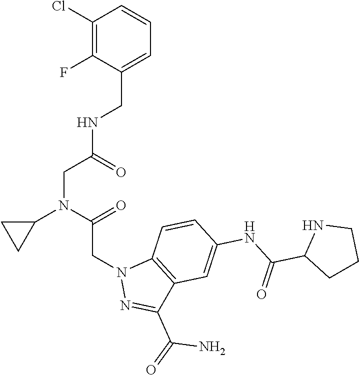 [SMILES: NC(=O)c1nn(CC(=O)N(CC(=O)NCc2cccc(Cl)c2F)C2CC2)c2ccc(NC(=O)C3CCCN3)cc12]

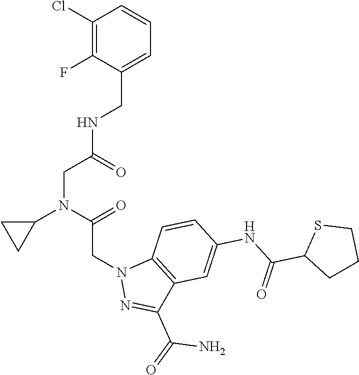 [SMILES: NC(=O)c1nn(CC(=O)N(CC(=O)NCc2cccc(Cl)c2F)C2CC2)c2ccc(NC(=O)C3CCCS3)cc12]

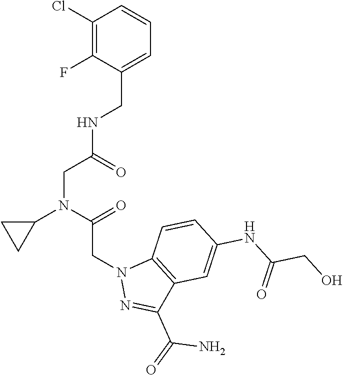 [SMILES: NC(=O)c1nn(CC(=O)N(CC(=O)NCc2cccc(Cl)c2F)C2CC2)c2ccc(NC(=O)CO)cc12]

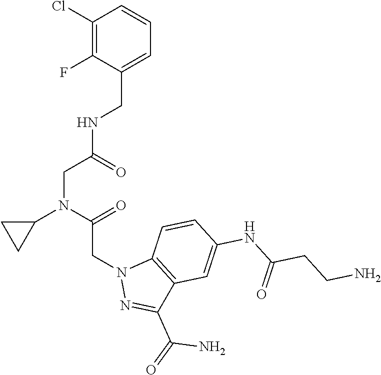 [SMILES: NCCC(=O)Nc1ccc2c(c1)c(C(N)=O)nn2CC(=O)N(CC(=O)NCc1cccc(Cl)c1F)C1CC1]

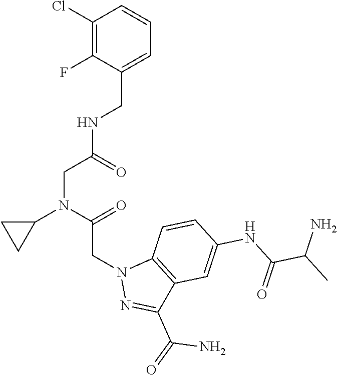 [SMILES: CC(N)C(=O)Nc1ccc2c(c1)c(C(N)=O)nn2CC(=O)N(CC(=O)NCc1cccc(Cl)c1F)C1CC1]

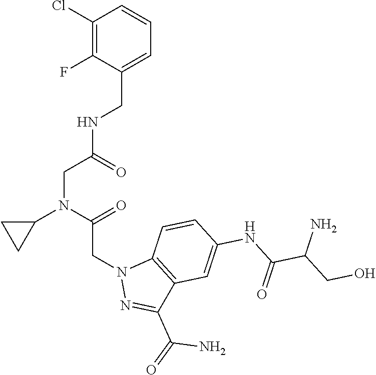 [SMILES: NC(=O)c1nn(CC(=O)N(CC(=O)NCc2cccc(Cl)c2F)C2CC2)c2ccc(NC(=O)C(N)CO)cc12]

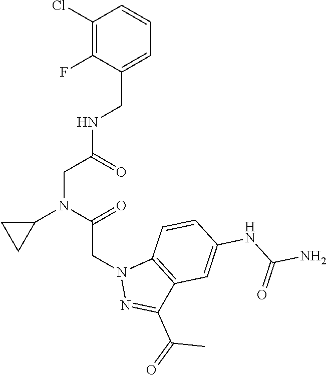 [SMILES: CC(=O)c1nn(CC(=O)N(CC(=O)NCc2cccc(Cl)c2F)C2CC2)c2ccc(NC(N)=O)cc12]

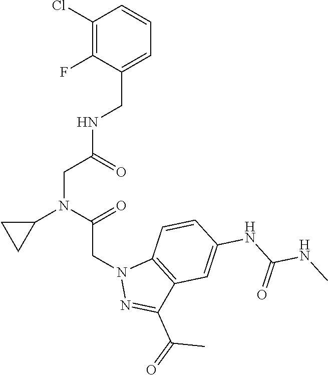 [SMILES: CNC(=O)Nc1ccc2c(c1)c(C(C)=O)nn2CC(=O)N(CC(=O)NCc1cccc(Cl)c1F)C1CC1]

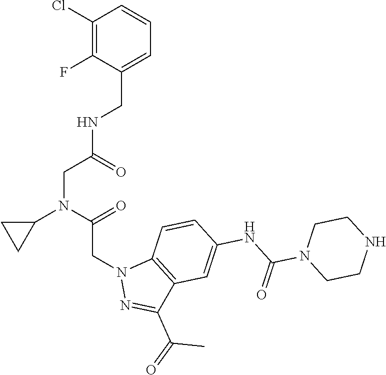 [SMILES: CC(=O)c1nn(CC(=O)N(CC(=O)NCc2cccc(Cl)c2F)C2CC2)c2ccc(NC(=O)N3CCNCC3)cc12]

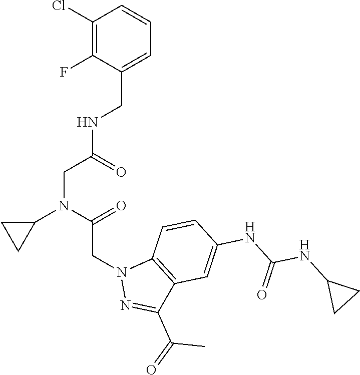 [SMILES: CC(=O)c1nn(CC(=O)N(CC(=O)NCc2cccc(Cl)c2F)C2CC2)c2ccc(NC(=O)NC3CC3)cc12]

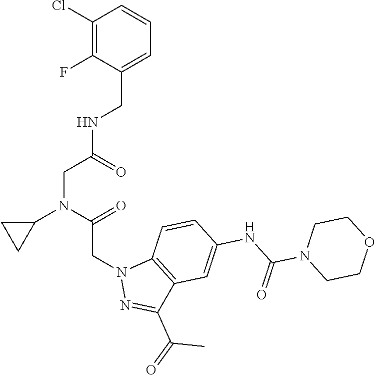 [SMILES: CC(=O)c1nn(CC(=O)N(CC(=O)NCc2cccc(Cl)c2F)C2CC2)c2ccc(NC(=O)N3CCOCC3)cc12]

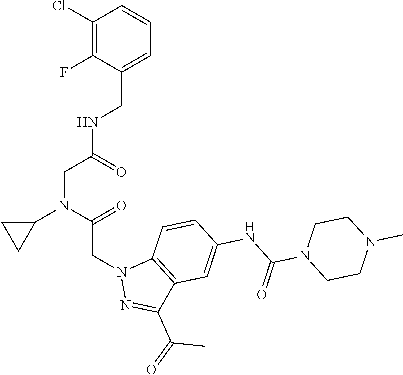 [SMILES: CC(=O)c1nn(CC(=O)N(CC(=O)NCc2cccc(Cl)c2F)C2CC2)c2ccc(NC(=O)N3CCN(C)CC3)cc12]

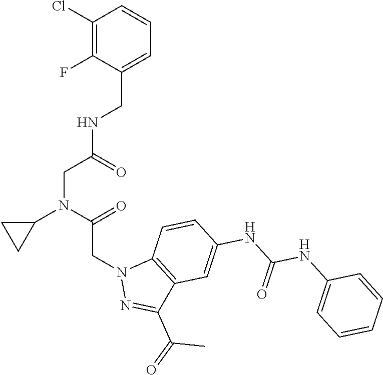 [SMILES: CC(=O)c1nn(CC(=O)N(CC(=O)NCc2cccc(Cl)c2F)C2CC2)c2ccc(NC(=O)Nc3ccccc3)cc12]

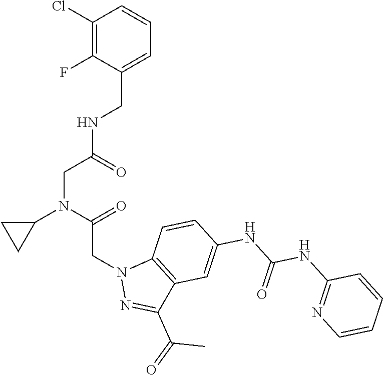 [SMILES: CC(=O)c1nn(CC(=O)N(CC(=O)NCc2cccc(Cl)c2F)C2CC2)c2ccc(NC(=O)Nc3ccccn3)cc12]

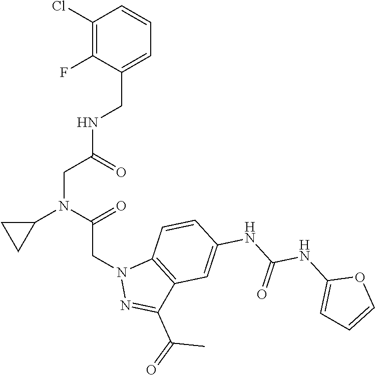 [SMILES: CC(=O)c1nn(CC(=O)N(CC(=O)NCc2cccc(Cl)c2F)C2CC2)c2ccc(NC(=O)Nc3ccco3)cc12]

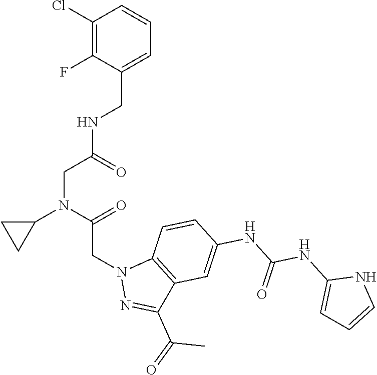 [SMILES: CC(=O)c1nn(CC(=O)N(CC(=O)NCc2cccc(Cl)c2F)C2CC2)c2ccc(NC(=O)Nc3ccc[nH]3)cc12]

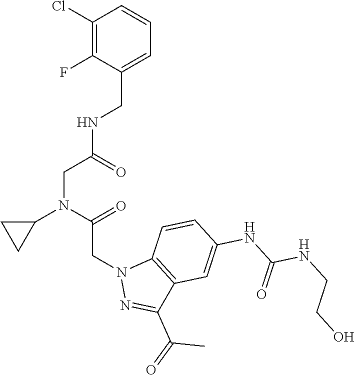 [SMILES: CC(=O)c1nn(CC(=O)N(CC(=O)NCc2cccc(Cl)c2F)C2CC2)c2ccc(NC(=O)NCCO)cc12]

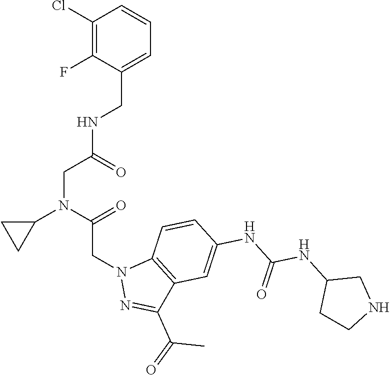 [SMILES: CC(=O)c1nn(CC(=O)N(CC(=O)NCc2cccc(Cl)c2F)C2CC2)c2ccc(NC(=O)NC3CCNC3)cc12]

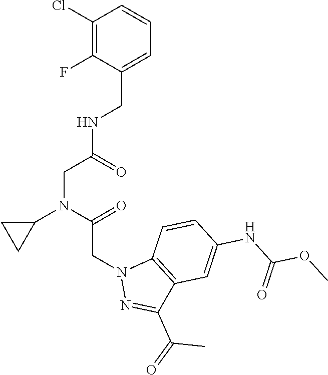 [SMILES: COC(=O)Nc1ccc2c(c1)c(C(C)=O)nn2CC(=O)N(CC(=O)NCc1cccc(Cl)c1F)C1CC1]

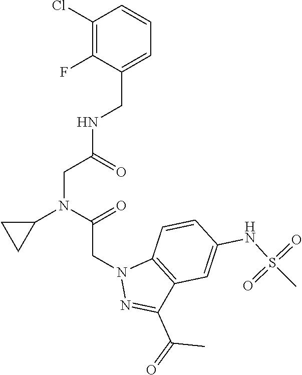 [SMILES: CC(=O)c1nn(CC(=O)N(CC(=O)NCc2cccc(Cl)c2F)C2CC2)c2ccc(NS(C)(=O)=O)cc12]

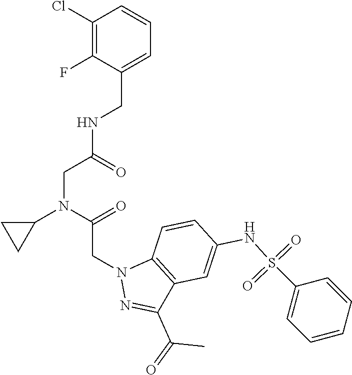 [SMILES: CC(=O)c1nn(CC(=O)N(CC(=O)NCc2cccc(Cl)c2F)C2CC2)c2ccc(NS(=O)(=O)c3ccccc3)cc12]

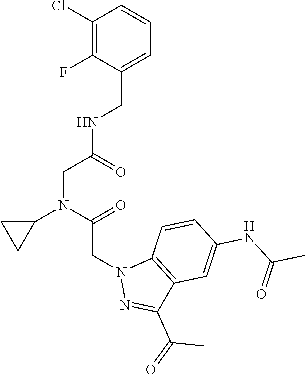 [SMILES: CC(=O)Nc1ccc2c(c1)c(C(C)=O)nn2CC(=O)N(CC(=O)NCc1cccc(Cl)c1F)C1CC1]

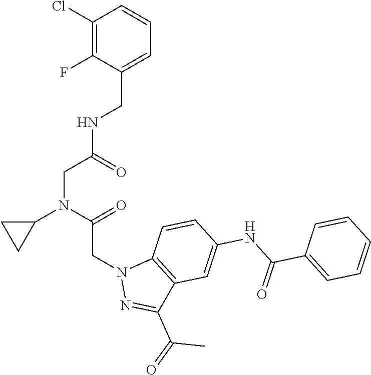 [SMILES: CC(=O)c1nn(CC(=O)N(CC(=O)NCc2cccc(Cl)c2F)C2CC2)c2ccc(NC(=O)c3ccccc3)cc12]

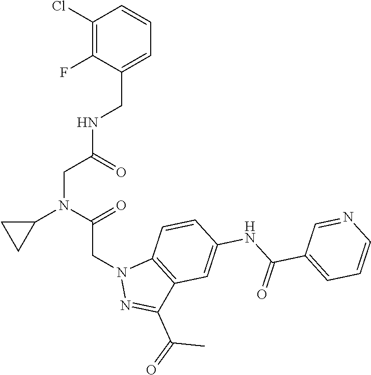 [SMILES: CC(=O)c1nn(CC(=O)N(CC(=O)NCc2cccc(Cl)c2F)C2CC2)c2ccc(NC(=O)c3cccnc3)cc12]

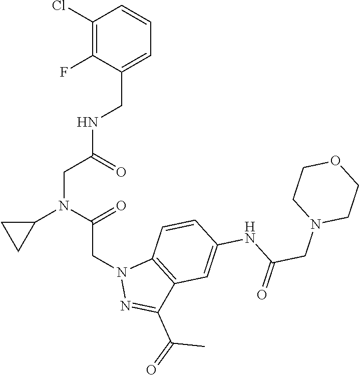 [SMILES: CC(=O)c1nn(CC(=O)N(CC(=O)NCc2cccc(Cl)c2F)C2CC2)c2ccc(NC(=O)CN3CCOCC3)cc12]

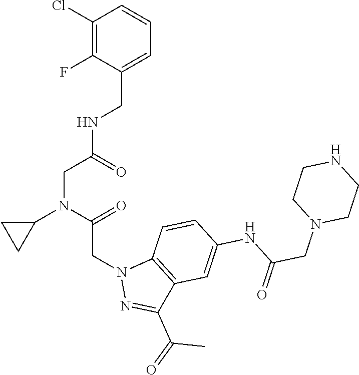 [SMILES: CC(=O)c1nn(CC(=O)N(CC(=O)NCc2cccc(Cl)c2F)C2CC2)c2ccc(NC(=O)CN3CCNCC3)cc12]

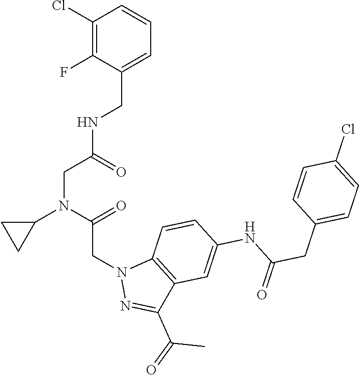 [SMILES: CC(=O)c1nn(CC(=O)N(CC(=O)NCc2cccc(Cl)c2F)C2CC2)c2ccc(NC(=O)Cc3ccc(Cl)cc3)cc12]

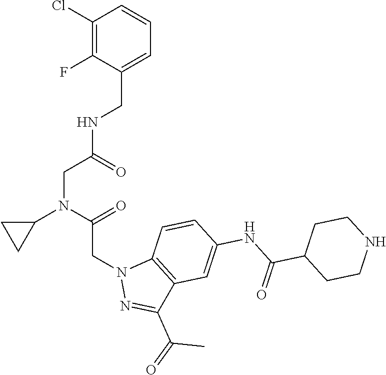 [SMILES: CC(=O)c1nn(CC(=O)N(CC(=O)NCc2cccc(Cl)c2F)C2CC2)c2ccc(NC(=O)C3CCNCC3)cc12]

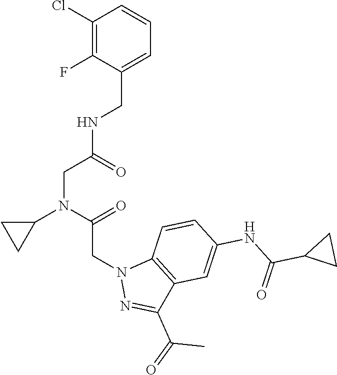 [SMILES: CC(=O)c1nn(CC(=O)N(CC(=O)NCc2cccc(Cl)c2F)C2CC2)c2ccc(NC(=O)C3CC3)cc12]

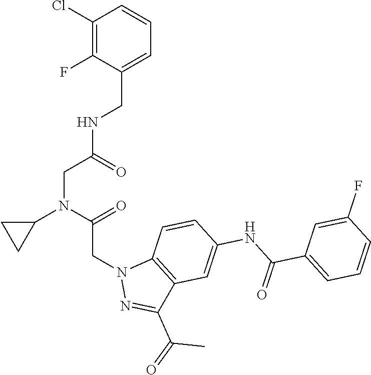 [SMILES: CC(=O)c1nn(CC(=O)N(CC(=O)NCc2cccc(Cl)c2F)C2CC2)c2ccc(NC(=O)c3cccc(F)c3)cc12]

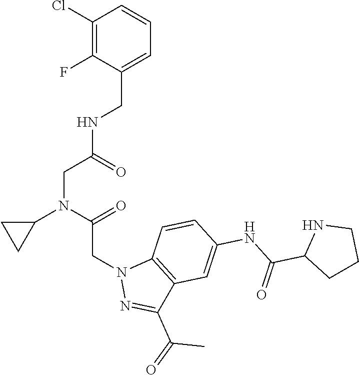 [SMILES: CC(=O)c1nn(CC(=O)N(CC(=O)NCc2cccc(Cl)c2F)C2CC2)c2ccc(NC(=O)C3CCCN3)cc12]

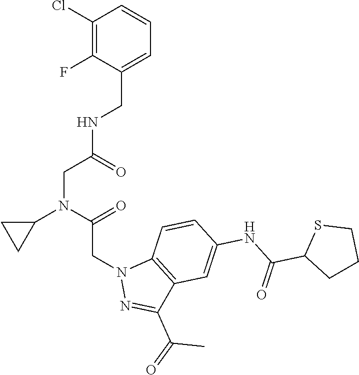 [SMILES: CC(=O)c1nn(CC(=O)N(CC(=O)NCc2cccc(Cl)c2F)C2CC2)c2ccc(NC(=O)C3CCCS3)cc12]

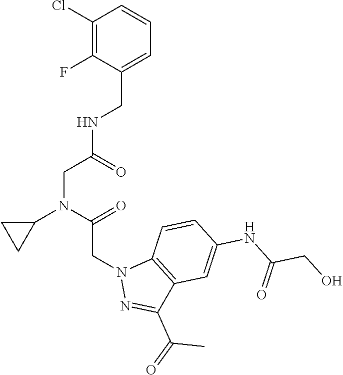 [SMILES: CC(=O)c1nn(CC(=O)N(CC(=O)NCc2cccc(Cl)c2F)C2CC2)c2ccc(NC(=O)CO)cc12]

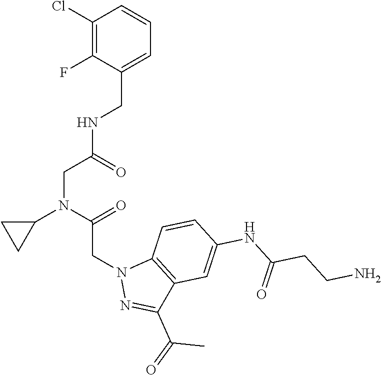 [SMILES: CC(=O)c1nn(CC(=O)N(CC(=O)NCc2cccc(Cl)c2F)C2CC2)c2ccc(NC(=O)CCN)cc12]

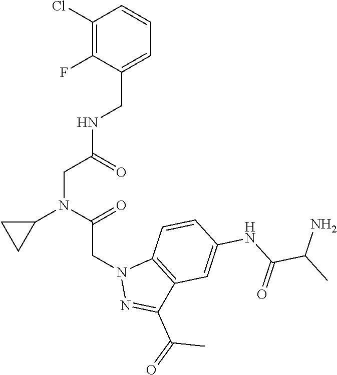 [SMILES: CC(=O)c1nn(CC(=O)N(CC(=O)NCc2cccc(Cl)c2F)C2CC2)c2ccc(NC(=O)C(C)N)cc12]

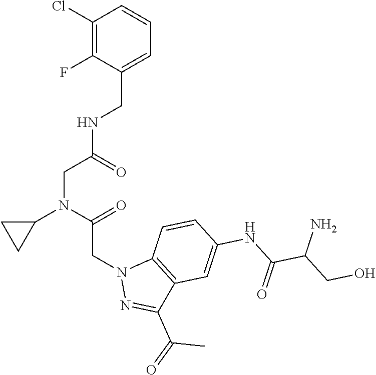 [SMILES: CC(=O)c1nn(CC(=O)N(CC(=O)NCc2cccc(Cl)c2F)C2CC2)c2ccc(NC(=O)C(N)CO)cc12]

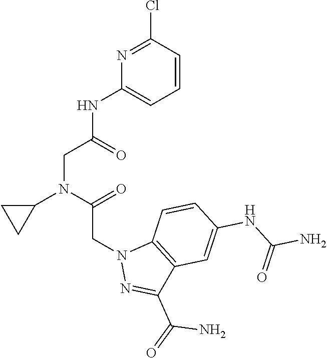 [SMILES: NC(=O)Nc1ccc2c(c1)c(C(N)=O)nn2CC(=O)N(CC(=O)Nc1cccc(Cl)n1)C1CC1]

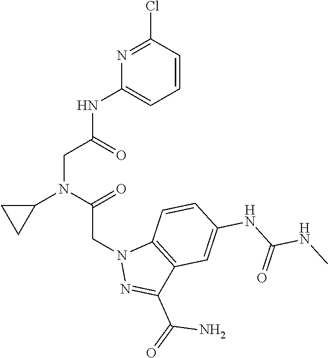 [SMILES: CNC(=O)Nc1ccc2c(c1)c(C(N)=O)nn2CC(=O)N(CC(=O)Nc1cccc(Cl)n1)C1CC1]

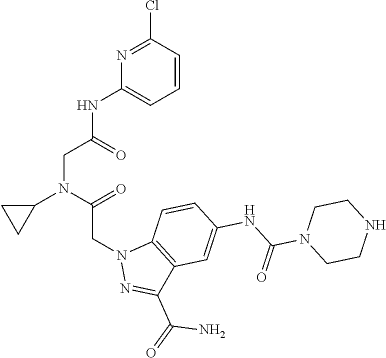 [SMILES: NC(=O)c1nn(CC(=O)N(CC(=O)Nc2cccc(Cl)n2)C2CC2)c2ccc(NC(=O)N3CCNCC3)cc12]

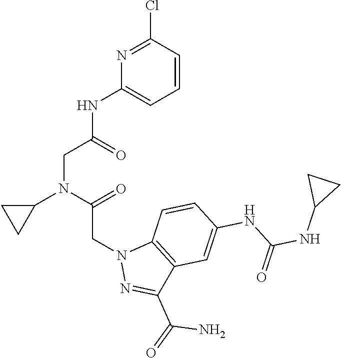 [SMILES: NC(=O)c1nn(CC(=O)N(CC(=O)Nc2cccc(Cl)n2)C2CC2)c2ccc(NC(=O)NC3CC3)cc12]

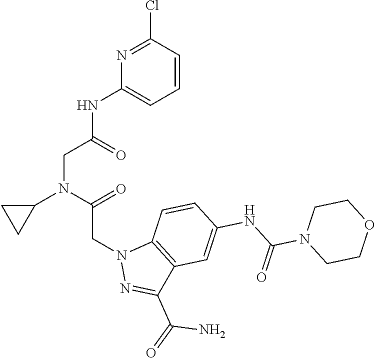 [SMILES: NC(=O)c1nn(CC(=O)N(CC(=O)Nc2cccc(Cl)n2)C2CC2)c2ccc(NC(=O)N3CCOCC3)cc12]

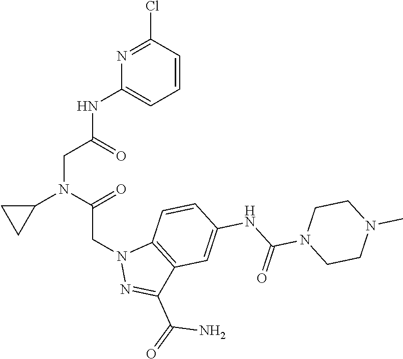 [SMILES: CN1CCN(C(=O)Nc2ccc3c(c2)c(C(N)=O)nn3CC(=O)N(CC(=O)Nc2cccc(Cl)n2)C2CC2)CC1]

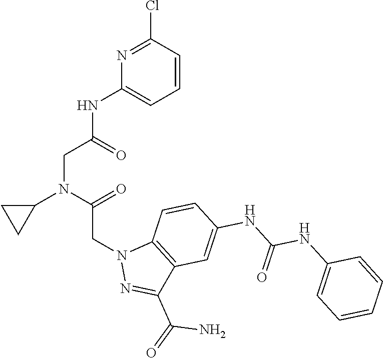 [SMILES: NC(=O)c1nn(CC(=O)N(CC(=O)Nc2cccc(Cl)n2)C2CC2)c2ccc(NC(=O)Nc3ccccc3)cc12]

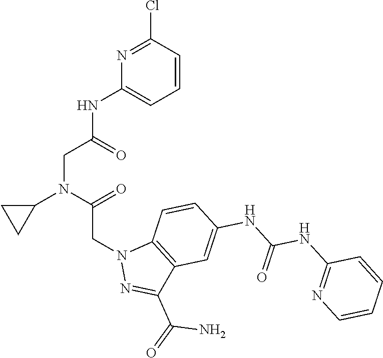 [SMILES: NC(=O)c1nn(CC(=O)N(CC(=O)Nc2cccc(Cl)n2)C2CC2)c2ccc(NC(=O)Nc3ccccn3)cc12]

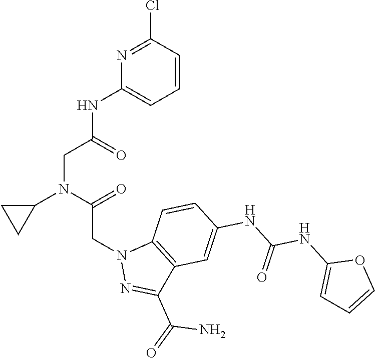 [SMILES: NC(=O)c1nn(CC(=O)N(CC(=O)Nc2cccc(Cl)n2)C2CC2)c2ccc(NC(=O)Nc3ccco3)cc12]

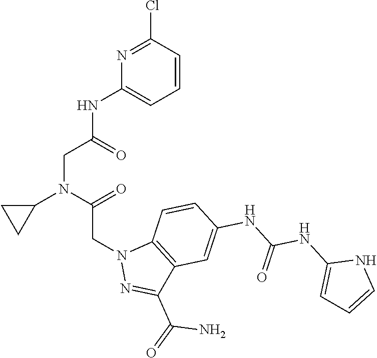 [SMILES: NC(=O)c1nn(CC(=O)N(CC(=O)Nc2cccc(Cl)n2)C2CC2)c2ccc(NC(=O)Nc3ccc[nH]3)cc12]

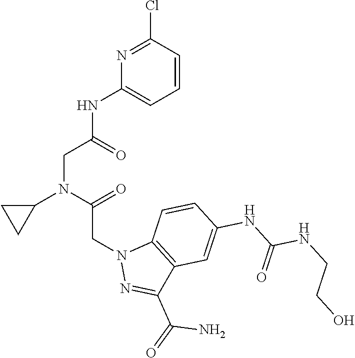 [SMILES: NC(=O)c1nn(CC(=O)N(CC(=O)Nc2cccc(Cl)n2)C2CC2)c2ccc(NC(=O)NCCO)cc12]

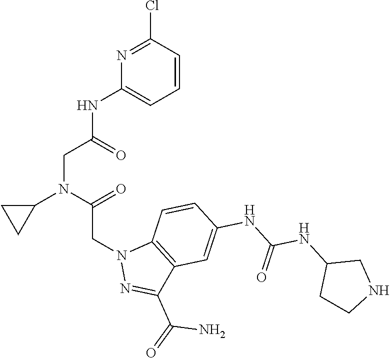 [SMILES: NC(=O)c1nn(CC(=O)N(CC(=O)Nc2cccc(Cl)n2)C2CC2)c2ccc(NC(=O)NC3CCNC3)cc12]

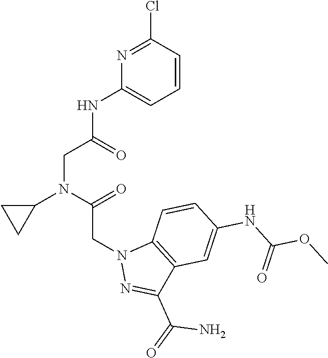 [SMILES: COC(=O)Nc1ccc2c(c1)c(C(N)=O)nn2CC(=O)N(CC(=O)Nc1cccc(Cl)n1)C1CC1]

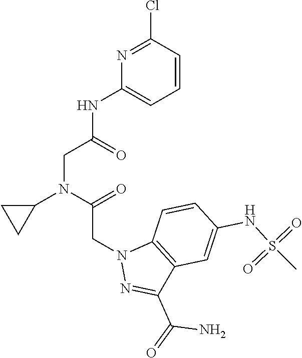 [SMILES: CS(=O)(=O)Nc1ccc2c(c1)c(C(N)=O)nn2CC(=O)N(CC(=O)Nc1cccc(Cl)n1)C1CC1]

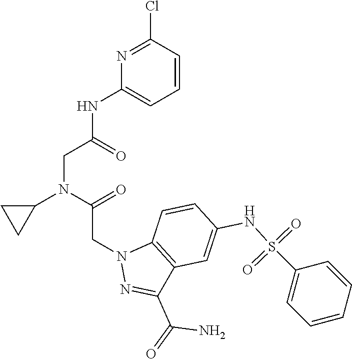 [SMILES: NC(=O)c1nn(CC(=O)N(CC(=O)Nc2cccc(Cl)n2)C2CC2)c2ccc(NS(=O)(=O)c3ccccc3)cc12]

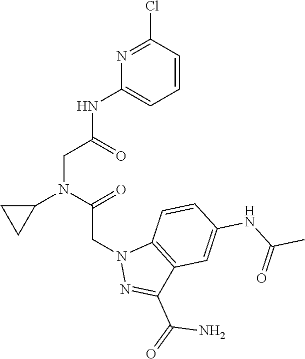 [SMILES: CC(=O)Nc1ccc2c(c1)c(C(N)=O)nn2CC(=O)N(CC(=O)Nc1cccc(Cl)n1)C1CC1]

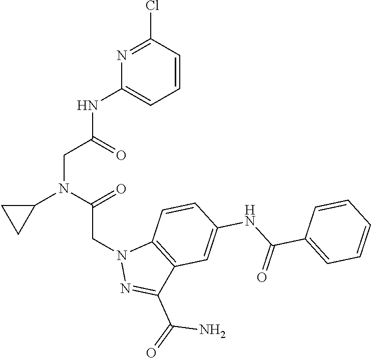 [SMILES: NC(=O)c1nn(CC(=O)N(CC(=O)Nc2cccc(Cl)n2)C2CC2)c2ccc(NC(=O)c3ccccc3)cc12]

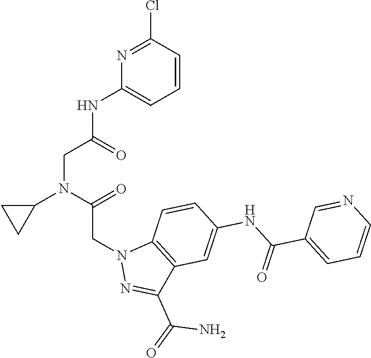 [SMILES: NC(=O)c1nn(CC(=O)N(CC(=O)Nc2cccc(Cl)n2)C2CC2)c2ccc(NC(=O)c3cccnc3)cc12]

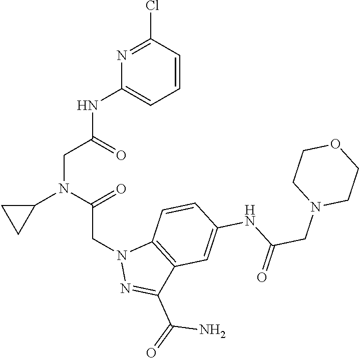 [SMILES: NC(=O)c1nn(CC(=O)N(CC(=O)Nc2cccc(Cl)n2)C2CC2)c2ccc(NC(=O)CN3CCOCC3)cc12]

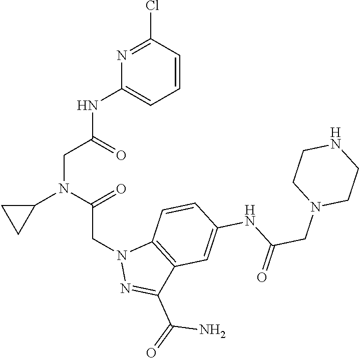 [SMILES: NC(=O)c1nn(CC(=O)N(CC(=O)Nc2cccc(Cl)n2)C2CC2)c2ccc(NC(=O)CN3CCNCC3)cc12]

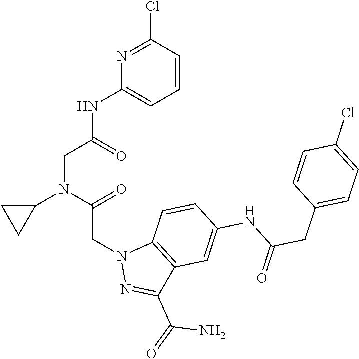 [SMILES: NC(=O)c1nn(CC(=O)N(CC(=O)Nc2cccc(Cl)n2)C2CC2)c2ccc(NC(=O)Cc3ccc(Cl)cc3)cc12]

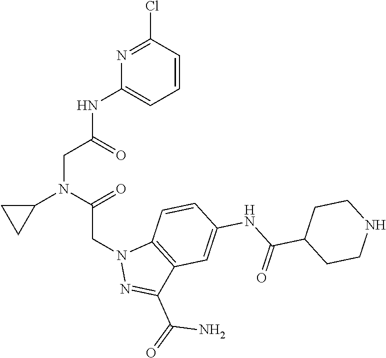 [SMILES: NC(=O)c1nn(CC(=O)N(CC(=O)Nc2cccc(Cl)n2)C2CC2)c2ccc(NC(=O)C3CCNCC3)cc12]

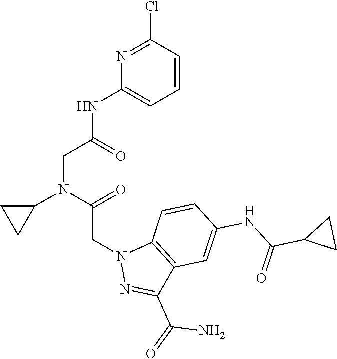 [SMILES: NC(=O)c1nn(CC(=O)N(CC(=O)Nc2cccc(Cl)n2)C2CC2)c2ccc(NC(=O)C3CC3)cc12]

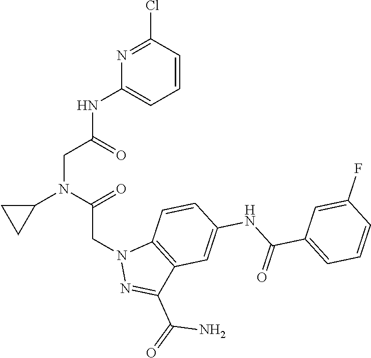 [SMILES: NC(=O)c1nn(CC(=O)N(CC(=O)Nc2cccc(Cl)n2)C2CC2)c2ccc(NC(=O)c3cccc(F)c3)cc12]

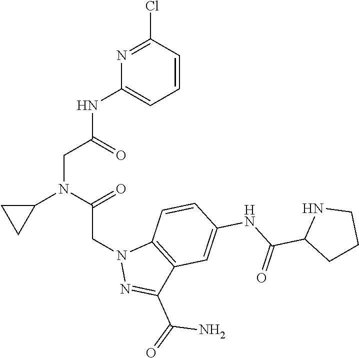 [SMILES: NC(=O)c1nn(CC(=O)N(CC(=O)Nc2cccc(Cl)n2)C2CC2)c2ccc(NC(=O)C3CCCN3)cc12]

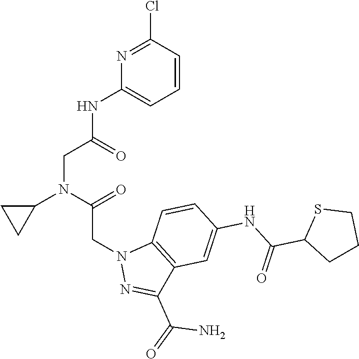 [SMILES: NC(=O)c1nn(CC(=O)N(CC(=O)Nc2cccc(Cl)n2)C2CC2)c2ccc(NC(=O)C3CCCS3)cc12]

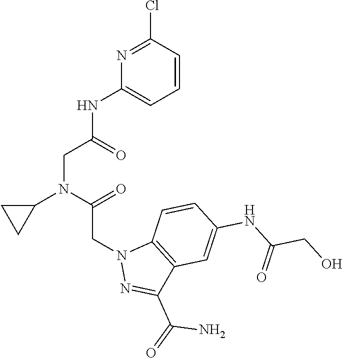 [SMILES: NC(=O)c1nn(CC(=O)N(CC(=O)Nc2cccc(Cl)n2)C2CC2)c2ccc(NC(=O)CO)cc12]

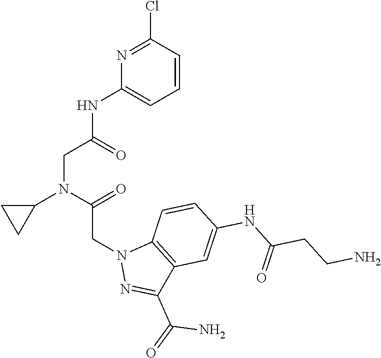 [SMILES: NCCC(=O)Nc1ccc2c(c1)c(C(N)=O)nn2CC(=O)N(CC(=O)Nc1cccc(Cl)n1)C1CC1]

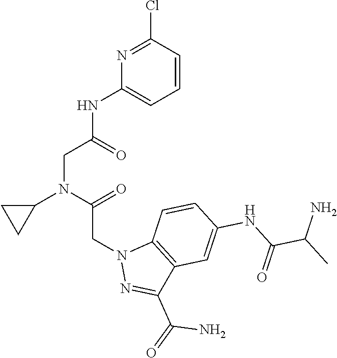 [SMILES: CC(N)C(=O)Nc1ccc2c(c1)c(C(N)=O)nn2CC(=O)N(CC(=O)Nc1cccc(Cl)n1)C1CC1]

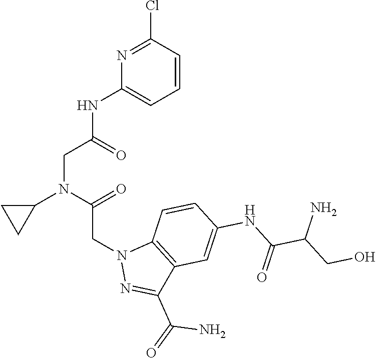 [SMILES: NC(=O)c1nn(CC(=O)N(CC(=O)Nc2cccc(Cl)n2)C2CC2)c2ccc(NC(=O)C(N)CO)cc12]

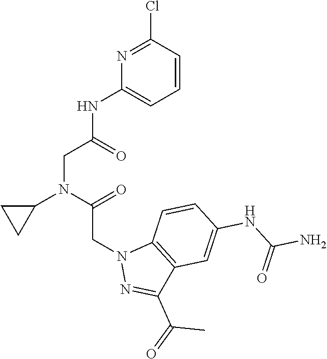 [SMILES: CC(=O)c1nn(CC(=O)N(CC(=O)Nc2cccc(Cl)n2)C2CC2)c2ccc(NC(N)=O)cc12]

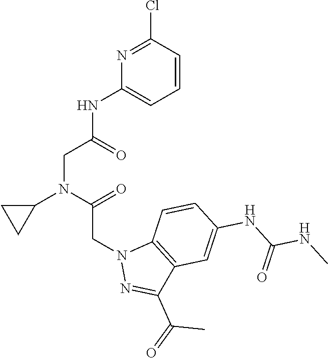 [SMILES: CNC(=O)Nc1ccc2c(c1)c(C(C)=O)nn2CC(=O)N(CC(=O)Nc1cccc(Cl)n1)C1CC1]

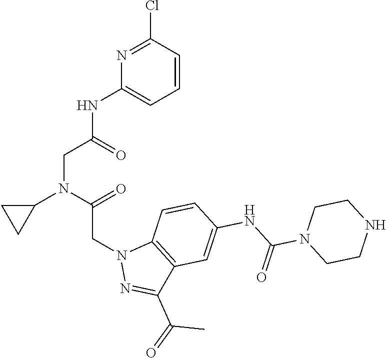 [SMILES: CC(=O)c1nn(CC(=O)N(CC(=O)Nc2cccc(Cl)n2)C2CC2)c2ccc(NC(=O)N3CCNCC3)cc12]

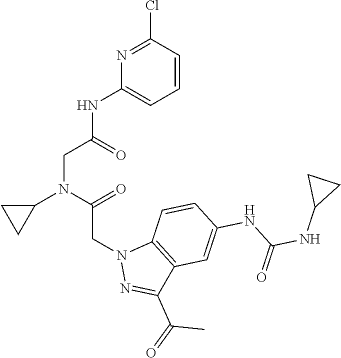 [SMILES: CC(=O)c1nn(CC(=O)N(CC(=O)Nc2cccc(Cl)n2)C2CC2)c2ccc(NC(=O)NC3CC3)cc12]

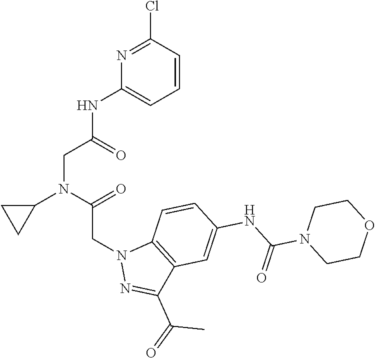 [SMILES: CC(=O)c1nn(CC(=O)N(CC(=O)Nc2cccc(Cl)n2)C2CC2)c2ccc(NC(=O)N3CCOCC3)cc12]

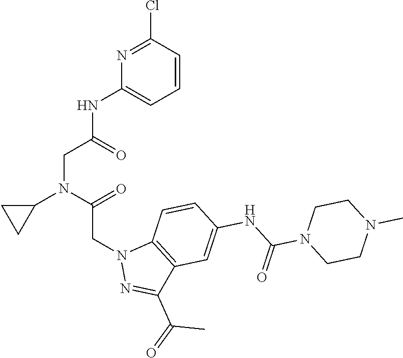 [SMILES: CC(=O)c1nn(CC(=O)N(CC(=O)Nc2cccc(Cl)n2)C2CC2)c2ccc(NC(=O)N3CCN(C)CC3)cc12]

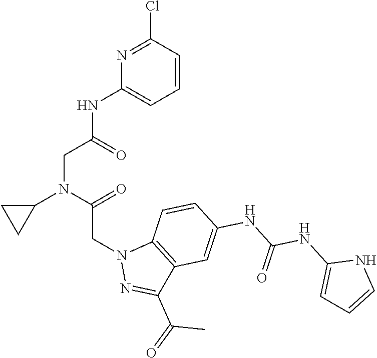 [SMILES: CC(=O)c1nn(CC(=O)N(CC(=O)Nc2cccc(Cl)n2)C2CC2)c2ccc(NC(=O)Nc3ccc[nH]3)cc12]

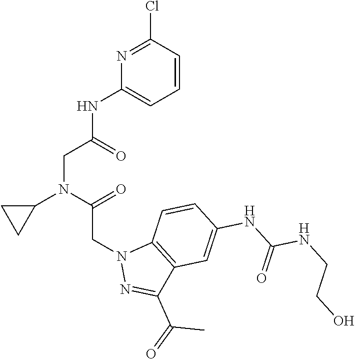 [SMILES: CC(=O)c1nn(CC(=O)N(CC(=O)Nc2cccc(Cl)n2)C2CC2)c2ccc(NC(=O)NCCO)cc12]

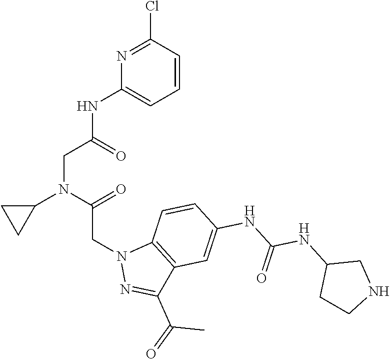 [SMILES: CC(=O)c1nn(CC(=O)N(CC(=O)Nc2cccc(Cl)n2)C2CC2)c2ccc(NC(=O)NC3CCNC3)cc12]

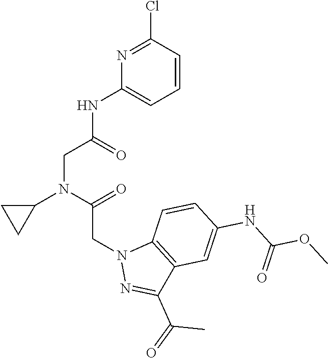 [SMILES: COC(=O)Nc1ccc2c(c1)c(C(C)=O)nn2CC(=O)N(CC(=O)Nc1cccc(Cl)n1)C1CC1]

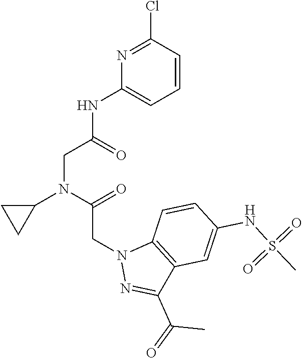 [SMILES: CC(=O)c1nn(CC(=O)N(CC(=O)Nc2cccc(Cl)n2)C2CC2)c2ccc(NS(C)(=O)=O)cc12]

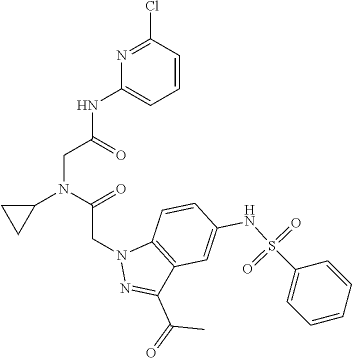 [SMILES: CC(=O)c1nn(CC(=O)N(CC(=O)Nc2cccc(Cl)n2)C2CC2)c2ccc(NS(=O)(=O)c3ccccc3)cc12]

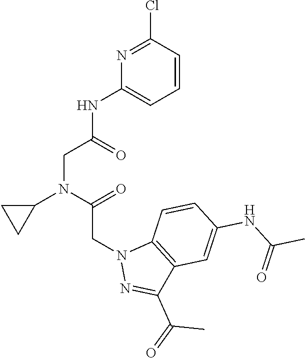 [SMILES: CC(=O)Nc1ccc2c(c1)c(C(C)=O)nn2CC(=O)N(CC(=O)Nc1cccc(Cl)n1)C1CC1]

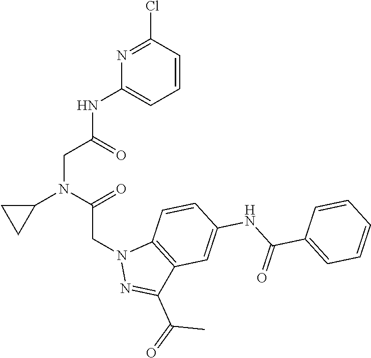 [SMILES: CC(=O)c1nn(CC(=O)N(CC(=O)Nc2cccc(Cl)n2)C2CC2)c2ccc(NC(=O)c3ccccc3)cc12]

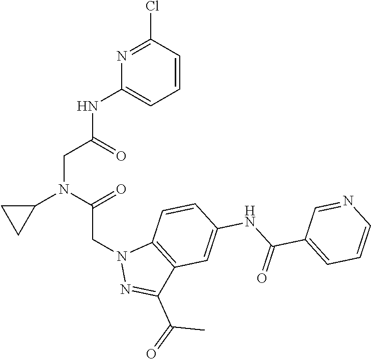 [SMILES: CC(=O)c1nn(CC(=O)N(CC(=O)Nc2cccc(Cl)n2)C2CC2)c2ccc(NC(=O)c3cccnc3)cc12]

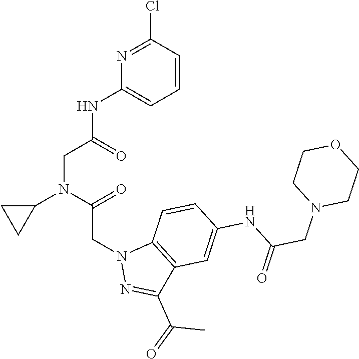 [SMILES: CC(=O)c1nn(CC(=O)N(CC(=O)Nc2cccc(Cl)n2)C2CC2)c2ccc(NC(=O)CN3CCOCC3)cc12]

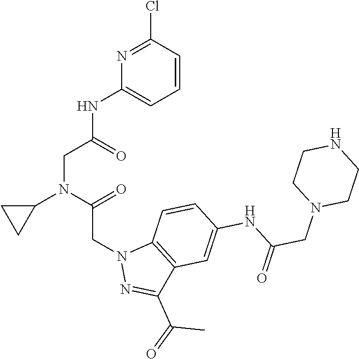 [SMILES: CC(=O)c1nn(CC(=O)N(CC(=O)Nc2cccc(Cl)n2)C2CC2)c2ccc(NC(=O)CN3CCNCC3)cc12]

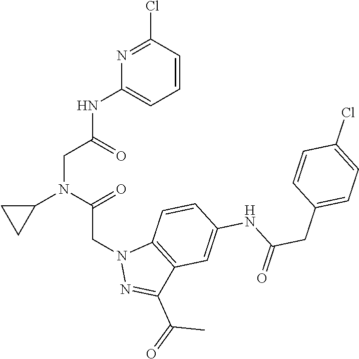 [SMILES: CC(=O)c1nn(CC(=O)N(CC(=O)Nc2cccc(Cl)n2)C2CC2)c2ccc(NC(=O)Cc3ccc(Cl)cc3)cc12]

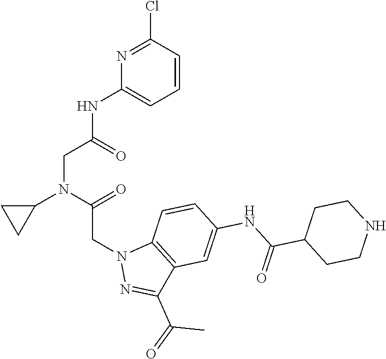 [SMILES: CC(=O)c1nn(CC(=O)N(CC(=O)Nc2cccc(Cl)n2)C2CC2)c2ccc(NC(=O)C3CCNCC3)cc12]

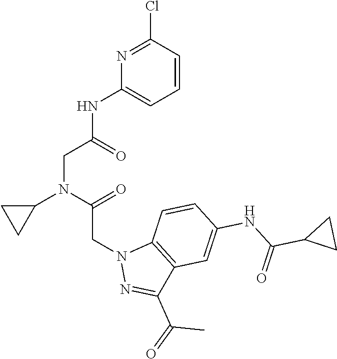 [SMILES: CC(=O)c1nn(CC(=O)N(CC(=O)Nc2cccc(Cl)n2)C2CC2)c2ccc(NC(=O)C3CC3)cc12]

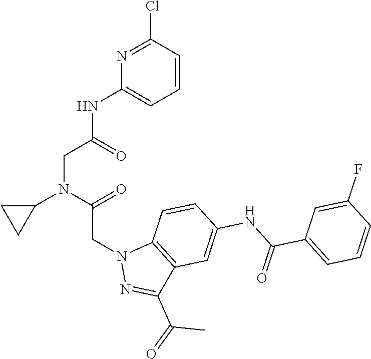 [SMILES: CC(=O)c1nn(CC(=O)N(CC(=O)Nc2cccc(Cl)n2)C2CC2)c2ccc(NC(=O)c3cccc(F)c3)cc12]

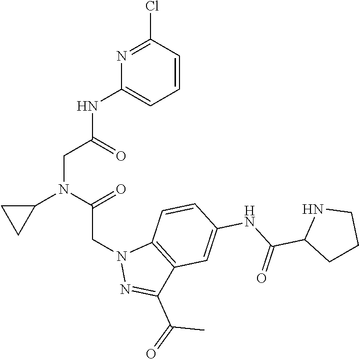 [SMILES: CC(=O)c1nn(CC(=O)N(CC(=O)Nc2cccc(Cl)n2)C2CC2)c2ccc(NC(=O)C3CCCN3)cc12]

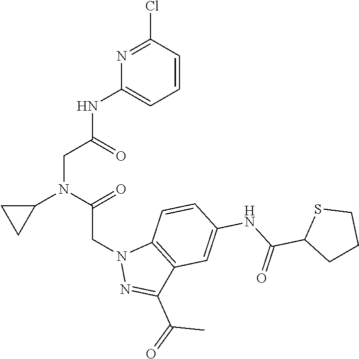 [SMILES: CC(=O)c1nn(CC(=O)N(CC(=O)Nc2cccc(Cl)n2)C2CC2)c2ccc(NC(=O)C3CCCS3)cc12]

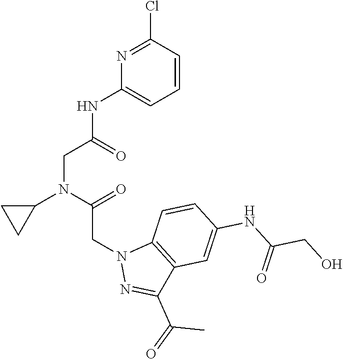 [SMILES: CC(=O)c1nn(CC(=O)N(CC(=O)Nc2cccc(Cl)n2)C2CC2)c2ccc(NC(=O)CO)cc12]

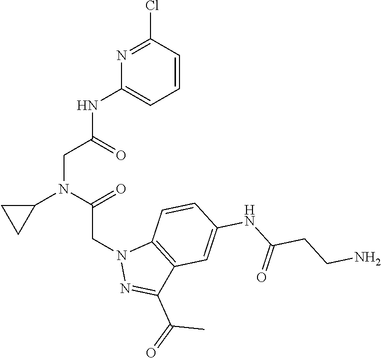 [SMILES: CC(=O)c1nn(CC(=O)N(CC(=O)Nc2cccc(Cl)n2)C2CC2)c2ccc(NC(=O)CCN)cc12]

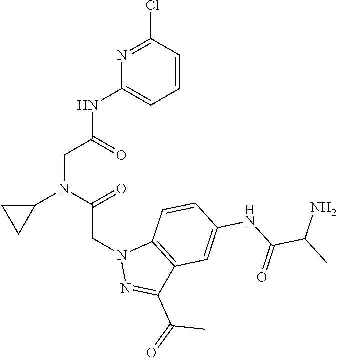 [SMILES: CC(=O)c1nn(CC(=O)N(CC(=O)Nc2cccc(Cl)n2)C2CC2)c2ccc(NC(=O)C(C)N)cc12]

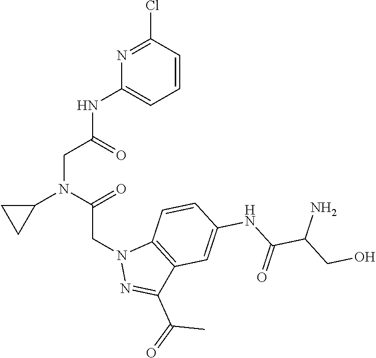 [SMILES: CC(=O)c1nn(CC(=O)N(CC(=O)Nc2cccc(Cl)n2)C2CC2)c2ccc(NC(=O)C(N)CO)cc12]

In some embodiments, the complement factor D inhibitory compound described by Formula (I) or Formula (Ia) has a structure provided in Table 3.

TABLE 3

| 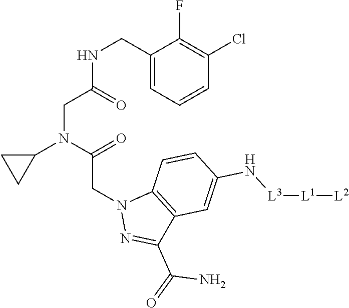 | | | 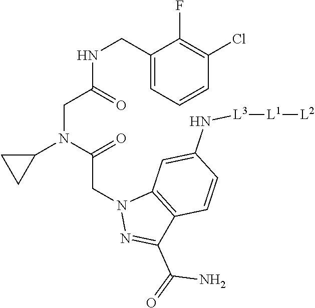 | | |
|---|---|---|---|---|---|
| —L³ | —L¹ | —L² | —L³ | —L¹ | —L² |
| C═O | absent | Ph | C═O | absent | Ph |
| C═O | absent | 3-pyridyl | C═O | absent | 3-pyridyl |
| C═O | absent | 2-pyridyl | C═O | absent | 2-pyridyl |
| C═O | absent | 4-pyridyl | C═O | absent | 4-pyridyl |
| C═O | absent | 5-pyrimidinyl | C═O | absent | 5-pyrimidinyl |
| C═O | absent | 4-pyrimidinyl | C═O | absent | 4-pyrimidinyl |
| C═O | absent | 2-pyrimidinyl | C═O | absent | 2-pyrimidinyl |
| C═O | absent | 2-chlorobenzyl | C═O | absent | 2-chlorobenzyl |
| C═O | absent | 3-chlorobenzyl | C═O | absent | 3-chlorobenzyl |
| C═O | absent | 4-chlorobenzyl | C═O | absent | 4-chlorobenzyl |
| C═O | absent | 2-fluorobenzyl | C═O | absent | 2-fluorobenzyl |
| C═O | absent | 3-fluorobenzyl | C═O | absent | 3-fluorobenzyl |
| C═O | absent | 4-fluorobenzyl | C═O | absent | 4-fluorobenzyl |
| C═O | absent | 2-cyanobenzyl | C═O | absent | 2-cyanobenzyl |
| C═O | absent | 3-cyanobenzyl | C═O | absent | 3-cyanobenzyl |
| C═O | absent | 4-cyanobenzyl | C═O | absent | 4-cyanobenzyl |
| C═O | absent | 2-methylbenzyl | C═O | absent | 2-methylbenzyl |
| C═O | absent | 3-methylbenzyl | C═O | absent | 3-methylbenzyl |
| C═O | absent | 4-methylbenzyl | C═O | absent | 4-methylbenzyl |
| C═O | absent | cyclopropyl | C═O | absent | cyclopropyl |
| C═O | absent | 2-piperidinyl | C═O | absent | 2-piperidinyl |
| C═O | absent | 3-piperidinyl | C═O | absent | 3-piperidinyl |
| C═O | absent | 2-piperidinyl | C═O | absent | 2-piperidinyl |
| C═O | absent | 2-morpholinyl | C═O | absent | 2-morpholinyl |
| C═O | absent | 3-morpholinyl | C═O | absent | 3-morpholinyl |
| C═O | absent | 4-morpholinyl | C═O | absent | 4-morpholinyl |
| C═O | absent | 2-tetrahydrofuryl | C═O | absent | 2-tetrahydrofuryl |
| C═O | absent | 3-tetrahydrofuryl | C═O | absent | 3-tetrahydrofuryl |
| C═O | absent | 2-pyrrolidinyl | C═O | absent | 2-pyrrolidinyl |
| C═O | absent | 3-pyrrolidinyl | C═O | absent | 3-pyrrolidinyl |
| C═O | absent | 2-tetrahydrothienyl | C═O | absent | 2-tetrahydrothienyl |
| C═O | absent | 3-tetrahydrothienyl | C═O | absent | 3-tetrahydrothienyl |
| C═O | absent | 1-piperazinyl | C═O | absent | 1-piperazinyl |
| C═O | absent | 2-piperazinyl | C═O | absent | 2-piperazinyl |
| C═O | absent | 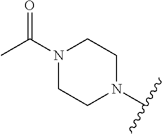 [SMILES: *N1CCN(C(C)=O)CC1] | C═O | absent | 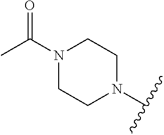 [SMILES: *N1CCN(C(C)=O)CC1] |
| C═O | absent | 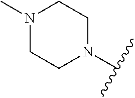 [SMILES: *N1CCN(C)CC1] | C═O | absent | 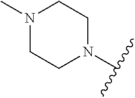 [SMILES: *N1CCN(C)CC1] |
| C═O | absent | Me | C═O | absent | Me |
| C═O | CH₂ | —OH | C═O | CH₂ | —OH |
| C═O | CH₂ | —OMe | C═O | CH₂ | —OMe |
| C═O | CH₂ | —NH₂ | C═O | CH₂ | —NH₂ |
| C═O | CH₂ | —NHMe | C═O | CH₂ | —NHMe |
| C═O | CH₂ | —CH₂OH | C═O | CH₂ | —CH₂OH |
| C═O | CH₂ | —CH₂OMe | C═O | CH₂ | —CH₂OMe |
| C═O | CH₂ | —CH₂NH₂ | C═O | CH₂ | —CH₂NH₂ |
| C═O | CH₂ | —CH₂NHMe | C═O | CH₂ | —CH₂NHMe |
| C═O | CH₂ | —CH₂OH | C═O | CH₂ | —CH₂OH |
| C═O | CH₂ | Ph | C═O | CH₂ | Ph |
| C═O | CH₂ | 3-pyridyl | C═O | CH₂ | 3-pyridyl |
| C═O | CH₂ | 2-pyridyl | C═O | CH₂ | 2-pyridyl |
| C═O | CH₂ | 4-pyridyl | C═O | CH₂ | 4-pyridyl |
| C═O | CH₂ | 5-pyrimidinyl | C═O | CH₂ | 5-pyrimidinyl |
| C═O | CH₂ | 4-pyrimidinyl | C═O | CH₂ | 4-pyrimidinyl |
| C═O | CH₂ | 2-pyrimidinyl | C═O | CH₂ | 2-pyrimidinyl |
| C═O | CH₂ | 2-chlorobenzyl | C═O | CH₂ | 2-chlorobenzyl |
| C═O | CH₂ | 3-chlorobenzyl | C═O | CH₂ | 3-chlorobenzyl |
| C═O | CH₂ | 4-chlorobenzyl | C═O | CH₂ | 4-chlorobenzyl |
| C═O | CH₂ | 2-fluorobenzyl | C═O | CH₂ | 2-fluorobenzyl |
| C═O | CH₂ | 3-fluorobenzyl | C═O | CH₂ | 3-fluorobenzyl |
| C═O | CH₂ | 4-fluorobenzyl | C═O | CH₂ | 4-fluorobenzyl |
| C═O | CH₂ | 2-cyanobenzyl | C═O | CH₂ | 2-cyanobenzyl |
| C═O | CH₂ | 3-cyanobenzyl | C═O | CH₂ | 3-cyanobenzyl |
| C═O | CH₂ | 4-cyanobenzyl | C═O | CH₂ | 4-cyanobenzyl |
| C═O | CH₂ | 2-methylbenzyl | C═O | CH₂ | 2-methylbenzyl |
| C═O | CH₂ | 3-methylbenzyl | C═O | CH₂ | 3-methylbenzyl |
| C═O | CH₂ | 4-methylbenzyl | C═O | CH₂ | 4-methylbenzyl |
| C═O | CH₂ | cyclopropyl | C═O | CH₂ | cyclopropyl |
| C═O | CH₂ | 2-piperidinyl | C═O | CH₂ | 2-piperidinyl |
| C═O | CH₂ | 3-piperidinyl | C═O | CH₂ | 3-piperidinyl |
| C═O | CH₂ | 2-piperidinyl | C═O | CH₂ | 2-piperidinyl |
| C═O | CH₂ | 2-morpholinyl | C═O | CH₂ | 2-morpholinyl |
| C═O | CH₂ | 3-morpholinyl | C═O | CH₂ | 3-morpholinyl |
| C═O | CH₂ | 4-morpholinyl | C═O | CH₂ | 4-morpholinyl |
| C═O | CH₂ | 1-piperazinyl | C═O | CH₂ | 1-piperazinyl |
| C═O | CH₂ | 2-piperazinyl | C═O | CH₂ | 2-piperazinyl |
| C═O | CH₂ | 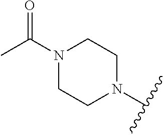 [SMILES: *N1CCN(C(C)=O)CC1] | C═O | CH₂ | 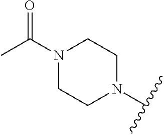 [SMILES: *N1CCN(C(C)=O)CC1] |
| C═O | CH₂ | 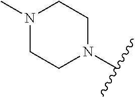 [SMILES: *N1CCN(C)CC1] | C═O | CH₂ | 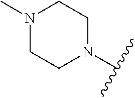 [SMILES: *N1CCN(C)CC1] |
| C═O | —CH(NH₂)— | Me | C═O | —CH(NH₂)— | Me |
| C═O | —CH(NH₂)— | Et | C═O | —CH(NH₂)— | Et |
| C═O | —CH(NH₂)— | iPr | C═O | —CH(NH₂)— | iPr |
| C═O | —CH(NH₂)— | iBu | C═O | —CH(NH₂)— | iBu |
| C═O | —CH(NH₂)— | —CH₂OH | C═O | —CH(NH₂)— | —CH₂OH |
| C═O | NH | Ph | C═O | NH | Ph |
| C═O | NH | 3-pyridyl | C═O | NH | 3-pyridyl |
| C═O | NH | 2-pyridyl | C═O | NH | 2-pyridyl |
| C═O | NH | 4-pyridyl | C═O | NH | 4-pyridyl |
| C═O | NH | 5-pyrimidinyl | C═O | NH | 5-pyrimidinyl |
| C═O | NH | 4-pyrimidinyl | C═O | NH | 4-pyrimidinyl |
| C═O | NH | 2-pyrimidinyl | C═O | NH | 2-pyrimidinyl |
| C═O | NH | 2-chlorobenzyl | C═O | NH | 2-chlorobenzyl |
| C═O | NH | 3-chlorobenzyl | C═O | NH | 3-chlorobenzyl |
| C═O | NH | 4-chlorobenzyl | C═O | NH | 4-chlorobenzyl |
| C═O | NH | 2-fluorobenzyl | C═O | NH | 2-fluorobenzyl |
| C═O | NH | 3-fluorobenzyl | C═O | NH | 3-fluorobenzyl |
| C═O | NH | 4-fluorobenzyl | C═O | NH | 4-fluorobenzyl |
| C═O | NH | 2-cyanobenzyl | C═O | NH | 2-cyanobenzyl |
| C═O | NH | 3-cyanobenzyl | C═O | NH | 3-cyanobenzyl |
| C═O | NH | 4-cyanobenzyl | C═O | NH | 4-cyanobenzyl |
| C═O | NH | 2-methylbenzyl | C═O | NH | 2-methylbenzyl |
| C═O | NH | 3-methylbenzyl | C═O | NH | 3-methylbenzyl |
| C═O | NH | 4-methylbenzyl | C═O | NH | 4-methylbenzyl |
| C═O | NH | cyclopropyl | C═O | NH | cyclopropyl |
| C═O | NH | 2-piperidinyl | C═O | NH | 2-piperidinyl |
| C═O | NH | 3-piperidinyl | C═O | NH | 3-piperidinyl |
| C═O | NH | 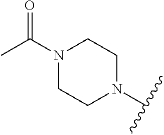 [SMILES: *N1CCN(C(C)=O)CC1] | C═O | NH | 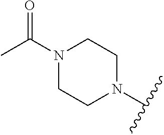 [SMILES: *N1CCN(C(C)=O)CC1] |
| C═O | NH | 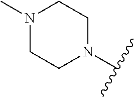 [SMILES: *N1CCN(C)CC1] | C═O | NH | 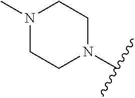 [SMILES: *N1CCN(C)CC1] |
| C═O | NH | 2-morpholinyl | C═O | NH | 2-morpholinyl |
| C═O | NH | 3-morpholinyl | C═O | NH | 3-morpholinyl |
| C═O | NH | 4-morpholinyl | C═O | NH | 4-morpholinyl |
| C═O | NH | 2-tetrahydrofuryl | C═O | NH | 2-tetrahydrofuryl |
| C═O | NH | 3-tetrahydrofuryl | C═O | NH | 3-tetrahydrofuryl |
| C═O | NH | 2-pyrrolidinyl | C═O | NH | 2-pyrrolidinyl |
| C═O | NH | 3-pyrrolidinyl | C═O | NH | 3-pyrrolidinyl |
| C═O | NH | 2-tetrahydrothienyl | C═O | NH | 2-tetrahydrothienyl |
| C═O | NH | 3-tetrahydrothienyl | C═O | NH | 3-tetrahydrothienyl |
| C═O | NH | 1-piperazinyl | C═O | NH | 1-piperazinyl |
| C═O | NH | 2-piperazinyl | C═O | NH | 2-piperazinyl |
| C═O | —NHCH₂— | —CH₂OH | C═O | —NHCH₂— | —CH₂OH |
| C═O | —NHCH₂— | —CH₂OMe | C═O | —NHCH₂— | —CH₂OMe |
| C═O | —NHCH₂— | —CH₂NH₂ | C═O | —NHCH₂— | —CH₂NH₂ |
| C═O | —NHCH₂— | —CH₂NHMe | C═O | —NHCH₂— | —CH₂NHMe |
| C═O | O | Me | C═O | O | Me |
| C═O | O | Et | C═O | O | Et |
| C═O | O | iPr | C═O | O | iPr |
| C═O | O | iBu | C═O | O | iBu |
| C═O | O | Ph | C═O | O | Ph |
| absent | SO₂ | Me | absent | SO₂ | Me |
| absent | SO₂ | Ph | absent | SO₂ | Ph |
| 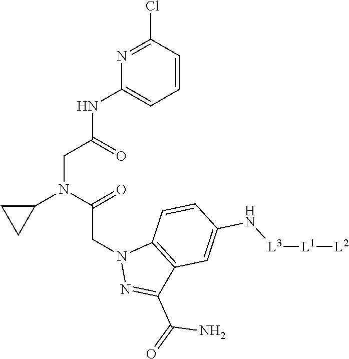 | | | 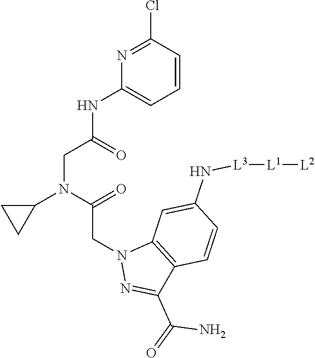 | | |
| —L³ | —L¹ | —L² | —L³ | —L¹ | —L² |
| C═O | absent | Ph | C═O | absent | Ph |
| C═O | absent | 3-pyridyl | C═O | absent | 3-pyridyl |
| C═O | absent | 2-pyridyl | C═O | absent | 2-pyridyl |
| C═O | absent | 4-pyridyl | C═O | absent | 4-pyridyl |
| C═O | absent | 5-pyrimidinyl | C═O | absent | 5-pyrimidinyl |
| C═O | absent | 4-pyrimidinyl | C═O | absent | 4-pyrimidinyl |
| C═O | absent | 2-pyrimidinyl | C═O | absent | 2-pyrimidinyl |
| C═O | absent | 2-chlorobenzyl | C═O | absent | 2-chlorobenzyl |
| C═O | absent | 3-chlorobenzyl | C═O | absent | 3-chlorobenzyl |
| C═O | absent | 4-chlorobenzyl | C═O | absent | 4-chlorobenzyl |
| C═O | absent | 2-fluorobenzyl | C═O | absent | 2-fluorobenzyl |
| C═O | absent | 3-fluorobenzyl | C═O | absent | 3-fluorobenzyl |
| C═O | absent | 4-fluorobenzyl | C═O | absent | 4-fluorobenzyl |
| C═O | absent | 2-cyanobenzyl | C═O | absent | 2-cyanobenzyl |
| C═O | absent | 3-cyanobenzyl | C═O | absent | 3-cyanobenzyl |
| C═O | absent | 4-cyanobenzyl | C═O | absent | 4-cyanobenzyl |
| C═O | absent | 2-methylbenzyl | C═O | absent | 2-methylbenzyl |
| C═O | absent | 3-methylbenzyl | C═O | absent | 3-methylbenzyl |
| C═O | absent | 4-methylbenzyl | C═O | absent | 4-methylbenzyl |
| C═O | absent | cyclopropyl | C═O | absent | cyclopropyl |
| C═O | absent | 2-piperidinyl | C═O | absent | 2-piperidinyl |
| C═O | absent | 3-piperidinyl | C═O | absent | 3-piperidinyl |
| C═O | absent | 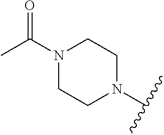 [SMILES: *N1CCN(C(C)=O)CC1] | C═O | absent | 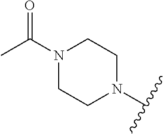 [SMILES: *N1CCN(C(C)=O)CC1] |
| C═O | absent | 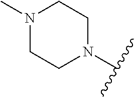 [SMILES: *N1CCN(C)CC1] | C═O | absent | 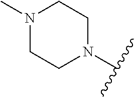 [SMILES: *N1CCN(C)CC1] |
| C═O | absent | 2-morpholinyl | C═O | absent | 2-morpholinyl |
| C═O | absent | 3-morpholinyl | C═O | absent | 3-morpholinyl |
| C═O | absent | 4-morpholinyl | C═O | absent | 4-morpholinyl |
| C═O | absent | 2-tetrahydrofuryl | C═O | absent | 2-tetrahydrofuryl |
| C═O | absent | 3-tetrahydrofuryl | C═O | absent | 3-tetrahydrofuryl |
| C═O | absent | 2-pyrrolidinyl | C═O | absent | 2-pyrrolidinyl |
| C═O | absent | 3-pyrrolidinyl | C═O | absent | 3-pyrrolidinyl |
| C═O | absent | 2-tetrahydrothienyl | C═O | absent | 2-tetrahydrothienyl |
| C═O | absent | 3-tetrahydrothienyl | C═O | absent | 3-tetrahydrothienyl |
| C═O | absent | 1-piperazinyl | C═O | absent | 1-piperazinyl |
| C═O | absent | 2-piperazinyl | C═O | absent | 2-piperazinyl |
| C═O | CH₂ | —OH | C═O | CH₂ | —OH |
| C═O | CH₂ | —OMe | C═O | CH₂ | —OMe |
| C═O | CH₂ | —NH₂ | C═O | CH₂ | —NH₂ |
| C═O | CH₂ | —NHMe | C═O | CH₂ | —NHMe |
| C═O | CH₂ | —CH₂OH | C═O | CH₂ | —CH₂OH |
| C═O | CH₂ | —CH₂OMe | C═O | CH₂ | —CH₂OMe |
| C═O | CH₂ | —CH₂NH₂ | C═O | CH₂ | —CH₂NH₂ |
| C═O | CH₂ | —CH₂NHMe | C═O | CH₂ | —CH₂NHMe |
| C═O | CH₂ | Me | C═O | CH₂ | Me |
| C═O | CH₂ | Et | C═O | CH₂ | Et |
| C═O | CH₂ | iPr | C═O | CH₂ | iPr |
| C═O | CH₂ | iBu | C═O | CH₂ | iBu |
| C═O | CH₂ | —CH₂OH | C═O | CH₂ | —CH₂OH |
| C═O | CH₂ | Ph | C═O | CH₂ | Ph |
| C═O | CH₂ | 3-pyridyl | C═O | CH₂ | 3-pyridyl |
| C═O | CH₂ | 2-pyridyl | C═O | CH₂ | 2-pyridyl |
| C═O | CH₂ | 4-pyridyl | C═O | CH₂ | 4-pyridyl |
| C═O | CH₂ | 5-pyrimidinyl | C═O | CH₂ | 5-pyrimidinyl |
| C═O | CH₂ | 4-pyrimidinyl | C═O | CH₂ | 4-pyrimidinyl |
| C═O | CH₂ | 2-pyrimidinyl | C═O | CH₂ | 2-pyrimidinyl |
| C═O | CH₂ | 2-chlorobenzyl | C═O | CH₂ | 2-chlorobenzyl |
| C═O | CH₂ | 3-chlorobenzyl | C═O | CH₂ | 3-chlorobenzyl |
| C═O | CH₂ | 4-chlorobenzyl | C═O | CH₂ | 4-chlorobenzyl |
| C═O | CH₂ | 2-fluorobenzyl | C═O | CH₂ | 2-fluorobenzyl |
| C═O | CH₂ | 3-fluorobenzyl | C═O | CH₂ | 3-fluorobenzyl |
| C═O | CH₂ | 4-fluorobenzyl | C═O | CH₂ | 4-fluorobenzyl |
| C═O | CH₂ | 2-cyanobenzyl | C═O | CH₂ | 2-cyanobenzyl |
| C═O | CH₂ | 3-cyanobenzyl | C═O | CH₂ | 3-cyanobenzyl |
| C═O | CH₂ | 4-cyanobenzyl | C═O | CH₂ | 4-cyanobenzyl |
| C═O | CH₂ | 2-methylbenzyl | C═O | CH₂ | 2-methylbenzyl |
| C═O | CH₂ | 3-methylbenzyl | C═O | CH₂ | 3-methylbenzyl |
| C═O | CH₂ | 4-methylbenzyl | C═O | CH₂ | 4-methylbenzyl |
| C═O | CH₂ | cyclopropyl | C═O | CH₂ | cyclopropyl |
| C═O | CH₂ | 2-piperidinyl | C═O | CH₂ | 2-piperidinyl |
| C═O | CH₂ | 3-piperidinyl | C═O | CH₂ | 3-piperidinyl |
| C═O | CH₂ | 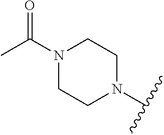 [SMILES: *N1CCN(C(C)=O)CC1] | C═O | CH₂ | 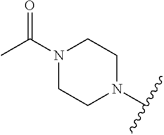 [SMILES: *N1CCN(C(C)=O)CC1] |
| C═O | CH₂ | 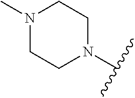 [SMILES: *N1CCN(C)CC1] | C═O | CH₂ | 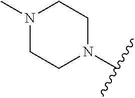 [SMILES: *N1CCN(C)CC1] |
| C═O | CH₂ | 2-morpholinyl | C═O | CH₂ | 2-morpholinyl |
| C═O | CH₂ | 3-morpholinyl | C═O | CH₂ | 3-morpholinyl |
| C═O | CH₂ | 4-morpholinyl | C═O | CH₂ | 4-morpholinyl |
| C═O | CH₂ | 1-piperazinyl | C═O | CH₂ | 1-piperazinyl |
| C═O | CH₂ | 2-piperazinyl | C═O | CH₂ | 2-piperazinyl |
| C═O | —CH(NH₂)— | Me | C═O | —CH(NH₂)— | Me |
| C═O | —CH(NH₂)— | Et | C═O | —CH(NH₂)— | Et |
| C═O | —CH(NH₂)— | iPr | C═O | —CH(NH₂)— | iPr |
| C═O | —CH(NH₂)— | iBu | C═O | —CH(NH₂)— | iBu |
| C═O | —CH(NH₂)— | —CH₂OH | C═O | —CH(NH₂)— | —CH₂OH |
| C═O | NH | Ph | C═O | NH | Ph |
| C═O | NH | 3-pyridyl | C═O | NH | 3-pyridyl |
| C═O | NH | 2-pyridyl | C═O | NH | 2-pyridyl |
| C═O | NH | 4-pyridyl | C═O | NH | 4-pyridyl |
| C═O | NH | 5-pyrimidinyl | C═O | NH | 5-pyrimidinyl |
| C═O | NH | 4-pyrimidinyl | C═O | NH | 4-pyrimidinyl |
| C═O | NH | 2-pyrimidinyl | C═O | NH | 2-pyrimidinyl |
| C═O | NH | 2-chlorobenzyl | C═O | NH | 2-chlorobenzyl |
| C═O | NH | 3-chlorobenzyl | C═O | NH | 3-chlorobenzyl |
| C═O | NH | 4-chlorobenzyl | C═O | NH | 4-chlorobenzyl |
| C═O | NH | 2-fluorobenzyl | C═O | NH | 2-fluorobenzyl |
| C═O | NH | 3-fluorobenzyl | C═O | NH | 3-fluorobenzyl |
| C═O | NH | 4-fluorobenzyl | C═O | NH | 4-fluorobenzyl |
| C═O | NH | 2-cyanobenzyl | C═O | NH | 2-cyanobenzyl |
| C═O | NH | 3-cyanobenzyl | C═O | NH | 3-cyanobenzyl |
| C═O | NH | 4-cyanobenzyl | C═O | NH | 4-cyanobenzyl |
| C═O | NH | 2-methylbenzyl | C═O | NH | 2-methylbenzyl |
| C═O | NH | 3-methylbenzyl | C═O | NH | 3-methylbenzyl |
| C═O | NH | 4-methylbenzyl | C═O | NH | 4-methylbenzyl |
| C═O | NH | cyclopropyl | C═O | NH | cyclopropyl |
| C═O | NH | 2-piperidinyl | C═O | NH | 2-piperidinyl |
| C═O | NH | 3-piperidinyl | C═O | NH | 3-piperidinyl |
| C═O | NH | 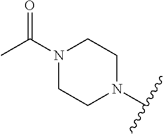 [SMILES: *N1CCN(C(C)=O)CC1] | C═O | NH | 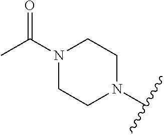 [SMILES: *N1CCN(C(C)=O)CC1] |
| C═O | NH | 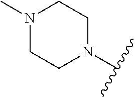 [SMILES: *N1CCN(C)CC1] | C═O | NH | 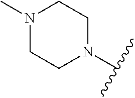 [SMILES: *N1CCN(C)CC1] |
| C═O | NH | 2-morpholinyl | C═O | NH | 2-morpholinyl |
| C═O | NH | 3-morpholinyl | C═O | NH | 3-morpholinyl |
| C═O | NH | 4-morpholinyl | C═O | NH | 4-morpholinyl |
| C═O | NH | 2-tetrahydrofuryl | C═O | NH | 2-tetrahydrofuryl |
| C═O | NH | 3-tetrahydrofuryl | C═O | NH | 3-tetrahydrofuryl |
| C═O | NH | 2-pyrrolidinyl | C═O | NH | 2-pyrrolidinyl |
| C═O | NH | 3-pyrrolidinyl | C═O | NH | 3-pyrrolidinyl |
| C═O | NH | 2-tetrahydrothienyl | C═O | NH | 2-tetrahydrothienyl |
| C═O | NH | 3-tetrahydrothienyl | C═O | NH | 3-tetrahydrothienyl |
| C═O | NH | 1-piperazinyl | C═O | NH | 1-piperazinyl |
| C═O | NH | 2-piperazinyl | C═O | NH | 2-piperazinyl |
| C═O | —NHCH₂— | —CH₂OH | C═O | —NHCH₂— | —CH₂OH |
| C═O | —NHCH₂— | —CH₂OMe | C═O | —NHCH₂— | —CH₂OMe |
| C═O | —NHCH₂— | —CH₂NH₂ | C═O | —NHCH₂— | —CH₂NH₂ |
| C═O | —NHCH₂— | —CH₂NHMe | C═O | —NHCH₂— | —CH₂NHMe |
| C═O | O | Me | C═O | O | Me |
| C═O | O | Et | C═O | O | Et |
| C═O | O | iPr | C═O | O | iPr |
| C═O | O | iBu | C═O | O | iBu |
| C═O | O | Ph | C═O | O | Ph |
| absent | SO₂ | Me | absent | SO₂ | Me |
| absent | SO₂ | Ph | absent | SO₂ | Ph |
| 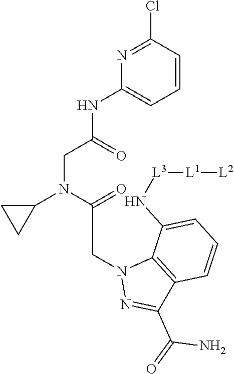 | | | 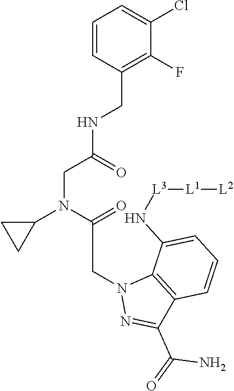 | | |
| —L³ | —L¹ | —L² | —L³ | —L¹ | —L² |
| C═O | absent | Ph | C═O | absent | Ph |
| C═O | absent | 3-pyridyl | C═O | absent | 3-pyridyl |
| C═O | absent | 2-pyridyl | C═O | absent | 2-pyridyl |
| C═O | absent | 4-pyridyl | C═O | absent | 4-pyridyl |
| C═O | absent | 5-pyrimidinyl | C═O | absent | 5-pyrimidinyl |
| C═O | absent | 4-pyrimidinyl | C═O | absent | 4-pyrimidinyl |
| C═O | absent | 2-pyrimidinyl | C═O | absent | 2-pyrimidinyl |
| C═O | absent | 2-chlorobenzyl | C═O | absent | 2-chlorobenzyl |
| C═O | absent | 3-chlorobenzyl | C═O | absent | 3-chlorobenzyl |
| C═O | absent | 4-chlorobenzyl | C═O | absent | 4-chlorobenzyl |
| C═O | absent | 2-fluorobenzyl | C═O | absent | 2-fluorobenzyl |
| C═O | absent | 3-fluorobenzyl | C═O | absent | 3-fluorobenzyl |
| C═O | absent | 4-fluorobenzyl | C═O | absent | 4-fluorobenzyl |
| C═O | absent | 2-cyanobenzyl | C═O | absent | 2-cyanobenzyl |
| C═O | absent | 3-cyanobenzyl | C═O | absent | 3-cyanobenzyl |
| C═O | absent | 4-cyanobenzyl | C═O | absent | 4-cyanobenzyl |
| C═O | absent | 2-methylbenzyl | C═O | absent | 2-methylbenzyl |
| C═O | absent | 3-methylbenzyl | C═O | absent | 3-methylbenzyl |
| C═O | absent | 4-methylbenzyl | C═O | absent | 4-methylbenzyl |
| C═O | absent | cyclopropyl | C═O | absent | cyclopropyl |
| C═O | absent | 2-piperidinyl | C═O | absent | 2-piperidinyl |
| C═O | absent | 3-piperidinyl | C═O | absent | 3-piperidinyl |
| C═O | absent | 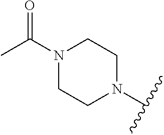 [SMILES: *N1CCN(C(C)=O)CC1] | C═O | absent | 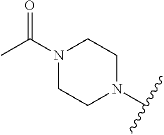 [SMILES: *N1CCN(C(C)=O)CC1] |
| C═O | absent | 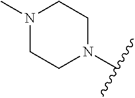 [SMILES: *N1CCN(C)CC1] | C═O | absent | 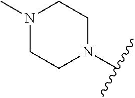 [SMILES: *N1CCN(C)CC1] |
| C═O | absent | 2-morpholinyl | C═O | absent | 2-morpholinyl |
| C═O | absent | 3-morpholinyl | C═O | absent | 3-morpholinyl |
| C═O | absent | 4-morpholinyl | C═O | absent | 4-morpholinyl |
| C═O | absent | 2-tetrahydrofuryl | C═O | absent | 2-tetrahydrofuryl |
| C═O | absent | 3-tetrahydrofuryl | C═O | absent | 3-tetrahydrofuryl |
| C═O | absent | 2-pyrrolidinyl | C═O | absent | 2-pyrrolidinyl |
| C═O | absent | 3-pyrrolidinyl | C═O | absent | 3-pyrrolidinyl |
| C═O | absent | 2-tetrahydrothienyl | C═O | absent | 2-tetrahydrothienyl |
| C═O | absent | 3-tetrahydrothienyl | C═O | absent | 3-tetrahydrothienyl |
| C═O | absent | 1-piperazinyl | C═O | absent | 1-piperazinyl |
| C═O | absent | 2-piperazinyl | C═O | absent | 2-piperazinyl |
| C═O | CH₂ | —OH | C═O | CH₂ | —OH |
| C═O | CH₂ | —OMe | C═O | CH₂ | —OMe |
| C═O | CH₂ | —NH₂ | C═O | CH₂ | —NH₂ |
| C═O | CH₂ | —NHMe | C═O | CH₂ | —NHMe |
| C═O | CH₂ | —CH₂OH | C═O | CH₂ | —CH₂OH |
| C═O | CH₂ | —CH₂OMe | C═O | CH₂ | —CH₂OMe |
| C═O | CH₂ | —CH₂NH₂ | C═O | CH₂ | —CH₂NH₂ |
| C═O | CH₂ | —CH₂NHMe | C═O | CH₂ | —CH₂NHMe |
| C═O | CH₂ | Me | C═O | CH₂ | Me |
| C═O | CH₂ | Et | C═O | CH₂ | Et |
| C═O | CH₂ | iPr | C═O | CH₂ | iPr |
| C═O | CH₂ | iBu | C═O | CH₂ | iBu |
| C═O | CH₂ | —CH₂OH | C═O | CH₂ | —CH₂OH |
| C═O | CH₂ | Ph | C═O | CH₂ | Ph |
| C═O | CH₂ | 3-pyridyl | C═O | CH₂ | 3-pyridyl |
| C═O | CH₂ | 2-pyridyl | C═O | CH₂ | 2-pyridyl |
| C═O | CH₂ | 4-pyridyl | C═O | CH₂ | 4-pyridyl |
| C═O | CH₂ | 5-pyrimidinyl | C═O | CH₂ | 5-pyrimidinyl |
| C═O | CH₂ | 4-pyrimidinyl | C═O | CH₂ | 4-pyrimidinyl |
| C═O | CH₂ | 2-pyrimidinyl | C═O | CH₂ | 2-pyrimidinyl |
| C═O | CH₂ | 2-chlorobenzyl | C═O | CH₂ | 2-chlorobenzyl |
| C═O | CH₂ | 3-chlorobenzyl | C═O | CH₂ | 3-chlorobenzyl |
| C═O | CH₂ | 4-chlorobenzyl | C═O | CH₂ | 4-chlorobenzyl |
| C═O | CH₂ | 2-fluorobenzyl | C═O | CH₂ | 2-fluorobenzyl |
| C═O | CH₂ | 3-fluorobenzyl | C═O | CH₂ | 3-fluorobenzyl |
| C═O | CH₂ | 4-fluorobenzyl | C═O | CH₂ | 4-fluorobenzyl |
| C═O | CH₂ | 2-cyanobenzyl | C═O | CH₂ | 2-cyanobenzyl |
| C═O | CH₂ | 3-cyanobenzyl | C═O | CH₂ | 3-cyanobenzyl |
| C═O | CH₂ | 4-cyanobenzyl | C═O | CH₂ | 4-cyanobenzyl |
| C═O | CH₂ | 2-methylbenzyl | C═O | CH₂ | 2-methylbenzyl |
| C═O | CH₂ | 3-methylbenzyl | C═O | CH₂ | 3-methylbenzyl |
| C═O | CH₂ | 4-methylbenzyl | C═O | CH₂ | 4-methylbenzyl |
| C═O | CH₂ | cyclopropyl | C═O | CH₂ | cyclopropyl |
| C═O | CH₂ | 2-piperidinyl | C═O | CH₂ | 2-piperidinyl |
| C═O | CH₂ | 3-piperidinyl | C═O | CH₂ | 3-piperidinyl |
| C═O | CH₂ | 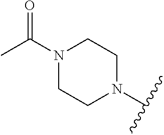 [SMILES: *N1CCN(C(C)=O)CC1] | | CH₂ | 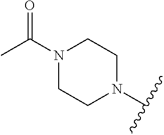 [SMILES: *N1CCN(C(C)=O)CC1] |
| C═O | CH₂ | 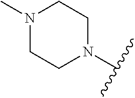 [SMILES: *N1CCN(C)CC1] | C═O | CH₂ | 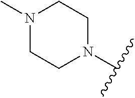 [SMILES: *N1CCN(C)CC1] |
| C═O | CH₂ | 2-morpholinyl | C═O | CH₂ | 2-morpholinyl |
| C═O | CH₂ | 3-morpholinyl | C═O | CH₂ | 3-morpholinyl |
| C═O | CH₂ | 4-morpholinyl | C═O | CH₂ | 4-morpholinyl |
| C═O | CH₂ | 1-piperazinyl | C═O | CH₂ | 1-piperazinyl |
| C═O | CH₂ | 2-piperazinyl | C═O | CH₂ | 2-piperazinyl |
| C═O | —CH(NH₂)— | Me | C═O | —CH(NH₂)— | Me |
| C═O | —CH(NH₂)— | Et | C═O | —CH(NH₂)— | Et |
| C═O | —CH(NH₂)— | iPr | C═O | —CH(NH₂)— | iPr |
| C═O | —CH(NH₂)— | iBu | C═O | —CH(NH₂)— | iBu |
| C═O | —CH(NH₂)— | —CH₂OH | C═O | —CH(NH₂)— | —CH₂OH |
| C═O | NH | Ph | C═O | NH | Ph |
| C═O | NH | 3-pyridyl | C═O | NH | 3-pyridyl |
| C═O | NH | 2-pyridyl | C═O | NH | 2-pyridyl |
| C═O | NH | 4-pyridyl | C═O | NH | 4-pyridyl |
| C═O | NH | 5-pyrimidinyl | C═O | NH | 5-pyrimidinyl |
| C═O | NH | 4-pyrimidinyl | C═O | NH | 4-pyrimidinyl |
| C═O | NH | 2-pyrimidinyl | C═O | NH | 2-pyrimidinyl |
| C═O | NH | 2-chlorobenzyl | C═O | NH | 2-chlorobenzyl |
| C═O | NH | 3-chlorobenzyl | C═O | NH | 3-chlorobenzyl |
| C═O | NH | 4-chlorobenzyl | C═O | NH | 4-chlorobenzyl |
| C═O | NH | 2-fluorobenzyl | C═O | NH | 2-fluorobenzyl |
| C═O | NH | 3-fluorobenzyl | C═O | NH | 3-fluorobenzyl |
| C═O | NH | 4-fluorobenzyl | C═O | NH | 4-fluorobenzyl |
| C═O | NH | 2-cyanobenzyl | C═O | NH | 2-cyanobenzyl |
| C═O | NH | 3-cyanobenzyl | C═O | NH | 3-cyanobenzyl |
| C═O | NH | 4-cyanobenzyl | C═O | NH | 4-cyanobenzyl |
| C═O | NH | 2-methylbenzyl | C═O | NH | 2-methylbenzyl |
| C═O | NH | 3-methylbenzyl | C═O | NH | 3-methylbenzyl |
| C═O | NH | 4-methylbenzyl | C═O | NH | 4-methylbenzyl |
| C═O | NH | cyclopropyl | C═O | NH | cyclopropyl |
| C═O | NH | 2-piperidinyl | C═O | NH | 2-piperidinyl |
| C═O | NH | 3-piperidinyl | C═O | NH | 3-piperidinyl |
| C═O | NH | 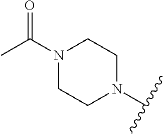 [SMILES: *N1CCN(C(C)=O)CC1] | C═O | NH | 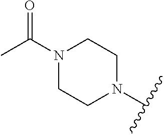 [SMILES: *N1CCN(C(C)=O)CC1] |
| C═O | NH | 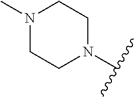 [SMILES: *N1CCN(C)CC1] | C═O | NH | 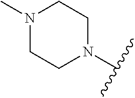 [SMILES: *N1CCN(C)CC1] |
| C═O | NH | 2-morpholinyl | C═O | NH | 2-morpholinyl |
| C═O | NH | 3-morpholinyl | C═O | NH | 3-morpholinyl |
| C═O | NH | 4-morpholinyl | C═O | NH | 4-morpholinyl |
| C═O | NH | 2-tetrahydrofuryl | C═O | NH | 2-tetrahydrofuryl |
| C═O | NH | 3-tetrahydrofuryl | C═O | NH | 3-tetrahydrofuryl |
| C═O | NH | 2-pyrrolidinyl | C═O | NH | 2-pyrrolidinyl |
| C═O | NH | 3-pyrrolidinyl | C═O | NH | 3-pyrrolidinyl |
| C═O | NH | 2-tetrahydrothienyl | C═O | NH | 2-tetrahydrothienyl |
| C═O | NH | 3-tetrahydrothienyl | C═O | NH | 3-tetrahydrothienyl |
| C═O | NH | 1-piperazinyl | C═O | NH | 1-piperazinyl |
| C═O | NH | 2-piperazinyl | C═O | NH | 2-piperazinyl |
| C═O | —NHCH₂— | —CH₂OH | C═O | —NHCH₂— | —CH₂OH |
| C═O | —NHCH₂— | —CH₂OMe | C═O | —NHCH₂— | —CH₂OMe |
| C═O | —NHCH₂— | —CH₂NH₂ | C═O | —NHCH₂— | —CH₂NH₂ |
| C═O | —NHCH₂— | —CH₂NHMe | C═O | —NHCH₂— | —CH₂NHMe |
| C═O | O | Me | C═O | O | Me |
| C═O | O | Et | C═O | O | Et |
| C═O | O | iPr | C═O | O | iPr |
| C═O | O | iBu | C═O | O | iBu |
| C═O | O | Ph | C═O | O | Ph |
| absent | SO₂ | Me | absent | SO₂ | Me |
| absent | SO₂ | Ph | absent | SO₂ | Ph |

### Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (American Chemical Society, Washington, D.C.). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the complement factor D inhibitory compound described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the complement factor D inhibitory compound as described herein is administered as a pure chemical. In other embodiments, the complement factor D inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Provided herein is a pharmaceutical composition comprising at least one complement factor D inhibitory compound, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable excipient and a compound of Formula (I) or (Ia), or a pharmaceutically acceptable salt thereof.

In certain embodiments, the complement factor D inhibitory compound as described by Formula (I) or (Ia) is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, Pa. (2005)).

The dose of the composition comprising at least one complement factor D inhibitory compound as described herein differ, depending upon the patient's (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Complement Factor D and Methods of Treatment

Complement Factor D (also referred to as C3 proactivator convertase, properdin factor D esterase, factor D (complement), CFD, or adipsin) is a protein which in humans is encoded by the CFD gene. Factor D is involved in the alternative complement pathway of the complement system where it cleaves factor B.

The complement factor D inhibitory compounds described herein function to modulate in vivo complement activation and/or the alternative complement pathway. In some embodiments, the complement factor D inhibitory compounds described herein function to inhibit in vivo complement activation and/or the alternative complement pathway. Accordingly, provided herein is a method of treating a disease or disorder associated with increased complement activity, the method comprising administering to a subject in need thereof a complement factor D inhibitory compound described herein. In some embodiments, the disease or disorder associated with increased complement activity is a disease or disorder associated with increased activity of the C3 amplification loop of the complement pathway.

Exemplary complement related diseases and disorders include, but are not limited to, autoimmune, inflammatory, and neurodegenerative diseases. In certain instances, the complement related diseases and disorder is paraoxysmal nocturnal hemoglobinuria. One embodiment provides a method for treating paraoxysmal nocturnal hemoglobinuria in a patient in need thereof, comprising administering to the patient a composition comprising a compound of Formula (I), or a pharmaceutically acceptable salt thereof. One embodiment provides a method for treating paraoxysmal nocturnal hemoglobinuria in a patient in need thereof, comprising administering to the patient a composition comprising a compound of Formula (Ia), or a pharmaceutically acceptable salt thereof. One embodiment provides a method for treating paraoxysmal nocturnal hemoglobinuria in a patient in need thereof, comprising administering to the patient a composition comprising a therapeutically effective amount of a compound of Formula (I), or a pharmaceutically acceptable salt thereof. One embodiment provides a method for treating paraoxysmal nocturnal hemoglobinuria in a patient in need thereof, comprising administering to the patient a composition comprising a therapeutically effective amount of a compound of Formula (Ia), or a pharmaceutically acceptable salt thereof.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (δ) and coupling constants, J are reported in Hertz. For proton spectra the solvent peak was used as the reference peak.

The following abbreviations and terms have the indicated meanings throughout:
AcOH=acetic acid
B₂pin₂=bis(pinacolato)diboron
Boc=tert-butoxycarbonyl
DCC=dicyclohexylcarbodiimide
DIEA=N,N-diisopropylethylamine
DMAP=4-dimethylaminopyridine
EDC=1-ethyl-3-(3-dimethylaminopropyl) carbodiimide
eq=equivalent(s)
Et=ethyl
EtOAc or EA=ethyl acetate
EtOH=ethanol
g=gram
h or hr=hour
HBTU=O-(benzotriazol-1-yl)-N,N,N′,N′-tetramethyluronium hexafluorophosphate
HOBt=hydroxybenzotriazole
HPLC=high pressure liquid chromatography
kg or Kg=kilogram
L or l=liter
LC/MS=LCMS=liquid chromatography-mass spectrometry
LRMS=low resolution mass spectrometry
m/z=mass-to-charge ratio
Me=methyl
MeOH=methanol
mg=milligram
min=minute
mL=milliliter
mmol=millimole
NaOAc=sodium acetate
PE=petroleum ether
Ph=phenyl
Prep=preparative
quant.=quantitative
RP-HPLC=reverse phase-high pressure liquid chromatography
rt or RT=room temperature
THF=tetrahydrofuran
UV=ultraviolet

### Preparation of 2-(3-carbamoyl-1H-indazol-1-yl)acetic acid

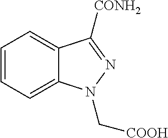 [SMILES: NC(=O)c1nn(CC(=O)O)c2ccccc12]

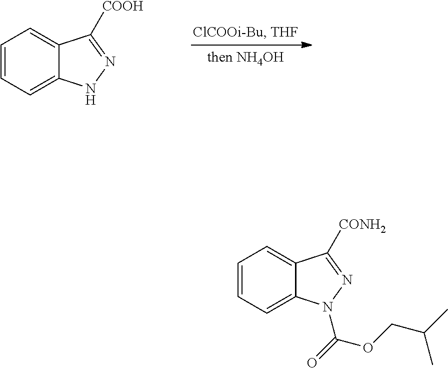 [SMILES: O=C(O)c1n[nH]c2ccccc12 ; CC(C)COC(=O)n1nc(C(N)=O)c2ccccc21]

To a solution of indazole 3-carboxylic acid (2.0 g, 12.4 mmol, 1.0 eq.) in anhydrous THF (30 mL) was added isobutyl chloroformate (2.6 g, 19.6 mmol, 1.5 eq.) and N-methylmorpholine (2.0 g, 19.6 mmol, 1.5 eq.) under nitrogen at −20° C. The mixture was stirred for 2 h, then 3.4 mL of NH₄OH was added. After the addition was complete, the mixture was stirred at rt for 1 h, then quenched by water. The mixture was extracted with EtOAc (2×50 mL). The combined organic layers were dried over anhydrous Na₂SO₄ and concentrated. The resulting residue was purified by column chromatography (CH₂Cl₂/MeOH=20:1) to provide isobutyl 3-carbamoyl-1H-indazole-1-carboxylate as a white solid (1.7 g, 52.4).

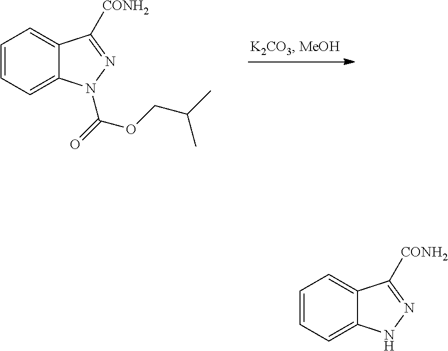 [SMILES: CC(C)COC(=O)n1nc(C(N)=O)c2ccccc21 ; NC(=O)c1n[nH]c2ccccc12]

To a solution of isobutyl 3-carbamoyl-1H-indazole-1-carboxylate (1.7 g, 6.5 mmol, 1.0 eq.) in MeOH (20 mL) was added K₂CO₃ (1.8 g, 13.0 mmol, 2.0 eq.). The mixture was stirred at 80° C. for 2 h, cooled, then quenched by water. The mixture was extracted with EtOAc (2×50 mL). The combined organic layers were dried over anhydrous Na₂SO₄ and concentrated. The residue was purified by column chromatography (CH₂Cl₂/MeOH=20:1) to provide 1H-indazole-3-carboxamide as a white solid (1.0 g, 94.8%).

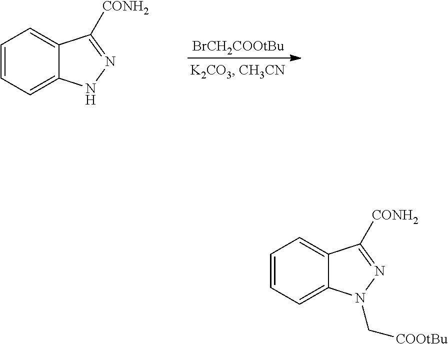

To a suspension of 1H-indazole-3-carboxamide (1.0 g, 6.2 mmol, 1.0 eq.) and potassium carbonate (2.1 g, 14.9 mmol, 2.4 eq.) in CH₃CN (30 mL) was added tert-butyl bromoacetate (1.1 mL, 7.4 mmol, 1.2 eq.) dropwise at rt. After the addition was complete, the resulting mixture was heated under reflux for 16 h, then cooled and filtered. The filtrate was concentrated in vacuum and the residue was purified by silica column chromatography (PE/EA=20:1) to provide tert-butyl 2-(3-carbamoyl-1H-indazol-1-yl)acetate (1.6 g, 93.6%).

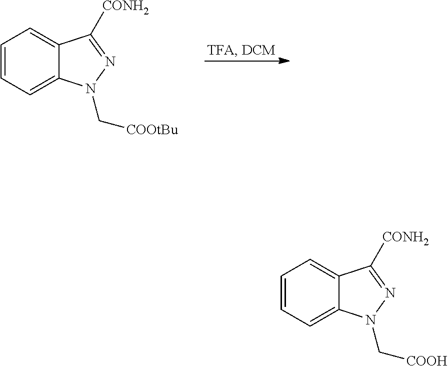

To a solution of tert-butyl 2-(3-carbamoyl-1H-indazol-1-yl)acetate (1.6 g, 5.8 mmol) in CH₂Cl₂ (16 mL) was added TFA (4 mL). The resulting mixture was stirred at rt for 16 h, then concentrated. The resulting residue was triturated in methanol and filtered to provide 2-(3-carbamoyl-1H-indazol-1-yl)acetic acid (1.0 g, 78.0%), which was used in the next step without further purification.

### Preparation of 2-(3-carbamoyl-1H-pyrazolo[3,4-c]pyridin-1-yl)acetic acid

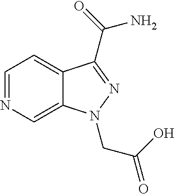 [SMILES: NC(=O)c1nn(CC(=O)O)c2cnccc12]

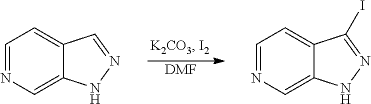

To a solution of 1H-pyrazolo[3,4-c]pyridine (4.0 g, 33.6 mmol, 1.0 eq.) in DMF (40 mL) were added K₂CO₃ (9.3 g, 100.8 mmol, 3.0 eq.) and I₂ (7.9 g, 33.6 mmol, 1.0 eq.). The resulting mixture was stirred at rt for 3 h, then diluted by H₂O and filtered. The solid was collected and dried to give 3-iodo-1H-pyrazolo[3,4-c]pyridine (6.0 g, 73.0% yield).

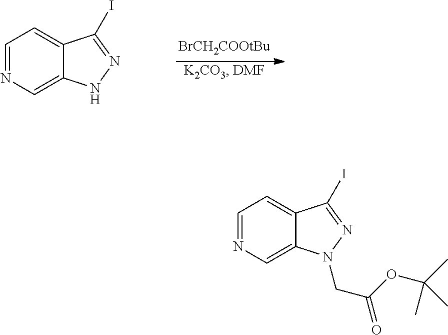

To a solution of 3-iodo-1H-pyrazolo[3,4-c]pyridine (6.0 g, 24.5 mmol, 1.0 eq.) and K₂CO₃ (4.0 g, 29.4 mmol, 1.2 eq.) in DMF (40 mL) was added tert-butyl 2-bromoacetate (4.78 g, 24.5 mmol, 1.0 eq.). The resulting mixture was stirred at rt for 2 h, poured into water (200 mL), and extracted with EtOAc (200 mL×3). The combined organic layers were dried and concentrated. The residue was purified by column chromatography (PE/EtOAc=3:1) to PROVIDE tert-butyl 2-(3-iodo-1H-pyrazolo[3,4-c]pyridin-1-yl)acetate as a yellow oil (6.0 g, 68.0% yield).

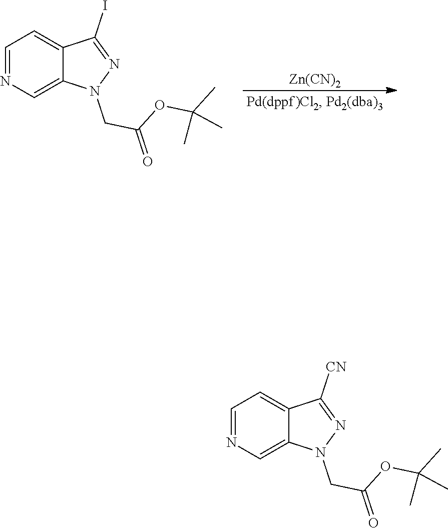

To a solution of tert-butyl 2-(3-iodo-1H-pyrazolo[3,4-c]pyridin-1-yl)acetate (6.0 g, 16.7 mmol, 1.0 eq.) and Zn(CN)₂ (2.3 g, 20.0 mmol, 1.2 eq.) in H₂O/DMF (5/35 ml) were added Pd(dppf)Cl2 (1.2 g, 1.6 mmol, 0.1 eq.), Pd2(dba)₃ (1.5 g, 1.6 mmol, 0.1 eq.). The resulting mixture was stirred at 80° C. for 1 h, cooled and poured into water (200 ml), and extracted with EtOAc (200 ml×3). The combined organic layers were dried over anhydrous Na₂SO₄ and concentrated. The residue was purified by column chromatography (PE/EtOAc=5:1) to give tert-butyl 2-(3-cyano-1H-pyrazolo[3,4-c]pyridin-1-yl)acetate (3.5 g, 81.0% yield).

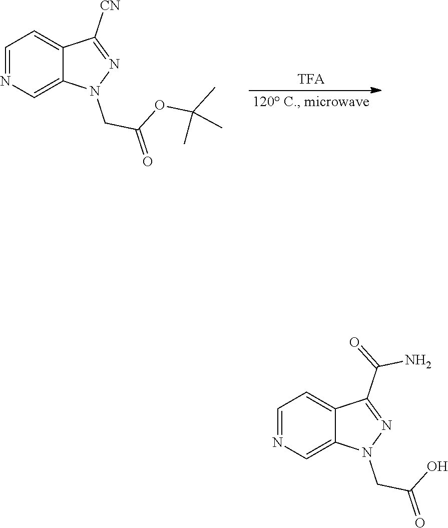 [SMILES: CC(C)(C)OC(=O)Cn1nc(C#N)c2ccncc21 ; "TFA" ; NC(=O)c1nn(CC(=O)O)c2cnccc12]

A solution of tert-butyl 2-(3-cyano-1H-pyrazolo[3,4-c]pyridin-1-yl)acetate (500 mg, 2.0 mmol) in TFA (2 mL) was stirred at 120° C. for 3 h under microwave irradiation, then cooled and concentrated under vacuum to provide crude 2-(3-carbamoyl-1H-pyrazolo[3,4-c]pyridin-1-yl)acetic acid (450 mg, ca. 100% yield) which was used in the next step without further purification.

### Example 1: Preparation of (S)-1-(2-((2-((3-chloro-2-fluorobenzyl)amino)-1-cyclopropyl-2-oxoethyl)amino)-2-oxoethyl)-1H-indazole-3-carboxamide

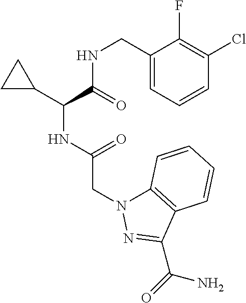 [SMILES: NC(=O)c1nn(CC(=O)N[C@H](C(=O)NCc2cccc(Cl)c2F)C2CC2)c2ccccc12]

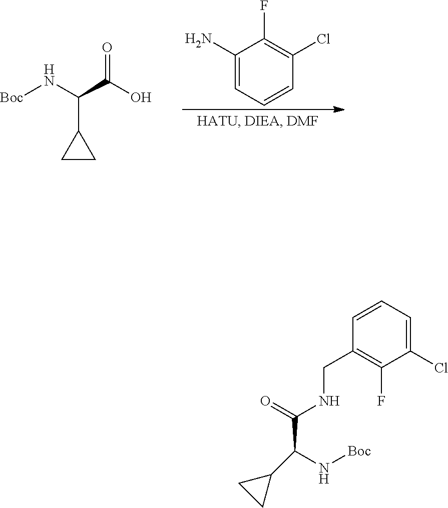 [SMILES: CC(C)(C)OC(=O)N[C@@H](C(=O)O)C1CC1 ; Nc1cccc(Cl)c1F ; CC(C)(C)OC(=O)N[C@H](C(=O)NCc1cccc(Cl)c1F)C1CC1]

To a solution of (S)-2-((tert-butoxycarbonyl)amino)-2-cyclopropylacetic acid (100.0 mg, 0.5 mmol, 1.0 eq.) and (3-chloro-2-fluorophenyl)methanamine (81.0 mg, 0.5 mmol, 1.0 eq.) in DMF (4 mL) were added HATU (264.0 mg, 0.7 mmol, 1.5 eq.) and DIEA (180.0 mg, 1.4 mmol, 3.0 eq.). The mixture was stirred at r.t. for 4 h. Ethyl acetate (50 mL) and water (50 mL) were added. The organic layer was separated and concentrated to provide crude (S)-tert-butyl (2-((3-chloro-2-fluorobenzyl)amino)-1-cyclopropyl-2-oxoethyl)carbamate (120.0 mg), which was used in the next step directly without further purification.

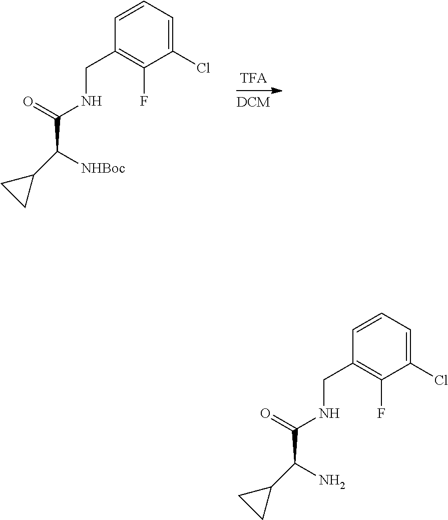 [SMILES: CC(C)(C)OC(=O)N[C@H](C(=O)NCc1cccc(Cl)c1F)C1CC1 ; N[C@H](C(=O)NCc1cccc(Cl)c1F)C1CC1]

To a solution of (S)-tert-butyl (2-((3-chloro-2-fluorobenzyl)amino)-1-cyclopropyl-2-oxoethyl)carbamate (48.0 mg) in dichloromethane (3.0 mL) was added TFA (1.0 mL). The mixture was stirred at r.t. for 3 h. The solution was concentrated and was added ethyl acetate (50.0 mL). The organic layer was washed with aqueous 15% NaHCO₃ solution (50 mL), dried over anhydrous anhydrous Na₂SO₄, filtered and concentrated to provide (S)-2-amino-N-(3-chloro-2-fluorobenzyl)-2-cyclopropylacetamide (30.0 mg, 87% yield in 2 steps).

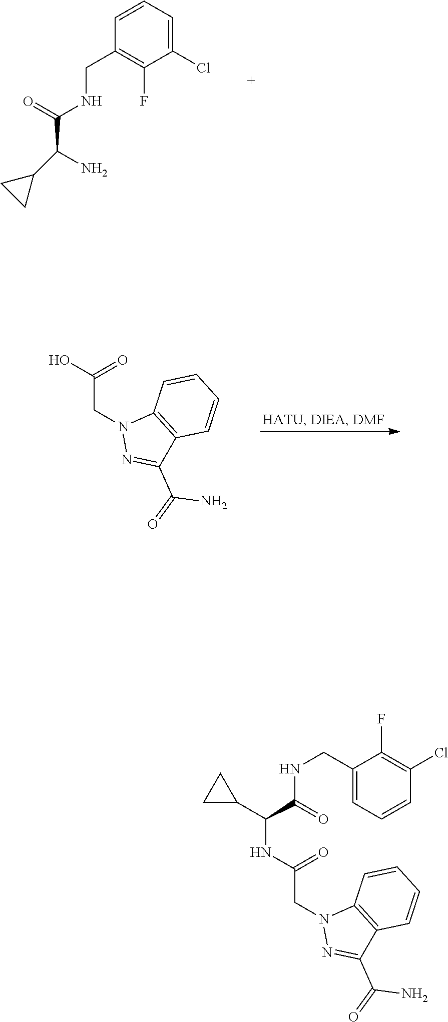 [SMILES: N[C@H](C(=O)NCc1cccc(Cl)c1F)C1CC1 ; NC(=O)c1nn(CC(=O)O)c2ccccc12 ; NC(=O)c1nn(CC(=O)N[C@H](C(=O)NCc2cccc(Cl)c2F)C2CC2)c2ccccc12]

To a solution of (S)-2-amino-N-(3-chloro-2-fluorobenzyl)-2-cyclopropylacetamide (30.0 mg, 0.1 mmol, 1.0 eq.) and 2-(3-carbamoyl-1H-indazol-1-yl)acetic acid (40.0 mg, 0.1 mmol, 1.0 eq.) in DMF (3.0 mL) were added HATU (66.0 mg, 0.2 mmol, 1.5 eq.) and DIEA (45.0 mg, 0.4 mmol, 3.0 eq.). The mixture was stirred at r.t. for 1 h. Water (50 mL) was added and the mixture was filtered. The filtrate was washed with dichloromethane/petroleum ether (1:5 10 mL) and evaporated. The residue was purified by flash chromatography (MeOH/PE=1/10) to provide 1-(2-((1-((3-chloro-2-fluorobenzyl)carbamoyl)cyclopropyl)amino)-2-oxoethyl)-1H-indazole-3-carboxamide (30.0 mg, 54% yield). ¹H NMR (DMSO-d₆, 400 MHz) δ=8.76 (d, 1H), 8.60 (s, 1H), 8.17 (d, 1H), 7.65 (t, 2H), 7.37-7.49 (m, 3H), 7.25 (t, 2H), 7.14 (t, 1H), 5.24 (d, 2H), 4.29-4.43 (m, 2H), 3.78 (t, 1H), 1.10-1.13 (m, 1H), 0.49 (d, 3H), 0.29 (d, 1H). LRMS (M+H⁺) m/z calculated 458.1, found 458.6.

### Example 2: Preparation of (S)-1-(2-((1-((3-chloro-2-fluorobenzyl)carbamoyl)cyclopropyl)amino)-2-oxoethyl)-1H-indazole-3-carboxamide

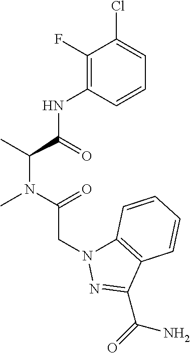 [SMILES: C[C@@H](C(=O)Nc1cccc(Cl)c1F)N(C)C(=O)Cn1nc(C(N)=O)c2ccccc21]

(S)-1-(2-((1-((3-chloro-2-fluorobenzyl)carbamoyl)cyclopropyl)amino)-2-oxoethyl)-1H-indazole-3-carboxamide (3.2 mg) was prepared as described for (S)-1-(2-((2-((3-chloro-2-fluorobenzyl)amino)-1-cyclopropyl-2-oxoethyl)amino)-2-oxoethyl)-1H-indazole-3-carboxamide. ¹H NMR (DMSO-d6, 400 MHz) δ 8.36-8.32 (m, 2H), 8.07-8.06 (m, 1H), 7.47-7.33 (m, 4H), 7.14-6.99 (m, 3H), 6.44 (s, 1H), 5.34 (s, 2H), 3.09-2.92 (s, 3H), 1.47-1.43 (m, 3H). LRMS (M+H⁺) m/z calculated 432.1, found 432.7.

### Example 3: Preparation of (S)-1-(2-((1-((3-chloro-2-fluorobenzyl)amino)-1-oxopropan-2-yl)(methyl)amino)-2-oxoethyl)-1H-indazole-3-carboxamide

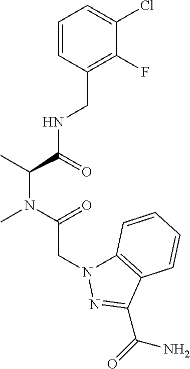 [SMILES: C[C@@H](C(=O)NCc1cccc(Cl)c1F)N(C)C(=O)Cn1nc(C(N)=O)c2ccccc21]

(S)-1-(2-((1-((3-chloro-2-fluorobenzyl)amino)-1-oxopropan-2-yl)(methyl)amino)-2-oxoethyl)-1H-indazole-3-carboxamide (74.3 mg) was prepared as described for (S)-1-(2-((2-((3-chloro-2-fluorobenzyl)amino)-1-cyclopropyl-2-oxoethyl)amino)-2-oxoethyl)-1H-indazole-3-carboxamide. ¹H NMR (CDCl₃, 400 MHz) δ 8.39-8.37 (m, 1H), 7.44-7.42 (m, 2H), 7.34-7.26 (m, 4H), 7.12 (s, 1H), 7.00 (s, 1H), 6.83 (s, 1H), 6.51 (s, 1H), 5.41 (s, 1H),5.27 (s, 2H). 5.11-5.09 (m, 1H). 4.47-4.38 (m, 2H). 3.06-3.02 (s, 3H). 2.05 (s, 1H). LRMS (M+H⁺) m/z calculated 446.1, found 446.6.

### Example 4: Preparation of 1-(2-((2-((3-chloro-2-fluorobenzyl)amino)-2-oxoethyl)(cyclopropyl)amino)-2-oxoethyl)-1H-indazole-3-carboxamide

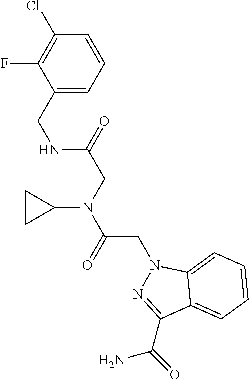 [SMILES: NC(=O)c1nn(CC(=O)N(CC(=O)NCc2cccc(Cl)c2F)C2CC2)c2ccccc12]

1-(2-((2-((3-chloro-2-fluorobenzyl)amino)-2-oxoethyl)(cyclopropyl)amino)-2-oxoethyl)-1H-indazole-3-carboxamide (57.0 mg) was prepared as described for (S)-1-(2-((2-((3-chloro-2-fluorobenzyl)amino)-1-cyclopropyl-2-oxoethyl)amino)-2-oxoethyl)-1H-indazole-3-carboxamide. ¹H NMR (DMSO-d6, 400 MHz) δ=8.48-8.47 (m, 1H), 8.19-7.17 (m, 1H), 7.70-7.66 (m, 1H), 7.65-7.63 (m, 1H), 7.48-7.37 (m, 3H), 7.28-7.13 (m, 2H), 7.11-7.10 (m, 1H), 5.66 (s, 2H), 4.33 (s, 2H), 3.98 (s, 2H), 3.07 (s, 1H), 1.00-0.91 (m, 4H). LRMS (M+H⁺) m/z calculated 458.1, found 458.6.

### Example 5: Preparation of 1-(2-((2-((3-chloro-2-fluorobenzyl)amino)-2-oxoethyl)(isopropyl)amino)-2-oxoethyl)-1H-indazole-3-carboxamide

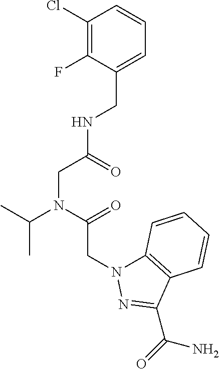 [SMILES: CC(C)N(CC(=O)NCc1cccc(Cl)c1F)C(=O)Cn1nc(C(N)=O)c2ccccc21]

1-(2-((2-((3-chloro-2-fluorobenzyl)amino)-2-oxoethyl)(isopropyl)amino)-2-oxoethyl)-1H-indazole-3-carboxamide (60.0 mg) was prepared as described for (S)-1-(2-((2-((3-chloro-2-fluorobenzyl)amino)-1-cyclopropyl-2-oxoethyl)amino)-2-oxoethyl)-1H-indazole-3-carboxamide. ¹H NMR (CD₃OD, 400 MHz) δ=8.23-8.21 (m, 1H), 7.61-7.56 (m, 1H), 7.46-7.38 (m, 2H), 7.33-7.22 (m, 2H), 7.13-7.98 (m, 1H), 5.58 (s, 1H), 5.46 (s, 1H), 4.71-4.37 (m, 3H), 4.22 (s, 1H), 3.97 (s, 1H), 1.28 (t, 3H), 1.09 (t, 3H). LRMS (M+H⁺) m/z calculated 460.1, found 460.2.

### Example 6: Preparation of 1-(2-((2-((3-chloro-2-fluorobenzyl)amino)-2-oxoethyl)(ethyl)amino)-2-oxoethyl)-1H-indazole-3-carboxamide

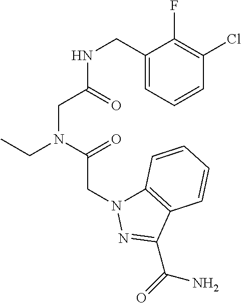 [SMILES: CCN(CC(=O)NCc1cccc(Cl)c1F)C(=O)Cn1nc(C(N)=O)c2ccccc21]

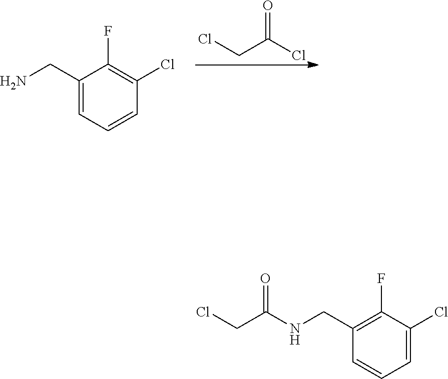 [SMILES: NCc1cccc(Cl)c1F ; O=C(Cl)CCl ; O=C(CCl)NCc1cccc(Cl)c1F]

To a solution of (3-chloro-2-fluorophenyl)methanamine (1.6 g, 10.0 mmol, 1.0 eq.) and Et₃N (1.51 g, 15.0 mmol, 1.0 eq.) in dichloromethane (100 mL) was added 2-chloroacetyl chloride (1.7 g, 15.0 mmol, 1.5 eq.). The resulting mixture was stirred at r.t. for 16 h. The mixture was washed with aq. 10% NaHCO₃ solution (100 mL), dried over anhydrous Na₂SO₄, filtered and concentrated to provide crude 2-chloro-N-(3-chloro-2-fluorobenzyl)acetamide (2.20 g, 93% yield).

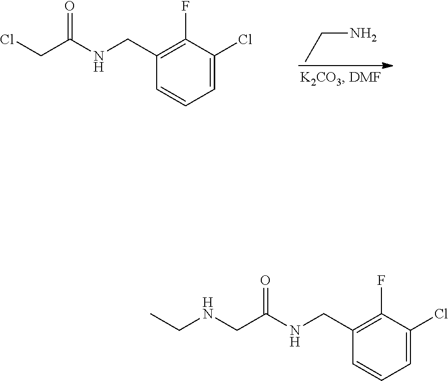

To a solution of 2-chloro-N-(3-chloro-2-fluorobenzyl)acetamide (150 mg, 0.6 mmol, 1.0 eq.) and K₂CO₃ (176 mg, 1.27 mmol, 2.0 eq.) in DMF (3 mL) was added a solution of ethanamine in methanol (6.0 N, 0.16 mL, 0.96 mmol, 1.5 eq.). The reaction was stirred at r.t. for 16 h. Ethyl acetate (50 mL) and water (50 mL) were added. The organic layer was separated and concentrated. The residue was purified by Prep-TLC (DCM/MeOH 10:1) to provide N-(3-chloro-2-fluorobenzyl)-2-(ethylamino)acetamide (44 mg, 28% yield).

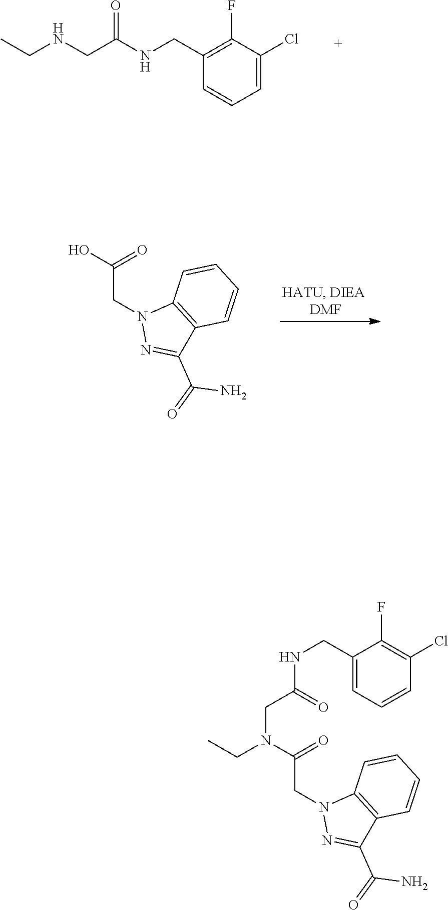 [SMILES: CCNCC(=O)NCc1cccc(Cl)c1F ; NC(=O)c1nn(CC(=O)O)c2ccccc12 ; CCN(CC(=O)NCc1cccc(Cl)c1F)C(=O)Cn1nc(C(N)=O)c2ccccc21]

To a solution of N-(3-chloro-2-fluorobenzyl)-2-(ethylamino)acetamide (25 mg, 0.1 mmol, 1.0 eq.) and 2-(3-carbamoyl-1H-indazol-1-yl)acetic acid (34 mg, 0.1 mmol, 1.0 eq.) in DMF (3 mL) were added HATU (58 mg, 0.15 mmol, 1.5 eq.) and DIEA (53 mg, 0.41 mmol, 4.0 eq.). The mixture was stirred at r.t. for 16 h. Ethyl acetate (50 mL) and water (50 mL) were added. The organic layer was separated and concentrated. The residue was purified by prep-TLC (DCM/MeOH=10:1) to provide 1-(2-((2-((3-chloro-2-fluorobenzyl)amino)-2-oxoethyl)(ethyl)amino)-2-oxoethyl)-1H-indazole-3-carboxamide (29 mg, 69% yield). ¹H NMR (MeOD, 400 MHz) δ=8.22 (d, 1H), 7.56 (d, 1H), 7.43-7.23 (m, 4H), 7.16-7.00 (m, 1H), 5.57 (s, 1H), 5.45 (s, 1H), 4.55 (s, 1H), 4.45 (s, 1H), 4.30 (s, 1H), 4.08 (s, 1H), 3.64 (q, 1H), 3.41 (q, 1H), 1.29 (t, 2H), 1.09 (t, 1H). LRMS (M+H⁺) m/z calculated 446.1, found 446.2.

### Example 7: Preparation of 1-(2-((2-((3-chloro-2-fluorophenyl)amino)-2-oxoethyl)(cyclopropyl)amino)-2-oxoethyl)-1H-indazole-3-carboxamide

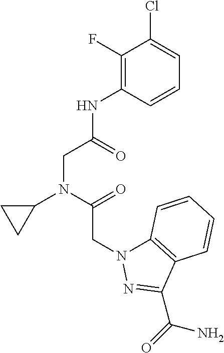 [SMILES: NC(=O)c1nn(CC(=O)N(CC(=O)Nc2cccc(Cl)c2F)C2CC2)c2ccccc12]

1-(2-((2-((3-chloro-2-fluorophenyl)amino)-2-oxoethyl)(cyclopropyl)amino)-2-oxoethyl)-1H-indazole-3-carboxamide (18.5 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ 8.39-8.37 (d, 1H), 8.11-8.09 (s, 2H), 7.44-7.26 (m, 4H), 7.19-7.05 (m, 3H), 6.84 (m, 1H), 5.54 (s, 2H), 4.24 (s, 2H), 1.10 (m, 4H). LRMS (M+H⁺) m/z calculated 444.1, found 444.5.

### Example 8: Preparation of 3-(2-((2-((3-chloro-2-fluorobenzyl)amino)-2-oxoethyl)(cyclopropyl)amino)-2-oxoethyl)-1H-indazole-1-carboxamide

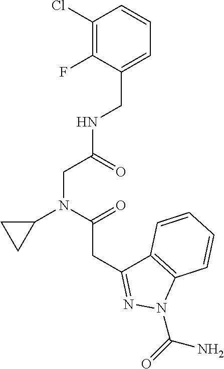 [SMILES: NC(=O)n1nc(CC(=O)N(CC(=O)NCc2cccc(Cl)c2F)C2CC2)c2ccccc21]

3-(2-((2-((3-chloro-2-fluorobenzyl)amino)-2-oxoethyl)(cyclopropyl)amino)-2-oxoethyl)-1H-indazole-1-carboxamide (18.9 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=8.48 (m, 1H), 8.18 (m, 1H), 7.70 (m, 1H), 7.63 (m, 1H), 7.42 (m, 3H), 7.20 (m, 2H), 7.10 (m, 1H), 5.66 (s, 2H), 4.33 (s, 2H), 3.98 (s, 2H), 3.07 (s, 1H), 2.31-2.28 (m, 1H), 0.95 (m, 4H). LRMS (M+H⁺) m/z calculated 458.1, found 458.6.

### Example 9: Preparation of 1-(2-(cyclopropyl(2-((5-hydroxy-3,3-dimethylcyclohexyl)amino)-2-oxoethyl)amino)-2-oxoethyl)-1H-indazole-3-carboxamide

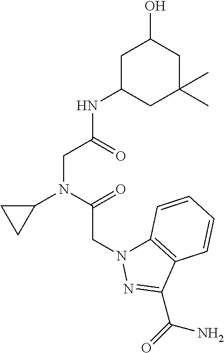 [SMILES: CC1(C)CC(O)CC(NC(=O)CN(C(=O)Cn2nc(C(N)=O)c3ccccc32)C2CC2)C1]

1-(2-(cyclopropyl(2-((5-hydroxy-3,3-dimethylcyclohexyl)amino)-2-oxoethyl)amino)-2-oxoethyl)-1H-indazole-3-carboxamide (7.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CD3OD, 400 MHz) δ=8.21 (d, 1H), 7.60 (d, 1H), 7.45 (t, 1H), 7.28 (t, 1H), 5.69 (s, 2H), 4.05 (s, 1H), 3.91 (s, 1H), 3.76 (s, 1H), 3.10 (s, 1H), 2.14 (s, 1H), 1.86 (d, 1H), 1.50-1.58 (m, 2H), 1.29-1.42 (m, 8H), 0.96-1.19 (m, 5H). LRMS (M+H⁺) m/z calculated 442.2, found 442.3.

### Example 10: Preparation of 1-(2-(cyclopropyl(2-(((1S,5S)-5-hydroxy-3,3-dimethylcyclohexyl)amino)-2-oxoethyl)amino)-2-oxoethyl)-1H-indazole-3-carboxamide

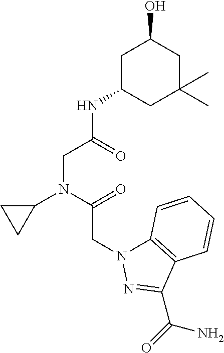 [SMILES: CC1(C)C[C@H](O)C[C@@H](NC(=O)CN(C(=O)Cn2nc(C(N)=O)c3ccccc32)C2CC2)C1]

1-(2-(cyclopropyl(2-(((1S,5S)-5-hydroxy-3,3-dimethylcyclohexyl)amino)-2-oxoethyl)amino)-2-oxoethyl)-1H-indazole-3-carboxamide (54.5 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CD3OD, 400 MHz) δ=8.22 (d, J=8.0, 1H), 7.60 (d, J=8.2, 1H), 7.44 (t, J=7.5, 1H), 7.29 (d, J=7.5, 1H), 5.70 (s, 2H), 4.05 (s, 1H), 3.91 (s, 1H), 3.76 (s, 1H), 3.10 (s, 1H), 2.14 (s, 1H), 1.51-1.66 (m, 2H), 0.96-1.04 (m, 14H). LRMS (M+H⁺) m/z calculated 442.2, found 442.3.

### Example 11: Preparation of 1-(2-((2-((3-chloro-2-fluorophenyl)amino)-2-oxoethyl)(isopropyl)amino)-2-oxoethyl)-1H-indazole-3-carboxamide

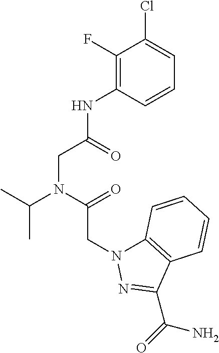 [SMILES: CC(C)N(CC(=O)Nc1cccc(Cl)c1F)C(=O)Cn1nc(C(N)=O)c2ccccc21]

1-(2-((2-((3-chloro-2-fluorophenyl)amino)-2-oxoethyl)(isopropyl)amino)-2-oxoethyl)-1H-indazole-3-carboxamide (18.5 mg) was prepared as described for 1-(2-((2-((3-chloro-2-fluorobenzyl) amino)-2-oxoethyl)(ethyl)amino)-2-oxoethyl)-1H-indazole-3-carboxamide. ¹H NMR (CDCl3, 400 MHz) δ 8.74 (s, 1H), 8.37 (s, 1H), 8.08 (s, 1H), 7.45-7.18 (m, 4H), 7.11-7.03 (m, 3H), 6.83 (m, 1H), 5.39 (m, 2H), 4.10 (m, 2H), 1.19-1.18 (m, 6H). LRMS (M+H⁺) m/z LRMS (M+H⁺) m/z calculated 446.1, found 446.5.

### Example 12: Preparation of 1-(2-((2-((6-chloropyridin-2-yl)amino)-2-oxoethyl)(isopropyl)amino)-2-oxoethyl)-1H-indazole-3-carboxamide

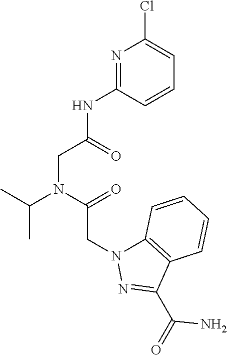 [SMILES: CC(C)N(CC(=O)Nc1cccc(Cl)n1)C(=O)Cn1nc(C(N)=O)c2ccccc21]

1-(2-((2-((6-chloropyridin-2-yl)amino)-2-oxoethyl)(isopropyl)amino)-2-oxoethyl)-1H-indazole-3-carboxamide (21.4 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ 11.18-10.78 (d, 1H), 8.19-8.13 (m, 1H), 7.92-7.60 (m, 3H), 7.46-7.18 (m, 4H), 5.63 (s, 1H), 5.46 (s, 1H), 4.606& 4.328 (m, 1H), 4.44 (s, 1H), 4.03 (s, 1H). 1.25-1.24 (m, 3H), 1.04-1.03 (m, 3H). LRMS (M+H⁺) m/z calculated 429.1, found 429.5.

### Example 13: Preparation of 1-(2-((2-((6-chloropyridin-2-yl)amino)-2-oxoethyl)(2-hydroxyethyl)amino)-2-oxoethyl)-1H-indazole-3-carboxamide

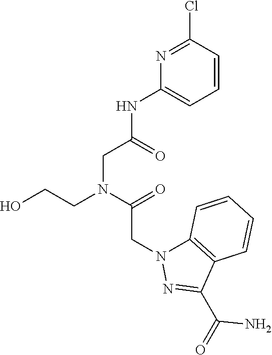 [SMILES: NC(=O)c1nn(CC(=O)N(CCO)CC(=O)Nc2cccc(Cl)n2)c2ccccc12]

1-(2-((2-((6-chloropyridin-2-yl)amino)-2-oxoethyl)(2-hydroxyethyl)amino)-2-oxoethyl)-1H-indazole-3-carboxamide (8 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CDCl₃+DMSO-d₆, 400 MHz) δ=8.25-8.15 (m, 2H), 7.78-7.68 (m, 1H), 7.56-7.50 (m, 1H), 7.40-7.37 (m, 1.5H), 7.24-7.19 (m, 1H), 7.13-7.05 (m, 1.5H), 5.67-5.16 (m, 2H), 4.56&4.22 (s, 2H), 3.74-3.47 (m, 4H). LRMS (M+H⁺) m/z calculated 431.1, found 431.6.

### Example 14: Preparation of 1-(2-((2-((6-chloropyridin-2-yl)amino)-2-oxoethyl)(methyl)amino)-2-oxoethyl)-1H-indazole-3-carboxamide

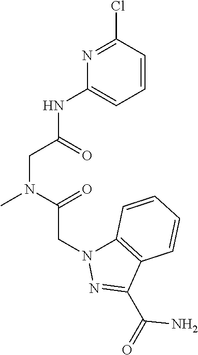 [SMILES: CN(CC(=O)Nc1cccc(Cl)n1)C(=O)Cn1nc(C(N)=O)c2ccccc21]

1-(2-((2-((6-chloropyridin-2-yl)amino)-2-oxoethyl)(methyl)amino)-2-oxoethyl)-1H-indazole-3-carboxamide (27 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CDCl₃+DMSO-d₆, 400 MHz) δ=11.09&10.76 (s, 1H), 8.29-8.26 (m, 1H), 8.17-8.06 (m, 1H), 7.70-7.67 (m, 1H), 7.54-7.42 (m, 2H), 7.27-7.20 (m, 2H), 7.11-7.04 (m, 1H), 6.85 (s, 1H), 5.47 (s, 2H), 4.45 (s, 2H), 3.27 (s, 3H). LRMS (M+H⁺) m/z calculated 401.1, found 401.5.

### Example 15: Preparation of 1-(2-((2-((6-chloropyridin-2-yl)amino)-2-oxoethyl)(ethyl)amino)-2-oxoethyl)-1H-indazole-3-carboxamide

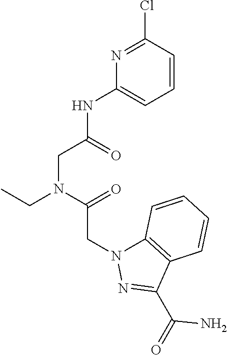 [SMILES: CCN(CC(=O)Nc1cccc(Cl)n1)C(=O)Cn1nc(C(N)=O)c2ccccc21]

1-(2-((2-((6-chloropyridin-2-yl)amino)-2-oxoethyl)(ethyl)amino)-2-oxoethyl)-1H-indazole-3-carboxamide (28 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=11.24 (s, 1H), 8.19-7.81 (m, 3H), 7.72-7.60 (m, 2H), 7.46-7.37 (m, 2H), 7.28-7.18 (m, 2H), 5.61&5.47 (s, 2H), 4.52 (s, 2H), 3.62-3.16 (m, 2H), 1.25&1.01 (t, 3H). LRMS (M+H⁺) m/z calculated 415.1, found 415.6.

### Example 16: Preparation of 1-(2-((2-((6-chloropyridin-2-yl)amino)-2-oxoethyl)(cyclopropyl)amino)-2-oxoethyl)-1H-indazole-3-carboxamide

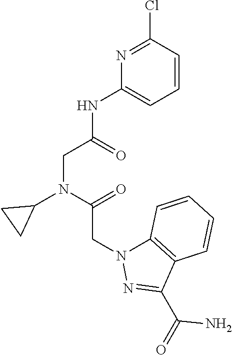 [SMILES: NC(=O)c1nn(CC(=O)N(CC(=O)Nc2cccc(Cl)n2)C2CC2)c2ccccc12]

1-(2-((2-((6-chloropyridin-2-yl)amino)-2-oxoethyl)(cyclopropyl)amino)-2-oxoethyl)-1H-indazole-3-carboxamide (20.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=10.92 (s, 1H), 8.17 (d, 1H), 8.00 (d, 1H), 7.86-7.83 (m, 1H), 7.70-7.65 (m, 2H), 7.43-7.26 (m, 2H), 7.21-7.19 (m, 2H), 5.69 (s, 2H), 4.18 (s, 2H), 3.12-3.11 (m, 1H), 1.01-0.95 (m, 4H). LRMS (M+H⁺) m/z calculated 427.1, found 427.5.

### Example 17: Preparation of 1-(2-((2-((6-chloropyridin-2-yl)amino)-2-oxoethyl)(piperidin-4-yl)amino)-2-oxoethyl)-1H-indazole-3-carboxamide

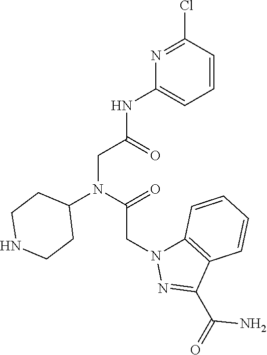 [SMILES: NC(=O)c1nn(CC(=O)N(CC(=O)Nc2cccc(Cl)n2)C2CCNCC2)c2ccccc12]

1-(2-((2-((6-chloropyridin-2-yl)amino)-2-oxoethyl)(piperidin-4-yl)amino)-2-oxoethyl)-1H-indazole-3-carboxamide (24.5 mg) was prepared as described for 1-(2-((2-((3-chloro-2-fluorobenzyl) amino)-2-oxoethyl)(ethyl)amino)-2-oxoethyl)-1H-indazole-3-carboxamide. ¹H NMR. (CD₃OD, 400 MHz) δ=8.20-8.23 (m, 1H), 7.97-8.14 (m, 1H), 7.68-7.81 (m, 1H), 7.56-7.64 (m, 1H), 7.42-7.48 (m, 1H), 7.29-7.30 (m, 1H), 7.07-7.08 (m, 1H), 5.33-5.78 (m, 3H), 4.43-4.53 (m, 2H), 3.75-4.16 (m, 1H), 3.41-3.51 (m, 2H), 2.87-3.23 (m, 2H), 1.93-2.13 (m, 3H). LCMS (M+H⁺) m/z calculated 470.2, found 470.7.

### Example 18: Preparation of 3-(2-((2-aminoethyl)(2-((6-chloropyridin-2-yl)amino)-2-oxoethyl)amino)-2-oxoethyl)-1H-indazole-1-carboxamide

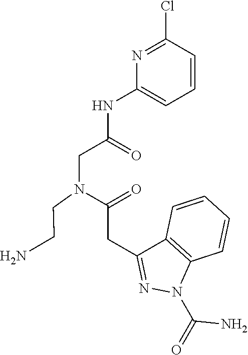 [SMILES: NCCN(CC(=O)Nc1cccc(Cl)n1)C(=O)Cc1nn(C(N)=O)c2ccccc12]

3-(2-((2-aminoethyl)(2-((6-chloropyridin-2-yl)amino)-2-oxoethyl)amino)-2-oxoethyl)-1H-indazole-1-carboxamide (40.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CD₃OD, 400 MHz) δ=11.16 (d, 1H), 8.18-8.10 (m, 1H), 7.95 (d, 2H), 7.69-7.64 (m, 4H), 7.46-7.40 (m, 2H), 7.29-7.27 (m, 2H), 5.54 (d, 2H), 4.33 (d, 2H), 3.51 (d, 2H), 2.96 (m, 2H). LRMS (M+H⁺) m/z calculated 430.1, found 430.6.

### Example 19: Preparation of 3-(2-((2-((6-chloropyridin-2-yl)amino)-2-oxoethyl)(cyclopentyl)amino)-2-oxoethyl)-1H-indazole-1-carboxamide

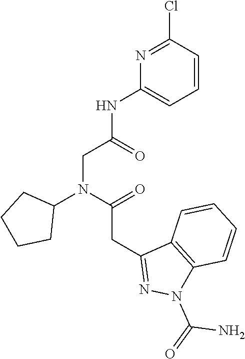 [SMILES: NC(=O)n1nc(CC(=O)N(CC(=O)Nc2cccc(Cl)n2)C2CCCC2)c2ccccc21]

3-(2-((2-((6-chloropyridin-2-yl)amino)-2-oxoethyl)(cyclopentyl)amino)-2-oxoethyl)-1H-indazole-1-carboxamide (50.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=11.76-10.78 (m, 1H), 8.19-7.79 (m, 3H), 7.69-7.59 (m, 2H), 7.47-7.28 (m, 2H), 7.25-7.26 (m, 2H), 5.51 (d, 2H), 4.23 (d, 2H), 1.99-1.97 (m, 1H), 1.69-1.24 (m, 8H). LRMS (M+H⁺) m/z calculated 455.2, found 455.6.

### Example 20: Preparation of 3-(2-((2-((6-chloropyridin-2-yl)amino)-2-oxoethyl)(cyclobutyl)amino)-2-oxoethyl)-1H-indazole-1-carboxamide

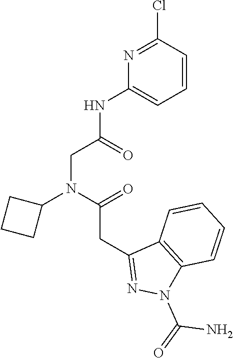 [SMILES: NC(=O)n1nc(CC(=O)N(CC(=O)Nc2cccc(Cl)n2)C2CCC2)c2ccccc21]

3-(2-((2-((6-chloropyridin-2-yl)amino)-2-oxoethyl)(cyclobutyl)amino)-2-oxoethyl)-1H-indazole-1-carboxamide (37 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=11.208-10.882 (d, 1H), 8.18-8.16 (m, 2H), 7.98-7.59 (m, 3H), 7.44-7.18 (m, 4H), 5.59 (d, 1H), 5.44 (d, 1H), 4.65 (m, 1H), 4.54 (d, 1H), 4.24 (d, 1H). 2.26-2.21 (m, 2H), 2.01-1.97 (m, 2H), 1.62-1.59 (m, 2H), LRMS (M+H⁺) m/z calculated 441.1, found 441.6.

### Example 21: Preparation of 3-(2-((2-((6-chloropyridin-2-yl)amino)-2-oxoethyl)(tetrahydro-2H-pyran-4-yl)amino)-2-oxoethyl)-1H-indazole-1-carboxamide

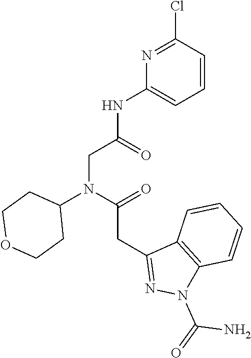 [SMILES: NC(=O)n1nc(CC(=O)N(CC(=O)Nc2cccc(Cl)n2)C2CCOCC2)c2ccccc21]

3-(2-((2-((6-chloropyridin-2-yl)amino)-2-oxoethyl)(tetrahydro-2H-pyran-4-yl)amino)-2-oxoethyl)-1H-indazole-1-carboxamide (6.4 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=11.20-10.78 (d, 1H), 7.96-7.90 (m, 1H), 7.81-7.60 (m, 3H), 7.45-7.38 (m, 2H), 7.29-7.16 (m, 2H), 5.72 (d, 1H), 5.50 (d, 1H), 4.49 (d, 1H), 4.09 (m, 1H). 3.90 (d, 1H), 3.87 (m, 2H), 3.46 (m, 1H),1.99 (d, 1H). 1.77-1.73 (m, 1H). 1.60-1.57 (m, 1H). 1.48-1.31 (m, 1H). 1.17-1.08 (m, 1H). LRMS (M+H⁺) m/z calculated 471.2, found 471.6.

### Example 22: Preparation of 1-(2-(azetidin-3-yl(2-((6-chloropyridin-2-yl)amino)-2-oxoethyl)amino)-2-oxoethyl)-1H-indazole-3-carboxamide

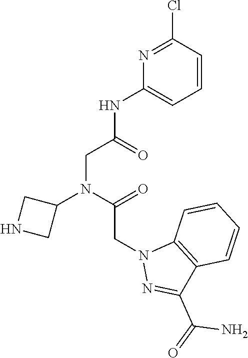 [SMILES: NC(=O)c1nn(CC(=O)N(CC(=O)Nc2cccc(Cl)n2)C2CNC2)c2ccccc12]

1-(2-(azetidin-3-yl(2-((6-chloropyridin-2-yl)amino)-2-oxoethyl)amino)-2-oxoethyl)-1H-indazole-3-carboxamide (20.2 mg) was prepared as described for tert-butyl 3-(2-(3-carbamoyl-1H-indazol-1-yl)-N-(2-((6-chloropyridin-2-yl)amino)-2-oxoethyl)acetamido)azetidine-1-carboxylate. ¹H NMR (CD₃OD, 400 MHz) δ=8.20 (d, 1H), 8.02 (d, 1H), 7.75-7.79 (m, 1H), 7.60 (d, 1H), 7.47 (d, 1H), 7.29 (d, 1H), 7.16 (d, 1H), 5.66 (s, 2H), 4.27-4.55 (m, 2H). LRMS (M+H⁺) m/z calculated 442.1, found 442.3.

### Example 23: Preparation of 1-(2-((2-((6-chloropyridin-2-yl)amino)-2-oxoethyl)(2-(methylamino)ethyl)amino)-2-oxoethyl)-1H-indazole-3-carboxamide

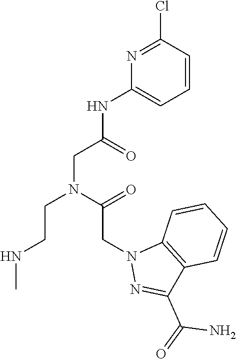 [SMILES: CNCCN(CC(=O)Nc1cccc(Cl)n1)C(=O)Cn1nc(C(N)=O)c2ccccc21]

1-(2-((2-((6-chloropyridin-2-yl)amino)-2-oxoethyl)(2-(methylamino)ethyl)amino)-2-oxoethyl)-1H-indazole-3-carboxamide (17.4 mg) was prepared as described for tert-butyl (2-(2-(3-carbamoyl-1H-indazol-1-yl)-N-(2-((6-chloropyridin-2-yl)amino)-2-oxoethyl)acetamido)ethyl)(methyl)carbamate. ¹H NMR (CD₃OD, 400 MHz) δ=8.19 (d, 1H), 8.06 (s, 1H), 7.76-7.80 (m, 1H), 7.58 (d, 1H), 7.45 (d, 1H), 7.26 (d, 1H), 7.18 (d, 1H), 5.65 (s, 2H), 4.60 (s, 2H), 3.31 (s, 2H), 3.22 (s, 2H), 2.69 (s, 3H). LRMS (M+H⁺) m/z calculated 444.1, found 444.1.

### Example 24: Preparation of 1-(2-((2-acetamidoethyl)(2-((6-chloropyridin-2-yl)amino)-2-oxoethyl)amino)-2-oxoethyl)-1H-indazole-3-carboxamide

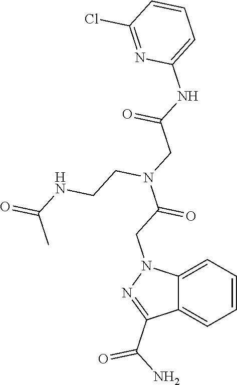 [SMILES: CC(=O)NCCN(CC(=O)Nc1cccc(Cl)n1)C(=O)Cn1nc(C(N)=O)c2ccccc21]

1-(2-((2-acetamidoethyl)(2-((6-chloropyridin-2-yl)amino)-2-oxoethyl)amino)-2-oxoethyl)-1H-indazole-3-carboxamide (3.5 mg) was prepared as described for 1-(2-((2-((3-chloro-2-fluorobenzyl) amino)-2-oxoethyl)(ethyl)amino)-2-oxoethyl)-1H-indazole-3-carboxamide. ¹H NMR (DMSO-d6, 400 MHz) δ=11.1 (d, 1H), 8.18-8.14 (m, 2H), 8.01-7.90 (m, 2H), 7.70-7.57 (m, 2H), 7.46-7.26 (m, 2H), 7.20-7.18 (m, 2H), 5.51 (d, 2H), 4.37 (d, 2H), 3.31 (d, 4H), 1.84 (d, 3H). LRMS (M+H⁺) m/z calculated 472.1, found 472.6.

### Example 25: Preparation of 1-(2-((2-((6-chloropyridin-2-yl)amino)-2-oxoethyl)(pyridin-4-ylmethyl)amino)-2-oxoethyl)-1H-indazole-3-carboxamide

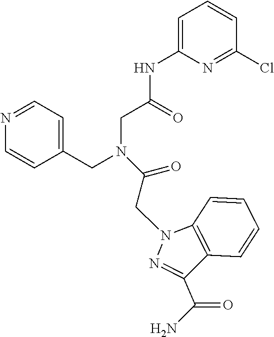 [SMILES: NC(=O)c1nn(CC(=O)N(CC(=O)Nc2cccc(Cl)n2)Cc2ccncc2)c2ccccc12]

1-(2-((2-((6-chloropyridin-2-yl)amino)-2-oxoethyl)(pyridin-4-ylmethyl)amino)-2-oxoethyl)-1H-indazole-3-carboxamide (39.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=11.00 (d, 1H), 8.52-8.64 (m, 2H), 8.10-8.20 (m, 2H), 7.84-8.00 (m, 1H), 7.69 (d, 2H), 7.25-7.49 (m, 6H), 5.59 (d, 2H), 4.17-4.94 (m, 4H). LRMS (M+H⁺) m/z calculated 478.1, found 478.1.

### Example 26: Preparation of 1-(2-((2-((6-chloropyridin-2-yl)amino)-2-oxoethyl)(pyrrolidin-3-yl)amino)-2-oxoethyl)-1H-indazole-3-carboxamide

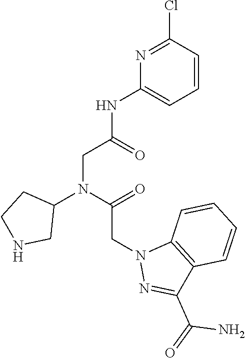 [SMILES: NC(=O)c1nn(CC(=O)N(CC(=O)Nc2cccc(Cl)n2)C2CCNC2)c2ccccc12]

1-(2-((2-((6-chloropyridin-2-yl)amino)-2-oxoethyl)(pyrrolidin-3-yl)amino)-2-oxoethyl)-1H-indazole-3-carboxamide (17.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CD₃OD, 400 MHz) δ=8.21 (d, 1H), 8.13-7.99 (q, 1H), 7.81-7.69 (m, 1H), 7.60 (d, 1H), 7.45 (t, 1H), 7.28 (t, 1H), 7.18-7.08 (m, 1H), 5.69-5.44 (m, 2H), 4.58-4.45 (m, 2H), 4.17 (s, 1H), 3.25-2.84 (m, 4H), 2.20-1.80 (m, 2H). LRMS (M+H⁺⁾m/z calculated 456.2, found 456.5.

### Example 27: Preparation of 1-(2-((2-((3-chloro-2-fluorobenzyl)amino)-2-oxoethyl)(thiophen-3-ylmethyl)amino)-2-oxoethyl)-1H-indazole-3-carboxamide

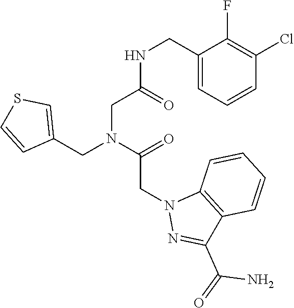 [SMILES: NC(=O)c1nn(CC(=O)N(CC(=O)NCc2cccc(Cl)c2F)Cc2ccsc2)c2ccccc12]

1-(2-((2-((3-chloro-2-fluorobenzyl)amino)-2-oxoethyl)(thiophen-3-ylmethyl)amino)-2-oxoethyl)-1H-indazole-3-carboxamide (27.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CD₃OD, 400 MHz) δ=8.22 (t, 1H), 7.62-7.22 (m, 7H), 7.16-6.97 (m, 2H), 5.62 (s, 1H), 5.53 (s, 1H), 4.59 (s, 1H), 4.47 (s, 1H), 4.44 (s, 1H), 4.25 (s, 1H), 4.11 (s, 1H). LRMS (M+H⁺) m/z calculated 514.1, found 514.5.

### Example 28: Preparation of 1-(2-((2-((3-chloro-2-fluorobenzyl)amino)-2-oxoethyl)(cyclobutyl)amino)-2-oxoethyl)-1H-indazole-3-carboxamide

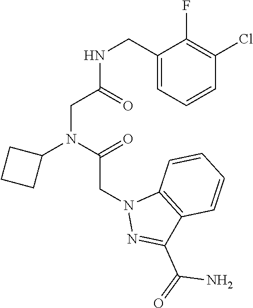 [SMILES: NC(=O)c1nn(CC(=O)N(CC(=O)NCc2cccc(Cl)c2F)C2CCC2)c2ccccc12]

1-(2-((2-((3-chloro-2-fluorobenzyl)amino)-2-oxoethyl)(cyclobutyl)amino)-2-oxoethyl)-1H-indazole-3-carboxamide (48.6 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=8.87-8.42 (s, 1H), 8.19-8.17 (d, 1H), 7.67-7.59 (d, 4H), 7.53-7.51 (m, 2H), 7.46-7.32 (m, 3H), 7.26-7.22 (m, 2H), 5.55&5.41 (d, 2H), 4.64-4.59 (m, 1H), 4.47 (m, 1H). 4.34-4.29 (m, 2H), 4.04 (s, 1H), 2.20-1.99 (m, 2H), 1.63-1.55 (m, 2H), 1.24 (s, 2H). ¹H NMR LRMS (M+H⁺) m/z calculated 472.2, found 472.2.

### Example 29: Preparation of 1-(2-((2-((3-chloro-2-fluorobenzyl)amino)-2-oxoethyl)(tetrahydro-2H-pyran-4-yl)amino)-2-oxoethyl)-1H-indazole-3-carboxamide

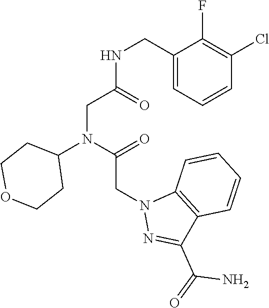 [SMILES: NC(=O)c1nn(CC(=O)N(CC(=O)NCc2cccc(Cl)c2F)C2CCOCC2)c2ccccc12]

1-(2-((2-((3-chloro-2-fluorobenzyl)amino)-2-oxoethyl)(tetrahydro-2H-pyran-4-yl)amino)-2-oxoethyl)-1H-indazole-3-carboxamide (21.7 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ 8.87-8.42 (s, 1H), 8.19-8.17 (d, 1H), 7.67-7.59 (d, 4H), 7.53-7.51 (m, 2H), 7.46-7.32 (m, 3H), 7.26-7.22 (m, 2H), 5.55&5.41 (d, 2H), 4.64-4.59 (m, 1H), 4.47 (m, 1H). 4.34-4.29 (m, 2H), 4.04 (s, 1H), 2.20-1.99 (m, 2H), 1.63-1.55 (m, 2H), 1.24 (s, 2H). LRMS (M+H⁺) m/z calculated 502.2, found 502.2.

### Example 30: Preparation of 1-(2-((2-((3-chloro-2-fluorobenzyl)amino)-2-oxoethyl)(cyclopentyl)amino)-2-oxoethyl)-1H-indazole-3-carboxamide

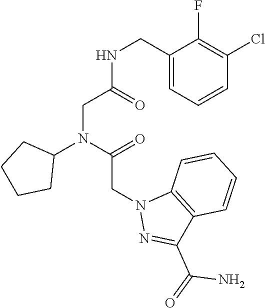 [SMILES: NC(=O)c1nn(CC(=O)N(CC(=O)NCc2cccc(Cl)c2F)C2CCCC2)c2ccccc12]

1-(2-((2-((3-chloro-2-fluorobenzyl)amino)-2-oxoethyl)(cyclopentyl)amino)-2-oxoethyl)-1H-indazole-3-carboxamide (45.5 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=8.82-8.37 (s, 1H), 8.19-8.17 (d, 1H), 7.67 (s, 1H), 7.61-7.52 (m, 1H), 7.49-7.44 (m, 1H), 7.41-7.36 (m, 3H), 7.26-7.22 (m, 2H), 5.62 (d, 1H), 5.44 (d, 1H). 4.58&4.39 (m, 1H), 4.47 (d, 1H), 4.32 (d, 1H), 4.19 (s, 1H), 3.82 (s, 1H), 1.95 (s, 1H), 1.67 (s, 1H), 1.62 (s, 1H), 1.55 (s, 1H), 1.45 (s, 1H), 1.36 (s, 1H), 1.23 (d, 2H). ¹H NMR LRMS (M+H⁺) m/z calculated 486.2, found 486.2.

### Example 31: Preparation of 1-(2-((2-aminoethyl)(2-((3-chloro-2-fluorobenzyl)amino)-2-oxoethyl)amino)-2-oxoethyl)-1H-indazole-3-carboxamide

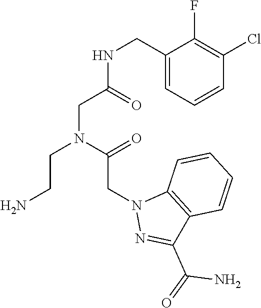 [SMILES: NCCN(CC(=O)NCc1cccc(Cl)c1F)C(=O)Cn1nc(C(N)=O)c2ccccc21]

1-(2-((2-aminoethyl)(2-((3-chloro-2-fluorobenzyl)amino)-2-oxoethyl)amino)-2-oxoethyl)-1H-indazole-3-carboxamide (45.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=8.82-8.75 (m, 1H), 8.19-8.17 (m, 1H), 8.03-7.93 (m, 1H), 7.71-7.54 (m, 4H), 7.51-7.40 (m, 3H), 7.30-7.20 (m, 2H), 5.55 (d, 2H), 4.49-4.30 (m, 4H), 3.62 (d, 2H), 3.21-3.18 (m, 1H), 2.93 (d, 1H). LRMS (M+H⁺) m/z calculated 461.1, found 461.5.

### Example 32: Preparation of 1-(2-((2-((3-chloro-2-fluorobenzyl)amino)-2-oxoethyl)(pyridin-4-ylmethyl)amino)-2-oxoethyl)-1H-indazole-3-carboxamide

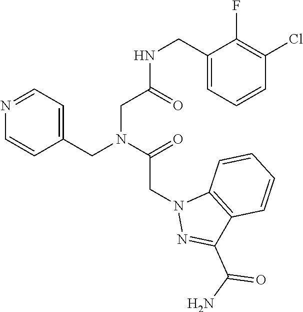 [SMILES: NC(=O)c1nn(CC(=O)N(CC(=O)NCc2cccc(Cl)c2F)Cc2ccncc2)c2ccccc12]

1-(2-((2-((3-chloro-2-fluorobenzyl)amino)-2-oxoethyl)(pyridin-4-ylmethyl)amino)-2-oxoethyl)-1H-indazole-3-carboxamide (38.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CD₃OD, 400 MHz) δ=8.45 (d, 2H), 8.16-8.24 (m, 1H), 7.04-7.62 (m, 8H), 5.56 (d, 2H), 4.96 (d, 1H), 4.65 (s, 1H), 4.46 (d, 2H), 4.31 (s, 1H), 4.11 (s, 1H). LRMS (M+H⁺) m/z calculated 509.1, found 509.6.

### Example 33: Preparation of 1-(2-((2-((3-chloro-2-fluorobenzyl)amino)-2-oxoethyl)(methyl)amino)-2-oxoethyl)-1H-indazole-3-carboxamide

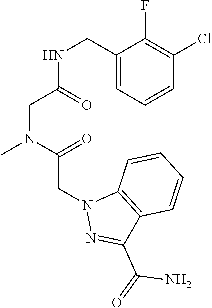 [SMILES: CN(CC(=O)NCc1cccc(Cl)c1F)C(=O)Cn1nc(C(N)=O)c2ccccc21]

1-(2-((2-((3-chloro-2-fluorobenzyl)amino)-2-oxoethyl)(methyl)amino)-2-oxoethyl)-1H-indazole-3-carboxamide (20.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=8.84-8.62 (m, 1H), 8.22-8.17 (m, 1H), 7.71-7.62 (m, 2H), 7.60-7.30 (m, 4H), 7.27-7.21 (m, 2H), 5.60-5.44 (m, 2H), 4.47-4.33 (m, 2H), 4.25-4.00 (d, 2H), 3.19-2.81 (m, 3H). LRMS (M+H⁺) m/z calculated 432.1, found 432.7.

### Example 34: Preparation of 1-(2-((2-((3-chloro-2-fluorobenzyl)amino)-2-oxoethyl)(piperidin-4-yl)amino)-2-oxoethyl)-1H-indazole-3-carboxamide

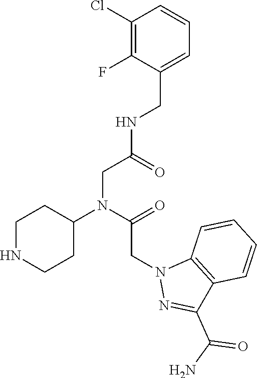 [SMILES: NC(=O)c1nn(CC(=O)N(CC(=O)NCc2cccc(Cl)c2F)C2CCNCC2)c2ccccc12]

1-(2-((2-((3-chloro-2-fluorobenzyl)amino)-2-oxoethyl)(piperidin-4-yl)amino)-2-oxoethyl)-1H-indazole-3-carboxamide (19.7 mg) was prepared as described for 1-(2-((2-((3-chloro-2-fluorobenzyl) amino)-2-oxoethyl)(ethyl)amino)-2-oxoethyl)-1H-indazole-3-carboxamide. ¹H NMR. (CD₃OD, 400 MHz) δ=8.22 (d, 1H), 7.54-7.60 (m, 1H), 7.36-7.44 (m, 2H), 7.21-7.33 (m, 2H), 6.97-7.14 (m, 1H), 5.65 (s, 1H), 5.41 (s, 1H), 4.55 (s, 1H), 4.30-4.41 (m, 3H), 3.98 (s, 1H), 3.49 (d, 1H), 3.49 (d, 1H), 3.14 (t, 1H), 3.00 (t, 1H), 1.88-2.02 (m, 4H). LCMS (M+H⁺) m/z calculated 501.2, found 501.7.

### Example 35: Preparation of 1-(2-((2-((3-chloro-2-fluorobenzyl)amino)-2-oxoethyl)(pyridin-3-ylmethyl)amino)-2-oxoethyl)-1H-indazole-3-carboxamide

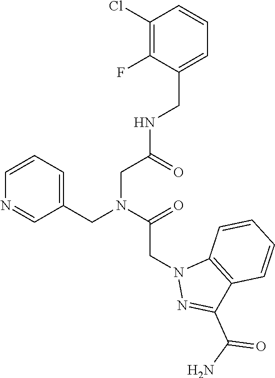 [SMILES: NC(=O)c1nn(CC(=O)N(CC(=O)NCc2cccc(Cl)c2F)Cc2cccnc2)c2ccccc12]

1-(2-((2-((3-chloro-2-fluorobenzyl)amino)-2-oxoethyl)(pyridin-3-ylmethyl)amino)-2-oxoethyl)-1H-indazole-3-carboxamide (3.8 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CD₃OD, 400 MHz) δ=8.26 (d, 1H), 8.10 (d, 1H), 7.90 (d, 1H), 7.65 (d, 1H), 7.51 (d, 1H), 7.38-7.48 (m, 3H), 7.04-7.14 (m, 2H), 5.53 (s, 2H), 4.78 (s, 2H), 4.64 (s, 4H). LRMS (M+H⁺) m/z calculated 509.1, found 509.1.

### Example 36: Preparation of 1-(2-((2-((3-chloro-2-fluorobenzyl)amino)-2-oxoethyl)(phenyl)amino)-2-oxoethyl)-1H-indazole-3-carboxamide

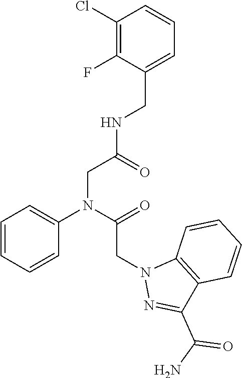 [SMILES: NC(=O)c1nn(CC(=O)N(CC(=O)NCc2cccc(Cl)c2F)c2ccccc2)c2ccccc12]

1-(2-((2-((3-chloro-2-fluorobenzyl)amino)-2-oxoethyl)(phenyl)amino)-2-oxoethyl)-1H-indazole-3-carboxamide (2.3 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=8.60-8.58 (m, 1H), 8.16-8.15 (m, 1H), 7.67-7.52 (m, 4H), 7.45-7.26 (m, 6H), 7.21-7.09 (m, 3H), 5.13 (s, 2H), 4.33-4.27 (d, 4H). LRMS (M+H⁺) m/z calculated 494.1, found 494.2.

### Example 37: Preparation of 1-(2-((2-((3-chloro-2-fluorobenzyl)amino)-2-oxoethyl)(pyrrolidin-3-yl)amino)-2-oxoethyl)-1H-indazole-3-carboxamide

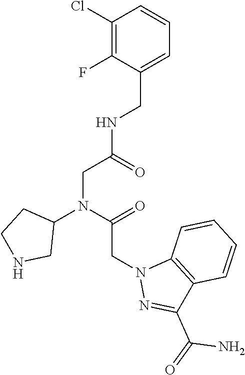 [SMILES: NC(=O)c1nn(CC(=O)N(CC(=O)NCc2cccc(Cl)c2F)C2CCNC2)c2ccccc12]

1-(2-((2-((3-chloro-2-fluorobenzyl)amino)-2-oxoethyl)(pyrrolidin-3-yl)amino)-2-oxoethyl)-1H-indazole-3-carboxamide (12.0 mg) was prepared as described for 1-(2-((2-((3-chloro-2-fluorobenzyl) amino)-2-oxoethyl)(ethyl)amino)-2-oxoethyl)-1H-indazole-3-carboxamide. ¹H NMR (CD₃OD, 400 MHz) δ=8.28 (d, 1H), 7.87 (s, 1H), 7.58 (d, 1H), 7.50 (t, 1H), 7.33 (t, 1H), 7.28-7.22 (m, 2H), 4.70 (t, 1H), 4.52-4.28 (m, 3H), 4.23 (s, 1H), 3.65-3.57 (m, 4H), 2.38 (t, 2H). LRMS (M+H⁺) m/z calculated 487.2, found 487.2.

### Example 38: Preparation of (R)-1-(2-((1-((3-chloro-2-fluorobenzyl)amino)-1-oxopropan-2-yl)(cyclopropyl)amino)-2-oxoethyl)-1H-indazole-3-carboxamide

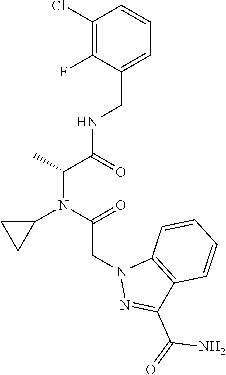 [SMILES: C[C@H](C(=O)NCc1cccc(Cl)c1F)N(C(=O)Cn1nc(C(N)=O)c2ccccc21)C1CC1]

(R)-1-(2-((1-((3-chloro-2-fluorobenzyl)amino)-1-oxopropan-2-yl)(cyclopropyl)amino)-2-oxoethyl)-1H-indazole-3-carboxamide (82.8 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (DMSO-d6, 400 MHz) δ=8.19-8.17 (m, 2H), 7.66-7.58 (m, 2H), 7.46-7.23 (m, 4H), 7.17-7.01 (m, 2H), 5.75-5.58 (m, 2H), 4.59-4.53 (m, 1H), 4.29 (brs, 2H), 2.95 (brs, 1H), 1.43 (d, 3H), 1.23-0.89 (m, 4H). LRMS (M+H⁺) m/z calculated 472.1, found 472.1.

### Example 39: Preparation of (S)-1-(2-((1-((3-chloro-2-fluorobenzyl)amino)-1-oxopropan-2-yl)(cyclopropyl)amino)-2-oxoethyl)-1H-indazole-3-carboxamide

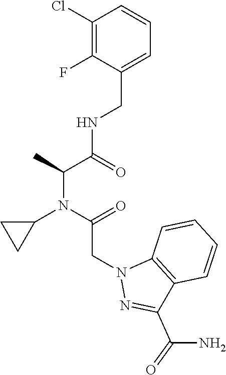 [SMILES: C[C@@H](C(=O)NCc1cccc(Cl)c1F)N(C(=O)Cn1nc(C(N)=O)c2ccccc21)C1CC1]

(S)-1-(2-((1-((3-chloro-2-fluorobenzyl)amino)-1-oxopropan-2-yl)(cyclopropyl)amino)-2-oxoethyl)-1H-indazole-3-carboxamide (30.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR. (DMSO-d6, 400 MHz) δ=8.18-8.20 (m, 2H), 7.62-7.66 (m, 2H), 7.41-7.43 (m, 3H), 7.25-7.29 (m, 1H), 7.18-7.20 (m, 1H), 7.00-7.08 (m, 1H), 5.64 (t, 2H), 4.55-7.59 (m, 1H), 4.28-4.32 (m, 2H), 2.96 (s, 1H), 1.44 (d, 3H), 0.96-1.02 (m, 4H). LRMS (M+Na⁺) m/z calculated 494.1, found 494.1.

### Example 40: Preparation of 1-(2-((2-((3-chloro-2-fluorobenzyl)amino)-2-oxoethyl)(cyclopropyl)amino)-2-oxoethyl)-1H-pyrazolo[3,4-c]pyridine-3-carboxamide

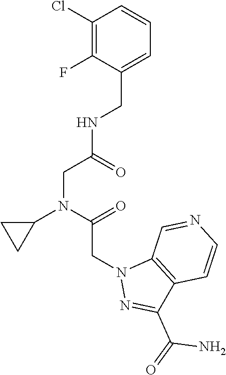 [SMILES: NC(=O)c1nn(CC(=O)N(CC(=O)NCc2cccc(Cl)c2F)C2CC2)c2cnccc12]

1-(2-((2-((3-chloro-2-fluorobenzyl)amino)-2-oxoethyl)(cyclopropyl)amino)-2-oxoethyl)-1H-pyrazolo[3,4-c]pyridine-3-carboxamide (10 mg) was prepared as described for 1-(2-((2-((3-chloro-2-fluorobenzyl)-2-oxoethyl)(ethyl(ethyl)amino)-2-oxoethyl)-1H-indazole-3-carboxamide. ¹H NMR (CD₃OD, 400 MHz) δ=9.66 (s, 1H), 8.67 (d, 1H), 8.57 (d, 1H), 7.33 (t, 1H), 7.25 (t, 1H), 7.03 (t, 1H), 6.04 (d, 2H), 4.45 (s, 2H), 4.16 (s, 2H), 3.13 (s, 1H), 1.07 (s, 4H). LRMS (M+H⁺) m/z calculated 459.1, found 459.1.

### Example 41: Preparation of 3-(2-((2-((3-chloro-2-fluorobenzyl)amino)-2-oxoethyl)(cyclopropyl)amino)-2-oxoethyl)-1H-indole-1-carboxamide

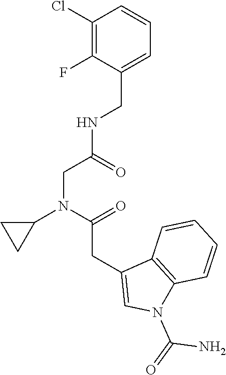 [SMILES: NC(=O)n1cc(CC(=O)N(CC(=O)NCc2cccc(Cl)c2F)C2CC2)c2ccccc21]

3-(2-((2-((3-chloro-2-fluorobenzyl)amino)-2-oxoethyl)(cyclopropyl)amino)-2-oxoethyl)-1H-indole-1-carboxamide (10 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CD₃OD, 400 MHz) δ=8.23 (d, 1H), 7.59 (s, 1H), 7.50 (d, 1H), 7.33 (t, 1H), 7.22 (d, 1H), 7.15 (t, 1H), 6.95 (t, 1H), 6.71 (t, 1H), 5.70 (s, 2H), 4.49-4.41 (m, 2H), 4.09 (s, 2H), 4.00 (s, 2H), 3.46 (d, 1H), 2.90-2.87 (m, 1H), 0.91-0.86 (m, 4H). LRMS (M+H⁺) m/z calculated 457.1, found 457.2.

### Example 42: Preparation of 3-(2-((2-((3-chloro-2-fluorobenzyl)amino)-2-oxoethyl)(cyclopropyl)amino)-2-oxoethyl)imidazo[1,5-a]pyridine-1-carboxamide

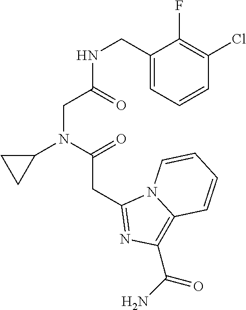 [SMILES: NC(=O)c1nc(CC(=O)N(CC(=O)NCc2cccc(Cl)c2F)C2CC2)n2ccccc12]

3-(2-((2-((3-chloro-2-fluorobenzyl)amino)-2-oxoethyl)(cyclopropyl)amino)-2-oxoethyl)imidazo[1,5-a]pyridine-1-carboxamide (16.0 mg) was prepared as described for 1-(2-((2-((3-chloro-2-fluorobenzyl)amino)-2-oxoethyl)(ethyl)amino)-2-oxoethyl)-1H-indazole-3-carboxamide. ¹H NMR (CD₃OD, 400 MHz) δ=8.12-8.19 (m, 2H), 7.33 (t, 1H), 7.24 (t, 1H), 7.11-7.15 (m, 1H), 7.03 (t, 1H), 6.81 (t, 1H), 4.52-4.56 (m, 2H), 4.40-4.46 (m, 2H), 4.10-4.14 (m, 2H), 3.06-3.12 (m, 1H), 1.00 (d, 1H). LRMS (M+H⁺) m/z calculated 458.1, found 458.7.

### Example 43: Preparation of 1-(2-((2-((3-chloro-2-fluorobenzyl)amino)-2-oxoethyl)(cyclopropyl)amino)-2-oxoethyl)-5-(trifluoromethyl)-1H-pyrazole-3-carboxamide

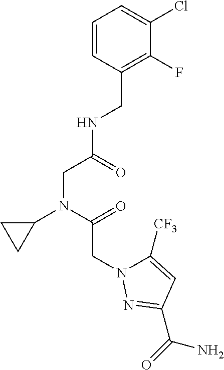 [SMILES: NC(=O)c1cc(C(F)(F)F)n(CC(=O)N(CC(=O)NCc2cccc(Cl)c2F)C2CC2)n1]

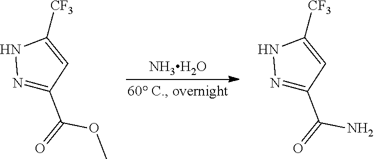

To a solution of methyl 5-(trifluoromethyl)-1H-pyrazole-3-carboxylate (150.0 mg, 0.8 mmol, 1.0 eq.) in NH₃.H₂O (3.0 mL) was added two drops of DMF. The mixture was stirred at 60° C. overnight, then concentrated under vacuum to provide crude 5-(trifluoromethyl)-1H-pyrazole-3-carboxamide (100.0 mg, 72% yield) as a white solid.

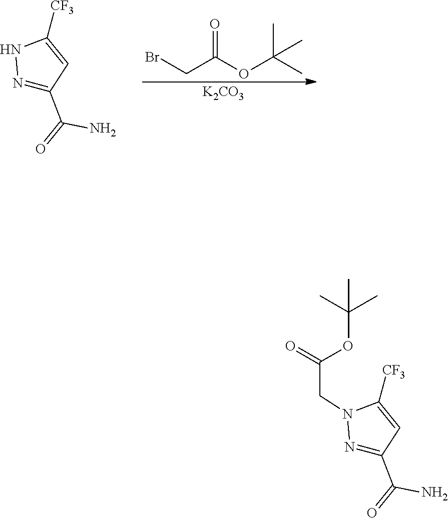

A solution of 5-(trifluoromethyl)-1H-pyrazole-3-carboxamide (100.0 mg, 0.6 mmol, 1.0 eq.), tert-butyl 2-bromoacetate (218.0 mg, 1.2 mmol, 2.0 eq), K₂CO₃ (0.2 g, 1.7 mmol, 3.0 eq.) in CH₃CN (10.0 mL) was stirred at 90° C. overnight. The reaction mixture was cooled and concentrated under vacuum.the resulting residue was purified by column chromatography (PE:EA=3/1) to provide tert-butyl 2-(3-carbamoyl-5-(trifluoromethyl)-1H-pyrazol-1-yl)acetate (100 mg, 60% yield) as a white solid.

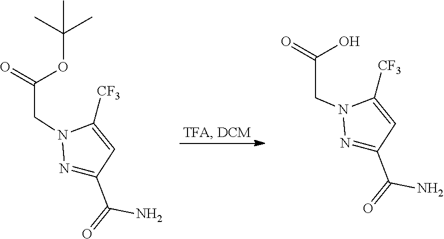 [SMILES: CC(C)(C)OC(=O)Cn1nc(C(N)=O)cc1C(F)(F)F ; NC(=O)c1cc(C(F)(F)F)n(CC(=O)O)n1]

To a solution of tert-butyl 2-(3-carbamoyl-5-(trifluoromethyl)-1H-pyrazol-1-yl)acetate (100.0 mg, 0.3 mmol, 1.0 eq.) in DCM (5.0 mL) was added TFA (2 mL). The mixture was stirred at r.t. for 2 h, and then concentrated under vacuum to provide crude 2-(3-carbamoyl-5-(trifluoromethyl)-1H-pyrazol-1-yl)acetic acid (80.0 mg, ca. 100% yield) as a yellow solid.

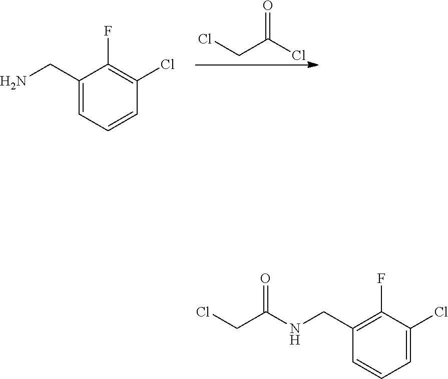 [SMILES: NCc1cccc(Cl)c1F ; O=C(Cl)CCl ; O=C(CCl)NCc1cccc(Cl)c1F]

To a solution of (3-chloro-2-fluorophenyl)methanamine (1.6 g, 10.0 mmol, 1.0 eq.) and Et₃N (1.51 g, 15.0 mmol, 1.0 eq.) in dichloromethane (100 mL) was added 2-chloroacetyl chloride (1.7 g, 15.0 mmol, 1.5 eq.). The mixture was stirred at r.t. for 16 h, and then washed with aq. 10% NaHCO₃ solution (100 mL), dried over anhydrous Na₂SO₄, filtered, and concentrated to provide crude 2-chloro-N-(3-chloro-2-fluorobenzyl)acetamide (2.2 g, 93% yield).

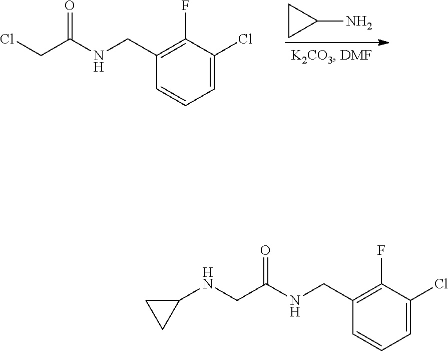

To a solution of 2-chloro-N-(3-chloro-2-fluorobenzyl)acetamide (3.0 g, 12.7 mmol, 1.0 eq.) and K₂CO₃ (3.5 g, 25.4 mmol, 2.0 eq.) in DMF (20 mL) was added cyclopropanamine (1.5 g, 25.4 mmol, 2.0 eq.). The reaction mixture was stirred at 60° C. for 16 h. Ethyl acetate (50 mL) and water (50 mL) were added. The organic layer was separated and concentrated. The residue was and purified by silica column chromatography (PE/EA=1/1) to provide N-(3-chloro-2-fluorobenzyl)-2-(ethylamino)acetamide (1.6 g, 49% yield).

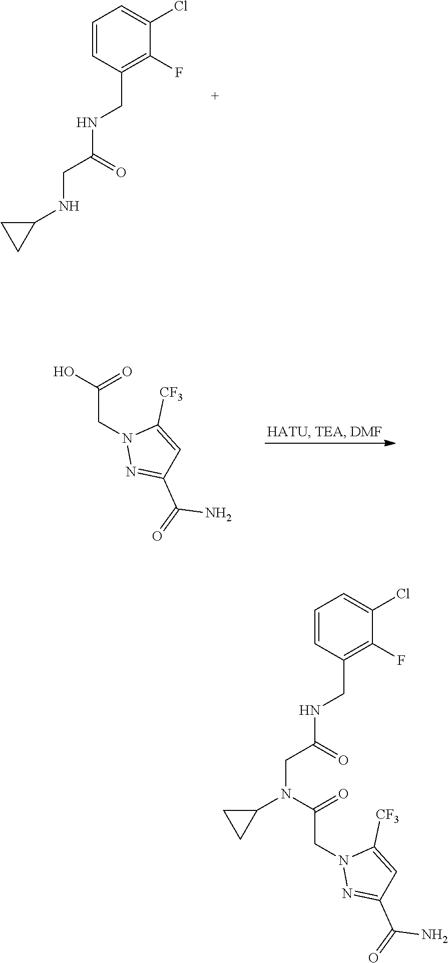 [SMILES: O=C(CNC1CC1)NCc1cccc(Cl)c1F ; NC(=O)c1cc(C(F)(F)F)n(CC(=O)O)n1 ; NC(=O)c1cc(C(F)(F)F)n(CC(=O)N(CC(=O)NCc2cccc(Cl)c2F)C2CC2)n1]

To a solution of N-(3-chloro-2-fluorobenzyl)-2-(cyclopropylamino)acetamide (100.0 mg, 0.4 mmol, 1.1 eq.) and 2-(3-carbamoyl-5-(trifluoromethyl)-1H-pyrazol-1-yl)acetic acid (80.0 mg, 0.35 mmol, 1.0 eq.) in DMF (3.0 mL) were added HATU (160.0 mg, 0.4 mmol, 1.2 eq.) and TEA (107.0 mg, 1.1 mmol, 3.0 eq.). The mixture was stirred at r.t. for 1 h. Water (50 mL) was added and the mixture was filtered. The filtrate was washed with dichloromethane/petroleum ether (1:5, 10 mL) and concentrated. The resulting residue was purified by Pre-HPLC to provide 1-(2-((2-((3-chloro-2-fluorobenzyl)amino)-2-oxoethyl)(cyclopropyl)amino)-2-oxoethyl)-5-(trifluoromethyl)-1H-pyrazole-3-carboxamide (34.0 mg, 21% yield). ¹H NMR (CD₃OD, 400 MHz) δ=7.36-7.23 (m, 2H), 7.19 (s, 1H), 7.07 (t, 1H), 5.56 (s, 2H), 4.43 (s, 2H), 4.11 (s, 2H), 3.02-2.98 (m, 1H), 1.00-0.94 (m, 4H). LRMS (M+H⁺) m/z calculated 476.1, found 476.2.

### Example 44: Preparation of 3-carbamoyl-1-(2-((2-((3-chloro-2-fluorobenzyl)amino)-2-oxoethyl)(cyclopropyl)amino)-2-oxoethyl)-1H-pyrazole-5-carboxylic acid

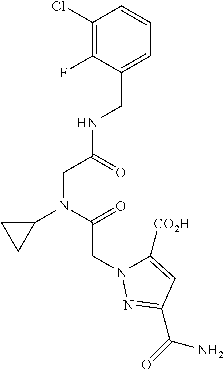 [SMILES: NC(=O)c1cc(C(=O)O)n(CC(=O)N(CC(=O)NCc2cccc(Cl)c2F)C2CC2)n1]

3-carbamoyl-1-(2-((2-((3-chloro-2-fluorobenzyl)amino)-2-oxoethyl)(cyclopropyl)amino)-2-oxoethyl)-1H-pyrazole-5-carboxylic acid (34.0 mg) was prepared as described for 1-(2-((2-((3-chloro-2-fluorobenzyl)amino)-2-oxoethyl)(cyclopropyl)amino)-2-oxoethyl)-5-(trifluoromethyl)-1H-pyrazole-3-carboxamide. ¹H NMR (CD₃OD, 400 MHz) δ=7.36-7.23 (m, 2H), 7.19 (s, 1H), 7.07 (t, 1H), 5.56 (s, 2H), 4.43 (s, 2H), 4.11 (s, 2H), 3.02-2.98 (m, 1H), 1.00-0.94 (m, 4H). LRMS (M+H⁺) m/z calculated 476.1, found 476.2.

### Example 45: Preparation of 1-(2-((2-((3-chloro-2-fluorobenzyl)amino)-2-oxoethyl)(cyclopropyl)amino)-2-oxoethyl)-1H-pyrazole-3,5-dicarboxamide

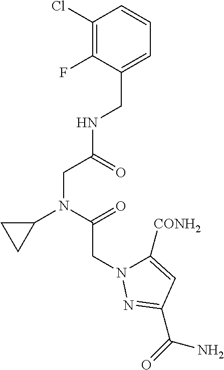 [SMILES: NC(=O)c1cc(C(N)=O)n(CC(=O)N(CC(=O)NCc2cccc(Cl)c2F)C2CC2)n1]

1-(2-((2-((3-chloro-2-fluorobenzyl)amino)-2-oxoethyl)(cyclopropyl)amino)-2-oxoethyl)-1H-pyrazole-3,5-dicarboxamide (9.0 mg) was prepared as described for 1-(2-((2-((3-chloro-2-fluorobenzyl)amino)-2-oxoethyl)(cyclopropyl)amino)-2-oxoethyl)-5-(trifluoromethyl)-1H-pyrazole-3-carboxamide. ¹H NMR. (DMSO-d6, 400 MHz) δ=8.37 (s, 1H), 8.09 (s, 1H), 7.55-7.45 (m, 3H), 7.39-7.16 (m, 4H), 5.65 (s, 2H), 4.34 (s, 2H), 3.95 (s, 2H), 2.98-2.90 (m, 1H), 0.94-086 (m, 4H). LRMS (M+H⁺) m/z calculated 451.1, found 451.2.

### Example 46: Preparation of 4-bromo-1-(2-((2-((3-chloro-2-fluorobenzyl)amino)-2-oxoethyl)(cyclopropyl)amino)-2-oxoethyl)-1H-pyrazole-3-carboxamide

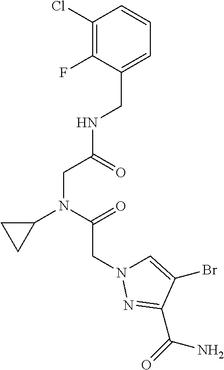 [SMILES: NC(=O)c1nn(CC(=O)N(CC(=O)NCc2cccc(Cl)c2F)C2CC2)cc1Br]

4-Bromo-1-(2-((2-((3-chloro-2-fluorobenzyl)amino)-2-oxoethyl)(cyclopropyl)amino)-2-oxoethyl)-1H-pyrazole-3-carboxamide (60.0 mg) was prepared as described for 1-(2-((2-((3-chloro-2-fluorobenzyl)amino)-2-oxoethyl)(cyclopropyl)amino)-2-oxoethyl)-5-(trifluoromethyl)-1H-pyrazole-3-carboxamide. ¹H NMR (DMSO-d6, 400 MHz) δ=8.45 (t, 1H), 7.95 (s, 1H), 7.50-7.45 (m, 2H), 7.28-7.24 (m, 2H), 7.20-7.18 (m, 1H), 5.35 (s, 2H), 4.34-4.33 (m, 2H), 3.98 (s, 2H), 2.92-2.91 (s, 1H), 0.88-0.86 (m, 4H). LRMS (M+H⁺) m/z calculated 486.0, found 486.6.

### Example 47: Preparation of 2-(3-acetyl-1H-indazol-1-yl)-N-(2-((3-chloro-2-fluorobenzyl)amino)-2-oxoethyl)-N-cyclopropylacetamide

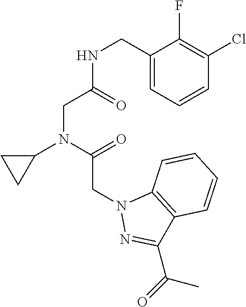 [SMILES: CC(=O)c1nn(CC(=O)N(CC(=O)NCc2cccc(Cl)c2F)C2CC2)c2ccccc12]

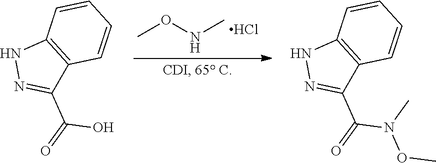

A solution of 1H-indazole-3-carboxylic acid (1.0 g, 6.17 mmol, 1.0 eq.) and CDI (1.1 g, 6.78 mmol, 1.1 eq.) in DMF (20 mL) was stirred at 65° C. for 2 h, then cooled down to r.t., N,O-dimethylhydroxylamine hydrochloride (661 mg, 6.78 mmol, 1.1 eq.) was added and the mixture was stirred at 65° C. for 12 h. The solvent of the mixture was removed under vacuum and the residue was purified by flash chromatography (elute: EA/PE=1/2) to give N-methoxy-N-methyl-1H-indazole-3-carboxamide (950 mg, 75.4% yield) as a yellow solid.

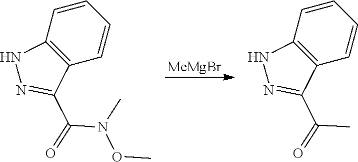

To a solution of N-methoxy-N-methyl-1H-indazole-3-carboxamide (950 mg, 4.63 mmol, 1.0 eq.) in dry THF, the mixture was cooled down to 0° C. Methylmagnesium bromide (7.7 ml, 23.17 mmol, 5.0 eq.) was added dropwise to the mixture at 0° C. The resulting mixture was stirred at r.t. for 12 h. Sat. NH₄Cl solution (5 ml) was added to the mixture. The solvent of the mixture was removed under vacuum, and the resulting residue was purified by column chromatography (EA/PE=1/2) to provide 1-(1H-indazol-3-yl)ethanone (610 mg, 82.3% yield) as yellow solid.

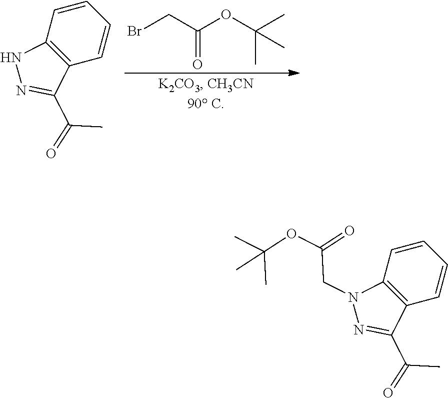

A solution of 1-(1H-indazol-3-yl)ethanone (610 mg, 3.8 mmol, 1.0 eq.), tert-butyl 2-bromoacetate (892 mg, 4.6 mmol, 1.2 eq) and K₂CO₃ (1.4 g, 9.9 mmol, 2.6 eq.) in CH₃CN (20 mL) was stirred at 90° C. for 12 h, and then cooled and concentrated under vacuum, the resulting residue was purified by column chromatography (EA/PE=1/5) to provide tert-butyl 2-(3-acetyl-1H-indazol-1-yl)acetate (980 mg, 93.9% yield).

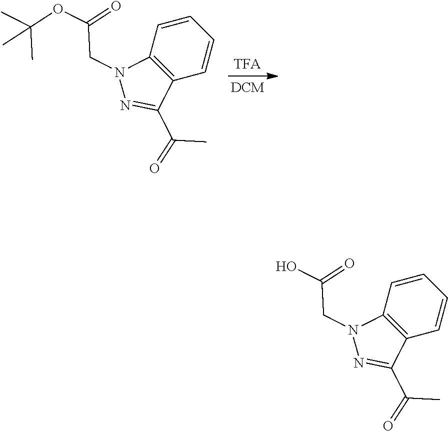 [SMILES: CC(=O)c1nn(CC(=O)OC(C)(C)C)c2ccccc12 ; CC(=O)c1nn(CC(=O)O)c2ccccc12]

To a solution of tert-butyl 2-(3-acetyl-1H-indazol-1-yl)acetate (980 mg, 3.6 mmol, 1.0 eq.) in DCM (10 mL) was added TFA (5 mL). The mixture was stirred at r.t. for 12 h., and then concentrated under vacuum to provide crude 2-(3-acetyl-1H-indazol-1-yl)acetic acid (780 mg, quant. yield).

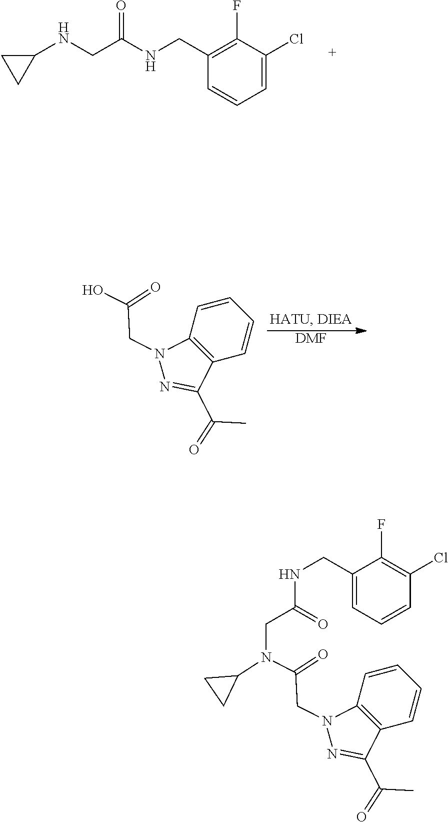

To a solution of N-(3-chloro-2-fluorobenzyl)-2-(ethylamino)acetamide (34 mg, 0.16 mmol, 1.0 eq.) and 2-(3-carbamoyl-1H-indazol-1-yl)acetic acid (40 mg, 0.16 mmol, 1.0 eq.) in DMF (3 mL) were added HATU (89 mg, 0.24 mmol, 1.5 eq.) and DIEA (61 mg, 0.47 mmol, 3.0 eq.). The mixture was stirred at r.t. for 2 h. Ethyl acetate (50 mL) and water (50 mL) were added. The organic layer was separated and concentrated. The resulting residue was purified by Prep-TLC (DCM/MeOH=10:1) to provide 2-(3-acetyl-1H-indazol-1-yl)-N-(2-((3-chloro-2-fluorobenzyl) amino)-2-oxoethyl)-N-cyclopropylacetamide (46.0 mg, yield 63.0%). ¹H NMR (CD3OD, 400 MHz) δ=8.25 (d, 1H), 7.60 (d, 1H), 7.44 (t, 1H), 7.33 (t, 2H), 7.24 (t, 1H), 7.00 (t, 1H), 5.75 (s, 2H), 4.44 (s, 2H), 4.14 (s, 2H), 3.13 (s, 1H), 2.66 (s, 3H), 1.04-1.07 (m, 4H). LRMS (M+H⁺) m/z calculated 456.1, found 457.6.

### Example 48: Preparation of 2-(1-acetylimidazo[1,5-a]pyridin-3-yl)-N-(2-((3-chloro-2-fluorobenzyl)amino)-2-oxoethyl)-N-cyclopropylacetamide

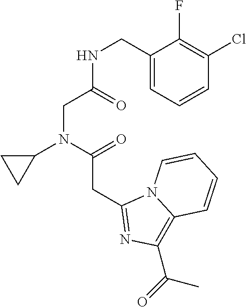 [SMILES: CC(=O)c1nc(CC(=O)N(CC(=O)NCc2cccc(Cl)c2F)C2CC2)n2ccccc12]

2-(1-acetylimidazo[1,5-a]pyridin-3-yl)-N-(2-((3-chloro-2-fluorobenzyl)amino)-2-oxoethyl)-N-cyclopropylacetamide (30 mg) was prepared as described for 2-(3-acetyl-1H-indazol-1-yl)-N-(2-((3-chloro-2-fluorobenzyl)amino)-2-oxoethyl)-N-cyclopropylacetamide. ¹H NMR. (MeOH-d4, 400 MHz) δ=8.24-8.31 (m, 2H), 7.32-7.35 (t, 2H), 7.22-7.26 (m, 1H), 7.00-7.04 (m, 1H), 6.93-6.96 (m, 1H), 7.62 (s, 2H), 7.45 (s, 2H), 4.13 (s, 2H), 3.09-3.15 (m, 1H), 2.59 (s, 3H), 1.02-1.03 (m, 4H). LRMS (M+H⁺) m/z calculated 457.1, found 457.6.

### Example 49: Preparation of N-(2-((3-chloro-2-fluorobenzyl)amino)-2-oxoethyl)-N-cyclopropyl-2-(3-(1-hydroxyethyl)-1H-indazol-1-yl)acetamide

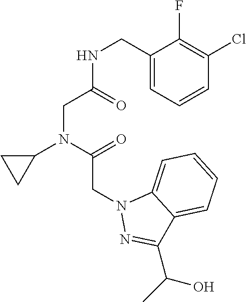 [SMILES: CC(O)c1nn(CC(=O)N(CC(=O)NCc2cccc(Cl)c2F)C2CC2)c2ccccc12]

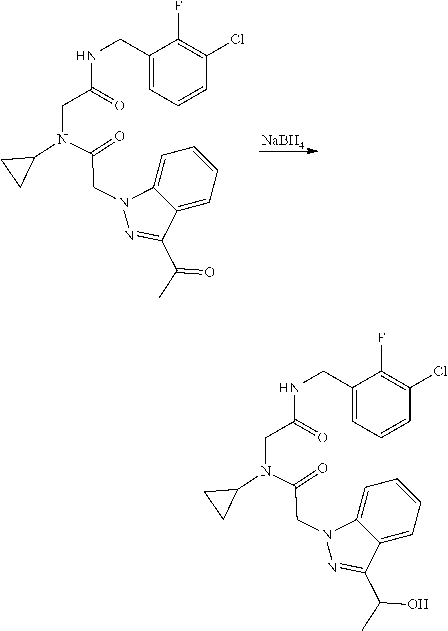 [SMILES: CC(=O)c1nn(CC(=O)N(CC(=O)NCc2cccc(Cl)c2F)C2CC2)c2ccccc12 ; "NaBH4" ; CC(O)c1nn(CC(=O)N(CC(=O)NCc2cccc(Cl)c2F)C2CC2)c2ccccc12]

NaBH₄ (4.0 mg, 0.1 mmol, 2.5 eq.) was added to a solution of 2-(3-acetyl-1H-indazol-1-yl)-N-(2-((3-chloro-2-fluorobenzyl)amino)-2-oxoethyl)-N-cyclopropylacetamide (20.0 mg, 0.04 mmol, 1.0 eq.) in MeOH (5 mL). The resulting mixture was stirred at r.t. for 2 h, and then cooled and concentrated under vacuum. The resulting residue was purified by column chromatography (DCM/MeOH=1:20) to provide N-(2-((3-chloro-2-fluorobenzyl)amino)-2-oxoethyl)-N-cyclopropyl-2-(3-(1-hydroxyethyl)-1H-indazol-1-yl)acetamide (16.0 mg, 80%). ¹H NMR. (CD₃OD, 400 MHz) δ=7.95 (s, 1H), 7.48 (d, 1H), 7.32-7.40 (m, 2H), 7.23 (t, 1H), 7.15 (t, 1H), 7.02 (t, 1H), 5.56 (s, 2H), 5.23 (d, 1H), 4.44 (s, 2H), 4.12 (s, 2H), 3.08 (t, 1H), 1.65 (d, 3H), 1.01-1.05 (m, 4H). LRMS (M+H⁺⁾m/z calculated 459.2, found 459.7.

### Example 50: Preparation of 3-(3-(2-((3-chloro-2-fluorobenzyl)amino)-2-oxoethyl)-3-isopropylureido)-1H-indole-1-carboxamide

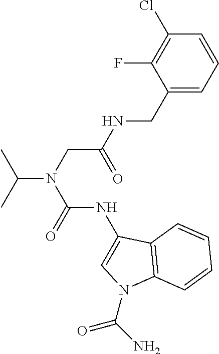 [SMILES: CC(C)N(CC(=O)NCc1cccc(Cl)c1F)C(=O)Nc1cn(C(N)=O)c2ccccc12]

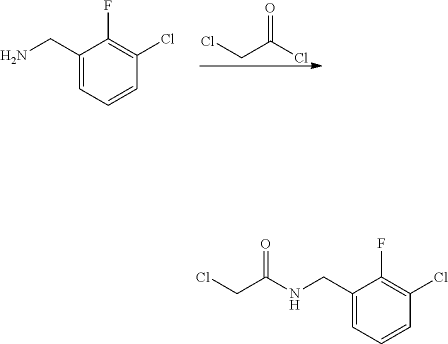 [SMILES: NCc1cccc(Cl)c1F ; O=C(Cl)CCl ; O=C(CCl)NCc1cccc(Cl)c1F]

2-Chloroacetyl chloride (142.0 mg, 1.25 mmol, 1.0 eq.) was added to a solution of (3-chloro-2-fluorophenyl) methanamine (200.0 mg, 1.25 mmol, 1.0 eq.) in dry toluene (10 mL). The resulting mixture was stirred at r.t. for 3 h, and then cooled and concentrated under vacuum. The resulting residue was purified by column chromatography (EA/PE=1:5) to provide 2-chloro-N-(3-chloro-2-fluorobenzyl)acetamide (200.0 mg, 67.8% yield).

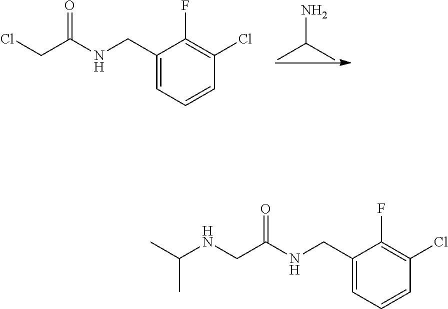

Propan-2-amine (30.0 mg, 0.51 mmol, 1.2 eq.) was added to a solution of 2-chloro-N-(3-chloro-2-fluorobenzyl)acetamide (100 mg, 0.43 mmol, 1.0 eq.) and K₂CO₃ (88 mg, 0.64 mmol, 1.5 eq.) in dry DMF (5 mL). The resulting mixture was stirred at r.t. overnight, then cooled, filtered and quenched by water. The mixture was extracted with EtOAc (2×50 mL). The combined organic layers were dried over anhydrous Na₂SO₄ and concentrated under vacuum. The resulting residue was purified by silica column chromatography (EA/PE=1:5) to provide to provide N-(3-chloro-2-fluorobenzyl)-2-(isopropylamino) acetamide (90 mg, 81.8% yield).

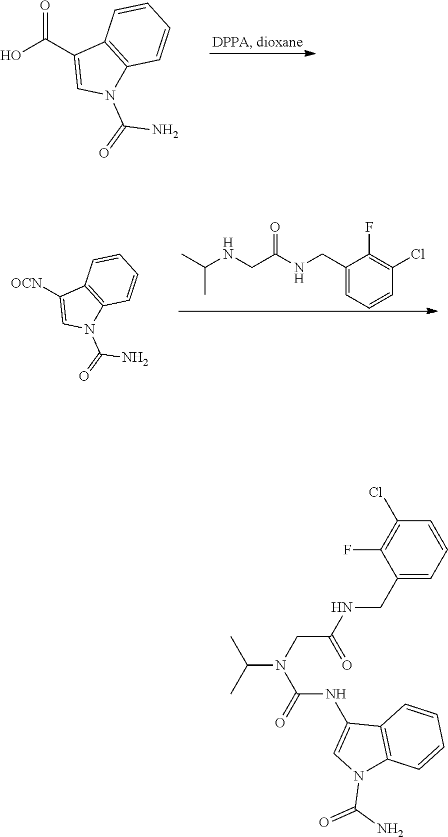

To a solution of 1-carbamoyl-1H-indole-3-carboxylic acid (24.0 mg, 0.12 mmol, 1.0 eq.) in 1,4-dioxane (4 mL) were added to DPPA (38.0 mg, 0.14 mmol, 1.2 eq.) and TEA (14.0 mg, 0.14 mmol, 1.2 eq.). The resulting mixture was stirred at r.t. for 2 h. The mixture was then added to N-(3-chloro-2-fluorobenzyl)-2-(isopropylamino) acetamide (30 mg, 0.12 mmol, 1.0 eq.). The mixture was stirred at 100° C. for 2 h, then cooled and evaporated. The residue was purified by Prep-HPLC to provide 3-(3-(2-((3-chloro-2-fluorobenzyl)amino)-2-oxoethyl)-3-isopropylureido)-1H-indole-1-carboxamide (18.0 mg, 34.0% yield). ¹H NMR (CD₃OD, 400 MHz) δ=8.23 (d, 1H), 7.74 (s, 1H), 7.58 (d, 1H), 7.36 (m, 2H), 7.29 (t, 1H), 7.19 (t, 1H), 7.08 (t, 1H), 4.45-4.53 (m, 3H), 4.03 (s, 2H), 1.19-1.23 (m, 6H). LRMS (M+H⁺) m/z calculated 460.0, found 460.1.

### Example 51: Preparation of 3-(3-(2-((3-chloro-2-fluorobenzyl)amino)-2-oxoethyl)-3-cyclopropylureido)-1H-indole-1-carboxamide

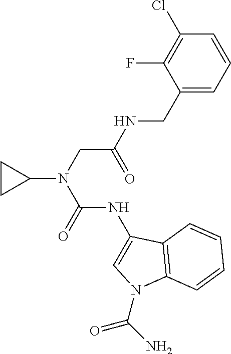 [SMILES: NC(=O)n1cc(NC(=O)N(CC(=O)NCc2cccc(Cl)c2F)C2CC2)c2ccccc21]

3-(3-(2-((3-chloro-2-fluorobenzyl)amino)-2-oxoethyl)-3-cyclopropylureido)-1H-indole-1-carboxamide (6.0 mg) was prepared as described for 3-(3-(2-((3-chloro-2-fluorobenzyl)amino)-2-oxoethyl)-3-isopropylureido)-1H-indole-1-carboxamide. ¹H NMR (CD₃OD, 400 MHz) δ=8.25 (d, 1H), 7.8 (s, 1H), 7.60 (d, 1H), 7.30-7.34 (m, 3H), 7.22 (t, 1H), 7.10 (t, 1H), 4.47 (s, 2H), 4.13 (s, 2H), 2.96 (s, 1H), 1.03 (d, 2H), 0.93 (s, 2H). LRMS (M+H⁺) m/z calculated 458.1, found 458.2.

### Example 52: Preparation of 3-(3-(2-((3-chloro-2-fluorobenzyl)amino)-2-oxoethyl)-3-(pyrrolidin-3-yl)ureido)-1H-indole-1-carboxamide

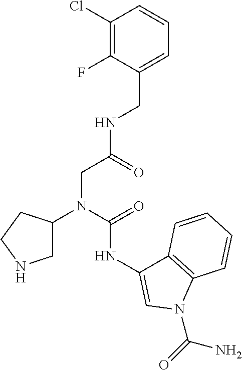 [SMILES: NC(=O)n1cc(NC(=O)N(CC(=O)NCc2cccc(Cl)c2F)C2CCNC2)c2ccccc21]

3-(3-(2-((3-chloro-2-fluorobenzyl)amino)-2-oxoethyl)-3-(pyrrolidin-3-yl)ureido)-1H-indole-1-carboxamide (16.0 mg) was prepared as described for 3-(3-(2-((3-chloro-2-fluorobenzyl)amino)-2-oxoethyl)-3-isopropylureido)-1H-indole-1-carboxamide. ¹H NMR (CD₃OD, 400 MHz) δ=8.21 (d, 1H), 7.54 (d, 1H), 7.45-7.39 (m, 4H), 7.14 (t, 1H), 5.40 (s, 2H), 4.55 (s, 2H), 4.40 (s, 2H), 4.20 (s, 1H), 3.54 (s, 1H), 3.31 (d, 2H). LRMS (M+H⁺) m/z calculated 487.2, found 487.1.

### Example 53: Preparation of 1-(3-(2-((3-chloro-2-fluorobenzyl)amino)-2-oxoethyl)-3-cyclopropylureido)imidazo[1,5-a]pyridine-3-carboxamide

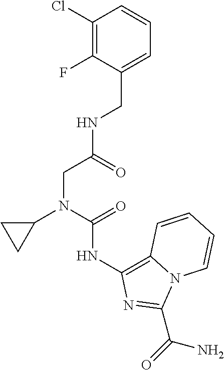 [SMILES: NC(=O)c1nc(NC(=O)N(CC(=O)NCc2cccc(Cl)c2F)C2CC2)c2ccccn12]

1-(3-(2-((3-chloro-2-fluorobenzyl)amino)-2-oxoethyl)-3-cyclopropylureido)imidazo[1,5-a]pyridine-3-carboxamide (21.0 mg) was prepared as described for 3-(3-(2-((3-chloro-2-fluorobenzyl)amino)-2-oxoethyl)-3-isopropylureido)-1H-indole-1-carboxamide. ¹H NMR (CD₃OD, 400 MHz) δ=9.36 (d, 1H), 7.66 (d, 1H), 7.31-7.38 (m, 2H), 7.09 (t, 1H), 7.02 (t, 1H), 6.93 (t, 1H), 4.48 (s, 2H), 4.12 (s, 2H), 2.94-2.97 (m, 1H), 0.96-1.00 (m, 4H). LRMS (M+H⁺) m/z calculated 459.1, found 459.2.

### Example 54: Preparation of 1-(2-((2-((3-chloro-2-fluorobenzyl)amino)-2-oxoethyl)(3-methylbutan-2-yl)amino)-2-oxoethyl)-1H-indazole-3-carboxamide

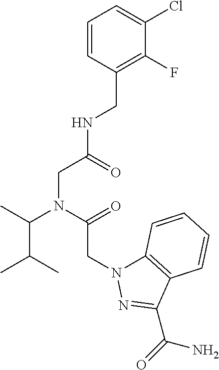 [SMILES: CC(C)C(C)N(CC(=O)NCc1cccc(Cl)c1F)C(=O)Cn1nc(C(N)=O)c2ccccc21]

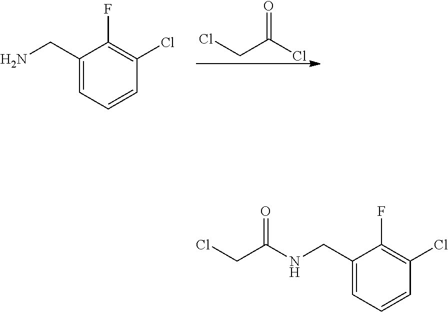

To a solution of (3-chloro-2-fluorophenyl)methanamine (1.6 g, 10.0 mmol, 1.0 eq) and Et₃N (1.51 g, 15.0 mmol, 1.0 eq) in dichloromethane (100.0 mL) was added 2-chloroacetyl chloride (1.7 g, 15.0 mmol, 1.5 eq). The mixture was stirred at r.t. for 16 h, then washed with aq. 10% NaHCO₃ solution (100.0 mL), dried over anhydrous Na₂SO₄, filtered and concentrated in vacuo to provide crude 2-chloro-N-(3-chloro-2-fluorobenzyl)acetamide (2.2 g, 93%).

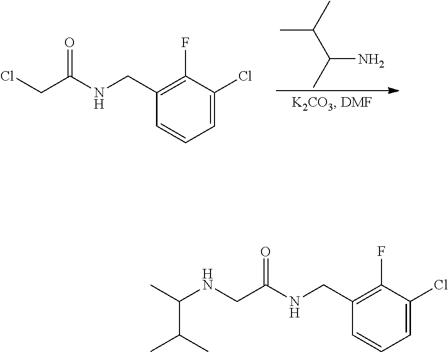

To a solution of 2-chloro-N-(3-chloro-2-fluorobenzyl)acetamide (150.0 mg, 0.63 mmol, 1.0 eq) and K₂CO₃ (220.0 mg, 1.59 mmol, 2.5 eq) in DMF (4.0 mL) was added a solution of 3-methylbutan-2-amine (66.0 mg, 0.76 mmol, 1.2 eq). The reaction was stirred at room temperature for 16 h. Ethyl acetate (50.0 mL) and water (50.0 mL) were added. The organic layer was separated, dried over anhydrous Na₂SO₄, filtered and concentrated in vacuo. The resulting residue was purified on column chromatography (EA/PE=1/3, v/v) to provide N-(3-chloro-2-fluorobenzyl)-2-((3-methylbutan-2-yl)amino)acetamide (95.0 mg, 52.7%).

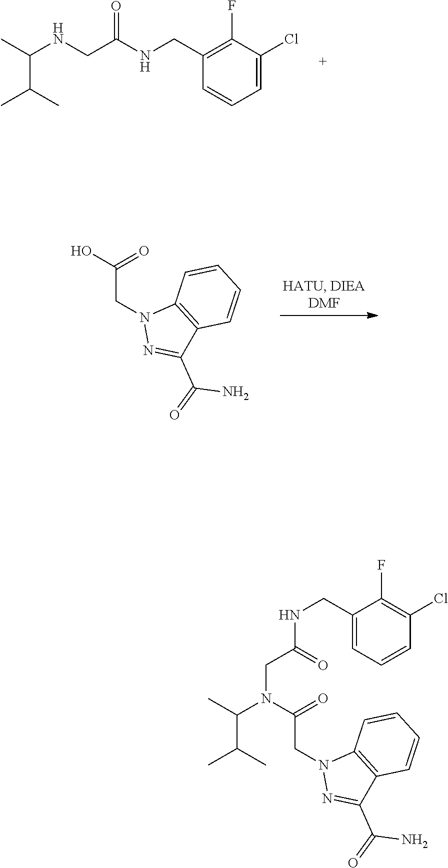 [SMILES: CC(C)C(C)NCC(=O)NCc1cccc(Cl)c1F ; NC(=O)c1nn(CC(=O)O)c2ccccc12 ; CC(C)C(C)N(CC(=O)NCc1cccc(Cl)c1F)C(=O)Cn1nc(C(N)=O)c2ccccc21]

To a solution of N-(3-chloro-2-fluorobenzyl)-2-((3-methylbutan-2-yl)acetic acid (110.0 mg, 0.33 mmol 1.0 eq) in DMF (3.0 mL) were added HATU (316.0 mg, 0.83 mmol, 2.5 eq) and DIEA (170.0 mg, 1.33 mmol, 4.0 eq). The mixture was stirred at rt for 16 h. Ethyl acetate (50.0 mL) and water (50.0 mL) were added. The organic layer was separated separated, dried over anhydrous Na₂SO₄, filtered and concentrated in vacuo. The resulting residue was purified by prep-HPLC to provide 1-(2-((2-((3-chloro-2-fluorobenzyl)amino)-2-oxyethyl(3-methylbutan-2-yl)amino)-2-oxoethyl)-1H-indazole-3-carboxamide (75.0 mg, 46.6%). ¹H NMR (CD₃MD, 400 MHz) δ 8.23-8.22 (m, 1H), 7.62-7.16 (m, 5H), 7.14-6.95 (m, 1H), 5.56-5.46 (m, 2H), 4.77 (s, 2H), 4.55-4.07 (m, 2H), 3.81-3.74 (m, 1H), 1.70-1.69 (m, 1H), 1.34-0.77 (m, 9H). LCMS (M+H⁺) m/z calculated 488.2, found 488.7.

### Example 55: Preparation of 1-(2-((2-((3-chloro-2-fluorobenzyl)amino)-2-oxoethyl)(1-cyclopropylethyl)amino)-2-oxoethyl)-H-indazole-3-carboxamide

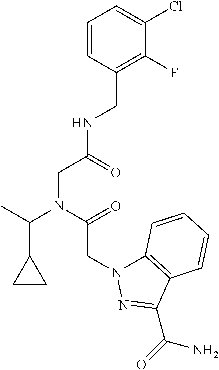 [SMILES: CC(C1CC1)N(CC(=O)NCc1cccc(Cl)c1F)C(=O)Cn1nc(C(N)=O)c2ccccc21]

1-(2-((2-((3-Chloro-2-fluorobenzyl)amino)-2-oxoethyl)(1-cyclopropylethyl)amino)-2-oxoethyl)-1H-indazole-3-carboxamide (107.4 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22-8.20 (m, 1H), 7.63-7.57 (m, 1H), 7.55-7.23 (m, 4H), 7.16-7.00 (m, 1H), 5.60-5.42 (m, 2H), 4.55-4.11 (m, 4H), 3.83-3.54 (m, 1H), 1.15-1.06 (m, 3H), 0.87-0.80 (m, 1H), 0.65-0.44 (m, 2H), 0.34-0.15 (m, 2H). LCMS (M+H⁺) m/z calculated 486.2, found 486.8.

### Example 56: Preparation of 1-(2-(sec-butyl(2-((3-chloro-2-fluorobenzyl)amino)-2-oxoethyl)amino)-2-oxoethyl)-1H-indazole-3-carboxamide

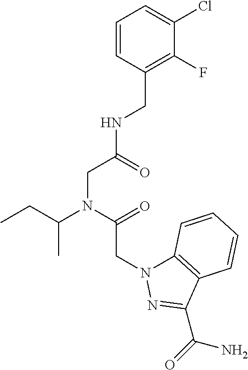 [SMILES: CCC(C)N(CC(=O)NCc1cccc(Cl)c1F)C(=O)Cn1nc(C(N)=O)c2ccccc21]

1-(2-(Sec-butyl(2-((3-chloro-2-fluorobenzyl)amino)-2-oxoethyl)amino)-2-oxoethyl)-1H-indazole-3-carboxamide (283.5 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CDCl₃, 400 MHz) δ 8.38 (d, 1H), 7.43 (t, 1H), 7.60-7.63 (m, 1H), 7.37-7.26 (m, 3H), 7.13 (t, 1H), 7.00-6.94 (m, 2H), 6.81 (s, 1H), 5.42 (s, 1H), 5.32 (s, 2H), 4.39 (d, 2H), 3.98-3.86 (m, 3H), 1.57 (s, 2H), 1.20 (d, 3H), 0.87 (t, 3H). LCMS (M+H⁺) m/z calculated 474.2, found 474.7.

### Example 57: Preparation of 1-(2-((2-((3-chloro-2-fluorobenzyl)amino)-2-oxoethyl)(4-hydroxybutan-2-yl)amino)-2-oxoethyl)-1H-indazole-3-carboxamide

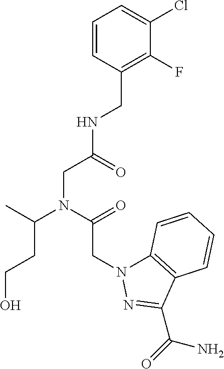 [SMILES: CC(CCO)N(CC(=O)NCc1cccc(Cl)c1F)C(=O)Cn1nc(C(N)=O)c2ccccc21]

1-(2-((2-((3-Chloro-2-fluorobenzyl)amino)-2-oxoethyl)(4-hydroxybutan-2-yl)amino)-2-oxoethyl)-1H-indazole-3-carboxamide (14.4 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD-d4, 400 MHz) δ 8.22 (d, 1H), 7.61-7.55 (m, 1H), 7.46-7.36 (m, 2H), 7.32-7.21 (m, 2H), 7.15-6.92 (m, 1H), 5.82-5.46 (m, 2H), 4.57 (d, 1H), 4.41 (s, 2H), 4.29-3.67 (m, 4H), 1.90-1.58 (m, 2H), 1.34-1.12 (m, 3H). LCMS (M+H⁺) m/z calculated 490.2, found 490.2.

### Example 58: Preparation of 1-(2-((2-((3-chloro-2-fluorobenzyl)amino)-2-oxoethyl)(1-hydroxypropan-2-yl)amino)-2-oxoethyl)-1H-indazole-3-carboxamide

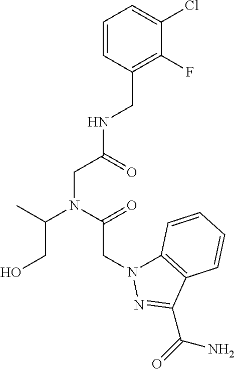 [SMILES: CC(CO)N(CC(=O)NCc1cccc(Cl)c1F)C(=O)Cn1nc(C(N)=O)c2ccccc21]

1-(2-((2-((3-Chloro-2-fluorobenzyl)amino)-2-oxoethyl)(1-hydroxypropan-2-yl)amino)-2-oxoethyl)-1H-indazole-3-carboxamide (13.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.23 (d, 1H), 7.48 (d, 1H), 7.39 (t, 1H), 7.26-7.31 (m, 2H), 7.21 (t, 1H), 6.91 (t, 1H), 5.59-5.71 (m, 2H), 4.42 (s, 2H), 4.28-4.36 (m, 1H), 3.98 (s, 2H), 3.53-3.66 (m, 2H), 1.20 (d, 3H). LCMS (M+H⁺) m/z calculated 476.2, found 476.2.

### Example 59: Preparation of methyl 2-(2-(3-carbamoyl-1H-indazol-1-yl)-N-(2-((3-chloro-2-fluorobenzyl)amino)-2-oxoethyl)acetamido)propanoate

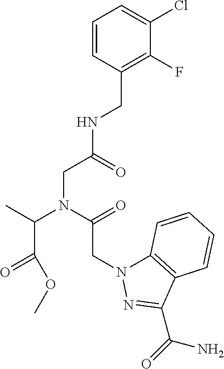 [SMILES: COC(=O)C(C)N(CC(=O)NCc1cccc(Cl)c1F)C(=O)Cn1nc(C(N)=O)c2ccccc21]

Methyl 2-(2-(3-carbamoyl-1H-indazol-1-yl)-N-(2-((3-chloro-2-fluorobenzyl)amino)-2-oxoethyl)acetamido)propanoate (13.2 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (DMSO-d6, 400 MHz) δ 8.79 (t, 1H), 8.17 (d, 1H), 7.69 (s, 1H), 7.50-7.35 (m, 4H), 7.27-7.19 (m, 3H), 5.56-5.45 (q, 2H), 4.48-4.41 (q, 2H), 3.72 (s, 1H), 3.57 (d, 2H), 3.16 (d, 2H), 1.51 (d, 1H), 1.25 (s, 1H), 1.23 (s, 2H). LCMS (M+H⁺) m/z calculated 504.1, found 504.2.

### Example 60: Preparation of 1-(2-((2-((3-chloro-2-fluorobenzyl)amino)-2-oxoethyl)(4-fluorobutan-2-yl)amino)-2-oxoethyl)-1H-indazole-3-carboxamide

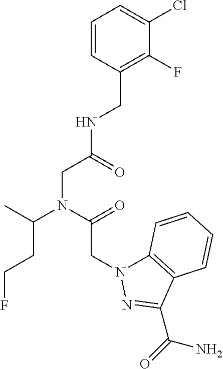 [SMILES: CC(CCF)N(CC(=O)NCc1cccc(Cl)c1F)C(=O)Cn1nc(C(N)=O)c2ccccc21]

1-(2-((2-((3-Chloro-2-fluorobenzyl)amino)-2-oxoethyl)(4-fluorobutan-2-yl)amino)-2-oxoethyl)-1H-indazole-3-carboxamide (5.8 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide.

H NMR (CD₃OD, 400 MHz) δ 8.22 (d, 1H), 7.61-7.55 (m, 1H), 7.46-7.36 (m, 2H), 7.32-7.21 (m, 2H), 7.15-6.92 (m, 1H), 5.82-5.46 (m, 2H), 4.55 (s, 1H), 4.41 (s, 2H), 4.29-3.92 (m, 2H), 2.03-1.90 (m, 2H), 1.34 (d, 2H), 1.30-1.15 (m, 3H). LCMS (M+H⁺) m/z calculated 492.2, found 492.2.

### Example 61: Preparation of 1-(2-((2-((3-chloro-2-fluorobenzyl)amino)-2-oxoethyl)(1-methoxybutan-2-yl)amino)-2-oxoethyl)-1H-indazole-3-carboxamide

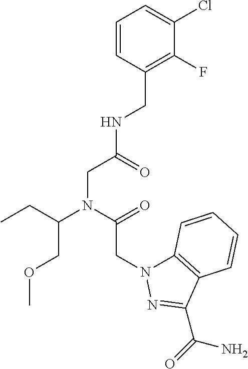 [SMILES: CCC(COC)N(CC(=O)NCc1cccc(Cl)c1F)C(=O)Cn1nc(C(N)=O)c2ccccc21]

1-(2-((2-((3-Chloro-2-fluorobenzyl)amino)-2-oxoethyl)(1-methoxybutan-2-yl)amino)-2-oxoethyl)-1H-indazole-3-carboxamide (6.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. 1H NMR (DMSO-d6, 400 MHz) δ 8.74-8.22 (m, 1H), 8.19-8.17 (d, 1H), 7.67 (s, 1H), 7.54-7.51 (t, 1H), 7.45-7.40 (m, 3H), 7.35-7.34 (d, 2 H), 7.27-7.22 (m, 1H), 5.61-5.45 (m, 2H), 4.47 (s, 1H), 4.38-4.36 (m, 1H), 4.31-4.27 (t, 1H), 4.23-4.21 (d, 1H). 4.07-3.82 (m, 1H), 3.11 (s, 2H), 1.61-1.37 (m, 2H), 1.24-1.16 (m, 1H), 1.01-0.97 (m, 1H), 0.78-0.74 (m, 2H). LCMS (M+H⁺) m/z calculated 504.1, found 504.2.

### Example 62: Preparation of 1-(2-((1-amino-1-oxopropan-2-yl)(2-((3-chloro-2-fluorobenzyl)amino)-2-oxoethyl)amino)-2-oxoethyl)-1H-indazole-3-carboxamide

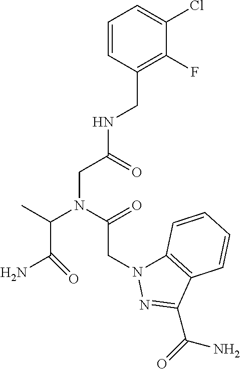 [SMILES: CC(C(N)=O)N(CC(=O)NCc1cccc(Cl)c1F)C(=O)Cn1nc(C(N)=O)c2ccccc21]

1-(2-((1-Amino-1-oxopropan-2-yl)(2-((3-chloro-2-fluorobenzyl)amino)-2-oxoethyl)amino)-2-oxoethyl)-1H-indazole-3-carboxamide (2.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22-8.20 (d, 1H), 7.52-7.50 (m, 1H), 7.44-7.36 (m, 2H), 7.30-7.26 (t, 2H), 7.14-7.11 (m, 1H), 6.96-6.92 (m, 1H), 5.56-5.28 (m, 2H), 4.56-4.52 (m, 4H), 1.60-1.59 (m, 1H), 1.39-1.37 (d, 2H), 1.29 (s, 1H). LCMS (M+H⁺) m/z calculated 489.1, found 489.2.

### Example 63: Preparation of 1-(2-((2-((3-chloro-2-fluorobenzyl)amino)-2-oxoethyl)(2-methylcyclopropyl)amino)-2-oxoethyl)-1H-indazole-3-carboxamide

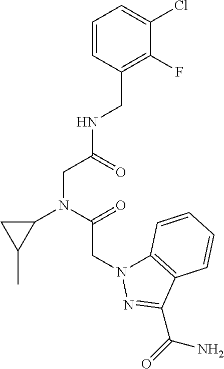 [SMILES: CC1CC1N(CC(=O)NCc1cccc(Cl)c1F)C(=O)Cn1nc(C(N)=O)c2ccccc21]

1-(2-((2-((3-Chloro-2-fluorobenzyl)amino)-2-oxoethyl)(2-methylcyclopropyl)amino)-2-oxoethyl)-1H-indazole-3-carboxamide (31.4 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.58 (d, 1H), 7.42 (t, 1H), 7.22-7.35 (m, 3H), 7.02 (t, 1H), 5.64 (s, 2H), 4.44 (s, 2H), 4.10 (s, 2H), 2.78-2.80 (m, 1H), 1.37-1.46 (m, 1H), 1.12-1.19 (m, 4H), 0.78-0.83 (m, 1H). LCMS (M+H⁺) m/z calculated 472.1, found 472.2.

### Example 64: Preparation of 1-(2-((2-((3-chloro-2-fluorobenzyl)amino)-2-oxoethyl)(3,3-dimethylbutan-2-yl)amino)-2-oxoethyl)-1H-indazole-3-carboxamide

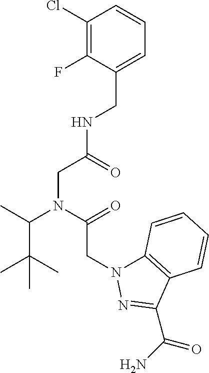 [SMILES: CC(N(CC(=O)NCc1cccc(Cl)c1F)C(=O)Cn1nc(C(N)=O)c2ccccc21)C(C)(C)C]

1-(2-((2-((3-Chloro-2-fluorobenzyl)amino)-2-oxoethyl)(3,3-dimethylbutan-2-yl)amino)-2-oxoethyl)-1H-indazole-3-carboxamide (280.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (DMSO-d6, 400 MHz) δ 8.81 (t, 1H), 8.17 (d, 1H), 7.65 (s, 1H), 7.56-7.48 (m, 2H), 7.44-7.34 (m, 3H), 7.27-7.19 (m, 2H), 5.62-5.50 (m, 2H), 5.36 (d, 1H), 4.47 (t, 2H), 4.41 (d, 1H), 4.13 (d, 1H), 1.03 (s, 1H), 0.98 (d, 2H), 0.83 (s, 6H), 0.821 (s, 3H). LCMS (M+H⁺) m/z calculated 502.2, found 502.2.

### Example 65: Preparation of 1-(2-((2-((3-chloro-2-fluorobenzyl)amino)-2-oxoethyl)(1-phenylethyl)amino)-2-oxoethyl)-1H-indazole-3-carboxamide

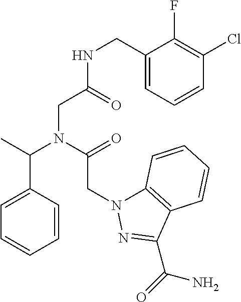 [SMILES: CC(c1ccccc1)N(CC(=O)NCc1cccc(Cl)c1F)C(=O)Cn1nc(C(N)=O)c2ccccc21]

1-(2-((2-((3-Chloro-2-fluorobenzyl)amino)-2-oxoethyl)(1-phenylethyl)amino)-2-oxoethyl)-1H-indazole-3-carboxamide (182.8 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃CD, 400 MHz) δ 8.19-8.24 (m, 1H), 6.97-7.66 (m, 11H), 5.88-5.90 (m, 1H), 5.47-5.66 (m, 2H), 4.31-4.41 (m, 2H), 3.74-4.16 (m, 2H), 1.45-1.71 (m, 3H). LCMS (M+H⁺) m/z calculated 522.1, found 522.2.

### Example 66: Preparation of 1-(2-((2-((3-chloro-2-fluorobenzyl)amino)-2-oxoethyl)(1-fluoropropan-2-yl)amino)-2-oxoethyl)-1H-indazole-3-carboxamide

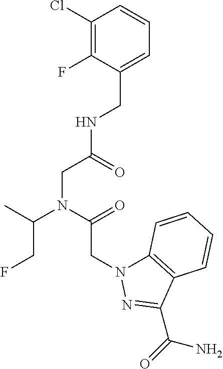 [SMILES: CC(CF)N(CC(=O)NCc1cccc(Cl)c1F)C(=O)Cn1nc(C(N)=O)c2ccccc21]

1-(2-((2-((3-Chloro-2-fluorobenzyl)amino)-2-oxoethyl)(1-fluoropropan-2-yl)amino)-2-oxoethyl)-1H-indazole-3-carboxamide (9.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.58 (d, 1H), 7.51 (d, 1H), 7.36-7.46 (m, 2H), 7.21-7.30 (m, 2H), 6.94-7.15 (m, 1H), 5.47-5.61 (m, 2H), 4.33-4.56 (m, 6H), 3.93-4.15 (m, 1H), 1.19-1.31 (m, 3H). LCMS (M+H⁺) m/z calculated 478.1, found 478.2.

### Example 67: Preparation of 1-(2-((2-((3-chloro-2-fluorobenzyl)amino)-2-oxoethyl)(1,1,1-trifluoropropan-2-yl)amino)-2-oxoethyl)-1H-indazole-3-carboxamide

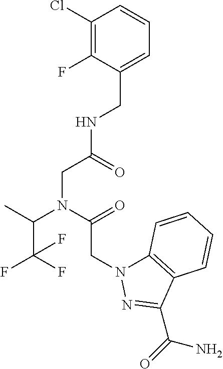 [SMILES: CC(N(CC(=O)NCc1cccc(Cl)c1F)C(=O)Cn1nc(C(N)=O)c2ccccc21)C(F)(F)F]

1-(2-((2-((3-Chloro-2-fluorobenzyl)amino)-2-oxoethyl)(1,1,1-trifluoropropan-2-yl)amino)-2-oxoethyl)-1H-indazole-3-carboxamide (8.1 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.56 (d, 1H), 7.50-7.19 (m, 4H), 7.15-6.92 (m, 1H), 5.75-5.47 (m, 2H), 5.36-5.00 (m, 1H), 4.61-4.49 (m, 2H), 4.42-3.93 (m, 2H), 1.60-1.34 (m, 3H). LCMS (M+H⁺) m/z calculated 514.1, found 514.2.

### Example 68: Preparation of 1-(2-((2-((3-chloro-2-fluorobenzyl)amino)-2-oxoethyl)(1-methoxypropan-2-yl)amino)-2-oxoethyl)-1H-indazole-3-carboxamide

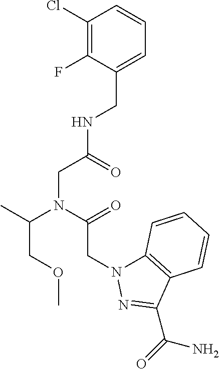 [SMILES: COCC(C)N(CC(=O)NCc1cccc(Cl)c1F)C(=O)Cn1nc(C(N)=O)c2ccccc21]

1-(2-((2-((3-Chloro-2-fluorobenzyl)amino)-2-oxoethyl)(1-methoxypropan-2-yl)amino)-2-oxoethyl)-1H-indazole-3-carboxamide (113.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (DMSO-d6, 400 MHz) δ 8.17-8.19 (m, 1H), 7.00-7.66 (m, 7H), 5.45-5.64 (m, 2H), 3.75-4.55 (m, 5H), 3.31-3.44 (m, 3H), 3.15-3.22 (m, 2H), 0.99-1.21 (m, 3H). LCMS (M+H⁺) m/z calculated 490.2, found 490.2.

### Example 69: Preparation of 1-(2-((2-((3-chloro-2-fluorobenzyl)amino)-2-oxoethyl)(1-cyclopropyl-2-hydroxyethyl)amino)-2-oxoethyl)-1H-indazole-3-carboxamide

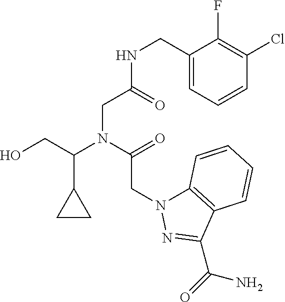 [SMILES: NC(=O)c1nn(CC(=O)N(CC(=O)NCc2cccc(Cl)c2F)C(CO)C2CC2)c2ccccc12]

1-(2-((2-((3-Chloro-2-fluorobenzyl)amino)-2-oxoethyl)(1-cyclopropyl-2-hydroxyethyl)amino)-2-oxoethyl)-1H-indazole-3-carboxamide (10.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.23-8.21 (m, 1H), 7.54-7.21 (m, 5H), 7.12-6.89 (m, 1H), 5.60-5.36 (m, 2H), 4.47 (s, 2H), 4.13 (s, 2H), 3.88-3.69 (m, 2H), 3.43-3.34 (m, 1H), 1.29-1.14 (m, 1H), 0.91-0.69 (m, 2H), 0.52-0.32 (m, 2H). LCMS (M+H⁺) m/z calculated 502.1, found 502.2.

### Example 70: Preparation of 1-(2-((2-((2-chloro-3-fluorobenzyl)amino)-2-oxoethyl)(1,3-difluoropropan-2-yl)amino)-2-oxoethyl)-1H-indazole-3-carboxamide

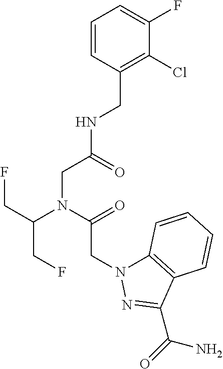 [SMILES: NC(=O)c1nn(CC(=O)N(CC(=O)NCc2cccc(F)c2Cl)C(CF)CF)c2ccccc12]

1-(2-((2-((2-Chloro-3-fluorobenzyl)amino)-2-oxoethyl)(1,3-difluoropropan-2-yl)amino)-2-oxoethyl)-1H-indazole-3-carboxamide (1.7 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD-d4, 400 MHz) δ 8.22 (d, 1H), 7.58-6.96 (m, 6H), 5.62-5.47 (m, 2H), 4.75-4.52 (m, 6H), 4.44 (s, 2H), 4.15 (s, 1H). LCMS (M+H⁺) m/z calculated 496.1, found 496.2.

### Example 71: Preparation of 1-(2-((4-aminobutan-2-yl)(2-((2-chloro-3-fluorobenzyl)amino)-2-oxoethyl)amino)-2-oxoethyl)-1H-indazole-3-carboxamide

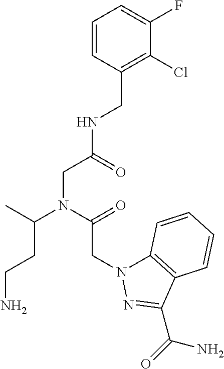 [SMILES: CC(CCN)N(CC(=O)NCc1cccc(F)c1Cl)C(=O)Cn1nc(C(N)=O)c2ccccc21]

1-(2-((4-Aminobutan-2-yl)(2-((2-chloro-3-fluorobenzyl)amino)-2-oxoethyl)amino)-2-oxoethyl)-1H-indazole-3-carboxamide (21.8 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.58-7.54 (m, 1H), 7.46-7.25 (m, 4H), 7.15-7.11 (m, 1H), 5.56-5.46 (m, 2H), 4.62-4.34 (m, 3H), 4.29 (s, 2H), 2.92 (t, 2H), 2.03-1.72 (m, 2H), 1.34-1.21 (m, 3H). LCMS (M+H⁺) m/z calculated 489.2, found 489.3.

### Example 72: Preparation of ethyl 3-(2-(3-carbamoyl-1H-indazol-1-yl)-N-(2-((3-chloro-2-fluorobenzyl)amino)-2-oxoethyl)acetamido)butanoate

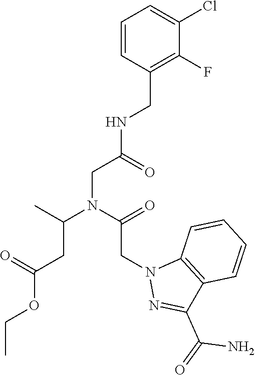 [SMILES: CCOC(=O)CC(C)N(CC(=O)NCc1cccc(Cl)c1F)C(=O)Cn1nc(C(N)=O)c2ccccc21]

Ethyl 3-(2-(3-carbamoyl-1H-indazol-1-yl)-N-(2-((3-chloro-2-fluorobenzyl)amino)-2-oxoethyl)acetamido)butanoate (3.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD-d4, 400 MHz) δ 8.23-8.203 (m, 1H), 7.61-7.56 (m, 1H), 7.43-7.21 (m, 4H), 7.13-6.92 (m, 1H), 5.94-5.41 (m, 2H), 4.80-4.68 (m, 2H), 4.40 (s, 2H), 4.24-3.95 (m, 3H), 2.82-2.49 (m, 2H), 1.32-1.16 (m, 6H). LCMS (M+H⁺) m/z calculated 532.2, found 532.2.

### Example 73: Preparation of 1-(2-((2-((3-chloro-2-fluorobenzyl)amino)-2-oxoethyl)(1-cyclopropyl-3-hydroxypropyl)amino)-2-oxoethyl)-1H-indazole-3-carboxamide

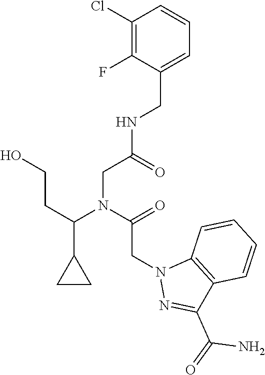 [SMILES: NC(=O)c1nn(CC(=O)N(CC(=O)NCc2cccc(Cl)c2F)C(CCO)C2CC2)c2ccccc12]

1-(2-((2-((3-Chloro-2-fluorobenzyl)amino)-2-oxoethyl)(1-cyclopropyl-3-hydroxypropyl)amino)-2-oxoethyl)-1H-indazole-3-carboxamide (10.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.25 (t, 1H), 7.66-7.53 (d, 1H), 7.53-7.38 (m, 2H), 7.35-7.24 (m, 2H), 7.18-6.94 (m, 1H), 5.68-5.49 (m, 2H), 4.59-4.43 (m, 2H), 4.43-4.03 (m, 2H), 3.89-3.74 (m, 1H), 3.65-3.44 (m, 2H), 2.05-1.75 (m, 2H), 1.08-0.90 (m, 1H), 0.70-0.47 (m, 2H), 0.31-0.18 (m, 2H). LCMS (M+Na⁺) m/z calculated 538.2, found 538.2.

### Example 74: Preparation of 3-(2-(3-carbamoyl-1H-indazol-1-yl)-N-(2-((3-chloro-2-fluorobenzyl)amino)-2-oxoethyl)acetamido)butanoic acid

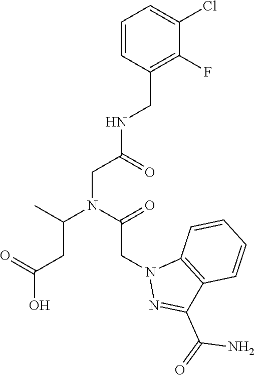 [SMILES: CC(CC(=O)O)N(CC(=O)NCc1cccc(Cl)c1F)C(=O)Cn1nc(C(N)=O)c2ccccc21]

3-(2-(3-Carbamoyl-1H-indazol-1-yl)-N-(2-((3-chloro-2-fluorobenzyl)amino)-2-oxoethyl)acetamido)butanoic acid (9.2 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.20 (d, 1H), 7.64 (d, 1H), 7.42-7.24 (m, 3H), 7.19-7.12 (m, 1H), 6.85 (t, 1H), 5.96-5.40 (m, 2H), 4.64 (s, 1H), 4.55-4.24 (m, 2H), 4.05-3.87 (m, 2H), 2.62-2.41 (m, 2H), 1.32-1.16 (m, 3H). LCMS (M+H⁺) m/z calculated 504.1, found 504.2.

### Example 75: Preparation of 1-(2-((3-amino-1-cyclopropyl-3-oxopropyl)(2-((3-chloro-2-fluorobenzyl)amino)-2-oxoethyl)amino)-2-oxoethyl)-1H-indazole-3-carboxamide

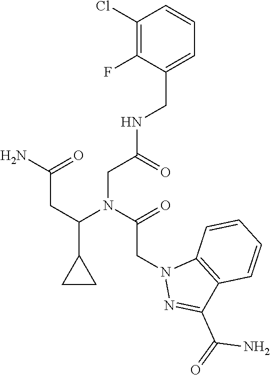 [SMILES: NC(=O)CC(C1CC1)N(CC(=O)NCc1cccc(Cl)c1F)C(=O)Cn1nc(C(N)=O)c2ccccc21]

1-(2-((3-Amino-1-cyclopropyl-3-oxopropyl)(2-((3-chloro-2-fluorobenzyl)amino)-2-oxoethyl)amino)-2-oxoethyl)-1H-indazole-3-carboxamide (27.7 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.20 (t, 1H), 7.56 (d, 1H), 7.45-7.37 (m, 2H), 7.31-6.90 (m, 3H), 5.67-5.41 (m, 2H), 4.56-4.36 (m, 2H), 4.17-4.04 (m, 2H), 3.90-3.76 (m, 1H), 2.85-2.48 (m, 2H), 1.15-0.98 (m, 1H), 0.70-0.51 (m, 2H), 0.40-0.16 (m, 2H). LCMS (M+H⁺) m/z calculated 529.2, found 529.2.

### Example 76: Preparation of 1-(2-((4-amino-4-oxobutan-2-yl)(2-((3-chloro-2-fluorobenzyl)amino)-2-oxoethyl)amino)-2-oxoethyl)-1H-indazole-3-carboxamide

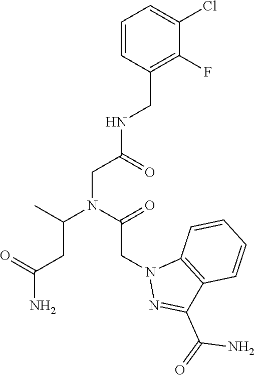 [SMILES: CC(CC(N)=O)N(CC(=O)NCc1cccc(Cl)c1F)C(=O)Cn1nc(C(N)=O)c2ccccc21]

1-(2-((4-Amino-4-oxobutan-2-yl)(2-((3-chloro-2-fluorobenzyl)amino)-2-oxoethyl)amino)-2-oxoethyl)-1H-indazole-3-carboxamide (8.8 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.21 (d, 1H), 7.64 (d, 1H), 7.59-7.54 (m, 1H), 7.41-7.25 (m, 3H), 7.21-7.11 (m, 1H) 6.91 (t, 1H), 5.85-5.38 (m, 2H), 4.74-4.56 (m, 2H), 4.41-4.26 (m, 2H), 4.03-3.87 (m, 1H), 2.70-2.37 (m, 2H), 1.35-1.20 (m, 3H). LCMS (M+H⁺) m/z calculated 503.2, found 503.2.

### Example 77: Preparation of ethyl 3-(2-(3-carbamoyl-1H-indazol-1-yl)-N-(2-((3-chloro-2-fluorobenzyl)amino)-2-oxoethyl)acetamido)-4,4,4-trifluorobutanoate

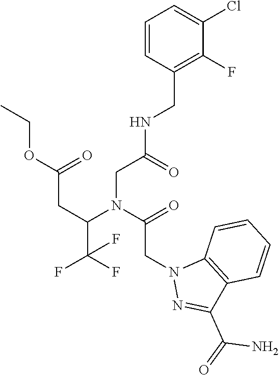 [SMILES: CCOC(=O)CC(N(CC(=O)NCc1cccc(Cl)c1F)C(=O)Cn1nc(C(N)=O)c2ccccc21)C(F)(F)F]

Ethyl 3-(2-(3-carbamoyl-1H-indazol-1-yl)-N-(2-((3-chloro-2-fluorobenzyl)amino)-2-oxoethyl)acetamido)-4,4,4-trifluorobutanoate (9.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.23 (t, 1H), 7.51-7.58 (m, 1H), 7.31-7.47 (m, 2H), 6.87-7.31 (m, 3H), 5.42-5.68 (m, 3H), 4.10-4.56 (m, 6H), 2.85-3.02 (m, 2H), 1.17-1.33 (m, 3H). LCMS (M+H⁺) m/z calculated 586.1, found 586.2.

### Example 78: Preparation of 1-(2-((2-((3-chloro-2-fluorobenzyl)amino)-2-oxoethyl)(cyclopropyl)amino)-2-oxoethyl)-5-fluoro-1H-indazole-3-carboxamide

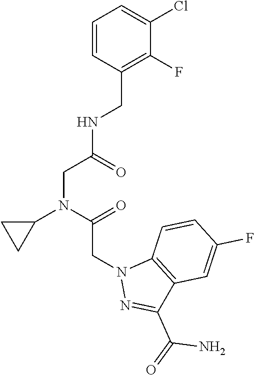 [SMILES: NC(=O)c1nn(CC(=O)N(CC(=O)NCc2cccc(Cl)c2F)C2CC2)c2ccc(F)cc12]

1-(2-((2-((3-Chloro-2-fluorobenzyl)amino)-2-oxoethyl)(cyclopropyl)amino)-2-oxoethyl)-5-fluoro-1H-indazole-3-carboxamide (74.8 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 7.81-7.83 (m, 1H), 7.57-7.59 (m, 1H), 7.31-7.34 (m, 1H), 7.22-7.24 (m, 2H), 6.99-7.03 (m, 1H), 5.67-5.68 (m, 2H), 4.42 (s, 2H), 4.11 (s, 2H), 3.08-3.09 (m, 1H), 1.03 (s, 4H). LCMS (M+H⁺) m/z calculated 476.1, found 476.2.

### Example 79: Preparation of 3-(2-(3-carbamoyl-1H-indazol-1-yl)-N-(2-((3-chloro-2-fluorobenzyl)amino)-2-oxoethyl)acetamido)-4,4,4-trifluorobutanoic acid

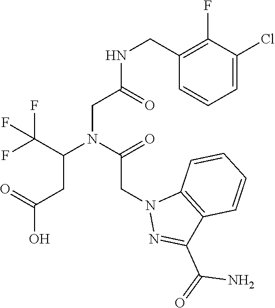 [SMILES: NC(=O)c1nn(CC(=O)N(CC(=O)NCc2cccc(Cl)c2F)C(CC(=O)O)C(F)(F)F)c2ccccc12]

3-(2-(3-Carbamoyl-1H-indazol-1-yl)-N-(2-((3-chloro-2-fluorobenzyl)amino)-2-oxoethyl)acetamido)-4,4,4-trifluorobutanoic acid (6.5 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.20-8.23 (m, 1H), 7.57 (t, 1H), 7.34-7.43 (m, 2H), 7.23-7.30 (m, 1H), 7.11-7.15 (m, 1H), 6.81 (t, 1H), 5.29-6.06 (m, 3H), 4.56 (d, 1H), 4.40 (t, 2H), 4.00-4.11 (m, 1H), 2.74-2.93 (m, 2H). LCMS (M+H⁺) m/z calculated 558.1, found 558.2.

### Example 80: Preparation of 5-chloro-1-(2-((2-((3-chloro-2-fluorobenzyl)amino)-2-oxoethyl)(cyclopropyl)amino)-2-oxoethyl)-1H-indazole-3-carboxamide

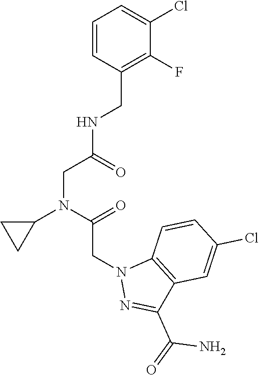 [SMILES: NC(=O)c1nn(CC(=O)N(CC(=O)NCc2cccc(Cl)c2F)C2CC2)c2ccc(Cl)cc12]

5-Chloro-1-(2-((2-((3-chloro-2-fluorobenzyl)amino)-2-oxoethyl)(cyclopropyl)amino)-2-oxoethyl)-1H-indazole-3-carboxamide (30.6 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.20 (d, 1H), 7.58 (d, 1H), 7.31-7.39 (m, 2H), 7.22 (t, 1H), 7.01 (t, 1H), 5.70 (s, 2H), 4.43 (s, 2H), 4.12 (s, 2H), 1.93 (s, 1H), 1.00-1.04 (m, 4H). LCMS (M+H⁺) m/z calculated 492.1, found 492.2.

### Example 81: Preparation of 7-chloro-1-(2-((2-((3-chloro-2-fluorobenzyl)amino)-2-oxoethyl)(cyclopropyl)amino)-2-oxoethyl)-1H-indazole-3-carboxamide

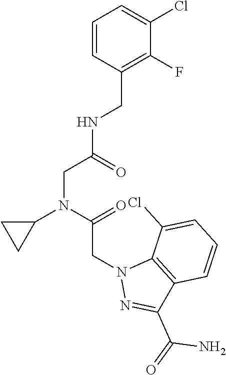 [SMILES: NC(=O)c1nn(CC(=O)N(CC(=O)NCc2cccc(Cl)c2F)C2CC2)c2c(Cl)cccc12]

7-Chloro-1-(2-((2-((3-chloro-2-fluorobenzyl)amino)-2-oxoethyl)(cyclopropyl)amino)-2-oxoethyl)-1H-indazole-3-carboxamide (47.1 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.42 (d, 1H), 7.33 (t, 1H), 7.23 (t, 2H), 7.01 (t, 1H), 5.97 (s, 2H), 4.42 (s, 2H), 4.12 (s, 2H), 3.10 (t, 1H), 1.03-1.04 (m, 4H). LCMS (M+Na⁺) m/z calculated 492.1, found 492.2.

### Example 82: Preparation of 1-(2-((2-((3-chloro-2,6-difluorobenzyl)amino)-2-oxoethyl)(cyclopropyl)amino)-2-oxoethyl)-1H-indazole-3-carboxamide

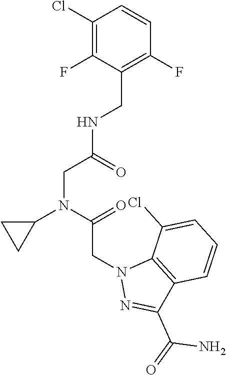 [SMILES: NC(=O)c1nn(CC(=O)N(CC(=O)NCc2c(F)ccc(Cl)c2F)C2CC2)c2c(Cl)cccc12]

1-(2-((2-((3-Chloro-2,6-difluorobenzyl)amino)-2-oxoethyl)(cyclopropyl)amino)-2-oxoethyl)-1H-indazole-3-carboxamide (52.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.21 (d, 1H), 7.58 (d, 1H), 7.400-7.44 (m, 2H), 7.28 (t, 1H), 6.98 (t, 1H), 5.68 (s, 2H), 4.48 (s, 2H), 4.07 (s, 2H), 3.04-3.09 (m, 1H), 1.00-1.01 (m, 4H). LCMS (M+Na⁺) m/z calculated 476.1, found 476.2.

### Example 83: Preparation of 1-(2-((2-((3-chloro-2,4-difluorobenzyl)amino)-2-oxoethyl)(cyclopropyl)amino)-2-oxoethyl)-1H-indazole-3-carboxamide

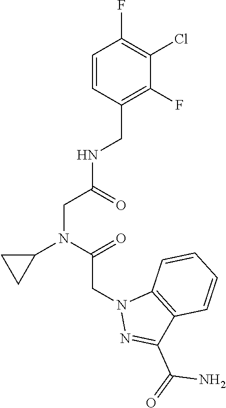 [SMILES: NC(=O)c1nn(CC(=O)N(CC(=O)NCc2ccc(F)c(Cl)c2F)C2CC2)c2ccccc12]

1-(2-((2-((3-Chloro-2,4-difluorobenzyl)amino)-2-oxoethyl)(cyclopropyl)amino)-2-oxoethyl)-1H-indazole-3-carboxamide (48.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.57 (d, 1H), 7.42 (t, 1H), 7.23-7.30 (m, 2H), 6.91-6.96 (m, 1H), 5.70 (s, 2H), 4.40 (s, 2H), 4.12 (s, 2H), 3.10-3.13 (m, 1H), 1.03-1.04 (m, 4H). LCMS (M+Na⁺) m/z calculated 476.1, found 476.2.

### Example 84: Preparation of 1-(2-((2-((3-chloro-2-fluorobenzyl)amino)-2-oxoethyl)(cyclopropyl)amino)-2-oxoethyl)-6-fluoro-1H-indazole-3-carboxamide

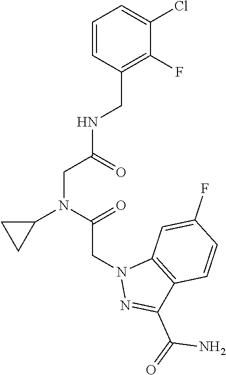 [SMILES: NC(=O)c1nn(CC(=O)N(CC(=O)NCc2cccc(Cl)c2F)C2CC2)c2cc(F)ccc12]

1-(2-((2-((3-Chloro-2-fluorobenzyl)amino)-2-oxoethyl)(cyclopropyl)amino)-2-oxoethyl)-6-fluoro-1H-indazole-3-carboxamide (67.5 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (DMSO-d₆, 400 MHz) δ 8.47 (d, 1H), 8.17 (d, 1H), 7.73 (s, 1H), 7.56-7.40 (m, 3H), 7.24-7.08 (m, 3H), 5.62 (s, 2H), 4.33 (d, 2H), 3.98 (s, 2H), 3.07-3.06 (m, 1H), 1.23 (brs, 2H), 0.98-0.94 (m, 1H), 0.90-0.87 (m, 1H). LCMS (M+H⁺) m/z calculated 476.1, found 476.1.

### Example 85: Preparation of 6-chloro-1-(2-((2-((3-chloro-2-fluorobenzyl)amino)-2-oxoethyl)(cyclopropyl)amino)-2-oxoethyl)-1H-indazole-3-carboxamide

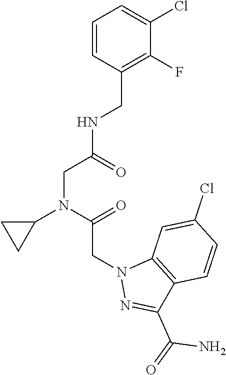 [SMILES: NC(=O)c1nn(CC(=O)N(CC(=O)NCc2cccc(Cl)c2F)C2CC2)c2cc(Cl)ccc12]

6-Chloro-1-(2-((2-((3-chloro-2-fluorobenzyl)amino)-2-oxoethyl)(cyclopropyl)amino)-2-oxoethyl)-1H-indazole-3-carboxamide (65.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (DMSO-d₆, 400 MHz) δ 8.48 (t, 1H), 8.16 (d, 1H), 7.88 (d, 1H), 7.77 (s, 1H), 7.46 (t, 2H), 7.21-7.29 (m, 2H), 7.11 (t, 1H), 5.66 (s, 2H), 4.34 (d, 2H), 3.99 (s, 2H), 3.06-3.08 (m, 1H), 1.00 (s, 2H), 0.91 (d, 2H). LCMS (M+Na⁺) m/z calculated 492.1, found 492.2.

### Example 86: Preparation of 1-(2-((2-((3-chloro-2-fluorobenzyl)amino)-2-oxoethyl)(cyclopropyl)amino)-2-oxoethyl)-6-cyano-1H-indazole-3-carboxamide

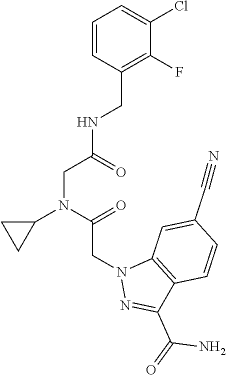 [SMILES: N#Cc1ccc2c(C(N)=O)nn(CC(=O)N(CC(=O)NCc3cccc(Cl)c3F)C3CC3)c2c1]

1-(2-((2-((3-Chloro-2-fluorobenzyl)amino)-2-oxoethyl)(cyclopropyl)amino)-2-oxoethyl)-6-cyano-1H-indazole-3-carboxamide (26.5 mg) was prepared as 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.39 (d, 1H), 8.20 (s, 1H), 7.53 (d, 1H), 7.34 (t, 1H), 7.25 (t, 1H), 7.02 (t, 1H), 5.79 (s, 2H), 4.42 (s, 2H), 4.14 (s, 2H), 1.29 (s, 1H), 1.06-1.03 (m, 4H). LCMS (M+H⁺) m/z calculated 483.1, found 483.4.

### Example 87: Preparation of 1-(2-((2-((3-chloro-6-cyano-2-fluorobenzyl)amino)-2-oxoethyl)(cyclopropyl)amino)-2-oxoethyl)-1H-indazole-3-carboxamide

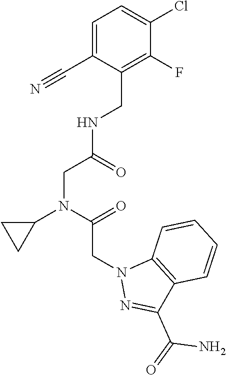 [SMILES: N#Cc1ccc(Cl)c(F)c1CNC(=O)CN(C(=O)Cn1nc(C(N)=O)c2ccccc21)C1CC1]

1-(2-((2-((3-Chloro-6-cyano-2-fluorobenzyl)amino)-2-oxoethyl)(cyclopropyl)amino)-2-oxoethyl)-1H-indazole-3-carboxamide (8.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.21 (d, 1H), 7.61-7.56 (m, 3H), 7.44 (t, 1H), 7.28 (t, 1H), 5.69 (s, 2H), 4.59 (s, 2H), 4.10 (s, 2H), 2.03 (s, 1H), 1.00-1.02 (m, 4H). LCMS (M+H⁺) m/z calculated 483.1, found 483.2.

### Example 88: Preparation of 1-(2-((2-((3-chloro-2-fluorobenzyl)amino)-2-oxoethyl)(cyclopropyl)amino)-2-oxoethyl)-7-fluoro-1H-indazole-3-carboxamide

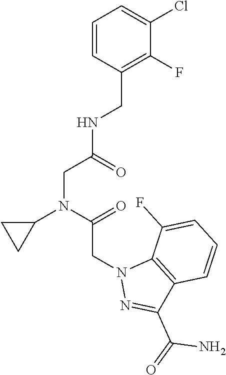 [SMILES: NC(=O)c1nn(CC(=O)N(CC(=O)NCc2cccc(Cl)c2F)C2CC2)c2c(F)cccc12]

1-(2-((2-((3-Chloro-2-fluorobenzyl)amino)-2-oxoethyl)(cyclopropyl)amino)-2-oxoethyl)-7-fluoro-1H-indazole-3-carboxamide (49.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹HNMR (CD₃OD, 400 MHz) δ 8.01-8.03 (m, 1H), 7.00-7.35 (m, 5H), 5.79 (s, 2H), 4.42 (s, 2H), 4.12 (s, 2H), 3.04-3.11 (m, 1H), 0.97-1.03 (m, 4H). LCMS (M+H⁺) m/z calculated 476.1, found 476.2.

### Example 89: Preparation of 6-(aminomethyl)-1-(2-((2-((3-chloro-2-fluorobenzyl)amino)-2-oxoethyl)(cyclopropyl)amino)-2-oxoethyl)-1H-indazole-3-carboxamide

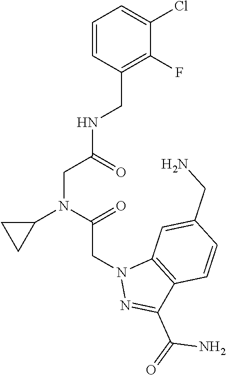 [SMILES: NCc1ccc2c(C(N)=O)nn(CC(=O)N(CC(=O)NCc3cccc(Cl)c3F)C3CC3)c2c1]

6-(Aminomethyl)-1-(2-((2-((3-chloro-2-fluorobenzyl)amino)-2-oxoethyl)(cyclopropyl)amino)-2-oxoethyl)-1H-indazole-3-carboxamide (1.9 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.16 (d, 1H), 7.55 (s, 1H), 7.33 (t, 1H), 7.29 (d, 1H), 7.22 (t, 1H), 6.97 (t, 1H), 5.70 (s, 2H), 4.44 (s, 2H), 4.14 (s, 2H), 3.92 (s, 2H), 3.14 (d, 1H), 1.05 (d, 4H). LCMS (M+H⁺) m/z calculated 487.2, found 487.2.

### Example 90: Preparation of 1-(2-((2-((3-chloro-5-cyano-2-fluorobenzyl)amino)-2-oxoethyl)(cyclopropyl)amino)-2-oxoethyl)-1H-indazole-3-carboxamide

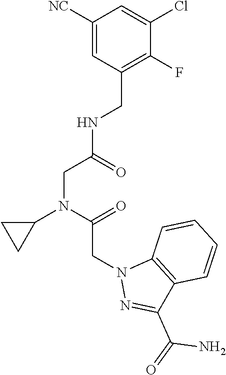 [SMILES: N#Cc1cc(Cl)c(F)c(CNC(=O)CN(C(=O)Cn2nc(C(N)=O)c3ccccc32)C2CC2)c1]

1-(2-((2-((3-Chloro-5-cyano-2-fluorobenzyl)amino)-2-oxoethyl)(cyclopropyl)amino)-2-oxoethyl)-1H-indazole-3-carboxamide (1.1 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.21 (d, 1H), 7.85-7.83 (m, 1H), 7.67 (t, 1H), 7.57 (d, 1H), 7.43 (t, 1H), 7.27 (t, 1H), 5.69 (s, 2H), 4.45 (s, 2H), 4.14 (s, 2H), 3.12-3.09 (m, 1H), 1.05-1.03 (m, 4H). LCMS (M+H⁺) m/z calculated 483.1, found 483.2.

### Example 91: Preparation of 1-(2-((2-(((6-chloro-1H-indazol-4-yl)methyl)amino)-2-oxoethyl)(cyclopropyl)amino)-2-oxoethyl)-1H-indazole-3-carboxamide

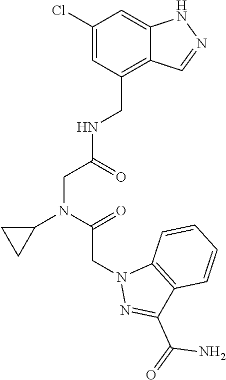 [SMILES: NC(=O)c1nn(CC(=O)N(CC(=O)NCc2cc(Cl)cc3[nH]ncc23)C2CC2)c2ccccc12]

1-(2-((2-(((6-Chloro-1H-indazol-4-yl)methyl)amino)-2-oxoethyl)(cyclopropyl)amino)-2-oxoethyl)-1H-indazole-3-carboxamide (37.1 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (DMSO-d₆, 400 MHz) δ 13.21 (s, 1H), 8.54 (s, 1H), 8.18 (d, 1H), 7.68-7.64 (m, 2H), 7.49-7.24 (m, 5H), 6.96 (s, 1H), 5.67 (s, 2H), 4.59 (d, 2H), 4.02 (s, 2H), 3.07 (s, 1H), 1.01-0.91 (m, 4H). LCMS (M+Na⁺) m/z calculated 480.1, found 480.1.

### Example 92: Preparation of 1-(2-((2-((3-chloro-2-fluorobenzyl)amino)-2-oxoethyl)(cyclopropyl)amino)-2-oxoethyl)-5-cyano-1H-indazole-3-carboxamide

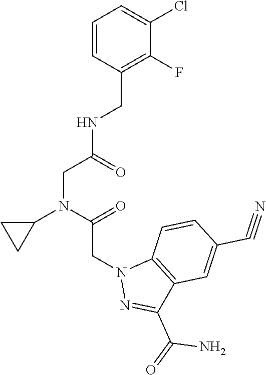 [SMILES: N#Cc1ccc2c(c1)c(C(N)=O)nn2CC(=O)N(CC(=O)NCc1cccc(Cl)c1F)C1CC1]

1-(2-((2-((3-Chloro-2-fluorobenzyl)amino)-2-oxoethyl)(cyclopropyl)amino)-2-oxoethyl)-5-cyano-1H-indazole-3-carboxamide (20.2 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.63 (s, 1H), 7.74-7.76 (m, 1H), 7.62-7.64 (m, 1H), 7.31-7.34 (m, 1H), 7.20-7.23 (m, 1H), 7.00 (t, 1H), 5.75 (s, 2H), 4.43 (s, 2H), 4.12 (s, 2H), 3.11-3.12 (m, 1H), 1.02-1.04 (m, 4H). LCMS (M+H⁺) m/z calculated 483.1, found 483.1.

### Example 93: Preparation of 1-(2-((2-((3-chloro-2-fluorobenzyl)amino)-2-oxoethyl)(cyclopropyl)amino)-2-oxoethyl)-1H-indazole-3,5-dicarboxamide

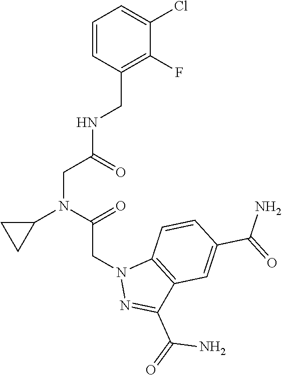 [SMILES: NC(=O)c1ccc2c(c1)c(C(N)=O)nn2CC(=O)N(CC(=O)NCc1cccc(Cl)c1F)C1CC1]

1-(2-((2-((3-Chloro-2-fluorobenzyl)amino)-2-oxoethyl)(cyclopropyl)amino)-2-oxoethyl)-1H-indazole-3,5-dicarboxamide (21.9 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.80 (s, 1H), 7.92-7.95 (m, 1H), 7.65 (d, 1H), 7.13-7.45 (m, 2H), 7.00 (t, 1H), 5.73 (s, 2H), 4.75 (s, 2H), 4.43 (s, 2H), 3.10-3.12 (m, 1H), 1.03-1.05 (m, 4H). LCMS (M+H⁺) m/z calculated 501.1, found 501.2.

### Example 94: Preparation of 1-(2-((2-((3-chloro-2-fluorobenzyl)amino)-2-oxoethyl)(cyclopropyl)amino)-2-oxoethyl)-6-(trifluoromethyl)-1H-indazole-3-carboxamide

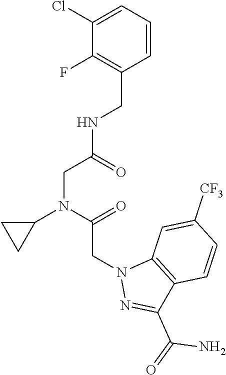 [SMILES: NC(=O)c1nn(CC(=O)N(CC(=O)NCc2cccc(Cl)c2F)C2CC2)c2cc(C(F)(F)F)ccc12]

1-(2-((2-((3-Chloro-2-fluorobenzyl)amino)-2-oxoethyl)(cyclopropyl)amino)-2-oxoethyl)-6-(trifluoromethyl)-1H-indazole-3-carboxamide (33.6 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃Cl, 400 MHz) δ 8.49-8.5 (d, 1H), 7.71 (s, 1H), 7.51-7.53 (d, 1H), 7.29-7.30 (m, 1H), 7.15 (t, 1H), 6.97 (t, 1H), 6.83 (s, 1H), 6.34 (s, 1H), 5.55 (s, 2H), 5.47 (s, 1H), 4.44-4.46 (d, 2H), 4.07 (s, 2H), 3.07 (m, 1H), 1.06-1.08 (m, 4H). LCMS (M+H⁺) m/z calculated 526.1, found 526.4.

### Example 95: Preparation of 1-(2-((2-((3-chloro-2-fluorobenzyl)amino)-2-oxoethyl)(cyclopropyl)amino)-2-oxoethyl)-5-(trifluoromethyl)-1H-indazole-3-carboxamide

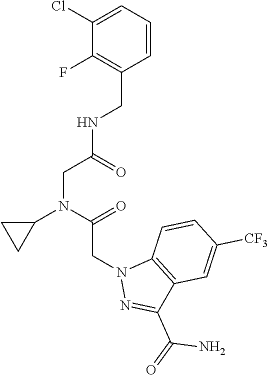 [SMILES: NC(=O)c1nn(CC(=O)N(CC(=O)NCc2cccc(Cl)c2F)C2CC2)c2ccc(C(F)(F)F)cc12]

1-(2-((2-((3-Chloro-2-fluorobenzyl)amino)-2-oxoethyl)(cyclopropyl)amino)-2-oxoethyl)-5-(trifluoromethyl)-1H-indazole-3-carboxamide (37.8 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃Cl, 400 MHz) δ 8.71 (s, 1H), 7.61-7.63 (d, 1H), 7.43-7.45 (d, 1H), 7.29-7.30 (m, 1H), 7.14 (t, 1H), 6.95 (t, 1H), 6.80 (s, 1H), 6.36 (s, 1H), 5.32 (s, 2H), 5.05 (m, 1H), 4.44-4.46 (d, 2H), 4.06 (s, 2H), 3.05 (m, 1H), 1.05-1.08 (m, 4H). LCMS (M+H⁺) m/z calculated 526.1, found 526.4.

### Example 96: Preparation of 1-(2-((2-((3-chloro-4-cyano-2-fluorobenzyl)amino)-2-oxoethyl)(cyclopropyl)amino)-2-oxoethyl)-1H-indazole-3-carboxamide

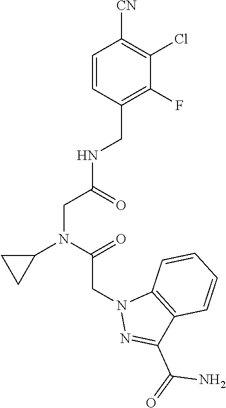 [SMILES: N#Cc1ccc(CNC(=O)CN(C(=O)Cn2nc(C(N)=O)c3ccccc32)C2CC2)c(F)c1Cl]

1-(2-((2-((3-Chloro-4-cyano-2-fluorobenzyl)amino)-2-oxoethyl)(cyclopropyl)amino)-2-oxoethyl)-1H-indazole-3-carboxamide (24.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.23 (d, 1H), 7.54 (d, 1H), 7.35-7.42 (m, 3H), 7.29 (t, 1H), 5.69 (s, 2H), 4.48 (s, 2H), 4.13 (s, 2H), 3.12-3.14 (m, 1H), 1.04-1.06 (m, 4H). LCMS (M+Na⁺) m/z calculated 483.1, found 483.2.

### Example 97: Preparation of 1-(2-((2-((3-chloro-2-fluorobenzyl)amino)-2-oxoethyl)(cyclopropyl)amino)-2-oxoethyl)-4-fluoro-1H-indazole-3-carboxamide

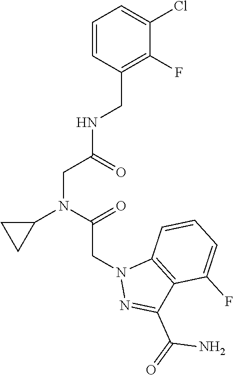 [SMILES: NC(=O)c1nn(CC(=O)N(CC(=O)NCc2cccc(Cl)c2F)C2CC2)c2cccc(F)c12]

1-(2-((2-((3-Chloro-2-fluorobenzyl)amino)-2-oxoethyl)(cyclopropyl)amino)-2-oxoethyl)-4-fluoro-1H-indazole-3-carboxamide (155.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹HNMR (CD₃OD, 400 MHz) δ 7.19-7.40 (m, 4H), 6.93-7.02 (m, 2H), 5.69 (s, 2H), 4.42 (s, 2H), 4.12 (s, 2H), 3.04-3.11 (m, 1H), 0.97-1.03 (m, 4H). LCMS (M+H⁺) m/z calculated 476.1, found 476.2.

### Example 98: Preparation of 2-(3-(1H-imidazol-2-yl)-1H-indazol-1-yl)-N-(2-((3-chloro-2-fluorobenzyl)amino)-2-oxoethyl)-N-cyclopropylacetamide

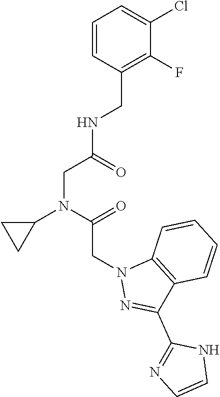 [SMILES: O=C(CN(C(=O)Cn1nc(-c2ncc[nH]2)c2ccccc21)C1CC1)NCc1cccc(Cl)c1F]

2-(3-(1H-imidazol-2-yl)-1H-indazol-1-yl)-N-(2-((3-chloro-2-fluorobenzyl)amino)-2-oxoethyl)-N-cyclopropylacetamide (30.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹HNMR (CD₃OD, 400 MHz) δ 8.12 (d, 1H), 7.74 (d, 1H), 7.67 (s, 2H), 7.56 (t, 1H), 7.42 (t, 1H), 7.21-7.31 (m, 2H), 6.98 (t, 1H), 5.83 (s, 2H), 4.43 (s, 2H), 4.16 (s, 2H), 3.14-3.16 (m, 1H), 1.03-1.05 (m, 4H). LCMS (M+H⁺) m/z calculated 481.1, found 481.4.

### Example 99: Preparation of 1-(2-((2-((3-chloro-2-fluoro-4-(hydroxymethyl)benzyl)amino)-2-oxoethyl)(cyclopropyl)amino)-2-oxoethyl)-1H-indazole-3-carboxamide

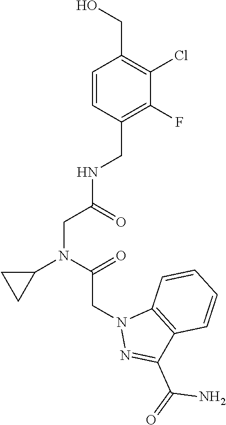 [SMILES: NC(=O)c1nn(CC(=O)N(CC(=O)NCc2ccc(CO)c(Cl)c2F)C2CC2)c2ccccc12]

1-(2-((2-((3-Chloro-2-fluoro-4-(hydroxymethyl)benzyl)amino)-2-oxoethyl)(cyclopropyl)amino)-2-oxoethyl)-1H-indazole-3-carboxamide (12.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.58 (d, 1H), 7.41-7.45 (m, 1H), 7.21-7.30 (m, 3H), 5.69 (s, 2H), 4.65 (s, 2H), 4.42 (s, 2H), 4.12 (s, 2H), 3.07-3.12 (m, 1H), 0.99-1.05 (m, 4H), LCMS (M+Na⁺) m/z calculated 488.1, found 488.2.

### Example 100: Preparation of 1-(2-((2-((3-chloro-2-fluorobenzyl)amino)-2-oxoethyl)(cyclopropyl)amino)-2-oxoethyl)-1H-indazole-3,6-dicarboxamide

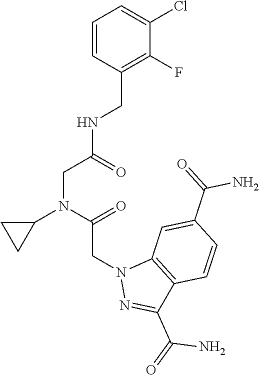 [SMILES: NC(=O)c1ccc2c(C(N)=O)nn(CC(=O)N(CC(=O)NCc3cccc(Cl)c3F)C3CC3)c2c1]

1-(2-((2-((3-Chloro-2-fluorobenzyl)amino)-2-oxoethyl)(cyclopropyl)amino)-2-oxoethyl)-1H-indazole-3,6-dicarboxamide (2.6 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.28 (d, 1H), 8.14 (s, 1H), 7.77 (d, 1H), 7.31 (t, 1H), 7.20 (t, 1H), 6.94 (t, 1H), 5.77 (s, 2H), 4.44 (s, 2H), 4.15 (s, 2H), 3.16 (s, 1H), 1.04-1.06 (m, 4H). LCMS (M+H⁺) m/z calculated 501.1, found 501.1.

### Example 101: Preparation of 1-(2-((2-((5-(aminomethyl)-3-chloro-2-fluorobenzyl)amino)-2-oxoethyl)(cyclopropyl)amino)-2-oxoethyl)-1H-indazole-3-carboxamide

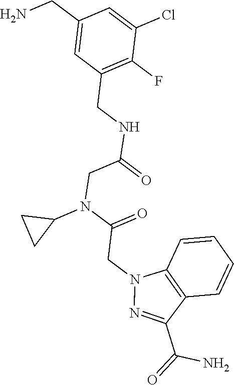 [SMILES: NCc1cc(Cl)c(F)c(CNC(=O)CN(C(=O)Cn2nc(C(N)=O)c3ccccc32)C2CC2)c1]

1-(2-((2-((5-(Aminomethyl)-3-chloro-2-fluorobenzyl)amino)-2-oxoethyl)(cyclopropyl)amino)-2-oxoethyl)-1H-indazole-3-carboxamide (2.6 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.23 (d, 1H), 7.56 (d, 1H), 7.40 (t, 1H), 7.28 (t, 2H), 7.17 (d, 1H), 5.70 (s, 2H), 4.43 (s, 2H), 4.14 (s, 2H), 3.48 (s, 2H), 3.14 (s, 1H), 1.04-1.07 (m, 4H). LCMS (M+H⁺⁾m/z calculated 487.2, found 487.2.

### Example 102: Preparation of 1-(2-((2-(((4-chloro-1H-indazol-6-yl)methyl)amino)-2-oxoethyl)(cyclopropyl)amino)-2-oxoethyl)-1H-indazole-3-carboxamide

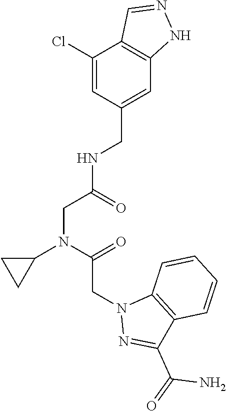 [SMILES: NC(=O)c1nn(CC(=O)N(CC(=O)NCc2cc(Cl)c3cn[nH]c3c2)C2CC2)c2ccccc12]

1-(2-((2-(((4-Chloro-1H-indazol-6-yl)methyl)amino)-2-oxoethyl)(cyclopropyl)amino)-2-oxoethyl)-1H-indazole-3-carboxamide (35.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹HNMR (CD₃OD, 400 MHz) δ 8.22 (d, 1H), 8.01 (s, 1H), 7.57 (d, 1H), 7.38-7.35 (m, 2H), 7.26 (t, 1H), 7.10 (s, 1H), 5.72 (s, 2H), 4.49 (s, 2H), 4.18 (s, 2H), 3.14-3.12 (m, 1H), 1.08-1.04 (m, 4H). LCMS (M+H⁺) m/z calculated 480.1, found 480.1.

### Example 103: Preparation of 1-(2-((2-((3-chloro-5-fluorobenzyl)amino)-2-oxoethyl)(cyclopropyl)amino)-2-oxoethyl)-1H-indazole-3-carboxamide

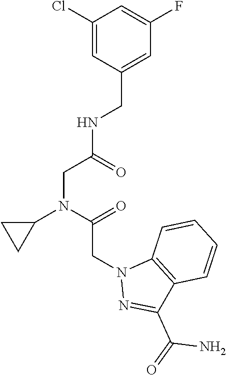 [SMILES: NC(=O)c1nn(CC(=O)N(CC(=O)NCc2cc(F)cc(Cl)c2)C2CC2)c2ccccc12]

1-(2-((2-((3-Chloro-5-fluorobenzyl)amino)-2-oxoethyl)(cyclopropyl)amino)-2-oxoethyl)-1H-indazole-3-carboxamide (61.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.59 (d, 1H), 7.43 (t, 1H), 7.28 (t, 1H), 7.13 (s, 1H), 7.04 (d, 1H), 6.97 (d, 1H), 5.70 (s, 2H), 4.36 (s, 2H), 4.14 (s, 2H), 3.10-3.13 (m, 1H), 1.04-1.05 (m, 4H). LCMS (M+Na⁺) m/z calculated 458.1, found 458.2.

### Example 104: Preparation of 1-(2-(cyclopropyl(2-((3,4-dichlorobenzyl)amino)-2-oxoethyl)amino)-2-oxoethyl)-1H-indazole-3-carboxamide

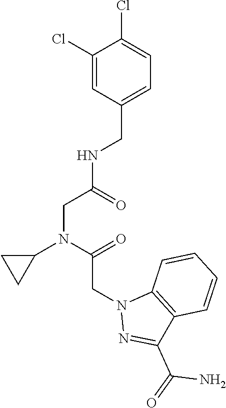 [SMILES: NC(=O)c1nn(CC(=O)N(CC(=O)NCc2ccc(Cl)c(Cl)c2)C2CC2)c2ccccc12]

1-(2-(Cyclopropyl(2-((3,4-dichlorobenzyl)amino)-2-oxoethyl)amino)-2-oxoethyl)-1H-indazole-3-carboxamide (61.6 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.58 (d, 1H), 7.42 (t, 2H), 7.37 (d, 1H), 7.28 (t, 1H), 7.17 (t, 1H), 5.70 (s, 2H), 4.34 (s, 2H), 4.13 (s, 2H), 3.09-3.14 (m, 1H), 1.04-1.05 (m, 4H). LCMS (M+Na⁺) m/z calculated 474.1, found 474.2.

### Example 105: Preparation of 1-(2-((2-((3-chloro-2-fluorobenzyl)amino)-2-oxoethyl)(cyclopropyl)amino)-2-oxoethyl)-7-cyano-1H-indazole-3-carboxamide

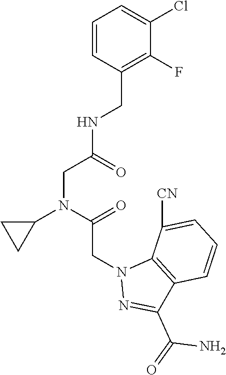 [SMILES: N#Cc1cccc2c(C(N)=O)nn(CC(=O)N(CC(=O)NCc3cccc(Cl)c3F)C3CC3)c12]

1-(2-((2-((3-Chloro-2-fluorobenzyl)amino)-2-oxoethyl)(cyclopropyl)amino)-2-oxoethyl)-7-cyano-1H-indazole-3-carboxamide (14.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.25 (d, 1H), 7.91 (d, 1H), 7.41 (t, 1H), 7.34 (t, 1H), 7.23 (t, 1H), 7.02 (t, 1H), 6.06 (s, 2H), 4.49 (s, 2H), 4.16 (s, 2H), 3.12-3.16 (m, 1H), 1.05-1.09 (m, 4H). LCMS (M+H⁺) m/z calculated 483.1, found 483.1.

### Example 106: Preparation of 1-(2-(cyclopropyl(2-((2,3-dichlorobenzyl)amino)-2-oxoethyl)amino)-2-oxoethyl)-1H-indazole-3-carboxamide

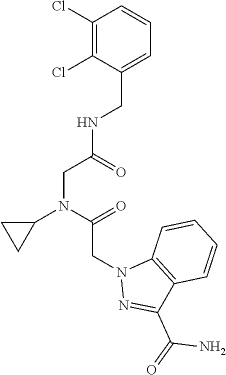 [SMILES: NC(=O)c1nn(CC(=O)N(CC(=O)NCc2cccc(Cl)c2Cl)C2CC2)c2ccccc12]

1-(2-(Cyclopropyl(2-((2,3-dichlorobenzyl)amino)-2-oxoethyl)amino)-2-oxoethyl)-1H-indazole-3-carboxamide (57.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.57 (d, 1H), 7.42 (t, 2H), 7.25-7.30 (m, 2H), 7.13 (t, 1H), 5.70 (s, 2H), 4.48 (s, 2H), 4.16 (s, 2H), 3.10-3.15 (m, 1H), 1.01-1.07 (m, 4H). LCMS (M+Na⁺) m/z calculated 474.1, found 474.2.

### Example 107: Preparation of 1-(2-((2-(((6-chloropyridin-2-yl)methyl)amino)-2-oxoethyl)(cyclopropyl)amino)-2-oxoethyl)-1H-indazole-3-carboxamide

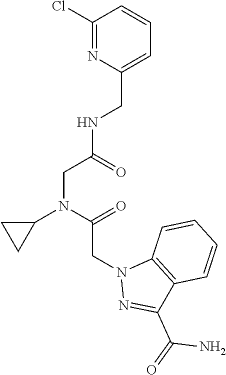 [SMILES: NC(=O)c1nn(CC(=O)N(CC(=O)NCc2cccc(Cl)n2)C2CC2)c2ccccc12]

1-(2-((2-(((6-Chloropyridin-2-yl)methyl)amino)-2-oxoethyl)(cyclopropyl)amino)-2-oxoethyl)-1H-indazole-3-carboxamide (22.6 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (t, 1H), 7.61-7.55 (m, 2H), 7.41 (d, 1H), 7.30-7.22 (m, 3H), 5.68 (s, 2H), 4.40 (s, 2H), 4.17 (s, 2H), 3.14-3.09 (m, 1H), 1.05-1.04 (m, 4H). LCMS (M+H⁺) m/z calculated 441.1, found 441.2.

### Example 108: Preparation of 1-(2-((2-((3-chloro-4-fluorobenzyl)amino)-2-oxoethyl)(cyclopropyl)amino)-2-oxoethyl)-1H-indazole-3-carboxamide

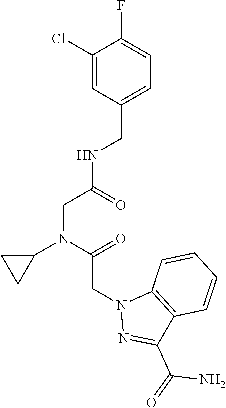 [SMILES: NC(=O)c1nn(CC(=O)N(CC(=O)NCc2ccc(F)c(Cl)c2)C2CC2)c2ccccc12]

1-(2-((2-((3-Chloro-4-fluorobenzyl)amino)-2-oxoethyl)(cyclopropyl)amino)-2-oxoethyl)-1H-indazole-3-carboxamide (10.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.58 (d, 1H), 7.42 (t, 1H), 7.36 (d, 1H), 7.28 (t, 1H), 7.17 (s, 1H), 7.10-7.06 (m, 1H), 5.68 (s, 2H), 4.32 (s, 2H), 4.11 (s, 2H), 3.14-3.09 (m, 1H), 1.05-1.04 (m, 4H). LCMS (M+H⁺) m/z calculated 458.1, found 458.2.

### Example 109: Preparation of 1-(2-((2-(((5-chloro-1H-indazol-7-yl)methyl)amino)-2-oxoethyl)(cyclopropyl)amino)-2-oxoethyl)-1H-indazole-3-carboxamide

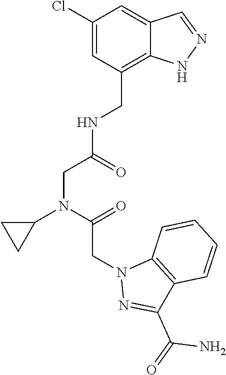 [SMILES: NC(=O)c1nn(CC(=O)N(CC(=O)NCc2cc(Cl)cc3cn[nH]c23)C2CC2)c2ccccc12]

1-(2-((2-(((5-Chloro-1H-indazol-7-yl)methyl)amino)-2-oxoethyl)(cyclopropyl)amino)-2-oxoethyl)-1H-indazole-3-carboxamide (37.7 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.23 (d, 1H), 8.04 (s, 1H), 7.68 (s, 1H), 7.59 (d, 1H), 7.43 (t, 1H), 7.29-7.16 (m, 2H), 5.71 (s, 2H), 4.64 (s, 2H), 4.17 (s, 2H), 3.15-3.08 (m, 1H), 1.09-0.99 (m, 4H). LCMS (M+H⁺) m/z calculated 480.1, found 480.5.

### Example 110: Preparation of 1-(2-((2-(((5-chlorobenzofuran-7-yl)methyl)amino)-2-oxoethyl)(cyclopropyl)amino)-2-oxoethyl)-1H-indazole-3-carboxamide

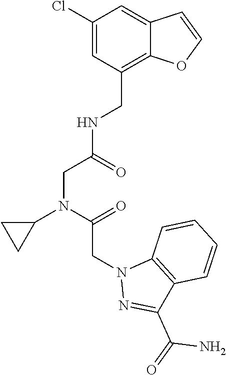 [SMILES: NC(=O)c1nn(CC(=O)N(CC(=O)NCc2cc(Cl)cc3ccoc23)C2CC2)c2ccccc12]

1-(2-((2-(((5-Chlorobenzofuran-7-yl)methyl)amino)-2-oxoethyl)(cyclopropyl)amino)-2-oxoethyl)-1H-indazole-3-carboxamide (36.5 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.21 (d, 1H), 7.79 (d, 1H), 7.59 (d, 1H), 7.50 (s, 1H), 7.27 (t, 1H), 7.20 (s, 1H), 6.82 (d, 1H), 5.71 (s, 2H), 4.66 (s, 2H), 4.16 (s, 2H), 3.15-3.08 (m, 1H), 1.09-0.99 (m, 4H). LCMS (M+H⁺) m/z calculated 480.1, found 480.2.

### Example 111: Preparation of 1-(2-((2-((3-chloro-2-fluorobenzyl)amino)-2-oxoethyl)(cyclopropyl)amino)-2-oxoethyl)-7-(trifluoromethyl)-1H-indazole-3-carboxamide

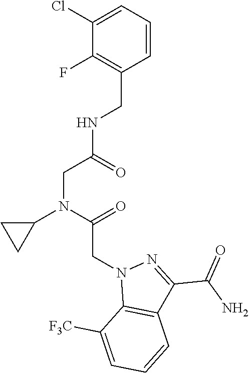 [SMILES: NC(=O)c1nn(CC(=O)N(CC(=O)NCc2cccc(Cl)c2F)C2CC2)c2c(C(F)(F)F)cccc12]

1-(2-((2-((3-Chloro-2-fluorobenzyl)amino)-2-oxoethyl)(cyclopropyl)amino)-2-oxoethyl)-7-(trifluoromethyl)-1H-indazole-3-carboxamide (40.1 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃Cl, 400 MHz) δ 8.72-8.70 (d, 1H), 7.77-7.79 (d, 1H), 7.35-7.39 (t, 1H), 7.29-7.26 (m, 1H), 7.10-7.14 (t, 1H), 6.89-6.93 (t, 1H), 6.89 (s, 1H), 6.46 (s, 1H), 5.67 (s, 2H), 5.51 (s, 1H), 4.41-4.42 (d, 2H),4.05 (s, 2H), 2.96-2.99 (m, 1H),1.04-1.06 (m, 4H). LCMS (M+H⁺) m/z calculated 526.1, found 526.2.

### Example 112: Preparation of 1-(2-((2-((3-chlorobenzyl)amino)-2-oxoethyl)(cyclopropyl)amino)-2-oxoethyl)-1H-indazole-3-carboxamide

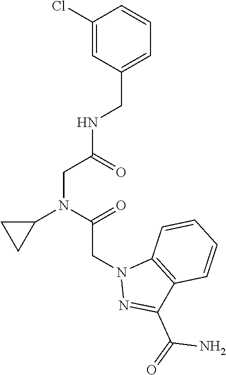 [SMILES: NC(=O)c1nn(CC(=O)N(CC(=O)NCc2cccc(Cl)c2)C2CC2)c2ccccc12]

1-(2-((2-((3-Chlorobenzyl)amino)-2-oxoethyl)(cyclopropyl)amino)-2-oxoethyl)-1H-indazole-3-carboxamide (65.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.59 (d, 1H), 7.43 (t, 1H), 7.17-7.30 (m, 5H), 5.70 (s, 2H), 4.36 (s, 2H), 4.14 (s, 2H), 3.08-3.14 (m, 1H), 1.03-1.05 (m, 4H). LCMS (M+Na⁺) m/z calculated 440.1, found 440.2.

### Example 113: Preparation of 1-(2-((2-((3-chloro-4-methylbenzyl)amino)-2-oxoethyl)(cyclopropyl)amino)-2-oxoethyl)-1H-indazole-3-carboxamide

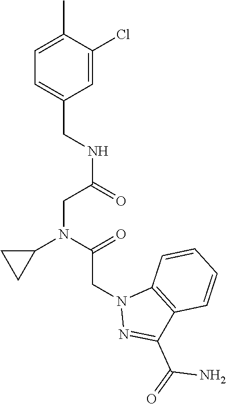 [SMILES: Cc1ccc(CNC(=O)CN(C(=O)Cn2nc(C(N)=O)c3ccccc32)C2CC2)cc1Cl]

1-(2-((2-((3-Chloro-4-methylbenzyl)amino)-2-oxoethyl)(cyclopropyl)amino)-2-oxoethyl)-1H-indazole-3-carboxamide (57.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.59 (d, 1H), 7.43 (t, 1H), 7.28 (t, 2H) 7.16 (d, 1H), 7.07 (d, 1H), 5.70 (s, 2H), 4.31 (s, 2H), 4.12 (s, 2H), 3.07-3.13 (m, 1H), 2.30 (s, 3H), 1.03-1.04 (m, 4H). LCMS (M+Na⁺) m/z calculated 454.1, found 454.2.

### Example 114: Preparation of 1-(2-((2-(((7-chloro-1H-indazol-5-yl)methyl)amino)-2-oxoethyl)(cyclopropyl)amino)-2-oxoethyl)-1H-indazole-3-carboxamide

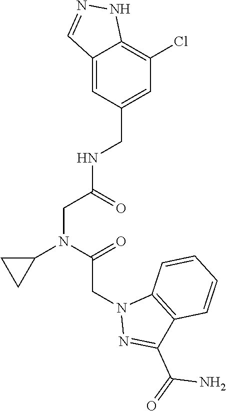 [SMILES: NC(=O)c1nn(CC(=O)N(CC(=O)NCc2cc(Cl)c3[nH]ncc3c2)C2CC2)c2ccccc12]

1-(2-((2-(((7-Chloro-1H-indazol-5-yl)methyl)amino)-2-oxoethyl)(cyclopropyl)amino)-2-oxoethyl)-1H-indazole-3-carboxamide (35.6 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.96 (s, 1H), 7.56-7.58 (m, 2H), 7.39 (t, 1H), 7.34 (s, 1H), 7.26 (t, 1H), 5.70 (s, 2H), 4.54 (s, 2H), 4.13 (s, 2H), 3.10-3.11 (m, 1H), 1.02-1.05 (m, 4H). LCMS (M+H⁺) m/z calculated 480.1, found 480.2.

### Example 115: Preparation of 1-(2-(cyclopropyl(2-((2-fluorobenzyl)amino)-2-oxoethyl)amino)-2-oxoethyl)-1H-indazole-3-carboxamide

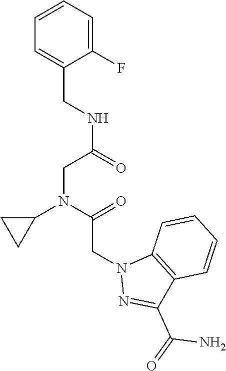 [SMILES: NC(=O)c1nn(CC(=O)N(CC(=O)NCc2ccccc2F)C2CC2)c2ccccc12]

1-(2-(Cyclopropyl(2-((2-fluorobenzyl)amino)-2-oxoethyl)amino)-2-oxoethyl)-1H-indazole-3-carboxamide (41.5 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.23 (d, 1H), 7.60 (d, 1H), 7.45 (t, 1H), 7.24-7.32 (m, 3H), 7.02-7.07 (m, 2H), 5.70 (s, 2H), 4.43 (s, 2H), 4.13 (s, 2H), 3.10-3.12 (m, 1H), 1.02-1.05 (m, 4H). LCMS (M+Na⁺) m/z calculated 424.2, found 424.2.

### Example 116: Preparation of 1-(2-((2-((3-chloro-2-methoxybenzyl)amino)-2-oxoethyl)(cyclopropyl)amino)-2-oxoethyl)-1H-indazole-3-carboxamide

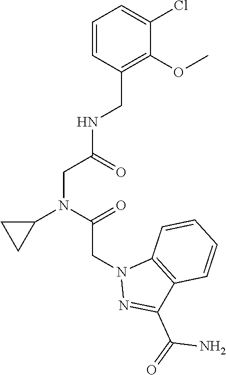 [SMILES: COc1c(Cl)cccc1CNC(=O)CN(C(=O)Cn1nc(C(N)=O)c2ccccc21)C1CC1]

1-(2-((2-((3-Chloro-2-methoxybenzyl)amino)-2-oxoethyl)(cyclopropyl)amino)-2-oxoethyl)-1H-indazole-3-carboxamide (51.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.23 (d, 1H), 7.59 (d, 1H), 7.43 (t, 1H), 7.28 (t, 2H), 7.19 (d, 1H), 6.97 (t, 1H), 5.70 (s, 2H), 4.43 (s, 2H), 4.13 (s, 2H), 3.83 (s, 3H), 3.10-3.12 (m, 1H), 1.04 (t, 4H). LCMS (M+Na⁺) m/z calculated 470.1, found 470.5.

### Example 117: Preparation of 1-(2-((2-((4-carbamoyl-2-fluorobenzyl)amino)-2-oxoethyl)(cyclopropyl)amino)-2-oxoethyl)-1H-indazole-3-carboxamide

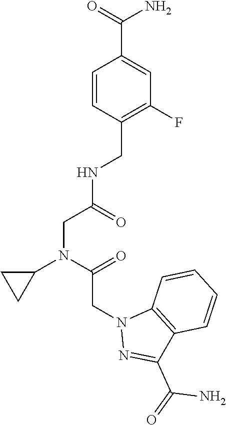 [SMILES: NC(=O)c1ccc(CNC(=O)CN(C(=O)Cn2nc(C(N)=O)c3ccccc32)C2CC2)c(F)c1]

1-(2-((2-((4-Carbamoyl-2-fluorobenzyl)amino)-2-oxoethyl)(cyclopropyl)amino)-2-oxoethyl)-1H-indazole-3-carboxamide (30.9 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.21 (d, 1H), 7.58-7.53 (m, 1H), 7.59 (d, 3H), 7.44-7.35 (m, 2H), 7.27 (t, 1H), 5.70 (s, 2H), 4.50 (s, 2H), 4.14 (s, 2H), 3.15-3.08 (m, 1H), 1.09-1.00 (m, 4H). LCMS (M+H⁺) m/z calculated 467.2, found 467.6.

### Example 118: Preparation of 1-(2-((2-((3-amino-2-fluorobenzyl)amino)-2-oxoethyl)(cyclopropyl)amino)-2-oxoethyl)-1H-indazole-3-carboxamide

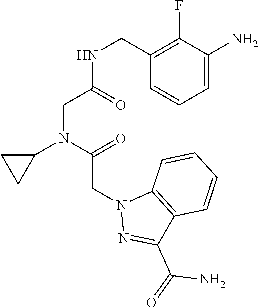 [SMILES: NC(=O)c1nn(CC(=O)N(CC(=O)NCc2cccc(N)c2F)C2CC2)c2ccccc12]

1-(2-((2-((3-Amino-2-fluorobenzyl)amino)-2-oxoethyl)(cyclopropyl)amino)-2-oxoethyl)-1H-indazole-3-carboxamide (108.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.59 (d, 1H), 7.44 (t, 1H), 7.28 (t, 1H), 6.70-6.81 (m, 2H), 6.57 (t, 1H), 5.70 (s, 2H), 4.38 (s, 2H), 4.12 (s, 2H), 3.08-3.13 (m, 1H), 1.02-1.05 (m, 4H). LCMS (M+Na⁺) m/z calculated 439.2, found 439.3.

### Example 119: Preparation of 1-(2-(cyclopropyl(2-((3,5-dichlorobenzyl)amino)-2-oxoethyl)amino)-2-oxoethyl)-1H-indazole-3-carboxamide

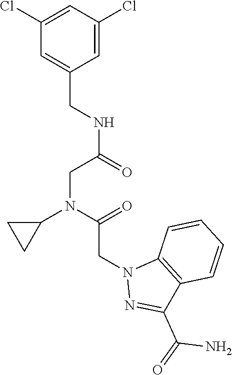 [SMILES: NC(=O)c1nn(CC(=O)N(CC(=O)NCc2cc(Cl)cc(Cl)c2)C2CC2)c2ccccc12]

1-(2-(Cyclopropyl(2-((3,5-dichlorobenzyl)amino)-2-oxoethyl)amino)-2-oxoethyl)-1H-indazole-3-carboxamide (10.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.20 (d, 1H), 7.60 (d, 1H), 7.42 (t, 1H), 7.23-7.29 (m, 4H), 5.70 (s, 2H), 4.34 (s, 2H), 4.13 (s, 2H), 3.11 (t, 1H), 0.98-1.05 (m, 4H). LCMS (M+Na⁺) m/z calculated 474.4, found 474.1.

### Example 120: Preparation of 1-(2-((2-((3-bromo-2-fluorobenzyl)amino)-2-oxoethyl)(cyclopropyl)amino)-2-oxoethyl)-1H-indazole-3-carboxamide

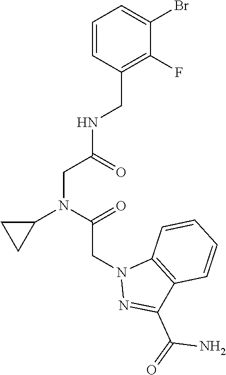 [SMILES: NC(=O)c1nn(CC(=O)N(CC(=O)NCc2cccc(Br)c2F)C2CC2)c2ccccc12]

1-(2-((2-((3-Bromo-2-fluorobenzyl)amino)-2-oxoethyl)(cyclopropyl)amino)-2-oxoethyl)-1H-indazole-3-carboxamide (25.5 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.58 (d, 1H), 7.41-7.49 (m, 2H), 7.26-7.30 (m, 2H), 6.95 (t, 1H), 5.70 (s, 2H), 4.44 (s, 2H), 4.13 (s, 2H), 3.08-3.12 (m, 1H), 1.03-1.04 (m, 4H). LCMS (M+Na⁺) m/z calculated 502.1, found 502.1.

### Example 121: Preparation of 1-(2-((2-((4-amino-2-fluorobenzyl)amino)-2-oxoethyl)(cyclopropyl)amino)-2-oxoethyl)-1H-indazole-3-carboxamide

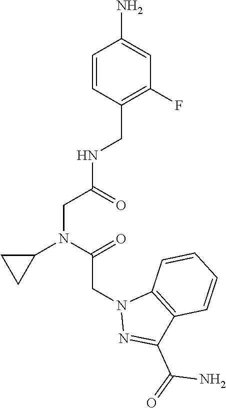 [SMILES: NC(=O)c1nn(CC(=O)N(CC(=O)NCc2ccc(N)cc2F)C2CC2)c2ccccc12]

1-(2-((2-((4-Amino-2-fluorobenzyl)amino)-2-oxoethyl)(cyclopropyl)amino)-2-oxoethyl)-1H-indazole-3-carboxamide (58.2 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.21 (d, 1H), 7.59 (d, 1H), 7.44 (t, 1H), 6.99 (t, 1H), 6.42-6.33 (m, 2H), 5.68 (s, 2H), 4.36 (s, 2H), 4.09 (s, 2H), 3.12-3.04 (m, 1H), 1.08-0.95 (m, 4H), LCMS (M+H⁺) m/z calculated 439.2, found 439.6.

### Example 122: Preparation of 1-(2-(cyclopropyl(2-((2-fluoro-3-methoxybenzyl)amino)-2-oxoethyl)amino)-2-oxoethyl)-1H-indazole-3-carboxamide

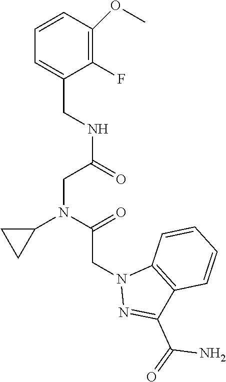 [SMILES: COc1cccc(CNC(=O)CN(C(=O)Cn2nc(C(N)=O)c3ccccc32)C2CC2)c1F]

1-(2-(Cyclopropyl(2-((2-fluoro-3-methoxybenzyl)amino)-2-oxoethyl)amino)-2-oxoethyl)-1H-indazole-3-carboxamide (63.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.21 (d, 1H), 7.57 (d, 1H), 7.41 (t, 1H), 7.26 (t, 1H), 6.96-6.98 (m, 2H), 6.82-6.86 (m, 1H), 5.69 (s, 2H), 4.41 (s, 2H), 4.12 (s, 2H), 3.83 (s, 3H), 3.08 (t, 1H), 1.03 (t, 4H). LCMS (M+Na⁺) m/z calculated 454.5, found 454.4.

### Example 123: Preparation of 1-(2-((2-((3-cyano-2-fluorobenzyl)amino)-2-oxoethyl)(cyclopropyl)amino)-2-oxoethyl)-1H-indazole-3-carboxamide

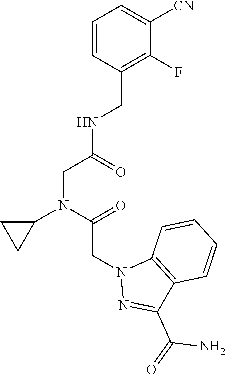 [SMILES: N#Cc1cccc(CNC(=O)CN(C(=O)Cn2nc(C(N)=O)c3ccccc32)C2CC2)c1F]

1-(2-((2-((3-Cyano-2-fluorobenzyl)amino)-2-oxoethyl)(cyclopropyl)amino)-2-oxoethyl)-1H-indazole-3-carboxamide (22.2 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.56-7.62 (m, 3H), 7.42 (t, 1H), 7.28 (t, 1H), 7.17 (t, 1H), 5.70 (s, 2H), 4.46 (s, 2H), 4.13 (s, 2H), 3.09-3.15 (m, 1H), 1.03-1.05 (m, 4H). LCMS (M+Na⁺) m/z calculated 449.2, found 449.3.

### Example 124: Preparation of 1-(2-((2-((3-chloro-5-(trifluoromethyl)benzyl)amino)-2-oxoethyl)(cyclopropyl)amino)-2-oxoethyl)-1H-indazole-3-carboxamide

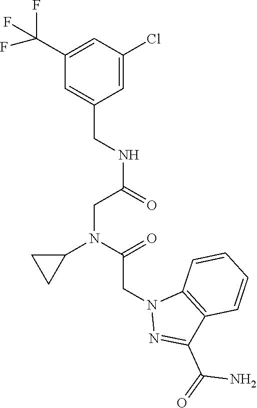 [SMILES: NC(=O)c1nn(CC(=O)N(CC(=O)NCc2cc(Cl)cc(C(F)(F)F)c2)C2CC2)c2ccccc12]

1-(2-((2-((3-Chloro-5-(trifluoromethyl)benzyl)amino)-2-oxoethyl)(cyclopropyl)amino)-2-oxoethyl)-1H-indazole-3-carboxamide (16.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.20 (d, 1H), 7.50-7.58 (m, 4H), 7.41 (t, 1H), 7.26 (t, 1H), 5.67 (s, 2H), 4.44 (s, 2H), 4.13 (s, 2H), 3.08 (t, 1H), 1.03 (t, 4H). LCMS (M+Na⁺) m/z calculated 508.4, found 508.1.

### Example 125: Preparation of 1-(2-((2-((3-chloro-4-methoxybenzyl)amino)-2-oxoethyl)(cyclopropyl)amino)-2-oxoethyl)-1H-indazole-3-carboxamide

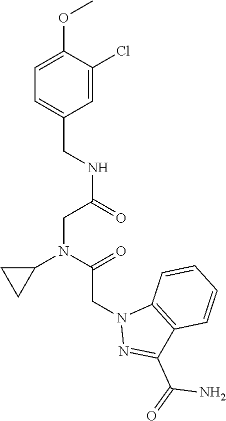 [SMILES: COc1ccc(CNC(=O)CN(C(=O)Cn2nc(C(N)=O)c3ccccc32)C2CC2)cc1Cl]

1-(2-((2-((3-Chloro-4-methoxybenzyl)amino)-2-oxoethyl)(cyclopropyl)amino)-2-oxoethyl)-1H-indazole-3-carboxamide (50.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.21 (d, 1H), 7.58 (d, 1H), 7.43 (t, 1H), 7.27 (d, 2H), 7.13 (d, 1H), 6.92 (d, 1H), 5.70 (s, 2H), 4.28 (s, 2H), 4.11 (s, 2H), 3.82 (s, 3H), 3.10-3.12 (m, 1H), 1.02 (d, 4H). LCMS (M+Na⁺) m/z calculated 470.2, found 470.6.

### Example 126: Preparation of 1-(2-(cyclopropyl(2-((2-fluoro-3-(hydroxymethyl)benzyl)amino)-2-oxoethyl)amino)-2-oxoethyl)-1H-indazole-3-carboxamide

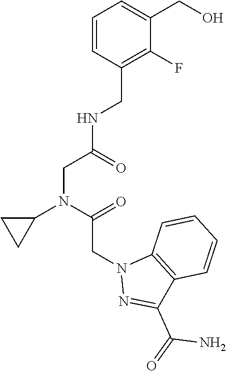 [SMILES: NC(=O)c1nn(CC(=O)N(CC(=O)NCc2cccc(CO)c2F)C2CC2)c2ccccc12]

1-(2-(Cyclopropyl(2-((2-fluoro-3-(hydroxymethyl)benzyl)amino)-2-oxoethyl)amino)-2-oxoethyl)-1H-indazole-3-carboxamide (41.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.24 (d, 1H), 7.61 (d, 1H), 7.46 (t, 1H), 7.36 (t, 1H), 7.31 (t, 1H), 7.25 (t, 1H), 7.07 (t, 1H), 5.72 (s, 2H), 4.67 (s, 2H), 4.45 (s, 2H), 4.15 (s, 2H), 3.10-3.14 (m, 1H), 1.05-1.07 (m, 4H). LCMS (M+Na⁺) m/z calculated 454.2, found 454.2.

### Example 127: Preparation of 1-(2-(cyclopropyl(2-((3-cyclopropyl-2-fluorobenzyl)amino)-2-oxoethyl)amino)-2-oxoethyl)-1H-indazole-3-carboxamide

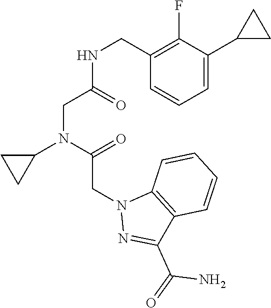 [SMILES: NC(=O)c1nn(CC(=O)N(CC(=O)NCc2cccc(C3CC3)c2F)C2CC2)c2ccccc12]

1-(2-(Cyclopropyl(2-((3-cyclopropyl-2-fluorobenzyl)amino)-2-oxoethyl)amino)-2-oxoethyl)-1H-indazole-3-carboxamide (11.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.59 (d, 1H), 7.43 (t, 1H), 7.28 (t, 1H), 7.07 (t, 1H), 6.93 (t, 1H), 6.83 (t, 1H), 5.70 (s, 2H), 4.42 (s, 2H), 4.13 (s, 2H), 3.08-3.13 (m, 1H), 2.02-2.08 (m, 1H), 0.99-1.05 (m, 4H), 0.93-0.98 (m, 2H), 0.66-0.70 (m, 2H). LCMS (M+Na⁺) m/z calculated 464.2, found 464.3.

### Example 128: Preparation of 1-(2-(cyclopropyl(2-((2-fluoro-3-methylbenzyl)amino)-2-oxoethyl)amino)-2-oxoethyl)-1H-indazole-3-carboxamide

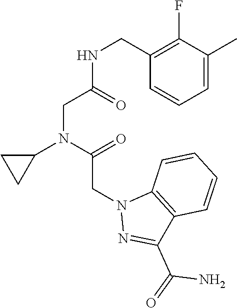 [SMILES: Cc1cccc(CNC(=O)CN(C(=O)Cn2nc(C(N)=O)c3ccccc32)C2CC2)c1F]

1-(2-(Cyclopropyl(2-((2-fluoro-3-methylbenzyl)amino)-2-oxoethyl)amino)-2-oxoethyl)-1H-indazole-3-carboxamide (43.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.59 (d, 1H), 7.44 (t, 1H), 7.28 (t, 1H), 7.10 (t, 2H), 6.93 (t, 1H), 5.70 (s, 2H), 4.41 (s, 2H), 4.13 (s, 2H), 3.08-3.15 (m, 1H), 2.24 (s, 3H), 1.02-1.05 (m, 4H). LCMS (M+Na⁺) m/z calculated 438.2, found 438.3.

### Example 129: Preparation of 1-(2-((2-((3-chloro-5-methylbenzyl)amino)-2-oxoethyl)(cyclopropyl)amino)-2-oxoethyl)-1H-indazole-3-carboxamide

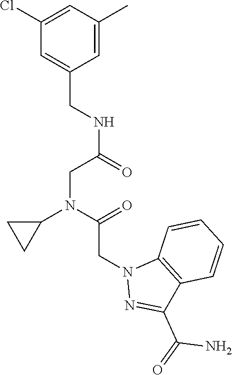 [SMILES: Cc1cc(Cl)cc(CNC(=O)CN(C(=O)Cn2nc(C(N)=O)c3ccccc32)C2CC2)c1]

1-(2-((2-((3-Chloro-5-methylbenzyl)amino)-2-oxoethyl)(cyclopropyl)amino)-2-oxoethyl)-1H-indazole-3-carboxamide (41.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.20 (d, 1H), 7.58 (d, 1H), 7.43 (t, 1H), 7.27 (t, 1H), 7.04 (t, 2H), 6.99 (d, 1H), 5.70 (s, 2H), 4.31 (s, 2H), 4.13 (s, 2H), 3.11 (d, 1H), 2.21 (s, 3H), 1.03 (d, 4H). LCMS (M+Na⁺) m/z calculated 454.1, found 454.3.

### Example 130: Preparation of 1-(2-((2-(((5-chloropyridin-3-yl)methyl)amino)-2-oxoethyl)(cyclopropyl)amino)-2-oxoethyl)-1H-indazole-3-carboxamide

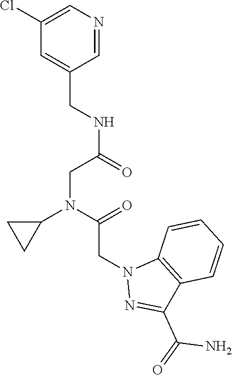 [SMILES: NC(=O)c1nn(CC(=O)N(CC(=O)NCc2cncc(Cl)c2)C2CC2)c2ccccc12]

1-(2-((2-(((5-Chloropyridin-3-yl)methyl)amino)-2-oxoethyl)(cyclopropyl)amino)-2-oxoethyl)-1H-indazole-3-carboxamide (51.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.39 (s, 2H), 8.20 (d, 1H), 7.77 (s, 1H), 7.57 (d, 1H), 7.42 (t, 1H), 7.27 (t, 1H), 5.69 (s, 2H), 4.40 (s, 2H), 4.13 (s, 2H), 3.11 (d, 1H), 1.03 (d, 4H). LCMS (M+Na⁺) m/z calculated 441.1, found 441.0.

### Example 131: Preparation of 1-(2-((2-((3-chloro-2-cyclopropylbenzyl)amino)-2-oxoethyl)(cyclopropyl)amino)-2-oxoethyl)-1H-indazole-3-carboxamide

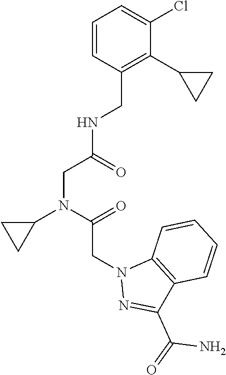 [SMILES: NC(=O)c1nn(CC(=O)N(CC(=O)NCc2cccc(Cl)c2C2CC2)C2CC2)c2ccccc12]

1-(2-((2-((3-Chloro-2-cyclopropylbenzyl)amino)-2-oxoethyl)(cyclopropyl)amino)-2-oxoethyl)-1H-indazole-3-carboxamide (35.8 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.58 (d, 1H), 7.43 (t, 1H), 7.28 (t, 2H), 7.22 (d, 1H), 7.14 (d, 1H), 7.06 (t, 1H), 5.70 (s, 2H), 4.63 (s, 2H), 4.14 (s, 2H), 3.15-3.08 (m, 1H), 1.80-1.69 (m, 1H), 1.12-0.96 (m, 6H), 0.67-0.60 (m, 2H). LCMS (M+H⁺) m/z calculated 480.2, found 480.3.

### Example 132: Preparation of 1-(2-(cyclopropyl(2-((2,3-difluorobenzyl)amino)-2-oxoethyl)amino)-2-oxoethyl)-1H-indazole-3-carboxamide

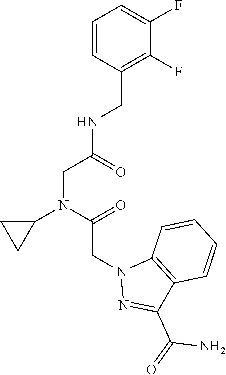 [SMILES: NC(=O)c1nn(CC(=O)N(CC(=O)NCc2cccc(F)c2F)C2CC2)c2ccccc12]

1-(2-(Cyclopropyl(2-((2,3-difluorobenzyl)amino)-2-oxoethyl)amino)-2-oxoethyl)-1H-indazole-3-carboxamide (39.1 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.58 (d, 1H), 7.43 (t, 1H), 7.28 (t, 1H), 7.14-7.00 (m, 3H), 5.70 (s, 2H), 4.45 (s, 2H), 4.13 (s, 2H), 3.12-3.10 (m, 1H), 1.05-1.03 (m, 4H). LCMS (M+H⁺) m/z calculated 442.2, found 442.2.

### Example 133: Preparation of 1-(2-((2-((3-chloro-5-cyanobenzyl)amino)-2-oxoethyl)(cyclopropyl)amino)-2-oxoethyl)-1H-indazole-3-carboxamide

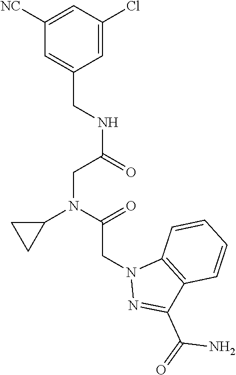 [SMILES: N#Cc1cc(Cl)cc(CNC(=O)CN(C(=O)Cn2nc(C(N)=O)c3ccccc32)C2CC2)c1]

1-(2-((2-((3-Chloro-5-cyanobenzyl)amino)-2-oxoethyl)(cyclopropyl)amino)-2-oxoethyl)-1H-indazole-3-carboxamide (50.0 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.21 (d, 1H), 7.59 (t, 4H), 7.42 (t, 1H), 7.27 (t, 1H), 5.69 (s, 2H), 4.39 (s, 2H), 4.14 (s, 2H), 3.11 (t, 1H), 1.04 (d, 4H). LCMS (M+Na⁺) m/z calculated 465.1, found 465.1.

### Example 134: Preparation of 1-(2-((2-((3-chloro-2-methylbenzyl)amino)-2-oxoethyl)(cyclopropyl)amino)-2-oxoethyl)-1H-indazole-3-carboxamide

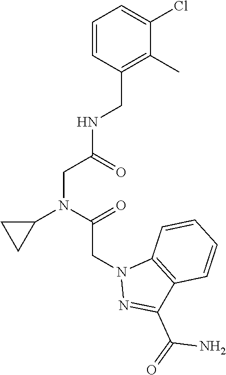 [SMILES: Cc1c(Cl)cccc1CNC(=O)CN(C(=O)Cn1nc(C(N)=O)c2ccccc21)C1CC1]

1-(2-((2-((3-Chloro-2-methylbenzyl)amino)-2-oxoethyl)(cyclopropyl)amino)-2-oxoethyl)-1H-indazole-3-carboxamide (9.7 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) 8.22 (d, 1H), 7.58 (d, 1H), 7.43 (t, 1H), 7.32-7.24 (m, 2H), 7.16 (d, 1H), 7.05 (t, 1H), 5.70 (s, 2H), 4.40 (s, 2H), 4.12 (s, 2H), 3.16-3.08 (m, 1H), 2.33 (s, 3H), 1.10-1.01 (m, 4H). LCMS (M+H⁺) m/z calculated 454.2, found 454.1.

### Example 135: Preparation of 1-(2-((2-((3-chloro-2-(trifluoromethyl)benzyl)amino)-2-oxoethyl)(cyclopropyl)amino)-2-oxoethyl)-1H-indazole-3-carboxamide

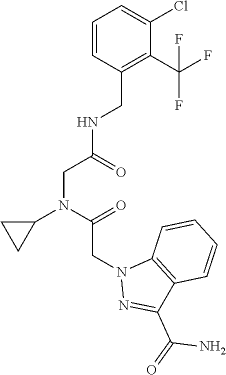 [SMILES: NC(=O)c1nn(CC(=O)N(CC(=O)NCc2cccc(Cl)c2C(F)(F)F)C2CC2)c2ccccc12]

1-(2-((2-((3-Chloro-2-(trifluoromethyl)benzyl)amino)-2-oxoethyl)(cyclopropyl)amino)-2-oxoethyl)-1H-indazole-3-carboxamide (48.3 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz) δ 8.22 (d, 1H), 7.57 (d, 1H), 7.48-7.26 (m, 5H), 5.70 (s, 2H), 4.57 (s, 2H), 4.16 (s, 2H), 3.15-3.09 (m, 1H), 1.12-1.00 (m, 4H). LCMS (M+H⁺) m/z calculated 508.1, found 508.2.

### Example 136: Preparation of 1-(2-(cyclopropyl(2-((2-fluoro-3-(trifluoromethyl)benzyl)amino)-2-oxoethyl)amino)-2-oxoethyl)-1H-indazole-3-carboxamide

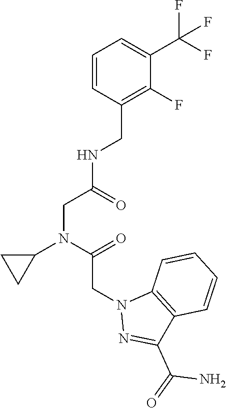 [SMILES: NC(=O)c1nn(CC(=O)N(CC(=O)NCc2cccc(C(F)(F)F)c2F)C2CC2)c2ccccc12]

1-(2-(cyclopropyl(2-((2-fluoro-3-(trifluoromethyl)benzyl)amino)-2-oxoethyl)amino)-2-oxoethyl)-1H-indazole-3-carboxamide (3.3 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR. (CD3OD, 400 MHz) δ 8.22 (d, 1H), 7.57 (t, 1H), 7.43 (t, 1H), 7.28 (t, 1H), 7.18 (t, 1H), 5.71 (s, 2H), 4.48 (s, 2H), 4.14 (s, 2H), 3.12-3.10 (m, 1H), 1.12-1.00 (m, 4H). LRMS (M+1) m/z calculated 492.2, found 492.1.

### Example 137: Preparation of 2-(3-acetylimidazo[1,5-a]pyridin-1-yl)-N-(2-((3-chloro-2-fluorobenzyl)amino)-2-oxoethyl)-N-cyclopropylacetamide

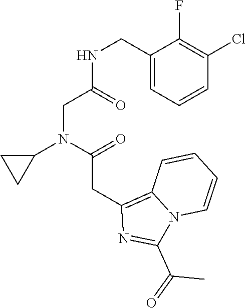 [SMILES: CC(=O)c1nc(CC(=O)N(CC(=O)NCc2cccc(Cl)c2F)C2CC2)c2ccccn12]

2-(3-Acetylimidazo[1,5-a]pyridin-1-yl)-N-(2-((3-chloro-2-fluorobenzyl)amino)-2-oxoethyl)-N-cyclopropylacetamide (19.3 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (400 MHz, CDCl₃) δ 9.57 (d, 1H), 7.69 (d, 1H), 7.08-7.15 (m, 2H), 6.90-6.98 (m, 2H), 7.40 (d, 1H), 7.27 (d, 1H), 6.87-6.91 (m, 1H), 6.79-6.82 (m, 1H), 4.43 (d, 2H), 4.30 (s, 2H), 4.09 (s, 2H), 3.04-3.10 (m, 1H), 2.68 (s, 3H), 0.98-1.04 (m, 4H). LCMS (M+H⁺) m/z calculated 457.1, found 457.2.

### Example 138: Preparation of 1-(2-((2-((3-chloro-2-fluorobenzyl)amino)-2-oxoethyl)(cyclopropyl)amino)-2-oxoethyl)imidazo[1,5-a]pyridine-3-carboxamide

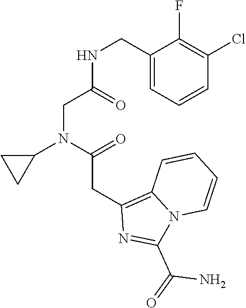 [SMILES: NC(=O)c1nc(CC(=O)N(CC(=O)NCc2cccc(Cl)c2F)C2CC2)c2ccccn12]

1-(2-((2-((3-Chloro-2-fluorobenzyl)amino)-2-oxoethyl)(cyclopropyl)amino)-2-oxoethyl)imidazo[1,5-a]pyridine-3-carboxamide (24.5 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. ¹H NMR (CD₃OD, 400 MHz,) δ 9.34 (d, 1H), 7.72 (d, 1H), 7.32 (t, 1H), 7.23 (t, 1H), 7.00-7.08 (m, 2H), 6.92 (d, 1H), 4.45 (s, 2H), 4.33 (s, 2H), 4.13 (s, 2H), 3.04-3.10 (m, 1H), 0.95-1.02 (m, 4H). LCMS (M+H⁺) m/z calculated 458.1, found 458.2.

### Example 139: Preparation of 2-(1-acetylimidazo[1,5-a]pyridin-3-yl)-N-(2-((3-chloro-2-fluorobenzyl)amino)-2-oxoethyl)-N-cyclopropylacetamide

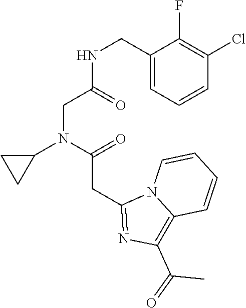 [SMILES: CC(=O)c1nc(CC(=O)N(CC(=O)NCc2cccc(Cl)c2F)C2CC2)n2ccccc12]

2-(1-Acetylimidazo[1,5-a]pyridin-3-yl)-N-(2-((3-chloro-2-fluorobenzyl)amino)-2-oxoethyl)-N-cyclopropylacetamide (2.8 mg) was prepared as described for 1-(2-((2-((3-chloro-2-fluorobenzyl)amino)-2-oxoethyl)(3-methylbutan-2-yl)amino)-2-oxoethyl)-1H-indazole-3-carboxamide. 1H NMR. (MeOH-d4, 400 MHz) δ=8.24-8.31 (m, 2H), 7.32-7.35 (t, 2H), 7.22-7.26 (m, 1H), 7.00-7.04 (m, 1H), 6.93-6.96 (m, 1H), 7.62 (s, 2H), 7.45 (s, 2H), 4.13 (s, 2H), 3.09-3.15 (m, 1H), 2.59 (s, 3H), 1.02-1.03 (m, 4H). LRMS (M+H⁺) m/z calculated 457.6, found 457.6.

### Example 140: Preparation of 3-(3-(2-((3-chloro-2-fluorobenzyl)amino)-2-oxoethyl)-3-cyclopropylureido)imidazo[1,5-a]pyridine-1-carboxamide

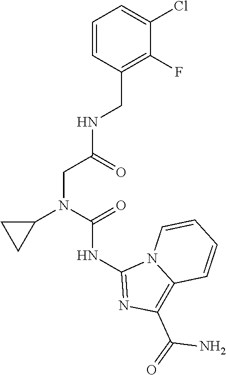 [SMILES: NC(=O)c1nc(NC(=O)N(CC(=O)NCc2cccc(Cl)c2F)C2CC2)n2ccccc12]

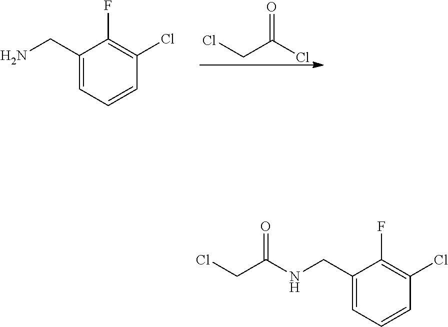 [SMILES: NCc1cccc(Cl)c1F ; O=C(Cl)CCl ; O=C(CCl)NCc1cccc(Cl)c1F]

2-Chloroacetyl chloride (142.0 mg, 1.25 mmol, 1.0 eq) was added to a solution of (3-chloro-2-fluorophenyl) methanamine (200.0 mg, 1.25 mmol, 1.0 eq) in dry toluene (10.0 mL). The resulting mixture was stirred at rt for 3 h, then cooled and concentrated in vacuo. The resulting residue was purified by column chromatography (EA/PE=1:5) to provide 2-chloro-N-(3-chloro-2-fluorobenzyl)acetamide (200.0 mg, 67.8%).

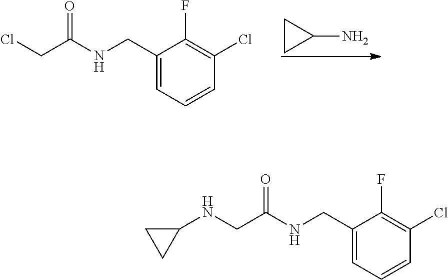

Cyclopropanamine (30.0 mg, 0.51 mmol, 1.2 eq) was added to a solution of 2-chloro-N-(3-chloro-2-fluorobenzyl)acetamide (100.0 mg, 0.43 mmol, 1.0 eq) and K₂CO₃ (88.0 mg, 0.64 mmol, 1.5 eq) in dry DMF (5.0 mL). The resulting mixture was stirred at rt overnight, then cooled, filtered and quenched by water. The mixture was extracted with EtOAc (50.0 mL×2). The combined organic layers were dried over anhydrous Na₂SO₄ and concentrated in vacuo. The residue was purified by column chromatography (EA/PE=1/5, v/v) to provide to provide N-(3-chloro-2-fluorobenzyl)-2-(cyclopropylamino)acetamide (90.0 mg, 81.8%).

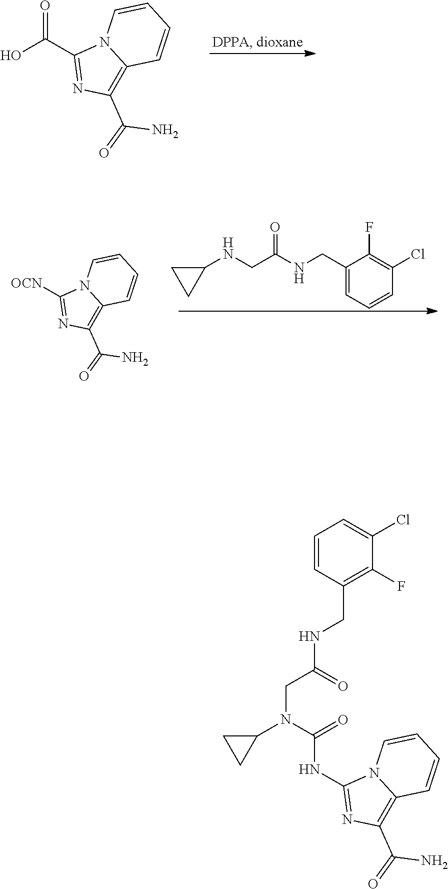

To a solution of 1-carbamoylimidazo[1,5-a]pyridine-3-carboxylic acid (100.0 mg, 0.31 mmol, 1.0 eq) in 1,4-dioxane (3.0 mL) were added to DPPA (103.0 mg, 0.38 mmol, 1.2 eq) and TEA (38.0 mg, 0.38 mmol, 1.2 eq). After the addition was completed, the resulting mixture was stirred at room temperature for 2.0 h. The mixture was then added to N-(3-chloro-2-fluorobenzyl)-2-(cyclopropylamino) acetamide (80.0 mg, 0.12 mmol, 1.0 eq). The mixture was stirred at 100° C. for 2.0 h, then cooled and concentrated in vacuo. The resulting residue was purified by prep-HPLC to provide 3-(3-(2-((3-chloro-2-fluorobenzyl)amino)-2-oxoethyl)-3-cyclopropylureido)imidazo[1,5-a]pyridine-1-carboxamide (1.5 mg, 0.7%). ¹H NMR (CD₃OD, 400 MHz) δ 8.15-8.09 (m, 2H), 7.38-7.29 (m, 2H), 7.19-7.05 (m, 2H), 8.84 (t, 1H), 4.49 (s, 2H), 4.12 (s, 2H), 2.97 (s, 1H), 0.99 (t, 4H). LCMS (M+H⁺) m/z calculated 459.1, found 459.1.

### Example 141: Preparation of 2-(3-(3-acetylimidazo[1,5-a]pyridin-1-yl)-1-cyclopropylureido)-N-(3-chloro-2-fluorobenzyl)acetamide

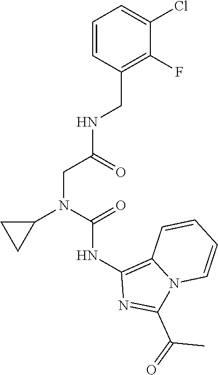 [SMILES: CC(=O)c1nc(NC(=O)N(CC(=O)NCc2cccc(Cl)c2F)C2CC2)c2ccccn12]

2-(3-(3-Acetylimidazo[1,5-a]pyridin-1-yl)-1-cyclopropylureido)-N-(3-chloro-2-fluorobenzyl)acetamide (3.6 mg) was prepared as described for 3-(3-(2-((3-chloro-2-fluorobenzyl)amino)-2-oxoethyl)-3-cyclopropylureido)imidazo[1,5-a]pyridine-1-carboxamide. ¹H NMR (CD₃OD, 400 MHz) δ 8.57 (d, 1H), 7.78-7.80 (m, 1H), 7.60-7.63 (m, 1H), 7.45-7.55 (m, 1H), 7.31-7.37 (m, 1H), 7.23 (t, 1H), 7.06-7.14 (m, 1H), 4.48 (s, 2H), 4.12 (s, 2H), 2.96-2.97 (m, 1H), 2.66 (s, 3H), 1.26-1.28 (m, 2H), 0.98-1.00 (m, 2H). LCMS (M+H⁺) m/z calculated 458.1, found 458.7.

### II. Biological Evaluation

### Example 1: In Vitro Enzyme Inhibition

The ability of the compounds disclosed herein to inhibit human complement factor D inhibitory activity was quantified according to the 12-step protocol provided below.
1. Prepare assay buffer: 50 mM Tris/HCl, pH 7.5, 1 M NaCl.
2. Dilute 10 mM Complement Factor D inhibitor Nafamostat Mesilate (Selleckchem, Catalog# S1386) solution from 10000 μM to 9.77 μM in 100% DMSO, 8 concentrations. Then dilute the serial concentrations of Nafamostat Mesilate 20-fold in assay buffer.
3. Add 10 μl diluted Nafamostat Mesilate duplicated into each of the inhibitor control well of a 96-well plate (Corning, Catalog#3599). Final concentrations are 50 μM, 25 μM, 12.5 μM, 6.25 μM, 3.125 μM, 0.781 μM, 0.195 μM and 0.049 μM. 0.5% DMSO was in each well finally.
4. Dilute 20 mM test compounds from 10000 μM to 35.72 μM in 100% DMSO, 6-fold dilution, 8 concentrations. Then dilute the serial concentrations of test compounds 20-fold in assay buffer.
5. Add 10 μl diluted test compounds duplicated into the 96-well plate. Final concentrations were 50 μM, 8.33 μM, 1.39 μM, 0.23 μM, 0.0386 μM, 0.0064 μM, 0.0011 μM and 0.0002 μM. 0.5% DMSO was in each well finally.
6. Dilute 20 mM substrate Z-Lys-SBzl (Bachem, Cat# M-1300) to 200 μM in assay buffer with 200 μM DTNB (Sigma, Catalog# D8130).
7. Dilute 738 ng/L Complement Factor D (R&D Systems, Catalog#1824-SE) to 6.25 ng/L in assay buffer. Add 40 μl diluted Complement Factor D in the 96-well plate.
8. Positive control well contains Complement Factor D without test compound. Negative control well contains neither Complement Factor D nor test compound. Using assay buffer, bring the total volume of all controls to 50 al.
9. Pre-incubate the plate for 5 min at room temperature.
10. Add 50 μl of diluted substrate/DTNB mixture into each well. Mix the reagents completely by shaking the plate gently for 30 sec.
11. For kinetic reading: Immediately start measuring absorbance (A_{405 nm}) continuously and record data every 30 sec for 60 min.
12. Data analysis
   Inhibition activity of compound was evaluated by IC₅₀. IC₅₀ was calculated according the dose-response curve of compound fitted using GraphPadPrism with “log(inhibitor)-response (variable slope)” equation.
   % inhibition was calculated by using following equation:

$$Inhibition \% = 100 - \frac{Sample value - Mean ( NC )}{Mean ( PC ) - Mean ( NC )} \times 100$$

Mean(NC): The average value of the negative control wells' A_{405 nm} values.
Mean(PC): The average value of the positive control wells' A_{405 nm} values.

The ability of the compounds disclosed herein to inhibit human complement factor D inhibitory activity was determined and the data is shown in Table 4.

TABLE 4

| | | CFD |
|---|---|---|
| Ex. | Name | IC₅₀ |
| 1 | (S)-1-(2-((2-((3-chloro-2-fluorobenzyl)amino)-1- | D |
| | cyclopropyl-2-oxoethyl)amino)-2-oxoethyl)-1H-indazole- | |
| | 3-carboxamide | |
| 2 | (S)-1-(2-((1-((3-chloro-2-fluorophenyl)amino)-1- | C |
| | oxopropan-2-yl)(methyl)amino)-2-oxoethyl)-1H-indazole- | |
| | 3-carboxamide | |
| 3 | (S)-1-(2-((1-((3-chloro-2-fluorobenzyl)amino)-1- | C |
| | oxopropan-2-yl)(methyl)amino)-2-oxoethyl)-1H-indazole- | |
| | 3-carboxamide | |
| 4 | 1-(2-((2-((3-chloro-2-fluorobenzyl)amino)-2- | A |
| | oxoethyl)(cyclopropyl)amino)-2-oxoethyl)-1H-indazole-3- | |
| | carboxamide | |
| 5 | 1-(2-((2-((3-chloro-2-fluorobenzyl)amino)-2- | A |
| | oxoethyl)(isopropyl)amino)-2-oxoethyl)-1H-indazole-3- | |
| | carboxamide | |
| 6 | 1-(2-((2-((3-chloro-2-fluorobenzyl)amino)-2- | A |
| | oxoethyl)(ethyl)amino)-2-oxoethyl)-1H-indazole-3- | |
| | carboxamide | |
| 7 | 1-(2-((2-((3-chloro-2-fluorophenyl)amino)-2- | B |
| | oxoethyl)(cyclopropyl)amino)-2-oxoethyl)-1H-indazole-3- | |
| | carboxamide | |
| 8 | 3-(2-((2-((3-chloro-2-fluorobenzyl)amino)-2- | A |
| | oxoethyl)(cyclopropyl)amino)-2-oxoethyl)-1H-indazole-1- | |
| | carboxamide | |
| 9 | 1-(2-(cyclopropyl(2-((5-hydroxy-3,3- | C |
| | dimethylcyclohexyl)amino)-2-oxoethyl)amino)-2- | |
| | oxoethyl)-1H-indazole-3-carboxamide | |
| 10 | 1-(2-(cyclopropyl(2-(((1S,5S)-5-hydroxy-3,3- | C |
| | dimethylcyclohexyl)amino)-2-oxoethyl)amino)-2- | |
| | oxoethyl)-1H-indazole-3-carboxamide | |
| 11 | 1-(2-((2-((3-chloro-2-fluorophenyl)amino)-2- | B |
| | oxoethyl)(isopropyl)amino)-2-oxoethyl)-1H-indazole-3- | |
| | carboxamide | |
| 12 | 1-(2-((2-((6-chloropyridin-2-yl)amino)-2- | B |
| | oxoethyl)(isopropyl)amino)-2-oxoethyl)-1H-indazole-3- | |
| | carboxamide | |
| 13 | 1-(2-((2-((6-chloropyridin-2-yl)amino)-2-oxoethyl)(2- | C |
| | hydroxyethyl)amino)-2-oxoethyl)-1H-indazole-3- | |
| | carboxamide | |
| 14 | 1-(2-((2-((6-chloropyridin-2-yl)amino)-2- | C |
| | oxoethyl)(methyl)amino)-2-oxoethyl)-1H-indazole-3- | |
| | carboxamide | |
| 15 | 1-(2-((2-((6-chloropyridin-2-yl)amino)-2- | B |
| | oxoethyl)(ethyl)amino)-2-oxoethyl)-1H-indazole-3- | |
| | carboxamide | |
| 16 | 1-(2-((2-((6-chloropyridin-2-yl)amino)-2- | B |
| | oxoethyl)(cyclopropyl)amino)-2-oxoethyl)-1H-indazole-3- | |
| | carboxamide | |
| 17 | 1-(2-((2-((6-chloropyridin-2-yl)amino)-2- | C |
| | oxoethyl)(piperidin-4-yl)amino)-2-oxoethyl)-1H-indazole- | |
| | 3-carboxamide | |
| 18 | 3-(2-((2-aminoethyl)(2-((6-chloropyridin-2-yl)amino)-2- | C |
| | oxoethyl)amino)-2-oxoethyl)-1H-indazole-1-carboxamide | |
| 19 | 3-(2-((2-((6-chloropyridin-2-yl)amino)-2- | B |
| | oxoethyl)(cyclopentyl)amino)-2-oxoethyl)-1H-indazole-1- | |
| | carboxamide | |
| 20 | 3-(2-((2-((6-chloropyridin-2-yl)amino)-2- | B |
| | oxoethyl)(cyclobutyl)amino)-2-oxoethyl)-1H-indazole-1- | |
| | carboxamide | |
| 21 | 3-(2-((2-((6-chloropyridin-2-yl)amino)-2- | C |
| | oxoethyl)(tetrahydro-2H-pyran-4-yl)amino)-2-oxoethyl)- | |
| | 1H-indazole-1-carboxamide | |
| 22 | 1-(2-(azetidin-3-yl(2-((6-chloropyridin-2-yl)amino)-2- | C |
| | oxoethyl)amino)-2-oxoethyl)-1H-indazole-3-carboxamide | |
| 23 | 1-(2-((2-((6-chloropyridin-2-yl)amino)-2-oxoethyl)(2- | C |
| | (methylamino)ethyl)amino)-2-oxoethyl)-1H-indazole-3- | |
| | carboxamide | |
| 24 | 1-(2-((2-acetamidoethyl)(2-((6-chloropyridin-2-yl)amino)- | C |
| | 2-oxoethyl)amino)-2-oxoethyl)-1H-indazole-3- | |
| | carboxamide | |
| 25 | 1-(2-((2-((6-chloropyridin-2-yl)amino)-2- | D |
| | oxoethyl)(pyridin-4-ylmethyl)amino)-2-oxoethyl)-1H- | |
| | indazole-3-carboxamide | |
| 26 | 1-(2-((2-((6-chloropyridin-2-yl)amino)-2- | C |
| | oxoethyl)(pyrrolidin-3-yl)amino)-2-oxoethyl)-1H- | |
| | indazole-3-carboxamide | |
| 27 | 1-(2-((2-((3-chloro-2-fluorobenzyl)amino)-2- | C |
| | oxoethyl)(thiophen-3-ylmethyl)amino)-2-oxoethyl)-1H- | |
| | indazole-3-carboxamide | |
| 28 | 1-(2-((2-((3-chloro-2-fluorobenzyl)amino)-2- | A |
| | oxoethyl)(cyclobutyl)amino)-2-oxoethyl)-1H-indazole-3- | |
| | carboxamide | |
| 29 | 1-(2-((2-((3-chloro-2-fluorobenzyl)amino)-2- | C |
| | oxoethyl)(tetrahydro-2H-pyran-4-yl)amino)-2-oxoethyl)- | |
| | 1H-indazole-3-carboxamide | |
| 30 | 1-(2-((2-((3-chloro-2-fluorobenzyl)amino)-2- | B |
| | oxoethyl)(cyclopentyl)amino)-2-oxoethyl)-1H-indazole-3- | |
| | carboxamide | |
| 31 | 1-(2-((2-aminoethyl)(2-((3-chloro-2-fluorobenzyl)amino)- | B |
| | 2-oxoethyl)amino)-2-oxoethyl)-1H-indazole-3- | |
| | carboxamide | |
| 32 | 1-(2-((2-((3-chloro-2-fluorobenzyl)amino)-2- | C |
| | oxoethyl)(pyridin-4-ylmethyl)amino)-2-oxoethyl)-1H- | |
| | indazole-3-carboxamide | |
| 33 | 1-(2-((2-((3-chloro-2-fluorobenzyl)amino)-2- | B |
| | oxoethyl)(methyl)amino)-2-oxoethyl)-1H-indazole-3- | |
| | carboxamide | |
| 34 | 1-(2-((2-((3-chloro-2-fluorobenzyl)amino)-2- | B |
| | oxoethyl)(piperidin-4-yl)amino)-2-oxoethyl)-1H-indazole- | |
| | 3-carboxamide | |
| 35 | 1-(2-((2-((3-chloro-2-fluorobenzyl)amino)-2- | C |
| | oxoethyl)(pyridin-3-ylmethyl)amino)-2-oxoethyl)-1H- | |
| | indazole-3-carboxamide | |
| 36 | 1-(2-((2-((3-chloro-2-fluorobenzyl)amino)-2- | C |
| | oxoethyl)(phenyl)amino)-2-oxoethyl)-1H-indazole-3- | |
| | carboxamide | |
| 37 | 1-(2-((2-((3-chloro-2-fluorobenzyl)amino)-2- | C |
| | oxoethyl)(pyrrolidin-3-yl)amino)-2-oxoethyl)-1H- | |
| | indazole-3-carboxamide | |
| 38 | (R)-1-(2-((1-((3-chloro-2-fluorobenzyl)amino)-1- | C |
| | oxopropan-2-yl)(cyclopropyl)amino)-2-oxoethyl)-1H- | |
| | indazole-3-carboxamide | |
| 39 | (S)-1-(2-((1-((3-chloro-2-fluorobenzyl)amino)-1- | D |
| | oxopropan-2-yl)(cyclopropyl)amino)-2-oxoethyl)-1H- | |
| | indazole-3-carboxamide | |
| 40 | 1-(2-((2-((3-chloro-2-fluorobenzyl)amino)-2- | A |
| | oxoethyl)(cyclopropyl)amino)-2-oxoethyl)-1H- | |
| | pyrazolo[3,4-c]pyridine-3-carboxamide | |
| 41 | 3-(2-((2-((3-chloro-2-fluorobenzyl)amino)-2- | B |
| | oxoethyl)(cyclopropyl)amino)-2-oxoethyl)-1H-indole-1- | |
| | carboxamide | |
| 42 | 3-(2-((2-((3-chloro-2-fluorobenzyl)amino)-2- | B |
| | oxoethyl)(cyclopropyl)amino)-2-oxoethyl)imidazo[1,5- | |
| | a]pyridine-1-carboxamide | |
| 43 | 1-(2-((2-((3-chloro-2-fluorobenzyl)amino)-2- | D |
| | oxoethyl)(cyclopropyl)amino)-2-oxoethyl)-5- | |
| | (trifluoromethyl)-1H-pyrazole-3-carboxamide | |
| 44 | 3-carbamoyl-1-(2-((2-((3-chloro-2-fluorobenzyl)amino)-2- | C |
| | oxoethyl)(cyclopropyl)amino)-2-oxoethyl)-1H-pyrazole-5- | |
| | carboxylic acid | |
| 45 | 1-(2-((2-((3-chloro-2-fluorobenzyl)amino)-2- | C |
| | oxoethyl)(cyclopropyl)amino)-2-oxoethyl)-1H-pyrazole- | |
| | 3,5-dicarboxamide | |
| 46 | 4-bromo-1-(2-((2-((3-chloro-2-fluorobenzyl)amino)-2- | C |
| | oxoethyl)(cyclopropyl)amino)-2-oxoethyl)-1H-pyrazole-3- | |
| | carboxamide | |
| 47 | 2-(3-acetyl-1H-indazol-1-yl)-N-(2-((3-chloro-2- | A |
| | fluorobenzyl)amino)-2-oxoethyl)-N-cyclopropylacetamide | |
| 48 | 2-(1-acetylimidazo[1,5-a]pyridin-3-yl)-N-(2-((3-chloro-2- | B |
| | fluorobenzyl)amino)-2-oxoethyl)-N-cyclopropylacetamide | |
| 49 | N-(2-((3-chloro-2-fluorobenzyl)amino)-2-oxoethyl)-N- | C |
| | cyclopropyl-2-(3-(1-hydroxyethyl)-1H-indazol-1- | |
| | yl)acetamide | |
| 50 | 3-(3-(2-((3-chloro-2-fluorobenzyl)amino)-2-oxoethyl)-3- | B |
| | isopropylureido)-1H-indole-1-carboxamide | |
| 51 | 3-(3-(2-((3-chloro-2-fluorobenzyl)amino)-2-oxoethyl)-3- | B |
| | cyclopropylureido)-1H-indole-1-carboxamide | |
| 52 | 3-(3-(2-((3-chloro-2-fluorobenzyl)amino)-2-oxoethyl)-3- | B |
| | (pyrrolidin-3-yl)ureido)-1H-indole-1-carboxamide | |
| 53 | 1-(3-(2-((3-chloro-2-fluorobenzyl)amino)-2-oxoethyl)-3- | B |
| | cyclopropylureido)imidazo[1,5-a]pyridine-3-carboxamide | |
| 54 | 1-(2-((2-((3-chloro-2-fluorobenzyl)amino)-2-oxoethyl)(3- | B |
| | methylbutan-2-yl)amino)-2-oxoethyl)-1H-indazole-3- | |
| | carboxamide | |
| 55 | 1-(2-((2-((3-chloro-2-fluorobenzyl)amino)-2-oxoethyl)(1- | B |
| | cyclopropylethyl)amino)-2-oxoethyl)-1H-indazole-3- | |
| | carboxamide | |
| 56 | 1-(2-(sec-butyl(2-((3-chloro-2-fluorobenzyl)amino)-2- | B |
| | oxoethyl)amino)-2-oxoethyl)-1H-indazole-3-carboxamide | |
| 57 | 1-(2-((2-((3-chloro-2-fluorobenzyl)amino)-2-oxoethyl)(4- | B |
| | hydroxybutan-2-yl)amino)-2-oxoethyl)-1H-indazole-3- | |
| | carboxamide | |
| 58 | 1-(2-((2-((3-chloro-2-fluorobenzyl)amino)-2-oxoethyl)(1- | B |
| | hydroxypropan-2-yl)amino)-2-oxoethyl)-1H-indazole-3- | |
| | carboxamide | |
| 59 | methyl 2-(2-(3-carbamoyl-1H-indazol-1-yl)-N-(2-((3- | C |
| | chloro-2-fluorobenzyl)amino)-2- | |
| | oxoethyl)acetamido)propanoate | |
| 60 | 1-(2-((2-((3-chloro-2-fluorobenzyl)amino)-2-oxoethyl)(4- | B |
| | fluorobutan-2-yl)amino)-2-oxoethyl)-1H-indazole-3- | |
| | carboxamide | |
| 61 | 1-(2-((2-((3-chloro-2-fluorobenzyl)amino)-2-oxoethyl)(1- | C |
| | methoxybutan-2-yl)amino)-2-oxoethyl)-1H-indazole-3- | |
| | carboxamide | |
| 62 | 1-(2-((1-amino-1-oxopropan-2-yl)(2-((3-chloro-2- | C |
| | fluorobenzyl)amino)-2-oxoethyl)amino)-2-oxoethyl)-1H- | |
| | indazole-3-carboxamide | |
| 63 | 1-(2-((2-((3-chloro-2-fluorobenzyl)amino)-2-oxoethyl)(2- | B |
| | methylcyclopropyl)amino)-2-oxoethyl)-1H-indazole-3- | |
| | carboxamide | |
| 64 | 1-(2-((2-((3-chloro-2-fluorobenzyl)amino)-2- | B |
| | oxoethyl)(3,3-dimethylbutan-2-yl)amino)-2-oxoethyl)-1H- | |
| | indazole-3-carboxamide | |
| 65 | 1-(2-((2-((3-chloro-2-fluorobenzyl)amino)-2-oxoethyl)(1- | B |
| | phenylethyl)amino)-2-oxoethyl)-1H-indazole-3- | |
| | carboxamide | |
| 66 | 1-(2-((2-((3-chloro-2-fluorobenzyl)amino)-2-oxoethyl)(1- | B |
| | fluoropropan-2-yl)amino)-2-oxoethyl)-1H-indazole-3- | |
| | carboxamide | |
| 67 | 1-(2-((2-((3-chloro-2-fluorobenzyl)amino)-2- | B |
| | oxoethyl)(1,1,1-trifluoropropan-2-yl)amino)-2-oxoethyl)- | |
| | 1H-indazole-3-carboxamide | |
| 68 | 1-(2-((2-((3-chloro-2-fluorobenzyl)amino)-2-oxoethyl)(1- | B |
| | methoxypropan-2-yl)amino)-2-oxoethyl)-1H-indazole-3- | |
| | carboxamide | |
| 69 | 1-(2-((2-((3-chloro-2-fluorobenzyl)amino)-2-oxoethyl)(1- | C |
| | cyclopropyl-2-hydroxyethyl)amino)-2-oxoethyl)-1H- | |
| | indazole-3-carboxamide | |
| 70 | 1-(2-((2-((2-chloro-3-fluorobenzyl)amino)-2- | C |
| | oxoethyl)(1,3-difluoropropan-2-yl)amino)-2-oxoethyl)-1H- | |
| | indazole-3-carboxamide | |
| 71 | 1-(2-((4-aminobutan-2-yl)(2-((2-chloro-3- | B |
| | fluorobenzyl)amino)-2-oxoethyl)amino)-2-oxoethyl)-1H- | |
| | indazole-3-carboxamide | |
| 72 | ethyl 3-(2-(3-carbamoyl-1H-indazol-1-yl)-N-(2-((3-chloro- | B |
| | 2-fluorobenzyl)amino)-2-oxoethyl)acetamido)butanoate | |
| 73 | 1-(2-((2-((3-chloro-2-fluorobenzyl)amino)-2-oxoethyl)(1- | C |
| | cyclopropyl-3-hydroxypropyl)amino)-2-oxoethyl)-1H- | |
| | indazole-3-carboxamide | |
| 74 | 3-(2-(3-carbamoyl-1H-indazol-1-yl)-N-(2-((3-chloro-2- | C |
| | fluorobenzyl)amino)-2-oxoethyl)acetamido)butanoic acid | |
| 75 | 1-(2-((3-amino-1-cyclopropyl-3-oxopropyl)(2-((3-chloro- | C |
| | 2-fluorobenzyl)amino)-2-oxoethyl)amino)-2-oxoethyl)- | |
| | 1H-indazole-3-carboxamide | |
| 76 | 1-(2-((4-amino-4-oxobutan-2-yl)(2-((3-chloro-2- | B |
| | fluorobenzyl)amino)-2-oxoethyl)amino)-2-oxoethyl)-1H- | |
| | indazole-3-carboxamide | |
| 77 | ethyl 3-(2-(3-carbamoyl-1H-indazol-1-yl)-N-(2-((3-chloro- | C |
| | 2-fluorobenzyl)amino)-2-oxoethyl)acetamido)-4,4,4- | |
| | trifluorobutanoate | |
| 78 | 1-(2-((2-((3-chloro-2-fluorobenzyl)amino)-2- | B |
| | oxoethyl)(cyclopropyl)amino)-2-oxoethyl)-5-fluoro-1H- | |
| | indazole-3-carboxamide | |
| 79 | 3-(2-(3-carbamoyl-1H-indazol-1-yl)-N-(2-((3-chloro-2- | C |
| | fluorobenzyl)amino)-2-oxoethyl)acetamido)-4,4,4- | |
| | trifluorobutanoic acid | |
| 80 | 5-chloro-1-(2-((2-((3-chloro-2-fluorobenzyl)amino)-2- | B |
| | oxoethyl)(cyclopropyl)amino)-2-oxoethyl)-1H-indazole-3- | |
| | carboxamide | |
| 81 | 7-chloro-1-(2-((2-((3-chloro-2-fluorobenzyl)amino)-2- | A |
| | oxoethyl)(cyclopropyl)amino)-2-oxoethyl)-1H-indazole-3- | |
| | carboxamide | |
| 82 | 1-(2-((2-((3-chloro-2,6-difluorobenzyl)amino)-2- | B |
| | oxoethyl)(cyclopropyl)amino)-2-oxoethyl)-1H-indazole-3- | |
| | carboxamide | |
| 83 | 1-(2-((2-((3-chloro-2,4-difluorobenzyl)amino)-2- | B |
| | oxoethyl)(cyclopropyl)amino)-2-oxoethyl)-1H-indazole-3- | |
| | carboxamide | |
| 84 | 1-(2-((2-((3-chloro-2-fluorobenzyl)amino)-2- | B |
| | oxoethyl)(cyclopropyl)amino)-2-oxoethyl)-6-fluoro-1H- | |
| | indazole-3-carboxamide | |
| 85 | 6-chloro-1-(2-((2-((3-chloro-2-fluorobenzyl)amino)-2- | B |
| | oxoethyl)(cyclopropyl)amino)-2-oxoethyl)-1H-indazole-3- | |
| | carboxamide | |
| 86 | 1-(2-((2-((3-chloro-2-fluorobenzyl)amino)-2- | B |
| | oxoethyl)(cyclopropyl)amino)-2-oxoethyl)-6-cyano-1H- | |
| | indazole-3-carboxamide | |
| 87 | 1-(2-((2-((3-chloro-6-cyano-2-fluorobenzyl)amino)-2- | B |
| | oxoethyl)(cyclopropyl)amino)-2-oxoethyl)-1H-indazole-3- | |
| | carboxamide | |
| 88 | 1-(2-((2-((3-chloro-2-fluorobenzyl)amino)-2- | B |
| | oxoethyl)(cyclopropyl)amino)-2-oxoethyl)-7-fluoro-1H- | |
| | indazole-3-carboxamide | |
| 89 | 6-(aminomethyl)-1-(2-((2-((3-chloro-2- | C |
| | fluorobenzyl)amino)-2-oxoethyl)(cyclopropyl)amino)-2- | |
| | oxoethyl)-1H-indazole-3-carboxamide | |
| 90 | 1-(2-((2-((3-chloro-5-cyano-2-fluorobenzyl)amino)-2- | C |
| | oxoethyl)(cyclopropyl)amino)-2-oxoethyl)-1H-indazole-3- | |
| | carboxamide | |
| 91 | 1-(2-((2-(((6-chloro-1H-indazol-4-yl)methyl)amino)-2- | B |
| | oxoethyl)(cyclopropyl)amino)-2-oxoethyl)-1H-indazole-3- | |
| | carboxamide | |
| 92 | 1-(2-((2-((3-chloro-2-fluorobenzyl)amino)-2- | B |
| | oxoethyl)(cyclopropyl)amino)-2-oxoethyl)-5-cyano-1H- | |
| | indazole-3-carboxamide | |
| 93 | 1-(2-((2-((3-chloro-2-fluorobenzyl)amino)-2- | B |
| | oxoethyl)(cyclopropyl)amino)-2-oxoethyl)-1H-indazole- | |
| | 3,5-dicarboxamide | |
| 94 | 1-(2-((2-((3-chloro-2-fluorobenzyl)amino)-2- | B |
| | oxoethyl)(cyclopropyl)amino)-2-oxoethyl)-6- | |
| | (trifluoromethyl)-1H-indazole-3-carboxamide | |
| 95 | 1-(2-((2-((3-chloro-2-fluorobenzyl)amino)-2- | C |
| | oxoethyl)(cyclopropyl)amino)-2-oxoethyl)-5- | |
| | (trifluoromethyl)-1H-indazole-3-carboxamide | |
| 96 | 1-(2-((2-((3-chloro-4-cyano-2-fluorobenzyl)amino)-2- | C |
| | oxoethyl)(cyclopropyl)amino)-2-oxoethyl)-1H-indazole-3- | |
| | carboxamide | |
| 97 | 1-(2-((2-((3-chloro-2-fluorobenzyl)amino)-2- | C |
| | oxoethyl)(cyclopropyl)amino)-2-oxoethyl)-4-fluoro-1H- | |
| | indazole-3-carboxamide | |
| 98 | 2-(3-(1H-imidazol-2-yl)-1H-indazol-1-yl)-N-(2-((3-chloro- | C |
| | 2-fluorobenzyl)amino)-2-oxoethyl)-N- | |
| | cyclopropylacetamide | |
| 99 | 1-(2-((2-((3-chloro-2-fluoro-4- | D |
| | (hydroxymethyl)benzyl)amino)-2- | |
| | oxoethyl)(cyclopropyl)amino)-2-oxoethyl)-1H-indazole-3- | |
| | carboxamide | |
| 100 | 1-(2-((2-((3-chloro-2-fluorobenzyl)amino)-2- | A |
| | oxoethyl)(cyclopropyl)amino)-2-oxoethyl)-1H-indazole- | |
| | 3,6-dicarboxamide | |
| 101 | 1-(2-((2-((5-(aminomethyl)-3-chloro-2- | C |
| | fluorobenzyl)amino)-2-oxoethyl)(cyclopropyl)amino)-2- | |
| | oxoethyl)-1H-indazole-3-carboxamide | |
| 102 | 1-(2-((2-(((4-chloro-1H-indazol-6-yl)methyl)amino)-2- | C |
| | oxoethyl)(cyclopropyl)amino)-2-oxoethyl)-1H-indazole-3- | |
| | carboxamide | |
| 103 | 1-(2-((2-((3-chloro-5-fluorobenzyl)amino)-2- | B |
| | oxoethyl)(cyclopropyl)amino)-2-oxoethyl)-1H-indazole-3- | |
| | carboxamide | |
| 104 | 1-(2-(cyclopropyl(2-((3,4-dichlorobenzyl)amino)-2- | C |
| | oxoethyl)amino)-2-oxoethyl)-1H-indazole-3-carboxamide | |
| 105 | 1-(2-((2-((3-chloro-2-fluorobenzyl)amino)-2- | C |
| | oxoethyl)(cyclopropyl)amino)-2-oxoethyl)-7-cyano-1H- | |
| | indazole-3-carboxamide | |
| 106 | 1-(2-(cyclopropyl(2-((2,3-dichlorobenzyl)amino)-2- | B |
| | oxoethyl)amino)-2-oxoethyl)-1H-indazole-3-carboxamide | |
| 107 | 1-(2-((2-(((6-chloropyridin-2-yl)methyl)amino)-2- | C |
| | oxoethyl)(cyclopropyl)amino)-2-oxoethyl)-1H-indazole-3- | |
| | carboxamide | |
| 108 | 1-(2-((2-((3-chloro-4-fluorobenzyl)amino)-2- | C |
| | oxoethyl)(cyclopropyl)amino)-2-oxoethyl)-1H-indazole-3- | |
| | carboxamide | |
| 109 | 1-(2-((2-(((5-chloro-1H-indazol-7-yl)methyl)amino)-2- | C |
| | oxoethyl)(cyclopropyl)amino)-2-oxoethyl)-1H-indazole-3- | |
| | carboxamide | |
| 110 | 1-(2-((2-(((5-chlorobenzofuran-7-yl)methyl)amino)-2- | B |
| | oxoethyl)(cyclopropyl)amino)-2-oxoethyl)-1H-indazole-3- | |
| | carboxamide | |
| 111 | 1-(2-((2-((3-chloro-2-fluorobenzyl)amino)-2- | A |
| | oxoethyl)(cyclopropyl)amino)-2-oxoethyl)-7- | |
| | (trifluoromethyl)-1H-indazole-3-carboxamide | |
| 112 | 1-(2-((2-((3-chlorobenzyl)amino)-2- | B |
| | oxoethyl)(cyclopropyl)amino)-2-oxoethyl)-1H-indazole-3- | |
| | carboxamide | |
| 113 | 1-(2-((2-((3-chloro-4-methylbenzyl)amino)-2- | C |
| | oxoethyl)(cyclopropyl)amino)-2-oxoethyl)-1H-indazole-3- | |
| | carboxamide | |
| 114 | 1-(2-((2-(((7-chloro-1H-indazol-5-yl)methyl)amino)-2- | C |
| | oxoethyl)(cyclopropyl)amino)-2-oxoethyl)-1H-indazole-3- | |
| | carboxamide | |
| 115 | 1-(2-(cyclopropyl(2-((2-fluorobenzyl)amino)-2- | C |
| | oxoethyl)amino)-2-oxoethyl)-1H-indazole-3-carboxamide | |
| 116 | 1-(2-((2-((3-chloro-2-methoxybenzyl)amino)-2- | C |
| | oxoethyl)(cyclopropyl)amino)-2-oxoethyl)-1H-indazole-3- | |
| | carboxamide | |
| 117 | 1-(2-((2-((4-carbamoyl-2-fluorobenzyl)amino)-2- | C |
| | oxoethyl)(cyclopropyl)amino)-2-oxoethyl)-1H-indazole-3- | |
| | carboxamide | |
| 118 | 1-(2-((2-((3-amino-2-fluorobenzyl)amino)-2- | C |
| | oxoethyl)(cyclopropyl)amino)-2-oxoethyl)-1H-indazole-3- | |
| | carboxamide | |
| 119 | 1-(2-(cyclopropyl(2-((3,5-dichlorobenzyl)amino)-2- | B |
| | oxoethyl)amino)-2-oxoethyl)-1H-indazole-3-carboxamide | |
| 120 | 1-(2-((2-((3-bromo-2-fluorobenzyl)amino)-2- | A |
| | oxoethyl)(cyclopropyl)amino)-2-oxoethyl)-1H-indazole-3- | |
| | carboxamide | |
| 121 | 1-(2-((2-((4-amino-2-fluorobenzyl)amino)-2- | C |
| | oxoethyl)(cyclopropyl)amino)-2-oxoethyl)-1H-indazole-3- | |
| | carboxamide | |
| 122 | 1-(2-(cyclopropyl(2-((2-fluoro-3-methoxybenzyl)amino)- | C |
| | 2-oxoethyl)amino)-2-oxoethyl)-1H-indazole-3- | |
| | carboxamide | |
| 123 | 1-(2-((2-((3-cyano-2-fluorobenzyl)amino)-2- | C |
| | oxoethyl)(cyclopropyl)amino)-2-oxoethyl)-1H-indazole-3- | |
| | carboxamide | |
| 124 | 1-(2-((2-((3-chloro-5-(trifluoromethyl)benzyl)amino)-2- | C |
| | oxoethyl)(cyclopropyl)amino)-2-oxoethyl)-1H-indazole-3- | |
| | carboxamide | |
| 125 | 1-(2-((2-((3-chloro-4-methoxybenzyl)amino)-2- | C |
| | oxoethyl)(cyclopropyl)amino)-2-oxoethyl)-1H-indazole-3- | |
| | carboxamide | |
| 126 | 1-(2-(cyclopropyl(2-((2-fluoro-3- | D |
| | (hydroxymethyl)benzyl)amino)-2-oxoethyl)amino)-2- | |
| | oxoethyl)-1H-indazole-3-carboxamide | |
| 127 | 1-(2-(cyclopropyl(2-((3-cyclopropyl-2- | C |
| | fluorobenzyl)amino)-2-oxoethyl)amino)-2-oxoethyl)-1H- | |
| | indazole-3-carboxamide | |
| 128 | 1-(2-(cyclopropyl(2-((2-fluoro-3-methylbenzyl)amino)-2- | B |
| | oxoethyl)amino)-2-oxoethyl)-1H-indazole-3-carboxamide | |
| 129 | 1-(2-((2-((3-chloro-5-methylbenzyl)amino)-2- | C |
| | oxoethyl)(cyclopropyl)amino)-2-oxoethyl)-1H-indazole-3- | |
| | carboxamide | |
| 130 | 1-(2-((2-(((5-chloropyridin-3-yl)methyl)amino)-2- | C |
| | oxoethyl)(cyclopropyl)amino)-2-oxoethyl)-1H-indazole-3- | |
| | carboxamide | |
| 131 | 1-(2-((2-((3-chloro-2-cyclopropylbenzyl)amino)-2- | D |
| | oxoethyl)(cyclopropyl)amino)-2-oxoethyl)-1H-indazole-3- | |
| | carboxamide | |
| 132 | 1-(2-(cyclopropyl(2-((2,3-difluorobenzyl)amino)-2- | B |
| | oxoethyl)amino)-2-oxoethyl)-1H-indazole-3-carboxamide | |
| 133 | 1-(2-((2-((3-chloro-5-cyanobenzyl)amino)-2- | C |
| | oxoethyl)(cyclopropyl)amino)-2-oxoethyl)-1H-indazole-3- | |
| | carboxamide | |
| 134 | 1-(2-((2-((3-chloro-2-methylbenzyl)amino)-2- | C |
| | oxoethyl)(cyclopropyl)amino)-2-oxoethyl)-1H-indazole-3- | |
| | carboxamide | |
| 135 | 1-(2-((2-((3-chloro-2-(trifluoromethyl)benzyl)amino)-2- | C |
| | oxoethyl)(cyclopropyl)amino)-2-oxoethyl)-1H-indazole-3- | |
| | carboxamide | |
| 136 | 1-(2-(cyclopropyl(2-((2-fluoro-3- | C |
| | (trifluoromethyl)benzyl)amino)-2-oxoethyl)amino)-2- | |
| | oxoethyl)-1H-indazole-3-carboxamide | |
| 137 | 2-(3-acetylimidazo[1,5-a]pyridin-1-yl)-N-(2-((3-chloro-2- | C |
| | fluorobenzyl)amino)-2-oxoethyl)-N-cyclopropylacetamide | |
| 138 | 1-(2-((2-((3-chloro-2-fluorobenzyl)amino)-2- | B |
| | oxoethyl)(cyclopropyl)amino)-2-oxoethyl)imidazo[1,5- | |
| | a]pyridine-3-carboxamide | |
| 139 | 2-(1-acetylimidazo[1,5-a]pyridin-3-yl)-N-(2-((3-chloro-2- | B |
| | fluorobenzyl)amino)-2-oxoethyl)-N-cyclopropylacetamide | |
| 140 | 3-(3-(2-((3-chloro-2-fluorobenzyl)amino)-2-oxoethyl)-3- | B |
| | cyclopropylureido)imidazo[1,5-a]pyridine-1-carboxamide | |
| 141 | 2-(3-(3-acetylimidazo[1,5-a]pyridin-1-yl)-1- | B |
| | cyclopropylureido)-N-(3-chloro-2-fluorobenzyl)acetamide | |

Note:
Biochemical assay IC₅₀ data are designated within the following ranges:
A: ≤0.10 μM
B: >0.10 μM to ≤1.0 μM
C: >1.0 μM to ≤10 μM
D: >10 μM

### Example 2: AP Hemolysis Inhibition Assay

The ability of the compounds disclosed herein to inhibit alternative pathway (AP) hemolytic activity was determined. Red blood cells (RBC), chicken or rabbit erythrocyctes (SbjBio), were washed three time using assay buffer containing 0.1% gelatin, 5 mM Veronal, 145 mM NaCl, 0.025% NaN₃, 10 mM Mg-EGTA pH 7.3. In 100 μL reaction system, 1300 to 1500 ng/L final concentration of Normal Human Serum (CompTech) was incubated with compound for 15 min at 37° C. Then 2×10⁶ cells/well of chicken or rabbit erythrocytes in assay buffer were added and incubated for an additional 60 min at 37° C. Positive control (100% lysis) consists of serum and RBC, and negative control (0% lysis) consists of assay buffer and RBC only. Samples were centrifuged at 2000 g for 5 min, and supernatants collected. Optical density of the supernatant is monitored at 414 nm using Synergy 2 (BioTek). Percentage lysis in each sample is calculated relative to positive control (100% lysis).

Table 5 discloses the inhibitory activity in the hemolysis assay of certain compounds provided herein.

TABLE 5

| Ex. | Name | EC₅₀ |
|---|---|---|
| 4 | 1-(2-((2-((3-chloro-2-fluorobenzyl)amino)-2- | B |
| | oxoethyl)(cyclopropyl)amino)-2-oxoethyl)-1H-indazole-3- | |
| | carboxamide | |
| 5 | 1-(2-((2-((3-chloro-2-fluorobenzyl)amino)-2- | B |
| | oxoethyl)(isopropyl)amino)-2-oxoethyl)-1H-indazole-3- | |
| | carboxamide | |
| 6 | 1-(2-((2-((3-chloro-2-fluorobenzyl)amino)-2- | B |
| | oxoethyl)(ethyl)amino)-2-oxoethyl)-1H-indazole-3- | |
| | carboxamide | |
| 8 | 3-(2-((2-((3-chloro-2-fluorobenzyl)amino)-2- | B |
| | oxoethyl)(cyclopropyl)amino)-2-oxoethyl)-1H-indazole-1- | |
| | carboxamide | |
| 12 | 1-(2-((2-((6-chloropyridin-2-yl)amino)-2- | B |
| | oxoethyl)(isopropyl)amino)-2-oxoethyl)-1H-indazole-3- | |
| | carboxamide | |
| 16 | 1-(2-((2-((6-chloropyridin-2-yl)amino)-2- | B |
| | oxoethyl)(cyclopropyl)amino)-2-oxoethyl)-1H-indazole-3- | |
| | carboxamide | |
| 20 | 3-(2-((2-((6-chloropyridin-2-yl)amino)-2- | B |
| | oxoethyl)(cyclobutyl)amino)-2-oxoethyl)-1H-indazole-1- | |
| | carboxamide | |
| 28 | 1-(2-((2-((3-chloro-2-fluorobenzyl)amino)-2- | B |
| | oxoethyl)(cyclobutyl)amino)-2-oxoethyl)-1H-indazole-3- | |
| | carboxamide | |
| 30 | 1-(2-((2-((3-chloro-2-fluorobenzyl)amino)-2- | B |
| | oxoethyl)(cyclopentyl)amino)-2-oxoethyl)-1H-indazole-3- | |
| | carboxamide | |
| 33 | 1-(2-((2-((3-chloro-2-fluorobenzyl)amino)-2- | B |
| | oxoethyl)(methyl)amino)-2-oxoethyl)-1H-indazole-3- | |
| | carboxamide | |
| 40 | 1-(2-((2-((3-chloro-2-fluorobenzyl)amino)-2- | B |
| | oxoethyl)(cyclopropyl)amino)-2-oxoethyl)-1H-pyrazolo[3,4- | |
| | c]pyridine-3-carboxamide trifluoroacetate salt | |
| 47 | 2-(3-acetyl-1H-indazol-1-yl)-N-(2-((3-chloro-2- | B |
| | fluorobenzyl)amino)-2-oxoethyl)-N-cyclopropylacetamide | |
| 50 | 3-(3-(2-((3-chloro-2-fluorobenzyl)amino)-2-oxoethyl)-3- | C |
| | isopropylureido)-1H-indole-1-carboxamide | |
| 53 | 1-(3-(2-((3-chloro-2-fluorobenzyl)amino)-2-oxoethyl)-3- | B |
| | cyclopropylureido)imidazo[1,5-a]pyridine-3-carboxamide | |

Note:
Hemolysis assay EC₅₀ data are designated within the following ranges:
A: ≤0.10 μM
B: >0.10 μM to ≤1.0 μM
C: >1.0 μM to ≤10 μM
D: >10 μM

### III. Preparation of Pharmaceutical Dosage Forms

### Example 1: Oral Tablet

A tablet is prepared by mixing 48% by weigh of a compound of Formula (I) or a pharmaceutically acceptable salt thereof, 45% by weight of microcrystalline cellulose, 5% by weight of low-substituted hydroxypropyl cellulose, and 2% by weight of magnesium stearate. Tablets are prepared by direct compression. The total weight of the compressed tablets is maintained at 250-500 mg.

We claim:

1. A compound, or a pharmaceutically acceptable salt thereof, having the structure of Formula (I): 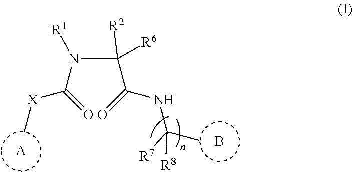
wherein
Ring A is an optionally substituted heteroaryl ring;
Ring B is an optionally substituted aryl, optionally substituted heteroaryl, optionally substituted 3-, 4-, 5-, or 6-membered cycloalkyl, or optionally substituted 3-, 4-, 5-, or 6-membered heterocyclyl;
X is —CR⁹R¹⁰— or —NR¹¹—;
R¹ is optionally substituted alkyl, optionally substituted 3-, 4-, 5-, or 6-membered cycloalkyl, optionally substituted carbocyclylalkyl, optionally substituted 3-, 4-, 5-, or 6-membered heterocyclyl, optionally substituted aryl, optionally substituted aralkyl, optionally substituted heteroaryl, optionally substituted heteroarylalkyl, or optionally substituted dicyclopropylmethyl;
R², R⁶, R⁹, R¹⁰, each R⁷, or each R⁸, is independently selected from hydrogen, halo, hydroxy, amino, —CO₂H, —S(O)—R²⁰, —S—R²⁰, —S(O)₂—R²⁰, optionally substituted alkoxy, optionally substituted aryloxy, optionally substituted heteroaryloxy, optionally substituted (heterocyclyl)-O—, optionally substituted alkyl, optionally substituted cycloalkyl, optionally substituted alkenyl, optionally substituted aryl, optionally substituted heteroaryl, optionally substituted heterocyclyl, optionally substituted alkylamino, optionally substituted dialkylamino, —CO—R²⁰, —CO₂—R²⁰, —CON(R²¹)₂, —SO₂N(R²¹)₂, —C(═NR²²)—N(R²¹)₂, optionally substituted alkynyl, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂;
each R²⁰ is independently optionally substituted alkyl, optionally substituted cycloalkyl, optionally substituted aryl, or optionally substituted heteroaryl;
each R²¹ is independently hydrogen, optionally substituted alkyl, optionally substituted cycloalkyl, optionally substituted aryl, or optionally substituted heteroaryl;
each R²² is independently hydrogen, optionally substituted alkyl, or optionally substituted cycloalkyl;
R¹¹ is hydrogen, or optionally substituted alkyl; and
n is 0, 1, 2, or 3.

2. The compound of claim 1, or a pharmaceutically acceptable salt thereof, wherein Ring A is 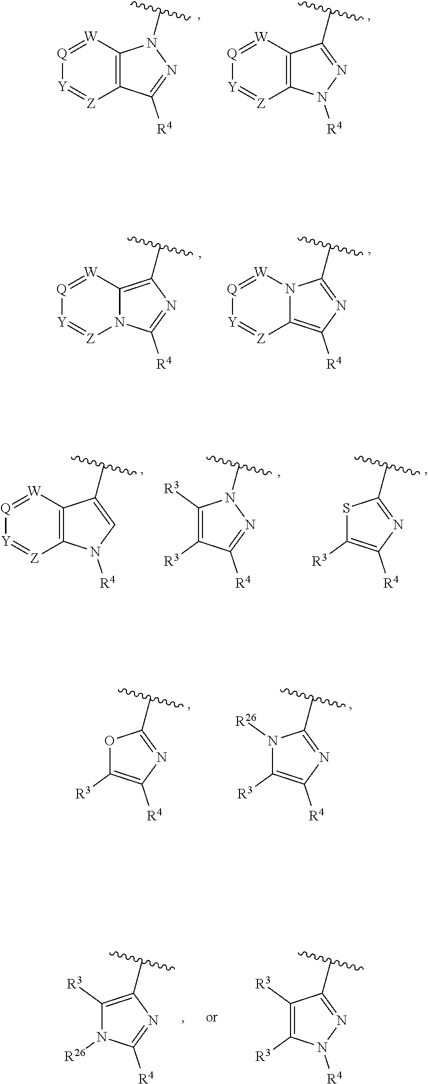
wherein
W, Q, Y, and Z are each independently selected from N or C—R³;
each R³ is independently selected from hydrogen, cyano, halo, hydroxy, azido, amino, nitro, —CO₂H, —S(O)—R²³, —S—R²³, —S(O)₂—R²³, optionally substituted alkoxy, optionally substituted aryloxy, optionally substituted heteroaryloxy, optionally substituted (heterocyclyl)-O—, optionally substituted alkyl, optionally substituted alkynyl, optionally substituted carbocyclyl, optionally substituted alkenyl, optionally substituted aryl, optionally substituted heteroaryl, optionally substituted heterocyclyl, optionally substituted aralkoxy, optionally substituted heteroarylalkoxy, optionally substituted (carbocyclyl)-O—, optionally substituted heterocyclylalkoxy, —N(R²⁴), —CO—R²³, —CO₂—R²³, CO(R⁴)₂, —NR²⁴CO—R²³, —NR²⁴CO₂—R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, —O—CO₂—R²³;
each R²³ is independently optionally substituted alkyl, optionally substituted cycloalkyl, optionally substituted aryl, or optionally substituted heteroaryl;
each R²⁴ is independently hydrogen, optionally substituted alkyl, optionally substituted cycloalkyl, optionally substituted aryl, or optionally substituted heteroaryl;
each R²⁵ is independently hydrogen, optionally substituted alkyl, or optionally substituted cycloalkyl;
each R²⁶ is independently hydrogen or optionally substituted alkyl;
R⁴ is optionally substituted alkyl, —C(═O)R⁵, or heteroaryl;
R⁵ is selected from NH₂, optionally substituted alkylamino, optionally substituted dialkylamino, optionally substituted alkyl, or optionally substituted cycloalkyl.

3. The compound of claim 2, or a pharmaceutically acceptable salt thereof, wherein Ring A is

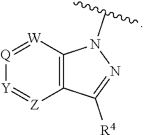

4. The compound of claim 2 or 3, or a pharmaceutically acceptable salt thereof, wherein W, Q, Y, and Z are C—R³.

5. The compound of claim 2 or 3, or a pharmaceutically acceptable salt thereof, wherein W, Q, Y, and Z are C—R³ and each R³ is independently selected from hydrogen, CO₂H, —S(O)—R²³, —S—R²³, —S(O)₂—R²³, —N(R²⁴), —CO—R²³, —CO₂—R²³, —CO(NR²⁴)₂, —NR²⁴CO—R²³, —NR²⁴CO₂—R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, or —O—CO₂—R²³.

6. The compound of claim 2 or 3, or a pharmaceutically acceptable salt thereof, wherein W, Q, Y, and Z are C—R³ and each R³ is independently selected from hydrogen, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

7. The compound of claim 2 or 3, or a pharmaceutically acceptable salt thereof, wherein W, Q, Y, and Z are C—R³ and each R³ is independently selected from hydrogen, halogen, optionally substituted alkyl, optionally substituted cycloalkyl, or optionally substituted alkoxy.

8. The compound of claim 2 or 3, or a pharmaceutically acceptable salt thereof, wherein W, Q, Y, and Z are C—R³ and each R³ is hydrogen.

9. The compound of claim 2 or 3, or a pharmaceutically acceptable salt thereof, wherein Q is N; W, Y, and Z are C—R³.

10. The compound of claim 2 or 3, or a pharmaceutically acceptable salt thereof, wherein Q is N; W, Y, and Z are C—R³; and each R³ is independently selected from hydrogen, CO₂H, —S(O)—R²³, —S—R²³, —S(O)₂—R²³, —N(R²⁴), —CO—R²³, —CO₂—R²³, —CO(NR²⁴)₂, —NR²⁴CO—R²³, —NR²⁴CO₂R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, or —O—CO₂—R²³.

11. The compound of claim 2 or 3, or a pharmaceutically acceptable salt thereof, wherein Q is N; W, Y, and Z are C—R³; and each R³ is independently selected from hydrogen, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

12. The compound of claim 2 or 3, or a pharmaceutically acceptable salt thereof, wherein Q is N; W, Y, and Z are C—R³; and each R³ is independently selected from hydrogen, halogen, optionally substituted alkyl, optionally substituted cycloalkyl, or optionally substituted alkoxy.

13. The compound of claim 2 or 3, or a pharmaceutically acceptable salt thereof, wherein Q is N; W, Y, and Z are C—R³; and each R³ is hydrogen.

14. The compound of claim 2 or 3, or a pharmaceutically acceptable salt thereof, wherein W is N; Q, Y, and Z are C—R³.

15. The compound of claim 2 or 3, or a pharmaceutically acceptable salt thereof, wherein W is N; Q, Y, and Z are C—R³; and each R³ is independently selected from hydrogen, CO₂H, —S(O)—R²³, —S—R²³, —S(O)₂—R²³, —N(R²⁴), —CO—R²³, —CO₂—R²³, —CO(NR²⁴)₂, —NR²⁴CO—R²³, —NR²⁴CO₂—R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂—NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, or —O—CO₂—R²³.

16. The compound of claim 2 or 3, or a pharmaceutically acceptable salt thereof, wherein W is N; Q, Y, and Z are C—R³; and each R³ is independently selected from hydrogen, —N(R²⁴), —NR²⁴CO—R²³, —NR²⁴CO₂—R²³, —NR²⁴CO₂—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

17. The compound of claim 2 or 3, or a pharmaceutically acceptable salt thereof, wherein W is N; Q, Y, and Z are C—R³; and each R³ is independently selected from hydrogen, halogen, optionally substituted alkyl, optionally substituted cycloalkyl, or optionally substituted alkoxy.

18. The compound of claim 2 or 3, or a pharmaceutically acceptable salt thereof, wherein W is N; Q, Y, and Z are C—R³; and each R³ is hydrogen.

19. The compound of claim 2 or 3, or a pharmaceutically acceptable salt thereof, wherein Y is N; W, Q, and Z are C—R³.

20. The compound of claim 2 or 3, or a pharmaceutically acceptable salt thereof, wherein Y is N; W, Q, and Z are C—R³; and each R³ is independently selected from hydrogen, CO₂H, —S(O)—R²³, —S—R²³, —S(O)₂—R²³, —N(R²⁴), —CO—R²³, —CO₂—R²³, —CO(NR²⁴)₂, —NR²⁴CO—R²³, —NR²⁴CO₂R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, or —O—CO₂—R²³.

21. The compound of claim 2 or 3, or a pharmaceutically acceptable salt thereof, wherein Y is N; W, Q, and Z are C—R³; and each R³ is independently selected from hydrogen, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

22. The compound of claim 2 or 3, or a pharmaceutically acceptable salt thereof, wherein Y is N; W, Q, and Z are C—R³; and each R³ is independently selected from hydrogen, halogen, optionally substituted alkyl, optionally substituted cycloalkyl, or optionally substituted alkoxy.

23. The compound of claim 2 or 3, or a pharmaceutically acceptable salt thereof, wherein Y is N; W, Q, and Z are C—R³; and each R³ is hydrogen.

24. The compound of claim 2 or 3, or a pharmaceutically acceptable salt thereof, wherein Z is N; W, Q, and Y are C—R³.

25. The compound of claim 2 or 3, or a pharmaceutically acceptable salt thereof, wherein Z is N; W, Q, and Y are C—R³; and each R³ is independently selected from hydrogen, CO₂H, —S(O)—R²³, —S—R²³, —S(O)₂—R²³, —N(R²⁴), —CO—R²³, —CO₂—R²³, —CO(NR²⁴)₂, —NR²⁴CO—R²³, —NR²⁴CO₂—R²³, —SO₂(NR²⁴)₂, —C(═NR²⁵)—(NR²⁴)₂, —NR²⁴CO—R²³, —NR²⁴CO₂—R²³, —NR²⁴CO—N(R²³)₂, —NR²⁴SO₂—N(R²³)₂—NR²⁴CO—R²³, —NR²⁴CO₂—R²³, —NR²⁴CO—N(R²⁴)₂, —NR²⁴SO₂—N(R²⁴)₂, —O—CO—R²³, —O—CO—R²³, —O—CO₂—R²³, or —O—CO₂—R²³.

26. The compound of claim 2 or 3, or a pharmaceutically acceptable salt thereof, wherein Z is N; W, Q, and Y are C—R³; and each R³ is independently selected from hydrogen, —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

27. The compound of claim 2 or 3, or a pharmaceutically acceptable salt thereof, wherein Z is N; W, Q, and Y are C—R³; and each R³ is independently selected from hydrogen, halogen, optionally substituted alkyl, optionally substituted cycloalkyl, or optionally substituted alkoxy.

28. The compound of claim 2 or 3, or a pharmaceutically acceptable salt thereof, wherein Z is N; W, Q, and Y are C—R³; and each R³ is hydrogen.

29. The compound of any one of claims 2-28, or a pharmaceutically acceptable salt thereof, wherein R⁴ is —CONH₂.

30. The compound of claim 2, or a pharmaceutically acceptable salt thereof, wherein Ring A is

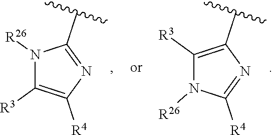

31. The compound of claim 2, or a pharmaceutically acceptable salt thereof, wherein Ring A is

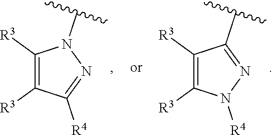

32. The compound of claim 2, or a pharmaceutically acceptable salt thereof, wherein Ring A is

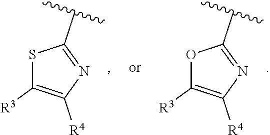

33. The compound of any one of claims 1-32, or a pharmaceutically acceptable salt thereof, wherein Ring B is optionally substituted aryl.

34. The compound of any one of claims 1-33, or a pharmaceutically acceptable salt thereof, wherein Ring B is optionally substituted phenyl.

35. The compound of any one of claims 1-32, or a pharmaceutically acceptable salt thereof, wherein Ring B is optionally substituted heteroaryl.

36. The compound of any one of claims 1-32, or a pharmaceutically acceptable salt thereof, wherein Ring B is optionally substituted pyridyl.

37. The compound of any one of claims 1-32, or a pharmaceutically acceptable salt thereof, wherein Ring B is optionally substituted 6-membered cycloalkyl.

38. The compound of any one of claims 1-37, or a pharmaceutically acceptable salt thereof, wherein Ring B is optionally substituted with at least one substituent selected from halogen, cyano, halo, hydroxy, azido, amino, nitro, —CO₂H, cycloalkyl, —CONH₂, —SO₂Me, or alkoxy.

39. The compound of any one of claims 1-38, or a pharmaceutically acceptable salt thereof, wherein Ring B is a phenyl substituted with at least one halogen and n is 1.

40. The compound of any one claims 1-38, or a pharmaceutically acceptable salt thereof, wherein n is 0.

41. The compound of any one claims 1-39, or a pharmaceutically acceptable salt thereof, wherein n is 1.

42. The compound of any one of claims 1-41, or a pharmaceutically acceptable salt thereof, wherein R¹ is optionally substituted alkyl.

43. The compound of any one of claims 1-41, or a pharmaceutically acceptable salt thereof, wherein R¹ is optionally substituted cycloalkyl.

44. The compound of any one of claims 1-41, or a pharmaceutically acceptable salt thereof, wherein R¹ is optionally substituted cyclopropyl.

45. The compound of any one of claims 1-41, or a pharmaceutically acceptable salt thereof, wherein R¹ is optionally substituted 3-, 4-, 5-, or 6-membered heterocyclyl, optionally substituted aryl, or optionally substituted heteroaryl.

46. The compound of any one of claims 1-41, or a pharmaceutically acceptable salt thereof, wherein R¹ is optionally substituted carbocyclylalkyl, optionally substituted aralkyl, or optionally substituted heteroarylalkyl.

47. The compound of any one of claims 1-46, or a pharmaceutically acceptable salt thereof, wherein R² is hydrogen.

48. The compound of any one of claims 1-46, or a pharmaceutically acceptable salt thereof, wherein R² is optionally substituted alkyl.

49. The compound of any one of claims 1-46, or a pharmaceutically acceptable salt thereof, wherein R² is —N(R²⁴), —NR²⁴CO₂—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

50. The compound of any one of claims 1-49, or a pharmaceutically acceptable salt thereof, wherein X is —CH₂—.

51. The compound of any one of claims 1-50, or a pharmaceutically acceptable salt thereof, wherein X is —NH—.

52. The compound of any one of claims 1-49, or a pharmaceutically acceptable salt thereof, wherein R⁹ or R¹⁰ is hydrogen or optionally substituted alkyl.

53. The compound of any one of claims 1-46, or a pharmaceutically acceptable salt thereof, wherein R⁹ or R¹⁰ is —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

54. The compound of any one of claims 1-49, or a pharmaceutically acceptable salt thereof, wherein R⁹ or R¹⁰ is hydrogen.

55. The compound of any one of claims 1-54, or a pharmaceutically acceptable salt thereof, wherein R⁶ is hydrogen.

56. The compound of any one of claims 1-46, or a pharmaceutically acceptable salt thereof, wherein R⁶ is —N(R²⁴), —NR²⁴CO₂—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂, —NR²⁴CO—R²³, —NR²⁴CO₂—R²³, —NR²⁴CO—N(R²⁴)₂, or —NR²⁴SO₂—N(R²⁴)₂.

57. The compound of any one of claims 1-56, or a pharmaceutically acceptable salt thereof, wherein R⁷ is hydrogen.

58. The compound of any one of claims 1-56, or a pharmaceutically acceptable salt thereof, wherein R⁷ and R⁸ are hydrogen.

59. The compound of any one of claims 1-46, or a pharmaceutically acceptable salt thereof, wherein R⁷ is —N(R²⁴), —NR²⁴CO—R²³, —NR²⁴CO₂—R²³, —NR²⁴CO—R²³, —NR²⁴CO₂—R²³, —NR²⁴CO—N(R²³)₂, —NR²⁴SO₂—N(R²³)₂—NR²⁴CO—R²³, —NR²⁴CO₂—R²³, —NR²⁴CO—N(R²⁴)₂, or —NR²⁴SO₂—N(R²⁴)₂.

60. The compound of any one of claims 1-59, or a pharmaceutically acceptable salt thereof, wherein Ring B is a pyridine substituted with at least one halogen and n is 0.

61. A compound, or pharmaceutically acceptable salt thereof, chosen from:
(S)-1-(2-((2-((3-chloro-2-fluorobenzyl)amino)-1-cyclopropyl-2-oxoethyl)amino)-2-oxoethyl)-1H-indazole-3-carboxamide;
(S)-1-(2-((1-((3-chloro-2-fluorophenyl)amino)-1-oxopropan-2-yl)(methyl)amino)-2-oxoethyl)-1H-indazole-3-carboxamide;
(S)-1-(2-((1-((3-chloro-2-fluorobenzyl)amino)-1-oxopropan-2-yl)(methyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(isopropyl)amino)-2-oxoethyl)-1H-indazole-3-carboxamide;
1-(2-((2-((3-chloro-2-fluorobenzyl)amino)-2-oxoethyl)(ethyl)amino)-2-oxoethyl)-1H-indazole-3-carboxamide;
1-(2-((2-((3-chloro-2-fluorophenyl)amino)-2-oxoethyl)(cyclopropyl)amino)-2-oxoethyl)-1H-indazole-3-carboxamide;
3-(2-((2-((3-chloro-2-fluorobenzyl)amino)-2-oxoethyl)(cyclopropyl)amino)-2-oxoethyl)-1H-indazole-1-carboxamide;
1-(2-(cyclopropyl(2-((5-hydroxy-3,3-dimethylcyclohexyl)amino)-2-oxoethyl)amino)-2-oxoethyl)-1H-indazole-3-carboxamide;
1-(2-(cyclopropyl(2-(((1S,5S)-5-hydroxy-3,3-dimethylcyclohexyl)amino)-2-oxoethyl)amino)-2-oxoethyl)-1H-indazole-3-carboxamide;
1-(2-((2-((3-chloro-2-fluorophenyl)amino)-2-oxoethyl)(isopropyl)amino)-2-oxoethyl)-1H-indazole-3-carboxamide;
1-(2-((2-((6-chloropyridin-2-yl)amino)-2-oxoethyl)(isopropyl)amino)-2-oxoethyl)-1H-indazole-3-carboxamide;
1-(2-((2-((6-chloropyridin-2-yl)amino)-2-oxoethyl)(2-hydroxyethyl)amino)-2-oxoethyl)-1H-indazole-3-carboxamide;
1-(2-((2-((6-chloropyridin-2-yl)amino)-2-oxoethyl)(methyl)amino)-2-oxoethyl)-1H-indazole-3-carboxamide;
1-(2-((2-((6-chloropyridin-2-yl)amino)-2-oxoethyl)(ethyl)amino)-2-oxoethyl)-1H-indazole-3-carboxamide;
1-(2-((2-((6-chloropyridin-2-yl)amino)-2-oxoethyl)(cyclopropyl)amino)-2-oxoethyl)-1H-indazole-3-carboxamide;
1-(2-((2-((6-chloropyridin-2-yl)amino)-2-oxoethyl)(piperidin-4-yl)amino)-2-oxoethyl)-1H-indazole-3-carboxamide hydrochloride;
3-(2-((2-aminoethyl)(2-((6-chloropyridin-2-yl)amino)-2-oxoethyl)amino)-2-oxoethyl)-1H-indazole-1-carboxamide;
3-(2-((2-((6-chloropyridin-2-yl)amino)-2-oxoethyl)(cyclopentyl)amino)-2-oxoethyl)-1H-indazole-1-carboxamide;
3-(2-((2-((6-chloropyridin-2-yl)amino)-2-oxoethyl)(cyclobutyl)amino)-2-oxoethyl)-1H-indazole-1-carboxamide;
3-(2-((2-((6-chloropyridin-2-yl)amino)-2-oxoethyl)(tetrahydro-2H-pyran-4-yl)amino)-2-oxoethyl)-1H-indazole-1-carboxamide;
1-(2-(azetidin-3-yl(2-((6-chloropyridin-2-yl)amino)-2-oxoethyl)amino)-2-oxoethyl)-1H-indazole-3-carboxamide;
1-(2-((2-((6-chloropyridin-2-yl)amino)-2-oxoethyl)(2-(methylamino)ethyl)amino)-2-oxoethyl)-1H-indazole-3-carboxamide;
1-(2-((2-acetamidoethyl)(2-((6-chloropyridin-2-yl)amino)-2-oxoethyl)amino)-2-oxoethyl)-1H-indazole-3-carboxamide;
1-(2-((2-((6-chloropyridin-2-yl)amino)-2-oxoethyl)(pyridin-4-ylmethyl)amino)-2-oxoethyl)-1H-indazole-3-carboxamide;
1-(2-((2-((6-chloropyridin-2-yl)amino)-2-oxoethyl)(pyrrolidin-3-yl)amino)-2-oxoethyl)-1H-indazole-3-carboxamide;
1-(2-((2-((3-chloro-2-fluorobenzyl)amino)-2-oxoethyl)(thiophen-3-ylmethyl)amino)-2-oxoethyl)-1H-indazole-3-carboxamide;
1-(2-((2-((3-chloro-2-fluorobenzyl)amino)-2-oxoethyl)(cyclobutyl)amino)-2-oxoethyl)-1H-indazole-3-carboxamide;
1-(2-((2-((3-chloro-2-fluorobenzyl)amino)-2-oxoethyl)(tetrahydro-2H-pyran-4-yl)amino)-2-oxoethyl)-1H-indazole-3-carboxamide;
1-(2-((2-((3-chloro-2-fluorobenzyl)amino)-2-oxoethyl)(cyclopentyl)amino)-2-oxoethyl)-1H-indazole-3-carboxamide;
1-(2-((2-aminoethyl)(2-((3-chloro-2-fluorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(pyridin-4-ylmethyl)amino)-2-oxoethyl)-1H-indazole-3-carboxamide;
1-(2-((2-((3-chloro-2-fluorobenzyl)amino)-2-oxoethyl)(methyl)amino)-2-oxoethyl)-1H-indazole-3-carboxamide;
1-(2-((2-((3-chloro-2-fluorobenzyl)amino)-2-oxoethyl)(piperidin-4-yl)amino)-2-oxoethyl)-1H-indazole-3-carboxamide;
1-(2-((2-((3-chloro-2-fluorobenzyl)amino)-2-oxoethyl)(pyridin-3-ylmethyl)amino)-2-oxoethyl)-1H-indazole-3-carboxamide;
1-(2-((2-((3-chloro-2-fluorobenzyl)amino)-2-oxoethyl)(phenyl)amino)-2-oxoethyl)-1H-indazole-3-carboxamide;
1-(2-((2-((3-chloro-2-fluorobenzyl)amino)-2-oxoethyl)(pyrrolidin-3-yl)amino)-2-oxoethyl)-1H-indazole-3-carboxamide;
(R)-1-(2-((1-((3-chloro-2-fluorobenzyl)amino)-1-oxopropan-2-yl)(cyclopropyl)amino)-2-oxoethyl)-1H-indazole-3-carboxamide;
(S)-1-(2-((1-((3-chloro-2-fluorobenzyl)amino)-1-oxopropan-2-yl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-1H-pyrazolo[3,4-c]pyridine-3-carboxamide;
3-(2-((2-((3-chloro-2-fluorobenzyl)amino)-2-oxoethyl)(cyclopropyl)amino)-2-oxoethyl)-1H-indole-1-carboxamide;
3-(2-((2-((3-chloro-2-fluorobenzyl)amino)-2-oxoethyl)(cyclopropyl)amino)-2-oxoethyl)imidazo[1,5-a]pyridine-1-carboxamide;
1-(2-((2-((3-chloro-2-fluorobenzyl)amino)-2-oxoethyl)(cyclopropyl)amino)-2-oxoethyl)-5-(trifluoromethyl)-1H-pyrazole-3-carboxamide;
3-carbamoyl-1-(2-((2-((3-chloro-2-fluorobenzyl)amino)-2-oxoethyl)(cyclopropyl)amino)-2-oxoethyl)-1H-pyrazole-5-carboxylic acid;
1-(2-((2-((3-chloro-2-fluorobenzyl)amino)-2-oxoethyl)(cyclopropyl)amino)-2-oxoethyl)-1H-pyrazole-3,5-dicarboxamide;
4-bromo-1-(2-((2-((3-chloro-2-fluorobenzyl)amino)-2-oxoethyl)(cyclopropyl)amino)-2-oxoethyl)-1H-pyrazole-3-carboxamide;
2-(3-acetyl-1H-indazol-1-yl)-N-(2-((3-chloro-2-fluorobenzyl)amino)-2-oxoethyl)-N-cyclopropylacetamide;
2-(1-acetylimidazo[1,5-a]pyridin-3-yl)-N-(2-((3-chloro-2-fluorobenzyl)amino)-2-oxoethyl)-N-cyclopropylacetamide;
N-(2-((3-chloro-2-fluorobenzyl)amino)-2-oxoethyl)-N-cyclopropyl-2-(3-(1-hydroxyethyl)-1H-indazol-1-yl)acetamide;
3-(3-(2-((3-chloro-2-fluorobenzyl)amino)-2-oxoethyl)-3-isopropylureido)-1H-indole-1-carboxamide;
3-(3-(2-((3-chloro-2-fluorobenzyl)amino)-2-oxoethyl)-3-cyclopropylureido)-1H-indole-1-carboxamide;
3-(3-(2-((3-chloro-2-fluorobenzyl)amino)-2-oxoethyl)-3-(pyrrolidin-3-yl)ureido)-1H-indole-1-carboxamide;
1-(3-(2-((3-chloro-2-fluorobenzyl)amino)-2-oxoethyl)-3-cyclopropylureido)imidazo[1,5-a]pyridine-3-carboxamide;
1-(2-((2-((3-chloro-2-fluorobenzyl)amino)-2-oxoethyl)(3-methylbutan-2-yl)amino)-2-oxoethyl)-1H-indazole-3-carboxamide;
1-(2-((2-((3-chloro-2-fluorobenzyl)amino)-2-oxoethyl)(1-cyclopropylethyl)amino)-2-oxoethyl)-1H-indazole-3-carboxamide;
1-(2-(sec-butyl(2-((3-chloro-2-fluorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(4-hydroxybutan-2-yl)amino)-2-oxoethyl)-1H-indazole-3-carboxamide;
1-(2-((2-((3-chloro-2-fluorobenzyl)amino)-2-oxoethyl)(1-hydroxypropan-2-yl)amino)-2-oxoethyl)-1H-indazole-3-carboxamide;
methyl 2-(2-(3-carbamoyl-1H-indazol-1-yl)-N-(2-((3-chloro-2-fluorobenzyl)amino)-2-oxoethyl)acetamido)propanoate;
1-(2-((2-((3-chloro-2-fluorobenzyl)amino)-2-oxoethyl)(4-fluorobutan-2-yl)amino)-2-oxoethyl)-1H-indazole-3-carboxamide;
1-(2-((2-((3-chloro-2-fluorobenzyl)amino)-2-oxoethyl)(1-methoxybutan-2-yl)amino)-2-oxoethyl)-1H-indazole-3-carboxamide;
1-(2-((1-amino-1-oxopropan-2-yl)(2-((3-chloro-2-fluorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(2-methylcyclopropyl)amino)-2-oxoethyl)-1H-indazole-3-carboxamide;
1-(2-((2-((3-chloro-2-fluorobenzyl)amino)-2-oxoethyl)(3,3-dimethylbutan-2-yl)amino)-2-oxoethyl)-1H-indazole-3-carboxamide;
1-(2-((2-((3-chloro-2-fluorobenzyl)amino)-2-oxoethyl)(1-phenylethyl)amino)-2-oxoethyl)-1H-indazole-3-carboxamide;
1-(2-((2-((3-chloro-2-fluorobenzyl)amino)-2-oxoethyl)(1-fluoropropan-2-yl)amino)-2-oxoethyl)-1H-indazole-3-carboxamide;
1-(2-((2-((3-chloro-2-fluorobenzyl)amino)-2-oxoethyl)(1,1,1-trifluoropropan-2-yl)amino)-2-oxoethyl)-1H-indazole-3-carboxamide;
1-(2-((2-((3-chloro-2-fluorobenzyl)amino)-2-oxoethyl)(1-methoxypropan-2-yl)amino)-2-oxoethyl)-1H-indazole-3-carboxamide;
1-(2-((2-((3-chloro-2-fluorobenzyl)amino)-2-oxoethyl)(1-cyclopropyl-2-hydroxyethyl)amino)-2-oxoethyl)-1H-indazole-3-carboxamide;
1-(2-((2-((2-chloro-3-fluorobenzyl)amino)-2-oxoethyl)(1,3-difluoropropan-2-yl)amino)-2-oxoethyl)-1H-indazole-3-carboxamide;
1-(2-((4-aminobutan-2-yl)(2-((2-chloro-3-fluorobenzyl)amino)-2-oxoethyl)amino)-2-oxoethyl)-1H-indazole-3-carboxamide;
ethyl 3-(2-(3-carbamoyl-1H-indazol-1-yl)-N-(2-((3-chloro-2-fluorobenzyl)amino)-2-oxoethyl)acetamido)butanoate;
1-(2-((2-((3-chloro-2-fluorobenzyl)amino)-2-oxoethyl)(1-cyclopropyl-3-hydroxypropyl)amino)-2-oxoethyl)-1H-indazole-3-carboxamide;
3-(2-(3-carbamoyl-1H-indazol-1-yl)-N-(2-((3-chloro-2-fluorobenzyl)amino)-2-oxoethyl)acetamido)butanoic acid;
1-(2-((3-amino-1-cyclopropyl-3-oxopropyl)(2-((3-chloro-2-fluorobenzyl)amino)-2-oxoethyl)amino)-2-oxoethyl)-1H-indazole-3-carboxamide;
1-(2-((4-amino-4-oxobutan-2-yl)(2-((3-chloro-2-fluorobenzyl)amino)-2-oxoethyl)amino)-2-oxoethyl)-1H-indazole-3-carboxamide;
ethyl 3-(2-(3-carbamoyl-1H-indazol-1-yl)-N-(2-((3-chloro-2-fluorobenzyl)amino)-2-oxoethyl)acetamido)-4,4,4-trifluorobutanoate;
1-(2-((2-((3-chloro-2-fluorobenzyl)amino)-2-oxoethyl)(cyclopropyl)amino)-2-oxoethyl)-5-fluoro-1H-indazole-3-carboxamide;
3-(2-(3-carbamoyl-1H-indazol-1-yl)-N-(2-((3-chloro-2-fluorobenzyl)amino)-2-oxoethyl)acetamido)-4,4,4-trifluorobutanoic acid;
5-chloro-1-(2-((2-((3-chloro-2-fluorobenzyl)amino)-2-oxoethyl)(cyclopropyl)amino)-2-oxoethyl)-1H-indazole-3-carboxamide;
7-chloro-1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-2,6-difluorobenzyl)amino)-2-oxoethyl)(cyclopropyl)amino)-2-oxoethyl)-1H-indazole-3-carboxamide;
1-(2-((2-((3-chloro-2,4-difluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-6-fluoro-1H-indazole-3-carboxamide;
6-chloro-1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-6-cyano-1H-indazole-3-carboxamide;
1-(2-((2-((3-chloro-6-cyano-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-7-fluoro-1H-indazole-3-carboxamide;
6-(aminomethyl)-1-(2-((2-((3-chloro-2-fluorobenzyl)amino)-2-oxoethyl)(cyclopropyl)amino)-2-oxoethyl)-1H-indazole-3-carboxamide;
1-(2-((2-((3-chloro-5-cyano-2-fluorobenzyl)amino)-2-oxoethyl)(cyclopropyl)amino)-2-oxoethyl)-1H-indazole-3-carboxamide;
1-(2-((2-(((6-chloro-1H-indazol-4-yl)methyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-5-cyano-1H-indazole-3-carboxamide;
1-(2-((2-((3-chloro-2-fluorobenzyl)amino)-2-oxoethyl)(cyclopropyl)amino)-2-oxoethyl)-1H-indazole-3,5-dicarboxamide;
1-(2-((2-((3-chloro-2-fluorobenzyl)amino)-2-oxoethyl)(cyclopropyl)amino)-2-oxoethyl)-6-(trifluoromethyl)-1H-indazole-3-carboxamide;
1-(2-((2-((3-chloro-2-fluorobenzyl)amino)-2-oxoethyl)(cyclopropyl)amino)-2-oxoethyl)-5-(trifluoromethyl)-1H-indazole-3-carboxamide;
1-(2-((2-((3-chloro-4-cyano-2-fluorobenzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-4-fluoro-1H-indazole-3-carboxamide;
2-(3-(1H-imidazol-2-yl)-1H-indazol-1-yl)-N-(2-((3-chloro-2-fluorobenzyl)amino)-2-oxoethyl)-N-cyclopropylacetamide;
1-(2-((2-((3-chloro-2-fluoro-4-(hydroxymethyl)benzyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-1H-indazole-3,6-dicarboxamide;
1-(2-((2-((5-(aminomethyl)-3-chloro-2-fluorobenzyl)amino)-2-oxoethyl)(cyclopropyl)amino)-2-oxoethyl)-1H-indazole-3-carboxamide;
1-(2-((2-(((4-chloro-1H-indazol-6-yl)methyl)amino)-2-oxoethyl)(cyclopropyl)amino)-2-oxoethyl)-1H-indazole-3-carboxamide;
1-(2-((2-((3-chloro-5-fluorobenzyl)amino)-2-oxoethyl)(cyclopropyl)amino)-2-oxoethyl)-1H-indazole-3-carboxamide;
1-(2-(cyclopropyl(2-((3,4-dichlorobenzyl)amino)-2-oxoethyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-7-cyano-1H-indazole-3-carboxamide;
1-(2-(cyclopropyl(2-((2,3-dichlorobenzyl)amino)-2-oxoethyl)amino)-2-oxoethyl)-1H-indazole-3-carboxamide;
1-(2-((2-(((6-chloropyridin-2-yl)methyl)amino)-2-oxoethyl)(cyclopropyl)amino)-2-oxoethyl)-1H-indazole-3-carboxamide;
1-(2-((2-((3-chloro-4-fluorobenzyl)amino)-2-oxoethyl)(cyclopropyl)amino)-2-oxoethyl)-1H-indazole-3-carboxamide;
1-(2-((2-(((5-chloro-1H-indazol-7-yl)methyl)amino)-2-oxoethyl)(cyclopropyl)amino)-2-oxoethyl)-1H-indazole-3-carboxamide;
1-(2-((2-(((5-chlorobenzofuran-7-yl)methyl)amino)-2-oxoethyl)(cyclopropyl)amino)-2-oxoethyl)-1H-indazole-3-carboxamide;
1-(2-((2-((3-chloro-2-fluorobenzyl)amino)-2-oxoethyl)(cyclopropyl)amino)-2-oxoethyl)-7-(trifluoromethyl)-1H-indazole-3-carboxamide;
1-(2-((2-((3-chlorobenzyl)amino)-2-oxoethyl)(cyclopropyl)amino)-2-oxoethyl)-1H-indazole-3-carboxamide;
1-(2-((2-((3-chloro-4-methylbenzyl)amino)-2-oxoethyl)(cyclopropyl)amino)-2-oxoethyl)-1H-indazole-3-carboxamide;
1-(2-((2-(((7-chloro-1H-indazol-5-yl)methyl)amino)-2-oxoethyl)(cyclopropyl)amino)-2-oxoethyl)-1H-indazole-3-carboxamide;
1-(2-(cyclopropyl(2-((2-fluorobenzyl)amino)-2-oxoethyl)amino)-2-oxoethyl)-1H-indazole-3-carboxamide;
1-(2-((2-((3-chloro-2-methoxybenzyl)amino)-2-oxoethyl)(cyclopropyl)amino)-2-oxoethyl)-1H-indazole-3-carboxamide;
1-(2-((2-((4-carbamoyl-2-fluorobenzyl)amino)-2-oxoethyl)(cyclopropyl)amino)-2-oxoethyl)-1H-indazole-3-carboxamide;
1-(2-((2-((3-amino-2-fluorobenzyl)amino)-2-oxoethyl)(cyclopropyl)amino)-2-oxoethyl)-1H-indazole-3-carboxamide;
1-(2-(cyclopropyl(2-((3,5-dichlorobenzyl)amino)-2-oxoethyl)amino)-2-oxoethyl)-1H-indazole-3-carboxamide;
1-(2-((2-((3-bromo-2-fluorobenzyl)amino)-2-oxoethyl)(cyclopropyl)amino)-2-oxoethyl)-1H-indazole-3-carboxamide;
1-(2-((2-((4-amino-2-fluorobenzyl)amino)-2-oxoethyl)(cyclopropyl)amino)-2-oxoethyl)-1H-indazole-3-carboxamide;
1-(2-(cyclopropyl(2-((2-fluoro-3-methoxybenzyl)amino)-2-oxoethyl)amino)-2-oxoethyl)-1H-indazole-3-carboxamide;
1-(2-((2-((3-cyano-2-fluorobenzyl)amino)-2-oxoethyl)(cyclopropyl)amino)-2-oxoethyl)-1H-indazole-3-carboxamide;
1-(2-((2-((3-chloro-5-(trifluoromethyl)benzyl)amino)-2-oxoethyl)(cyclopropyl)amino)-2-oxoethyl)-1H-indazole-3-carboxamide;
1-(2-((2-((3-chloro-4-methoxybenzyl)amino)-2-oxoethyl)(cyclopropyl)amino)-2-oxoethyl)-1H-indazole-3-carboxamide;
1-(2-(cyclopropyl(2-((2-fluoro-3-(hydroxymethyl)benzyl)amino)-2-oxoethyl)amino)-2-oxoethyl)-1H-indazole-3-carboxamide;
1-(2-(cyclopropyl(2-((3-cyclopropyl-2-fluorobenzyl)amino)-2-oxoethyl)amino)-2-oxoethyl)-1H-indazole-3-carboxamide;
1-(2-(cyclopropyl(2-((2-fluoro-3-methylbenzyl)amino)-2-oxoethyl)amino)-2-oxoethyl)-1H-indazole-3-carboxamide;
1-(2-((2-((3-chloro-5-methylbenzyl)amino)-2-oxoethyl)(cyclopropyl)amino)-2-oxoethyl)-1H-indazole-3-carboxamide;
1-(2-((2-(((5-chloropyridin-3-yl)methyl)amino)-2-oxoethyl)(cyclopropyl)amino)-2-oxoethyl)-1H-indazole-3-carboxamide;
1-(2-((2-((3-chloro-2-cyclopropylbenzyl)amino)-2-oxoethyl)(cyclopropyl)amino)-2-oxoethyl)-1H-indazole-3-carboxamide;
1-(2-(cyclopropyl(2-((2,3-difluorobenzyl)amino)-2-oxoethyl)amino)-2-oxoethyl)-1H-indazole-3-carboxamide;
1-(2-((2-((3-chloro-5-cyanobenzyl)amino)-2-oxoethyl)(cyclopropyl)amino)-2-oxoethyl)-1H-indazole-3-carboxamide;
1-(2-((2-((3-chloro-2-methylbenzyl)amino)-2-oxoethyl)(cyclopropyl)amino)-2-oxoethyl)-1H-indazole-3-carboxamide;
1-(2-((2-((3-chloro-2-(trifluoromethyl)benzyl)amino)-2-oxoethyl)(cyclopropyl)amino)-2-oxoethyl)-1H-indazole-3-carboxamide;
1-(2-(cyclopropyl(2-((2-fluoro-3-(trifluoromethyl)benzyl)amino)-2-oxoethyl)amino)-2-oxoethyl)-1H-indazole-3-carboxamide;
2-(3-acetylimidazo[1,5-a]pyridin-1-yl)-N-(2-((3-chloro-2-fluorobenzyl)amino)-2-oxoethyl)-N-cyclopropylacetamide;
1-(2-((2-((3-chloro-2-fluorobenzyl)amino)-2-oxoethyl)(cyclopropyl)amino)-2-oxoethyl)imidazo[1,5-a]pyridine-3-carboxamide;
2-(1-acetylimidazo[1,5-a]pyridin-3-yl)-N-(2-((3-chloro-2-fluorobenzyl)amino)-2-oxoethyl)-N-cyclopropylacetamide;
3-(3-(2-((3-chloro-2-fluorobenzyl)amino)-2-oxoethyl)-3-cyclopropylureido)imidazo[1,5-a]pyridine-1-carboxamide; or
2-(3-(3-acetylimidazo[1,5-a]pyridin-1-yl)-1-cyclopropylureido)-N-(3-chloro-2-fluorobenzyl)acetamide.

62. A pharmaceutical composition comprising a pharmaceutically acceptable excipient and a compound of any one of claims 1-61, or a pharmaceutically acceptable salt thereof.

63. A method of treating an autoimmune, inflammatory, or neurodegenerative disease in a patient in need thereof comprising administering to the patient a pharmaceutical composition comprising a compound of any one of claims 1-61, or a pharmaceutically acceptable salt thereof.

64. The method of claim 63, wherein the autoimmune, inflammatory, or neurodegenerative disease is paraoxysmal nocturnal hemoglobinuria.
